(19) United States
(12) Patent Application Publication
(10) Pub. No.: US 2020/0063214 A1
(43) Pub. Date: Feb. 27, 2020

(54) METHODS OF IDENTIFYING PATIENTS LIKELY TO BENEFIT FROM TREATMENT WITH A TELOMERASE INHIBITOR

(71) Applicant: Geron Corporation, Menlo Park, CA (US)

(72) Inventors: Jacqueline Cirillo BUSSOLARI, Skillman, NJ (US); Fei HUANG, Spring House, PA (US)

(21) Appl. No.: 16/525,026

(22) Filed: Jul. 29, 2019

Related U.S. Application Data
(60) Provisional application No. 62/772,849, filed on Nov. 29, 2018.
(60) Provisional application No. 62/712,841, filed on Jul. 31, 2018.

(51) Int. Cl. C12Q 1/6886; G16H 50/30; G16B 40/10; C12Q 1/6827; A61K 9/00; G16H 20/10; A61K 31/7105; A61K 47/54

(52) U.S. Cl. CPC C12Q 1/6886; G16H 50/30; G16B 40/10; A61K 47/543; A61K 9/0019; G16H 20/10; A61K 31/7105; C12Q 1/6827

(57) ABSTRACT

This disclosure provides methods of identifying or selecting a patient most likely to benefit from treatment with a telomerase inhibitor, such as e.g. imetelstat, by testing a patient for: a lack of a mutation in each of JAK2, CALR, and MPL; and/or a high-molecular risk (HMR), based on the presence of a mutation in at least one of the following genes: ASXL1, EZH2, SRSF2, and IDH1/2. The patient may be suffering from myelofibrosis. The disclosure also provides methods of treating myelofibrosis, which include identifying such patients.

### CROSS-REFERENCE TO RELATED APPLICATIONS

Pursuant to 35 U.S.C. § 119(e), this application claims the benefit of priority to the filing dates of U.S. Provisional Patent Application Ser. No. 62/712,841 filed on Jul. 31, 2018 and U.S. Provisional Patent Application Ser. No. 62/772,849 filed on Nov. 29, 2018; the disclosures of which application are herein incorporated by reference.

### SEQUENCE LISTING

The instant application contains a Sequence Listing, which has been filed electronically in ASCII format and is hereby incorporated by reference in its entirety. Said ASCII copy is named Sequence Listing.txt and is 356 KB in size.

### FIELD OF THE INVENTION

The present application relates to methods of identifying a patient most likely to benefit from treatment of a telomerase inhibitor by identifying a patient: lacking a mutation in each of JAK2, CALR, and MPL; and/or having a high-molecular risk (HMR), based on the presence of a mutation in at least one of the following genes: ASXL1, EZH2, SRSF2, and IDH1/2. The invention also relates to methods of treating myelofibrosis in a subject in need thereof (i.e. patient) with a telomerase inhibitor.

### INTRODUCTION

Myelofibrosis (MF) is one of the classical BCR-ABL1-negative chronic myeloproliferative neoplasms (MPNs), characterized by clonal myeloproliferation, dysregulated kinase signaling. Cervantes, Blood, 124(17):2635-2642 (2014). It is also characterized by cytopenias, constitutional symptoms, splenomegaly, and may transform into acute myeloid leukemia. Kuykendall et al., Annals of Hematology, 97: 435-431 (2018). MF is a poor-prognosis Philadelphia chromosome negative myeloproliferative neoplasm, for which the JAK1/JAK2 inhibitor ruxolitinib is currently an approved therapy. Ruxolitinib, a Janus kinase (JAK)-1 and JAK-2 inhibitor, is the first-in-class drug to be licensed in the United States for the treatment of high- and intermediate-risk myelofibrosis (MF). Pardanani, et al. Blood Cancer J.; 4(12): e268 (2014). Several other JAK inhibitors are in development with some currently undergoing phase-3 clinical trial testing. Id. Other treatment options for MF include allo-SCT, hydroxyurea, interferon, lenalidomide (Revlimid®) and thalidomide. Currently, there are ongoing clinical trials in MF to evaluate selective JAK inhibitors, histone deacetylase/DNA methyltransferase inhibitors, PI3K-inhibitors, Hedgehog/mammalian target of rapamycin (MTOR) inhibitors, anti-fibrotic agents, immunomodulators, monoclonal antibodies, and immune checkpoint inhibitors. Shreenivas, et al., Expert Opin Emerg Drugs, 23(1):37-49 (2018).

Other MPNs include essential thrombocythemia (ET) and polycythemia vera (PV). Cervantes (infra). MF may appear de novo (primary MF [PMF]) or following previous ET or PV (post-ET or post-PVMF). Id. According to Cervantes, MF is a clonal proliferation of a pluripotent hematopoietic stem cell in which the abnormal cell population releases several cytokines and growth factors in the bone marrow that lead to marrow fibrosis and stroma changes and colonizes extramedullary organs such as the spleen and liver. Id. Myelofibrosis has been associated with mutations in the Janus kinase (JAK) 2 gene (such as a V617F mutation), mutations in thrombopoietin receptor gene (MPL) and mutations in the calreticulin gene (CALR). Id. It mainly affects the elderly, and, according to Cervantes, “[a]t present, there is no curative treatment other than allogeneic hemopoietic stem cell transplantation (allo-SCT), which can be applied to a minority of patients.” Id.

In fact, according to Langabeer, “[t]he majority of patients with classical myeloproliferative neoplasms (MPN) of polycythemia vera, essential thrombocythemia, and primary myelofibrosis harbor distinct disease driving mutations within the JAK2, CALR, or MPL genes.” Langabeer, JAK-STAT, 5: e1248011 (2016). These mutations are so-called driver mutations. Exemplary driver mutations include JAK2 V617F, mutations with JAK2 exon 12, MPL exon 10, and CALR exon 9. Id.

According to Spiegel, in myelofibrosis (MF), driver mutations in JAK2, MPL, or CALR impact survival and progression to blast phase, with the greatest risk conferred by triple-negative status (i.e. nonmutated JAK2, MPL, and CALR). Spiegel et al., Blood Adv., 1(20):1729-1738 (2017). In fact, the absence of JAK2/MPL/CALR mutations (that is, triple negative) is associated with the most unfavorable outcome. Pardanani, et al. Blood Cancer J.; 4(12): e268 (2014); see also Tefferi et al., Blood, 124(16):2507-13 (2014). Furthermore, mutations in high-molecular risk (HAIR) genes, such as ASXL1, EZH2, IDH1/2, and SRSF2 have also been associated with inferior prognosis. Spiegel et al. The presence of increasing number of prognostically detrimental/“high molecular risk” mutations (that is, ASXL1, EZH2, SRSF2, and/or IDH-1/2 genes) conferred a progressively worse survival outcome, independent of traditional risk factors. Guglielmelli et al., Leukemia, 28(9):1804-10 (2014).

Driver mutations in JAK2, MPL, or CALR, either alone or in combination with subclonal mutations in genes, such as ASXL1, have been associated with differences in overall survival (OS). Spiegel et al. Triple-negative patients, who lack canonical mutations in JAK2,MPL, or CALR, have an increased risk of leukemic transformation as well as shortened OS. Spiegel observed that for patients suffering from myelofibrosis that were treated with ruxolitinib or momelotinib (JAK 1/2 inhibitors), these mutations were associated with a shorter time to treatment failure. Id. Similarly, “[c]omparing the clinical characteristics of JAK2-positive, CALR-positive, MPL-positive, and TN MF patients, those with CALR mutations had significantly lower hemoglobin (mean, 8.6 vs 10.7 g/dL; P 5.001) and white blood cell counts (mean, 11.0 vs 25 g/dL; P 5.033), trends that have been reported in other MPN cohorts.” Patel et al., Blood; 126(6):790-797 (2015). Patel et al. observed that patients treated with ruxolitinib, harboring ≥3 mutations exhibited inverse correlation with spleen response and time to treatment discontinuation. Driver mutations or triple negative (JAK2, MPL, CALR) status are found in myelofibrosis patient who discontinue treatment with JAK inhibitors. See e.g. Kuykendall et al.

### SUMMARY

The invention provides methods of identifying or selecting a patient most likely to benefit from treatment with a telomerase inhibitor, such as e.g. imetelstat, by testing a patient for: a lack of a mutation in each of Janus kinase 2 (JAK2), calreticulin (CALR), and thrombopoietin receptor (MPL) genes; and/or a high-molecular risk (HMR), based on the presence of a mutation in at least one of the following genes: additional sex combs like 1 (ASXL1), enhancer of zeste homolog 2 (EZH2), serine and arginine rich splicing factor 2 (SRSF2), and isocitrate dehydrogenase 1/2 (IDH1/2). The patient in need of treatment may be suffering from myelofibrosis. The invention also provides methods of treating myelofibrosis in a patient in need of such treatment, which include the step of identifying such a patient.

One embodiment of the invention is a method of identifying a myelofibrosis patient most likely to benefit from treatment with a telomerase inhibitor comprising: (a) testing a patient for the following: (i) triple negative status, based on the absence of a mutation in each of JAK2, CALR, and MPL genes, and/or (ii) a mutation in at least one of the following genes: ASXL1, EZH2, SRSF2, and IDH1/2; and (b) selecting the patient if the patient has: (i) triple negative status, based on the lack of mutation in each of JAK2, CALR, and MPL genes, and/or (ii) high molecular risk (HMR) based on the presence of a mutation in at least one of the following genes: ASXL1, EZH2, SRSF2, and IDH1/2, wherein the selected patient is most likely to benefit from treatment with a telomerase inhibitor.

An alternate embodiment of the invention is a method of identifying a patient most likely to benefit from treatment with a telomerase inhibitor comprising: (a) testing a patient for: triple negative status, based on the absence of a mutation in each of JAK2, CALR, and MPL genes; and (b) selecting the patient if the patient has: triple negative status, wherein the selected patient is most likely to benefit from treatment with a telomerase inhibitor. An alternate embodiment of the invention is a method of identifying a patient most likely to benefit from treatment with a telomerase inhibitor comprising (a) testing the patient for high molecular risk (HMR) based on the presence of a mutation in at least one of the following genes: ASXL1, EZH2, SRSF2, and IDH1/2 and (b) selecting a patient that is high molecular risk (HMR) based on the presence of a mutation in at least one of the following genes: ASXL1, EZH2, SRSF2, and IDH1/2. The invention further provides for methods of treating myelofibrosis in a patient that is triple negative and/or HMR with a telomerase inhibitor, such as e.g. imetelstat.

Another embodiment of the invention is a method of identifying a patient that has myelofibrosis most likely to benefit from treatment with a telomerase inhibitor comprising: (a) obtaining a DNA sample from a patient; (b) testing the DNA sample from such patient for (i) triple negative status, based on the absence of a mutation in each of JAK2, CALR, and MPL genes; and/or (ii) a high-molecular risk (HMR), based on the presence of a mutation in at least one of the following genes: ASXL1, EZH2, SRSF2, and IDH1/2; and (c) selecting the patient if the patient has (i) triple negative status, based on the absence of a mutation in each of JAK2, CALR, and MPL genes, and/or (ii) a high-molecular risk (HMR), based on the presence of a mutation in at least one of the following genes: ASXL1, EZH2, SRSF2, and IDH1/2, wherein the selected patient is most likely to benefit from treatment with a telomerase inhibitor. In certain embodiments of the method, the DNA sample is obtained from bone marrow, peripheral blood, or both.

The DNA sample may be obtained by first obtaining a bone marrow sample, a peripheral blood sample, or both and then isolating the DNA from the bone marrow sample, the peripheral blood sample, or both. In one embodiment, the step of obtaining a DNA sample from a patient comprises: obtaining a bone marrow sample from the patient, isolating cells from the bone marrow sample, and extracting DNA from the isolated cells. In another embodiment, the step of obtaining a DNA sample from a patient comprises: obtaining a peripheral blood sample from the patient; isolating cells from the peripheral blood sample (e.g. granulocytes); and extracting DNA from the isolated cells.

Yet another embodiment of the invention is a method of identifying a patient that has myelofibrosis most likely to benefit from treatment with a telomerase inhibitor comprising testing a patient for: (a) triple negative status, based on the absence of any mutation in JAK2, CALR, and MPL genes; (b) a high-molecular risk (HMR), based on the presence of a mutation in at least one of the following genes: ASXL1, EZH2, SRSF2, and IDH1/2; or (c) both; wherein the presence of (a), (b) or (c) is indicative of a patient most likely to benefit from treatment with a telomerase inhibitor.

In any of these methods, the patient may be suffering from myelofibrosis. The myelofibrosis may be: primary myelofibrosis; myelofibrosis that develops post-polycythemia vera (post-PV MF); or myelofibrosis that develops post essential thrombocythemia (post-ET MF). In certain embodiments, the patient has not previously received JAK-inhibitor therapy. In other embodiments, the patient has previously received JAK-inhibitor therapy and has “failed” JAK-inhibitor therapy (i.e., the disease was resistant or the patient was refractory to the therapy or although initially responsive to treatment, the disease has relapsed). In other embodiments, the patient has received JAK-inhibitor therapy and has discontinued JAK-inhibitor therapy due to treatment-related toxicities or intolerance.

The methods may also include the step of administering the telomerase inhibitor once such a patient has been identified. In certain embodiments, the telomerase inhibitor is imetelstat or a pharmaceutically acceptable salt thereof. In other embodiments, the imetelstat is imetelstat sodium.

When imetelstat is used to treat patients identified by these methods, imetelstat is administered for 1, 2, 3, 4, 5, 6, 7, 8, or more than 8 dosage cycles, each cycle comprising: intravenous administration of about 7-10 mg/kg imetelstat once every three weeks; intravenous administration of about 7-10 mg/kg imetelstat once weekly for three weeks; intravenous administration of about 2.5-10 mg/kg imetelstat once every three weeks; or intravenous administration of about 0.5-9.4 mg/kg imetelstat once every three weeks. In one embodiment, each dosage cycle comprises intravenous administration of about 7-10 mg/kg imetelstat once every three weeks. In another embodiment, each dosage cycle comprises intravenous administration of about 9.4 mg/kg imetelstat once every three weeks.

When imetelstat sodium is used to treat patients identified by these methods, imetelstat sodium is administered for 1, 2, 3, 4, 5, 6, 7, 8, or more than 8 dosage cycles, each cycle comprising: intravenous administration of about 7-10 mg/kg imetelstat sodium once every three weeks; intravenous administration of about 7-10 mg/kg imetelstat sodium once weekly for three weeks; intravenous administration of about 2.5-10 mg/kg imetelstat sodium once every three weeks; or intravenous administration of about 0.5-9.4 mg/kg imetelstat sodium once every three weeks. In one embodiment, each dosage cycle comprises intravenous administration of about 7-10 mg/kg imetelstat sodium once every three weeks. In another embodiment, each dosage cycle comprises intravenous administration of about 9.4 mg/kg imetelstat sodium once every three weeks.

Another embodiment of the invention is a method of treating a patient that has myelofibrosis with a telomerase inhibitor, such as imetelstat or imetelstat sodium, comprising:
(i) screening the patient to determine if such patient is: triple negative status, based on the absence of a mutation in each of JAK2, CALR, and MPL, and/or a high-molecular risk (HMR), based on the presence of a mutation in at least one of the following genes: ASXL1, EZH2, SRSF2, and IDH1/2; and
(ii) administering the telomerase inhibitor to the patient if such patient is triple negative status, based on the absence of a mutation in any of JAK2, CALR, and MPL, and/or is high-molecular risk (HMR) based on the presence of a mutation in at least one of the following genes: ASXL1, EZH2, SRSF2, and IDH1/2. The myelofibrosis may be: primary myelofibrosis, myelofibrosis that develops post-polycythemia vera (post-PV MF), or myelofibrosis that develops post essential thrombocythemia (post-ET MF). In certain embodiments, the patient has not previously received JAK-inhibitor therapy. In other embodiment, the patient has previously received JAK-inhibitor therapy and has failed JAK-inhibitor therapy,or has previously received JAK-inhibitor therapy and has discontinued JAK-inhibitor therapy due to treatment-related toxicities or intolerance.

In certain embodiments of the method of treating, the telomerase inhibitor is imetelstat and is administered for 1, 2, 3, 4, 5, 6, 7, 8, or more than 8 dosage cycles, each cycle comprising: intravenous administration of about 7-10 mg/kg imetelstat once every three weeks; intravenous administration of about 7-10 mg/kg imetelstat once weekly for three weeks; intravenous administration of about 2.5-10 mg/kg imetelstat once every three weeks; or intravenous administration of about 0.5-9.4 mg/kg imetelstat once every three weeks. In certain embodiments, each dosage cycle comprises intravenous administration of about 7-10 mg/kg imetelstat once every three weeks. In other embodiments, each dosage cycle comprises intravenous administration of about 9.4 mg/kg imetelstat once every three weeks.

In some embodiments of the methods of identifying or selecting a patient most likely to benefit from treatment with a telomerase inhibitor, the method further comprises determining average relative telomere length by analyzing the relative length of telomeric nucleic acids in target cells present in a biological sample from the patient. In some embodiments of the methods of identifying or selecting a patient most likely to benefit from treatment with a telomerase inhibitor, the method further comprises selecting the patient identified as having an average relative telomere length in target cells present in a biological sample from the patient determined to be in the 50th percentile or less of a relative telomere length range determined from one or more known standards. In certain embodiments, the telomerase inhibitor is imetelstat or a pharmaceutically acceptable salt thereof. In other embodiments, the imetelstat is imetelstat sodium.

The present disclosure provides a method of treating a patient that has myelofibrosis with a telomerase inhibitor comprising: administering the telomerase inhibitor to the patient if such patient is triple negative status, based on the absence of a mutation in each of JAK2, CALR, and MPL. In certain embodiments, the telomerase inhibitor is imetelstat or a pharmaceutically acceptable salt thereof. In other embodiments, the imetelstat is imetelstat sodium.

The present disclosure provides a method of treating a patient that has myelofibrosis with a telomerase inhibitor comprising: administering the telomerase inhibitor to the patient if such patient is triple negative status, based on the absence of a mutation in each of JAK2, CALR, and MPL, and/or is high-molecular risk (HMR) based on the presence of a mutation in at least one of the following genes: ASXL1, EZH2, SRSF2, and IDH1/2. In certain embodiments, the telomerase inhibitor is imetelstat or a pharmaceutically acceptable salt thereof. In other embodiments, the imetelstat is imetelstat sodium.

The present disclosure provides a method of treating a patient that has myelofibrosis with a telomerase inhibitor comprising: administering the telomerase inhibitor to the patient if such patient has one or more of the following characteristics:

(a) average relative telomere length in target cells present in a biological sample from the individual that is determined to be in the 50th percentile or less of a relative telomere length range determined from one or more known standards;

(b) triple negative status, based on the absence of a mutation in each of JAK2, CALR, and MPL; and

(c) high-molecular risk (HMR) based on the presence of a mutation in at least one of the following genes: ASXL1, EZH2, SRSF2, and IDH1/2. In certain embodiments, the telomerase inhibitor is imetelstat or a pharmaceutically acceptable salt thereof. In other embodiments, the imetelstat is imetelstat sodium.

The present disclosure provides a method of identifying a subject with myelofibrosis (MF) for treatment with a telomerase inhibitor, the method comprising: measuring hTERT expression level in a biological sample obtained from the patient after administration of a telomerase inhibitor; and comparing the hTERT expression level in the biological sample to a baseline hTERT expression level prior to administration of the telomerase inhibitor; wherein a reduction in hTERT expression level in the biological sample identifies a patient who has an increased likelihood of benefiting from treatment with the telomerase inhibitor.

The present disclosure provides a method of treating myelofibrosis (MF), the method comprising: administering to a subject in need thereof an effective amount of a telomerase inhibitor; and assessing hTERT expression level in a biological sample obtained from the patient after administration of the telomerase inhibitor. In certain embodiments, the telomerase inhibitor is imetelstat or a pharmaceutically acceptable salt thereof. In other embodiments, the imetelstat is imetelstat sodium.

The present disclosure provides a method of monitoring therapeutic efficacy in a subject with myelofibrosis (MF), the method comprising: measuring hTERT expression level in a biological sample obtained from the patient after administration of a telomerase inhibitor; and comparing the hTERT expression level in the biological sample to a baseline hTERT expression level prior to administration of the telomerase inhibitor; wherein a 50% or greater reduction in hTERT expression level in the biological sample identifies a subject who has an increased likelihood of benefiting from treatment with the telomerase inhibitor. In certain embodiments, the telomerase inhibitor is imetelstat or a pharmaceutically acceptable salt thereof. In other embodiments, the imetelstat is imetelstat sodium.

The present disclosure provides a method of selecting a patient most likely to benefit from treatment with a telomerase inhibitor comprising: testing a patient for average relative telomere length, by analyzing the relative length of telomeric nucleic acids in target cells present in a biological sample from the patient; and selecting the patient if the patient has average relative telomere length in target cells present in a biological sample from the patient that is determined to be in the 50th percentile or less of a relative telomere length range determined from one or more known standards, wherein the selected patient is most likely to benefit from treatment with a telomerase inhibitor.

The present disclosure provides a method of identifying a patient most likely to benefit from treatment with a telomerase inhibitor comprising: obtaining a biological sample from a patient; determining average relative telomere length by analyzing the relative length of telomeric nucleic acids in target cells present in the biological sample from the patient; and identifying the patient if the patient has average relative telomere length in target cells present in the biological sample from the patient that is determined to be in the 50th percentile or less of a relative telomere length range determined from one or more known standards, wherein the identified patient is most likely to benefit from treatment with a telomerase inhibitor.

The present disclosure provides a method of treating a patient that has myelofibrosis with a telomerase inhibitor comprising: administering the telomerase inhibitor to the patient if such patient has average relative telomere length in target cells present in a biological sample from the patient that is determined to be in the 50th percentile or less of a relative telomere length range determined from one or more known standards. In certain embodiments, the telomerase inhibitor is imetelstat or a pharmaceutically acceptable salt thereof. In other embodiments, the imetelstat is imetelstat sodium.

The present disclosure provides a method of monitoring therapeutic efficacy in a subject with myelofibrosis (MF), the method comprising measuring hTERT expression level in a biological sample obtained from the patient after administration of a telomerase inhibitor; and comparing the hTERT expression level in the biological sample to a baseline hTERT expression level prior to administration of the telomerase inhibitor; wherein a 50% or greater reduction in hTERT expression level in the biological sample identifies a subject who has an increased likelihood of benefiting from treatment with the telomerase inhibitor. In certain embodiments, the hTERT expression level measured or assessed is hTERT RNA expression level. In certain embodiments, the telomerase inhibitor is imetelstat or a pharmaceutically acceptable salt thereof. In other embodiments, the imetelstat is imetelstat sodium.

The present disclosure provides a method of identifying a patient with myelofibrosis (MF) for treatment with a telomerase inhibitor, the method comprising: measuring hTERT expression level in a biological sample obtained from the patient after administration of a telomerase inhibitor; and comparing the hTERT expression level in the biological sample to a baseline hTERT expression level prior to administration of the telomerase inhibitor; wherein a reduction in hTERT expression level in the biological sample identifies a patient who has an increased likelihood of benefiting from treatment with the telomerase inhibitor.

The present disclosure provides a method of monitoring therapeutic efficacy in a subject with myelofibrosis (MF), the method comprising: measuring telomerase activity level in a biological sample obtained from the patient after administration of a telomerase inhibitor; and comparing the telomerase activity level in the biological sample to a baseline telomerase activity level prior to administration of the telomerase inhibitor; wherein a 50% or greater reduction in telomerase activity level in the biological sample identifies a subject who has an increased likelihood of benefiting from treatment with the telomerase inhibitor. In certain embodiments, the telomerase inhibitor is imetelstat or a pharmaceutically acceptable salt thereof. In other embodiments, the imetelstat is imetelstat sodium.

### DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown.

FIG. 1 shows a waterfall plot of spleen volume reduction (SVR) at week 24 for the 4.7 mg/kg and 9.4 mg/kg treatment arms in Example 1. The SVR is shown as a percent change from baseline.

FIG. 2 shows a waterfall plot of total symptom score reduction (TSS) at week 24 for the 4.7 mg/kg and 9.4 mg/kg treatment arms in Example 1. The TSS is shown as a percent change from baseline.

FIG. 3 shows a Kaplan-Meier Plot of Overall Survival Grouped by Mutation Status of JAK2/MPL/CALR Genes: TN vs. Non-TN (MUT) for the 4.7 mg/kg arm. Specifically, FIG. 3 shows the survival probability for patients having triple negative status (TN) and patients having at least one mutation (MUT) as a function of time.

FIG. 4 shows a Kaplan-Meier Plot of Overall Survival Grouped by Mutation Status of JAK2/MPL/CALR Genes: TN vs. Non-TN (MUT) for the 9.4 mg/kg arm. Specifically, FIG. 4 shows the survival probability for patients having triple negative status (TN) and patients having at least one mutation (MUT) as a function of time for the 9.4 mg/kg arm.

FIG. 5 shows a Kaplan-Meier Plot of Overall Survival as a function of time grouped according to patients in the 9.4 mg/kg versus 4.7 mg/kg arm.

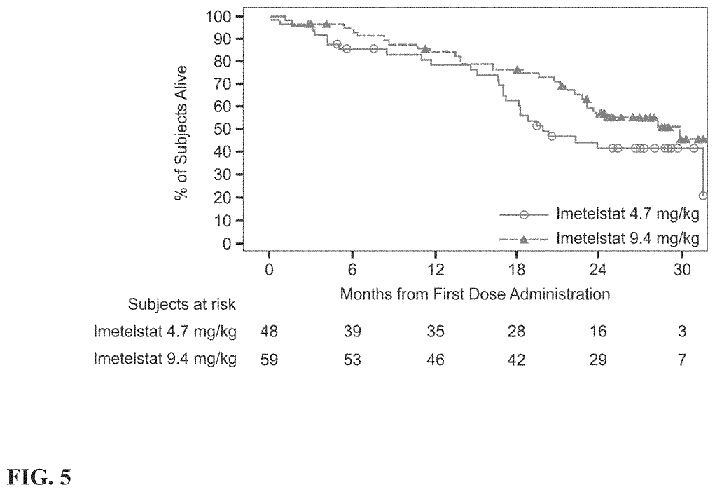

FIG. 6 shows a Kaplan-Meier Plot of Overall Survival (OS) Grouped by Mutation Status of JAK2/MPL/CALR Genes: TN vs. Non-TN for the 9.4 mg/kg arm. Specifically, FIG. 6 shows the survival probability for patients having triple negative status (TN) and patients having at least one mutation (non-TN) as a function of time for the 9.4 mg/kg arm.

FIG. 7 shows a Kaplan-Meier Plot of Overall Survival (OS) Grouped by Mutation Status of JAK2/MPL/CALR Genes: TN vs. Non-TN for the 4.7 mg/kg arm. Specifically, FIG. 7 shows the survival probability for patients having triple negative status (TN) and patients having at least one mutation (non-TN) as a function of time for the 4.7 mg/kg arm.

### DETAILED DESCRIPTION

This application is based on the discovery that patients that have myelofibrosis, that are triple negative (i.e. the absence of a mutation in each of JAK2, CALR, and MPL), and/or that are in a high-molecular risk (HMR) category, based on the presence of a mutation in at least one of the following genes: ASXL1, EZH2, SRSF2, and IDH1/2, are able to benefit from treatment with a telomerase inhibitor, such as imetelstat or imetelstat sodium. Patients having mutations in ASXL1, EZH1, IDH1/2, and SRSF2 genes have an elevated risk for early death or leukemic transformation. These patients typically do not benefit from treatment using conventional therapies, such as JAK inhibitors. Gisslinger et al., Blood, 128: 1931 (2016). Thus, the fact that these patients benefit from treatment with a telomerase inhibitor is unexpected and surprising.

Accordingly, this application provides for methods of identifying a patient most likely to benefit from treatment with a telomerase inhibitor, such as imetelstat. The methods comprise testing or identifying a patient to determine if the patient has triple negative status, based on the absence of a mutation in each of JAK2, CALR, and MPL; and/or is high-molecular risk (HMR), based on the presence of a mutation in at least one of the following genes: ASXL1, EZH2, SRSF2, and IDH1/2. The application also provides for methods of treating myelofibrosis with telomerase inhibitor, such as imetelstat, which involve identifying a patient that has: triple negative status, based on the absence of a mutation in each of JAK2, CALR, and MPL; and/or a high-molecular risk (HMR), based on the presence of a mutation in at least one of the following genes: ASXL1, EZH2, SRSF2, and IDH1/2. Such patients are most likely to benefit from treatment with a telomerase inhibitor. The telomerase inhibitor (e.g. imetelstat) is then administered to the patient. For clarity of disclosure, and not by way of limitation, the detailed description of the invention is divided into subsections that describe or illustrate certain features, embodiments, or applications of the present invention.

### A. Definitions

As used herein, a mutation in additional sex combs like 1 (ASXL1), enhancer of zeste homolog 2 (EZH2), serine and arginine rich splicing factor 2 (SRSF2), and isocitrate dehydrogenase 1/2 (IDH1/2) shall include any mutation in these genes that impacts survival and disease progression in a patient having myelofibrosis. Furthermore, as used herein IDH1/2 shall include IDH1 and IHD2. Exemplary mutations may be found in the following publications, the disclosure of each of which is being incorporated as it pertains to disclosing genetic mutations associated with myelofibrosis: Langabeer, JAK-STAT, 5: e1248011 (2016); Cervantes, Blood; 124(17):2635-2642 (2014); Patel et al., Blood; 126(6):790-797 (2015); Spiegel et al., Blood Adv., 1(20):1729-1738 (2017); Newburry et al., Blood,130(9):1125-1131 (2017); Kuykendall et al., Annals of Hematology, 97: 435-431 (2018). Exemplary sequences are as follows: high-molecular risk (HMR) may be determined based on the presence of a mutation in at least one of the following genes: a ASXL1 gene having, for example, the nucleic acid sequence of SEQ ID NO: 5, a EZH2 gene having, for example, the nucleic acid sequence of SEQ ID NO: 6, a SRSF2 gene having, for example, the nucleic acid sequence of SEQ ID NO: 7, an IDH1 gene having, for example, the nucleic acid sequence of SEQ ID NO: 8, an IDH2 gene having the nucleic acid sequence of SEQ ID NO: 9, and combinations thereof.

In some embodiments, mutations of interest in the ASXL1 gene include mutations of Q575, Q588, Y591, Q592, 5604, L614, Q623, A627, E635, T638, A640, G646, G658, R678, C687, D690, R693, Y700, G704, E705, Q708, G710, L721, E727, V751, P763, Q780, W796, V807, T822, K825, 5846, D855, C856, L857, L885, L890, S903, 5970, Y974, R965, G967, V962, L992, 51028, Q1039, R1073, E1102, H1153, S1209, 51231, A1312, F1305, P1377, R1415 and 11436. In some embodiments, the mutation is a Q575X mutation, a Q588X, a Y591X mutation, a Y591N mutation, a Q592X mutation, a 5604F mutation, a L614F mutation, a Q623X mutation, a A627G mutation, a E635R mutation, a T638V mutation, a A640G mutation, a G646W mutation, a G658X mutation, a R678K mutation, a C687R mutation, a C687V mutation, a D690G mutation, a R693X mutation, a Y700X mutation, a G704R mutation, a G704W mutation, a E705X mutation, a Q708X mutation, a G710E mutation, a L721C mutation, a E727X mutation, a V751L mutation, a P763R mutation, a Q780X mutation, a W796X mutation, a W796G mutation, a V807F mutation, a T822H mutation, a K825X mutation, a S846Q mutation, a D855A mutation, a C856X mutation, a L857R mutation, a L885X mutation, a L890F mutation, a S903I mutation, a 5970N mutation, a Y974X mutation, a R965X mutation, a G967del mutation, a V962A mutation, a L992Q mutation, a 51028R mutation, a Q1039L mutation, a R1073C mutation, a E1102D mutation, a H1153R mutation, a S12091 mutation, a 51231F mutation, a A1312V mutation, a F1305W mutation, a P13775 mutation, a R1415Q mutation and/or a I1436M mutation.

In some embodiments, mutations of interest in the EZH2 gene include mutations of W60, R63, P312, F145, N182, R288, Q328, Q553, R566, T573, R591, R659, D677, V679, R690, A702, V704, E726, D730 and/or Y733. In some embodiments, the mutation is a W60X mutation, a R63X mutation, a P3125 mutation, a F145S mutation, a N182D mutation, a R288Q mutation, a Q328X mutation, a Q553X mutation, a R566H mutation, a T573I mutation, a R591H mutation, a R659K mutation, a D677H mutation, a V679M mutation, a R690H mutation, a A702V mutation, a V704L mutation, a E726V mutation, a D730X mutation and/or a Y733X mutation.

In some embodiments, mutations of interest in the SRSF2 gene include mutations of P95. In some embodiments, the mutation is a P95H mutation, a P95L mutation or a P95R mutation.

In some embodiments, mutations of interest in the IDH1/2 gene include mutations of R132 and/or R140. In some embodiments, the mutation is a R132G mutation, a R132H mutation or a R140Q mutation.

In certain embodiment, mutations of interest include those set forth below:

| Gene | Protein | Change |
|---|---|---|
| ASXL1 | p.Tyr591* | Nonsense |
| ASXL1 | p.Gln592* | Nonsense |
| ASXL1 | p.Ile617* | Nonsense |
| ASXL1 | p.Glu635Argfs*15 | Frameshift |
| ASXL1 | p.Gly646Trpfs*12 | Frameshift |
| ASXL1 | p.Asp667Trpfs*2 | Frameshift |
| ASXL1 | p.Gln692* | Nonsense |
| ASXL1 | p.Arg693* | Nonsense |
| ASXL1 | p.Arg693Ter | Nonsense |
| ASXL1 | p.Tyr700Ilefs*3 | Nonsense |
| ASXL1 | p.Lys726* | Nonsense |
| ASXL1 | p.Gln760Hisfs*13 | Frameshift |
| ASXL1 | p.Leu775* | Nonsense |
| ASXL1 | p.Trp796* | Frameshift |
| ASXL1 | p.Pro808fs | Frameshift |
| ASXL1 | p.Pro808His | Missense |
| ASXL1 | p.Pro808Leufs*10 | Frameshift |
| ASXL1 | p.Leu823* | Nonsense |
| ASXL1 | p.Gly826Glufs*12 | Frameshift |
| ASXL1 | p.Ala861Aspfs*6 | Frameshift |
| ASXL1 | p.Pro938* | Nonsense |
| ASXL1 | p.Asn986Ser | Missense |
| ASXL1 | p.Gln1234* | Nonsense |
| ASXL1 | p.Thr1388Serfs*5 | Frameshift |
| EZH2 | p.Lys400_Glu401del | In-Frame Deletion |
| EZH2 | p.Cys457Tyr | Missense |
| EZH2 | p.Ser480Argfs*3 | Frameshift |
| EZH2 | p.Cys552Tyr | Missense |
| EZH2 | p.Leu56Phefs*2 | Frameshift |
| EZH2 | p.Ser669Arg | Missense |
| EZH2 | p.Asp681_Lys685del | In-Frame Deletion |
| EZH2 | p.Arg684Cys | Missense |
| EZH2 | p.Tyr733* | Nonsense |
| EZH2 | p.Glu745Lys | Missense |
| EZH2 | p.Ile223Phefs*18 | Frameshift |
| SRSF2 | p.Pro95Ala | Missense |
| SRSF2 | p.Pro95His | Missense |
| SRSF2 | p.Pro95Arg | Missense |
| SRSF2 | p.Pro95Leu | Missense |
| SRSF2 | p.Pro107His | Missense |
| IDH1 | p.Arg132His | Missense |
| IDH2 | p.Arg140Gln | Missense |

As used herein, “triple negative status”, “triple negative” or “TN” shall refer to patients having the absence of a mutation in each of Janus kinase 2 (JAK2), calreticulin (CALR), and thrombopoietin receptor (MPL) genes. Triple negative status may be determined based on the absence of a mutation in each of a JAK2 gene having, for example, the nucleic acid sequence of SEQ ID NO: 2, a CALR gene having, for example, the nucleic acid sequence of SEQ ID NO: 3, and a MPL gene having, for example, the nucleic acid sequence of SEQ ID NO: 4.

In certain embodiments, triple negative status includes an absence of a mutation in the JAK2 gene, such as a mutation at G335, F556, G571, V617 and/or V625. For example, triple negative status may include the absence in the JAK2 gene of a G335D mutation, a F556V mutation, a G5715 mutation, a V617F mutation and/or a V625S mutation.

In certain embodiments, triple negative status includes an absence of a mutation in the MPL gene, such as a mutation at T119, S204, P222, E230, V285, R321, S505, W515, Y591 and/or R592. For example, triple negative status may include the absence in the MPL gene of a T119I mutation, a S204F mutation, a S204P mutation, a P222S mutation, a E230G mutation, a V285E mutation, a R321W mutation, a S505N mutation, a W515R mutation, a W515L mutation, a Y591N mutation and/or a R592Q mutation.

In certain embodiments, triple negative status includes an absence of a mutation in the CALR gene, such as a mutation at L367, K368, E381, K385 and/or E396. For example, triple negative status may include the absence in the CALR gene of a L367T mutation, a K368R mutation, a K385N mutation, a E381A mutation and/or a E396del mutation.

In certain embodiments, mutations of interest include those set forth below:

| Gene | Protein | Change |
|---|---|---|
| JAK2 | p.Val617Phe | Missense |
| MPL | p.Ser505Asn | Missense |
| MPL | p.Trp515Lys | Missense |
| MPL | p.Trp515Leu | Missense |
| CALR | p.Leu367Thrfs*46 | Frameshift |
| CALR | p.Leu367fs | Frameshift |
| CALR | p.Glu381Ala | Missense |
| CALR | p.Lys385Asnfs*47 | Frameshift |
| CALR | p.Glu396del | In-Frame Deletion |
| CALR | p.Lys368Argfs*51 | Frameshift |

As used herein, a patient has “failed” JAK inhibitor therapy when the disease was resistant or the patient was refractory to the therapy or although initially responsive to treatment, the disease has relapsed.

As used herein, the term “about” when referring to a measurable value such as an amount, a temporal duration, and the like, is meant to encompass variations of between ±20% and ±0.1%, preferably ±20% or ±10%, more preferably ±5%, even more preferably ±1%, and still more preferably ±0.1% from the specified value, as such variations are appropriate to perform the disclosed methods.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, and the like; and when the molecule contains a basic functionality, salts of organic or inorganic acids, such as hydrochloride, and the like. Pharmaceutically acceptable salts of interest include, but are not limited to, aluminum, ammonium, arginine, barium, benzathine, calcium, cholinate, ethylenediamine, lysine, lithium, magnesium, meglumine, procaine, potassium, sodium, tromethamine, N-methylglucamine, N,N′-dibenzylethylene-diamine, chloroprocaine, diethanolamine, ethanolamine, piperazine, zinc, diisopropylamine, diisopropylethylamine, triethylamine and triethanolamine salts.

The term “salt(s) thereof” means a compound formed when a proton of an acid is replaced by a cation, such as a metal cation or an organic cation and the like. Preferably, the salt is a pharmaceutically acceptable salt. By way of example, salts of the present compounds include those wherein the compound is protonated by an inorganic or organic acid to form a cation, with the conjugate base of the inorganic or organic acid as the anionic component of the salt. Salts of interest include, but are not limited to, aluminum, ammonium, arginine, barium, benzathine, calcium, cesium, cholinate, ethylenediamine, lithium, magnesium, meglumine, procaine, N-methylglucamine, piperazine, potassium, sodium, tromethamine, zinc, N,N′-dibenzylethylene-diamine, chloroprocaine, diethanolamine, ethanolamine, piperazine, diisopropylamine, diisopropylethylamine, triethylamine and triethanolamine salts. It is understood that for any of the oligonucleotide structures depicted herein that include a backbone of internucleoside linkages, such oligonucleotides may also include any convenient salt forms. In some embodiments, acidic forms of the internucleoside linkages are depicted for simplicity. In some instances, the salt of the subject compound is a monovalent cation salt. In certain instances, the salt of the subject compound is a divalent cation salt. In some instances, the salt of the subject compound is a trivalent cation salt. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include for example cis-trans isomers, E and Z isomers, enantiomers, and diastereomers. As to any of the groups disclosed herein which contain one or more substituents, it is understood, of course, that such groups do not contain any substitution or substitution patterns which are sterically impractical and/or synthetically non-feasible. All stereoisomers are intended to be included within the scope of the present disclosure.

A person of ordinary skill in the art would recognize that other tautomeric arrangements of the groups described herein are possible. It is understood that all tautomeric forms of a subject compound are encompassed by a structure where one possible tautomeric arrangement of the groups of the compound is described, even if not specifically indicated.

It is intended to include a solvate of a pharmaceutically acceptable salt of a tautomer of a stereoisomer of a subject compound. These are intended to be included within the scope of the present disclosure.

Before certain embodiments are described in greater detail, it is to be understood that this invention is not limited to certain embodiments described, and as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements,or use of a “negative” limitation.

Each of the individual embodiments described and illustrated herein have discrete components and features which may be readily separated from or combined with the features of any of the other several embodiments without departing from the scope or spirit of the present invention. Any recited method may be carried out in the order of events recited or in any other order which is logically possible.

B. Identifying a Patient most Likely to Benefit from Treatment with a Telomerase Inhibitor

In one aspect, the present disclosure provides for methods of identifying or selecting a patient having myelofibrosis most likely to benefit from treatment with a telomerase inhibitor. The methods rely on identifying triple negative status patients (patients having the absence of a mutation in each of JAK2, CALR, and MPL genes) or patients that are high-molecular risk (HMR) based on the presence of a mutation in at least one of the following genes: ASXL1, EZH2, SRSF2, and IDH1/2. These triple negative patients or HMR patients are most likely to benefit from treatment with a telomerase inhibitor, such as imetelstat or imetelstat sodium.

The myelofibrosis may be primary myelofibrosis, myelofibrosis that develops post-polycythemia vera (post-PV MF), or myelofibrosis that develops post essential thrombocythemia (post-ET MF). In certain embodiments, the patient has not previously received JAK-inhibitor therapy. In other embodiments, the patient has previously received JAK-inhibitor therapy and has failed JAK-inhibitor therapy (i.e., the disease was resistant or the patient was refractory to the therapy or although initially responsive to treatment, the disease has relapsed). In other embodiments, the patient has previously received JAK-inhibitor therapy and has discontinued JAK-inhibitor therapy due to treatment-related toxicities or intolerance. In yet alternate embodiments, the patient has previously received JAK-inhibitor therapy and has discontinued JAK-inhibitor therapy.

In one embodiment, the patient has received JAK-inhibitor therapy and the myelofibrosis was resistant to JAK-inhibitor therapy. In another embodiment, the patient has received JAK-inhibitor therapy and the patient was refractory to JAK-inhibitor therapy. In another embodiments, the patient has received JAK-inhibitor therapy and the patient is relapsed. In an alternate embodiment, the patient has received JAK-inhibitor therapy and discontinued JAK-inhibitor therapy due to treatment-related toxicities or intolerance.

In one embodiment, the invention provides for methods of selecting a patient most likely to benefit from treatment with a telomerase inhibitor by testing for one or more of: triple negative status, based on the absence of a mutation in each of JAK2, CALR, and MPL genes (i.e. the lack of any mutation). In that embodiment, the patient may also be tested for a high-molecular risk (HMR), based on the presence of a mutation in at least one of the following genes: ASXL1, EZH2, SRSF2, and IDH1/2. In another embodiment, the invention provides for methods of selecting a patient most likely to benefit from treatment with a telomerase inhibitor by testing for: triple negative status, based on the absence of a mutation in each of JAK2, CALR, and MPL genes (i.e. the lack of any mutation); and/or a high-molecular risk (HMR), based on the presence of a mutation in at least one of the following genes: ASXL1, EZH2, SRSF2, and IDH1/2. In another embodiment, the invention provides for a method of identifying a patient most likely to benefit from treatment with a telomerase inhibitor comprising testing a patient for: (a) triple negative status, based on the absence of any mutation in JAK2, CALR, and MPL genes; (b) a high-molecular risk (HMR), based on the presence of a mutation in at least one of the following genes: ASXL1, EZH2, SRSF2, and IDH1/2; or (c) both. In this embodiment, the presence of (a), (b), or (c) is indicative of a patient most likely to benefit from treatment with a telomerase inhibitor.

Another embodiment of the invention is a method of identifying a patient most likely to benefit from treatment with a telomerase inhibitor comprising:

a. testing a patient for the following:
i. triple negative status, based on the absence of a mutation in each of JAK2, CALR, and MPL genes, and/or
ii. a high-molecular risk (HMR), based on the presence of a mutation in at least one of the following genes: ASXL1, EZH2, SRSF2, and IDH1/2,

b. selecting the patient if the patient has the following:
i. triple negative status, based on no mutation in each of JAK2, CALR, and MPL genes, and/or
ii. a high-molecular risk (HMR), based on the presence of a mutation in at least one of the following genes: ASXL1, EZH2, SRSF2, and IDH1/2,
   wherein the selected patient is most likely to benefit from treatment with a telomerase inhibitor.

Yet another embodiment of the invention is a method of identifying a patient most likely to benefit from treatment with a telomerase inhibitor comprising: testing a patient for triple negative status, based on the absence of a mutation in each of JAK2, CALR, and MPL genes; and selecting the patient if the patient has: triple negative status, based on the absence of a mutation in each of JAK2, CALR, and MPL genes, wherein the selected patient is most likely to benefit from treatment with a telomerase inhibitor. In one embodiment, this method also includes testing a patient for a high-molecular risk (HMR), based on the presence of a mutation in at least one of the following genes: ASXL1, EZH2, SRSF2, and IDH1/2 and selecting the patient if the patient has a HMR.

In certain embodiments of any of these methods, the triple negative patients lack a mutation in the coding region (exon) of the JAK2, CALR, and MPL genes.

Furthermore, in other embodiments of any of these methods, a high-molecular risk (HMR) is determined by the presence of a mutation in the coding region (exon) of at least one of the ASXL1, EZH2, SRSF2, and IDH1/2 genes.

In certain embodiment, a high-molecular risk (HMR) is determined by detecting the presence of a mutation in ASXL1, EZH2, SRSF2 or IDH1/2 or combination thereof. In some embodiments, methods include detecting the presence of a mutation in ASXL1. In some embodiments, methods include detecting the presence of a mutation in EZH2. In some embodiments, methods include detecting the presence of a mutation in SRSF2. In some embodiments, methods include detecting the presence of a mutation in IDH1/2. In some embodiments, methods include detecting the presence of a mutation in ASXL1 and EZH2. In some embodiments, methods include detecting the presence of a mutation in ASXL1 and SRSF2. In some embodiments, methods include detecting the presence of a mutation in ASXL1 and IDH1/2. In some embodiments, methods include detecting the presence of a mutation in EZH2, SRSF2. In some embodiments, methods include detecting the presence of a mutation in EZH2 and IDH1/2. In some embodiments, methods include detecting the presence of a mutation in SRSF2 and IDH1/2. In some embodiments, methods include detecting the presence of a mutation in ASXL1, EZH2 and SRSF2. In some embodiments, methods include detecting the presence of a mutation in ASXL1, EZH2 and IDH1/2. In some embodiments, methods include detecting the presence of a mutation in EZH2, SRSF2 and IDH1/2. In some embodiments, methods include detecting the presence of a mutation in ASXL1, EZH2, SRSF2 and IDH1/2. In yet another embodiment of the invention, the invention provides for a method of identifying or selecting a patient most likely to benefit from treatment with a telomerase inhibitor in a patient population. In this method, the patient population is screened for patients having mutations in each of JAK2, CALR, and MPL genes to identify triple negative patients in the population. In alternate embodiments, the methods rely on identifying triple negative patients lacking a canonical mutation in each of JAK2, MPL, and CALR.

In certain embodiments, the methods also include a step of taking a patient DNA sample. The patient sample may be collected from a DNA sample obtained from bone marrow, peripheral blood or both. Accordingly, in certain embodiments, the methods of invention include obtaining a patient blood sample and isolating (extracting) the DNA from the patient blood sample. The methods may also include a step of isolating cells (e.g. granulocytes) from the patient blood sample. Similarly, the methods of the invention include obtaining a bone marrow sample and isolating (extracting) the DNA from the bone marrow sample. The method may also include the step of isolating cells from the patient bone sample.

The patient DNA sample is tested for the presence or absence of mutations in each of the JAK2, CALR, and MPL genes using conventional techniques. Alternatively, the patient DNA sample is tested for the presence of a mutation in at least one of the following genes: ASXL1, EZH2, SRSF2, and IDH1/2 using conventional techniques. In certain embodiments, patient DNA sample is tested for: (i) the presence or absence of mutations in each of the JAK2, CALR, and MPL genes; and (ii) the presence of a mutation in at least one of the following genes: ASXL1, EZH2, SRSF2, and IDH1/2.

In certain embodiments, the testing of the DNA sample may be next generation sequencing assay using the Illumina MiSeq platform as disclosed in Patel et al., Blood; 126(6):790-797 (2015), the disclosure of which as it pertains to DNA sample testing is incorporated herein.

### C. Pharmacodynamics (PD)

The present disclosure is based in part on a pharmacodynamic effect demonstrating an association between response to telomerase inhibition therapy in subjects with myelofibrosis and a decrease in telomerase hTERT expression levels in the subjects from baseline levels. In some cases, a higher % of subjects achieve a 50% or more decrease in hTERT RNA expression levels in subjects who achieved clinical response (spleen or symptom) to telomerase inhibition therapy at week 24 than those who did not achieve response.

The present disclosure provides for stratification and selection of patients likely to benefit from telomerase inhibition therapy for myelofibrosis, and provides methods of monitoring response, relapse, and prognosis in subjects undergoing treatment.

Aspects of the present disclosure include methods selecting subjects with myelofibrosis (MF) for treatment with a telomerase inhibitor, and methods of treating MF. Methods of monitoring therapeutic efficacy in a subject with MF are also provided. In some case, the pharmacodynamic effect on which an embodiment of the subject methods is based is reduction of hTERT RNA expression by 50% or more, such as 60% or more, 70% or more, 80% or more, or 90% or more.

The telomerase ribonucleoprotein consists of components or subunits, two of these being telomerase RNA template (hTR), and telomerase reverse transcriptase protein (hTERT). hTERT expression levels can be assessed, determined and/or measured using any convenient methods. A variety of methods can be applied for the amplification, detection and measurement of mRNA of telomerase components or related proteins in bodily fluids. Methods and assays of interest which may be adapted for use in the subject methods include, but are not limited to, real-time quantitative RT-PCR assays, e.g., based on based on TaqMan fluorescence methodology, immunohistochemistry methods for protein expression, and methods described by U.S. Pat. No. 6,607,898, Bieche et al., Clin. Cancer Res Feb. 1 2000 (6) (2) 452-459, Terrin et al. (“Telomerase expression in B-cell chronic lymphocytic leukemia predicts survival and delineates subgroups of patients with the same igVH mutation status and different outcome.” Leukemia 2007; 21: 965-972), and Palma et al. (“Telomere length and expression of human telomerase reverse transcriptase splice variants in chronic lymphocytic leukemia.” Experimental Hematology 2013; 41: 615-626).

The hTERT expression levels can be assessed or measured in any convenient target cells or biological samples. Target cells can be any convenient cells of the patient, including but not limited to, cells of the bone marrow or peripheral blood of the patient. In some cases, the target cells are isolated from a bone marrow sample of the patient. In some cases, the target cells are isolated from a peripheral blood sample of the patient. The target cells can be granulocytes.

hTERT RNA expression levels can be assessed or measured in a RNA sample using any convenient methods. A RNA sample may be obtained by first obtaining a bone marrow sample, a peripheral blood sample, or both and then isolating the RNA from the bone marrow sample, the peripheral blood sample, or both. In one embodiment, the step of obtaining a sample from a patient comprises: obtaining a bone marrow sample from the patient, isolating cells from the bone marrow sample, and extracting RNA and/or DNA from the isolated cells. In another embodiment, the step of obtaining a RNA sample from a patient comprises: obtaining a peripheral blood sample from the patient; isolating cells from the peripheral blood sample (e.g. granulocytes); and extracting RNA and/or DNA from the isolated cells.

### D. Treatment

Aspects of the present disclosure include methods of treating myelofibrosis in a subject in need thereof (i.e. patient) having: triple negative status, based on the absence of any mutation in JAK2, CALR, and MPL genes (i.e. no mutation in these genes or these genes lacking mutation); and/or high-molecular risk (HMR), based on the presence of a mutation in at least one of the following genes: ASXL1, EZH2, SRSF2, and IDH1/2. One embodiment of the invention is a method of treating myelofibrosis in a subject in need thereof (i.e. patient) having: triple negative status, based on the absence of any mutation in JAK2, CALR, and MPL genes (i.e. no mutation in these genes or lacking a mutation). In one embodiment, the myelofibrosis is primary myelofibrosis. In another embodiment, the myelofibrosis is myelofibrosis that develops post-polycythemia vera (post-PV MF). In an alternate embodiment, the myelofibrosis is myelofibrosis that develops post essential thrombocythemia (post-ET MF).

In certain embodiments of the treatment methods, the patient has not previously received JAK-inhibitor therapy. In other embodiments, the patient has previously received JAK-inhibitor therapy and has “failed” JAK-inhibitor therapy (i.e., the disease was resistant or the patient was refractory to the therapy or although initially responsive to treatment, the disease has relapsed). In alternate embodiments of the treatment methods, the patient has received JAK-inhibitor therapy and has discontinued JAK-inhibitor therapy, due to treatment-related toxicities or intolerance. In certain embodiments, the methods of treating further include premedication with diphenhydramine (25 to 50 mg) and hydrocortisone (100 to 200 mg), or equivalent thereof.

A subject is a mammal in need of treatment for cancer. Generally, the subject is a human patient. In some embodiments of the invention, the subject can be a non-human mammal such as a non-human primate, an animal model (e.g., animals such as mice and rats used in screening, characterization, and evaluation of medicaments) and other mammals. As used herein, the terms patient, subject and individual are used interchangeably.

As used herein, and as well understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

### E. Telomerase Inhibitors

The methods of the invention may be used to identify a patient most likely to benefit from treatment with any convenient telomerase inhibitors. Furthermore, any convenient telomerase inhibitors can find use in the subject treatment methods. In some embodiments, the telomerase inhibitor is an oligonucleotide with telomerase inhibiting activity, in particular an oligonucleotide as defined in WO 2005/023994 and/or WO 2014/088785, the disclosures of which are herein incorporated by reference in their entirety. In some cases, one or more than one telomerase inhibitor (e.g., two or three telomerase inhibitors) can be administered to a mammal to treat a hematological malignancy.

Imetelstat

In certain embodiments, the telomerase inhibitor is imetelstat, including tautomers thereof and salts thereof, e.g., pharmaceutically acceptable salts. Imetelstat is a novel, first-in-class telomerase inhibitor with clinical activity in hematologic malignancies (Baerlocher et al., NEJM 2015; 373:920-928; Tefferi et al., NEJM 2015; 373:908-919) (shown below):

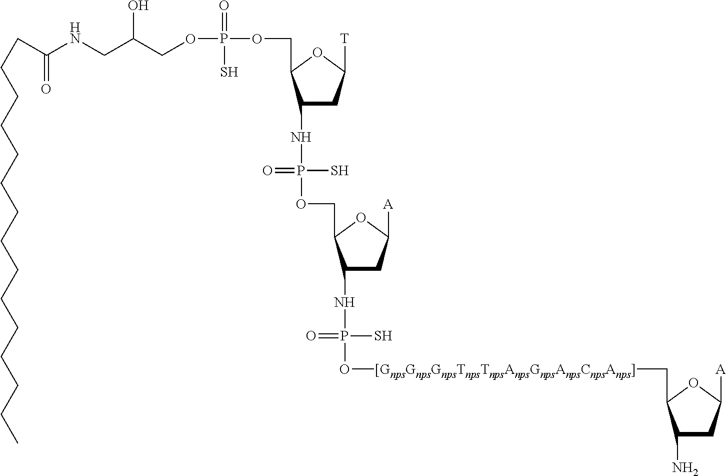

where “nps” represents a thiophosphoramidate linkage —NH—P(═O)(SH)—O—, connecting the 3′-carbon of one nucleoside to the 5′-carbon of the adjacent nucleoside.

In certain instances, the telomerase inhibitor is imetelstat sodium including tautomers thereof. Imetelstat sodium is the sodium salt of imetelstat, which is a synthetic lipid-conjugated, 13-mer oligonucleotide N3′→P5′-thio-phosphoramidate. Imetelstat sodium is a telomerase inhibitor that is a covalently-lipidated 13-mer oligonucleotide (shown below) complimentary to the human telomerase RNA (hTR) template region. The chemical name for imetelstat sodium is: DNA, d(3′-amino-3′-deoxy-P-thio) (T-A-G-G-G-T-T-A-G-A-C-A-A), 5′-[O-[2-hydroxy-3-(hexadecanoylamino)propyl] phosphorothioate], sodium salt (1:13) (SEQ ID NO: 1). Imetelstat sodium does not function through an anti-sense mechanism and therefore lacks the side effects commonly observed with such therapies.

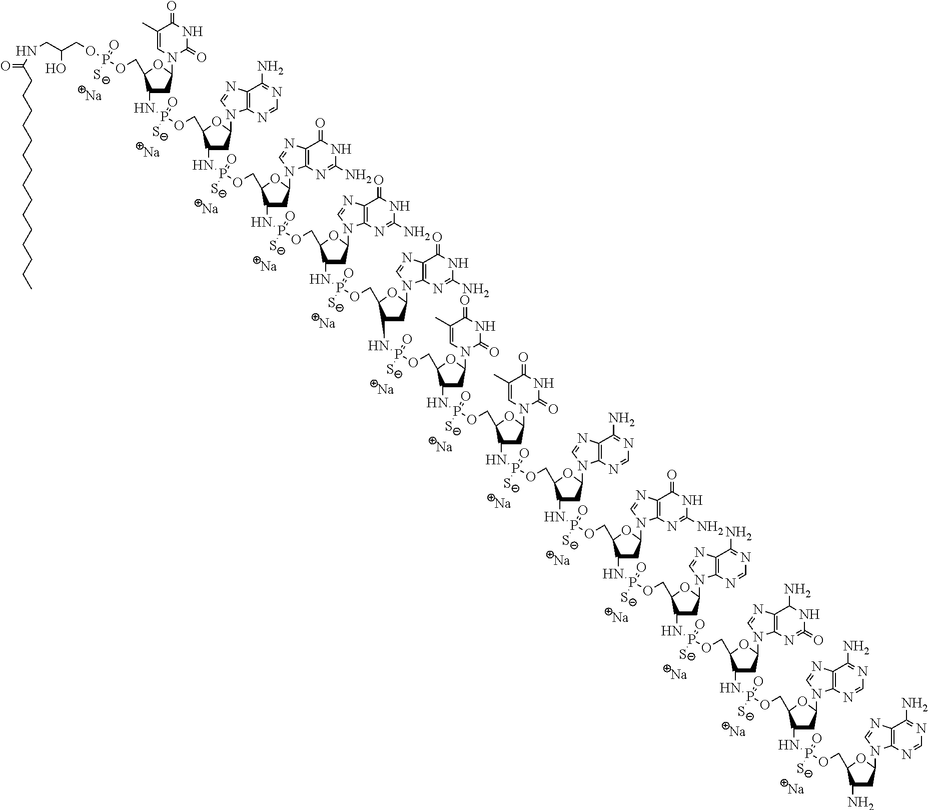

Unless otherwise indicated or clear from the context, references herein to imetelstat also include tautomers thereof and salts thereof, e.g., pharmaceutically acceptable salts. As mentioned, imetelstat sodium in particular is the sodium salt of imetelstat. Unless otherwise indicated or clear from the context, references herein to imetelstat sodium also include all tautomers thereof.

Imetelstat and imetelstat sodium can be produced, formulated, or obtained as described elsewhere (see e.g. Asai et al., Cancer Res., 63:3931-3939 (2003), Herbert et al., Oncogene, 24:5262-5268 (2005), and Gryaznov, Chem. Biodivers., 7:477-493 (2010)). Unless otherwise indicated or clear from the context, references herein to imetelstat also include salts thereof. As mentioned, imetelstat sodium in particular is the sodium salt of imetelstat.

Imetelstat targets the RNA template of telomerase and inhibits telomerase activity and cell proliferation in various cancer cell lines and tumor xenografts in mice. Phase 1 studies involving patients with breast cancer, non-small-cell lung cancer and other solid tumors, multiple myeloma, or chronic lymphocytic leukemia have provided information on drug pharmacokinetics and pharmacodynamics. A subsequent phase 2 study involving patients with essential thrombocythemia showed platelet-lowering activity accompanied by a significant reduction in JAK2 V617F and CALR mutant allele burdens. Imetelstat sodium is routinely administered intravenously; it is contemplated that in the practice of the subject methods other administration routes also can be used, such as intrathecal administration, intratumoral injection, oral administration and others. Imetelstat sodium can be administered at doses comparable to those routinely utilized clinically. In certain embodiments, imetelstat sodium is administered as described elsewhere herein.

A particular embodiment is according to any one of the other embodiments, wherein imetelstat is limited to imetelstat sodium.

### F. Pharmaceutical Compositions

For ease of administration, the telomerase inhibitor (e.g., as described herein) may be formulated into various pharmaceutical forms for administration purposes. In some cases, the telomerase inhibitor is administered as a pharmaceutical composition. The carrier or diluent of the pharmaceutical composition must be “acceptable” in the sense of being compatible with the other ingredients of the composition and not deleterious to the recipients thereof. The pharmaceutical composition may be in unitary dosage form suitable, in particular, for administration orally, rectally, percutaneously, by parenteral injection or by inhalation. In some cases, administration can be via intravenous injection. For example, in preparing the composition in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions, for example, may be prepared in which the carrier comprises saline solution, glucose solution or a mixture of saline and glucose solution. Injectable solutions containing the telomerase inhibitor described herein may be formulated in oil for prolonged action. Appropriate oils for this purpose are, for example, peanut oil, sesame oil, cottonseed oil, corn oil, soybean oil, synthetic glycerol esters of long chain fatty acids and mixtures of these and other oils. Injectable suspensions may also be prepared in which case appropriate liquid carriers, suspending agents and the like may be employed. Also included are solid form preparations that are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired composition. The composition may be administered in various ways, e.g., as a transdermal patch, as a spot-on, as an ointment.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

In order to enhance the solubility and/or the stability of the drug described herein in pharmaceutical compositions, it can be advantageous to employ α-, β- or γ-cyclodextrins or their derivatives, in particular hydroxyalkyl substituted cyclodextrins, e.g. 2 hydroxypropyl-β-cyclodextrin or sulfobutyl-β-cyclodextrin. Also, co-solvents such as alcohols may improve the solubility and/or the stability of the telomerase inhibitor in pharmaceutical compositions.

Depending on the mode of administration, the pharmaceutical composition will preferably comprise from 0.05 to 99% by weight, more preferably from 0.1 to 70% by weight, even more preferably from 0.1 to 50% by weight of the telomerase inhibitor described herein, and from 1 to 99.95% by weight, more preferably from 30 to 99.9% by weight, even more preferably from 50 to 99.9% by weight of a pharmaceutically acceptable carrier, all percentages being based on the total weight of the composition.

### G. Administration and Administration Regimens

The frequency of administration can be any frequency that reduces the severity of a symptom of myelofibrosis without producing significant toxicity to the subject. For example, the frequency of administration can be from about once every two months to about once a week, alternatively from about once a month to about twice a month, alternatively about once every six weeks, about once every 5 weeks, alternatively about once every 4 weeks, alternatively about once every 3 weeks, alternatively about once every 2 weeks or alternatively about once a week. The frequency of administration can remain constant or can be variable during the duration of treatment. A course of treatment with a composition containing one or more telomerase inhibitors can include rest periods. For example, a composition containing a telomerase inhibitor can be administered weekly over a three-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the myelofibrosis and related symptoms may require an increase or decrease in administration frequency.

An effective duration for administering a composition containing a telomerase inhibitor (e.g., imetelstat or imetelstat sodium) can be any duration that reduces the severity of a symptom of myelofibrosis (e.g., as described herein) without producing significant toxicity to the subject. Thus, the effective duration can vary from one month to several months or years (e.g., one month to two years, one month to one year, three months to two years, three months to ten months, or three months to 18 months). In general, the effective duration for the treatment of myelofibrosis can range in duration from two months to twenty months. In some cases, an effective duration can be for as long as an individual subject is alive. Multiple factors can influence the actual effective duration used for a particular treatment. For example, an effective duration can vary with the frequency of administration, effective amount, use of multiple treatment agents, route of administration, and severity of the myelofibrosis and related symptoms.

In certain instances, a course of treatment and the severity of one or more symptoms related to myelofibrosis can be monitored. Any method can be used to determine whether or not the severity of a symptom of myelofibrosis is reduced. For example, the severity of a symptom of myelofibrosis (e.g., as described herein) can be assessed using biopsy techniques.

Telomerase inhibitors as used in the subject methods can be administered at any dose that is therapeutically effective, such as doses comparable to those routinely utilized clinically. Specific dose regimens for known and approved anti-cancer agents (e.g., the recommended effective dose) are known to physicians and are given, for example, in the product descriptions found in the PHYSICIANS″ DESK REFERENCE, 2003, 57th Ed., Medical Economics Company, Inc., Oradell, N.J.; Goodman & Gilman's THE PHARMACOLOGICAL BASIS OF THERAPEUTICS″ 2001, 10th Edition, McGraw-Hill, New York; and/or are available from the Federal Drug Administration and/or are discussed in the medical literature.

In some aspects, the dose of a telomerase inhibitor, imetelstat sodium, administered to the subject is about 1.0 mg/kg to about 13.0 mg/kg. In other aspects, the dose of a telomerase inhibitor is about 4.5 mg/kg to about 11.7 mg/kg or about 6.0 mg/kg to about 11.7 mg/kg or about 6.5 mg/kg to about 11.7 mg/kg. In some embodiments, the dose of a telomerase inhibitor includes at least about any of 4.5 mg/kg, 4.6 mg/kg, 4.7 mg/kg, 4.8 mg/kg, 4.9 mg/kg, 5.0 mg/kg, 5.5 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 10.1 mg/kg, 10.2 mg/kg, 10.3 mg/kg, 10.4 mg/kg, 10.5 mg/kg, 10.6 mg/kg, 10.7 mg/kg, 10.8 mg/kg, 10.9 mg/kg, 11 mg/kg, 11.1 mg/kg, 11.2 mg/kg, 11.3 mg/kg, 11.4 mg/kg, 11.5 mg/kg, 11.6 mg/kg, 11.7 mg/kg, 11.8 mg/kg, 11.9 mg/kg, 12 mg/kg, 12.1 mg/kg, 12.2 mg/kg, 12.3 mg/kg, 12.4 mg/kg, 12.5 mg/kg, 12.6 mg/kg, 12.7 mg/kg, 12.8 mg/kg, 12.9 mg/kg, or 13 mg/kg.

In some embodiments, the effective amount of a telomerase inhibitor administered to the individual includes at least about any of 1 mg/kg, 2.5 mg/kg, 3.5 mg/kg, 4.7 mg/kg, 5 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.4 mg/kg, 10 mg/kg, 15 mg/kg, or 20 mg/kg. In some embodiments, the effective amount of a telomerase inhibitor administered to the individual is about any of 1 mg/kg, 2.5 mg/kg, 3.5 mg/kg, 4.7 mg/kg, 5 mg/kg, 6.5 mg/kg, 7.5 mg/kg, 9.4 mg/kg, 10 mg/kg, 15 mg/kg, or 20 mg/kg. In various embodiments, the effective amount of a telomerase inhibitor administered to the individual includes less than about any of 350 mg/kg, 300 mg/kg, 250 mg/kg, 200 mg/kg, 150 mg/kg, 100 mg/kg, 50 mg/kg, 30 mg/kg, 25 mg/kg, 20 mg/kg, 10 mg/kg, 7.5 mg/kg, 6.5 mg/kg, 5 mg/kg, 3.5 mg/kg, 2.5 mg/kg, 1 mg/kg, or 0.5 mg/kg of a telomerase inhibitor.

Exemplary dosing frequencies for the pharmaceutical composition including a telomerase inhibitor include, but are not limited to, daily; every other day; twice per week; three times per week; weekly without break; weekly, three out of four weeks; once every three weeks; once every two weeks; weekly, two out of three weeks. In some embodiments, the pharmaceutical composition is administered about once every week, once every 2 weeks, once every 3 weeks, once every 4 weeks, once every 5 weeks, once every 6 weeks, once every 7 weeks or once every 8 weeks. In some embodiments, the composition is administered at least about any of 1×, 2×, 3×, 4×, 5×, 6×, or 7× (i.e., daily) a week, or three times daily, two times daily. In some embodiments, the intervals between each administration are less than about any of 6 months, 3 months, 1 month, 20 days, 15 days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

Telomerase inhibitors such as imetelstat (e.g., imetelstat sodium) can be administered using any appropriate method. For example, telomerase inhibitors such as imetelstat (e.g., imetelstat sodium) can be administered intravenously once every 4 weeks over a period of time (e.g., one, two, three, four, or five hours). In some embodiments, imetelstat is administered intravenously once weekly over a period of about 2 hours at 7-10 mg/kg. In certain embodiments, imetelstat is administered intravenously once every 3 weeks over a period of about 2 hours about 0.5-9.4 mg/kg. In an embodiment, imetelstat is administered intravenously for a period of about 2 hours once every 4 weeks at 0.5-5 mg/kg. In an embodiment, imetelstat is administered intravenously once every 3 weeks over a period of about 2 hours at about 2.5-10 mg/kg. Alternatively, imetelstat is administered intravenously for a period of about 2 hours once every 4 weeks at about 0.5-9.4 mg/kg.

In certain embodiments of the method, imetelstat is administered for 1, 2, 3, 4, 5, 6, 7, 8 or more than 8 dosage cycles, each cycle comprising: intravenous administration of about 7-10 mg/kg imetelstat once every three weeks, intravenous administration of about 7-10 mg/kg imetelstat once weekly for three weeks, intravenous administration of about 2.5-10 mg/kg imetelstat once every three weeks, or intravenous administration of about 0.5-9.4 mg/kg imetelstat once every three weeks. In certain instance, each dosage cycle comprises intravenous administration of about 7-10 mg/kg imetelstat once every three weeks. In some cases, each dosage cycle comprises intravenous administration of about 9.4 mg/kg imetelstat about once every three weeks.

In one embodiment of the invention, imetelstat is administered intravenously at a dosage of about 7-10 mg/kg imetelstat once every three weeks following premedication with an antihistamine, corticosteroid, or both. In other embodiments, imetelstat is administered intravenously at a dosage of about 9.4 mg/kg, alternatively from about 7.0 mg/kg to about 9.8 mg/kg, imetelstat once every three weeks following premedication with an antihistamine, corticosteroid, or both.

In certain embodiments, imetelstat is administrated at a dosage about 7.5 mg/kg, alternatively from about 7.0 mg/kg to about 7.7 mg/kg, once every three weeks for at least three cycles and then the dosage is increased. In certain embodiments, the dosage of imetelstat may be increased to about 9.4 mg/kg, alternatively from about 8.8 mg/kg to about 9.6 kg/mg, provided ANC and platelet nadir have not dropped between about 1.5×10⁹/L and about 75×10⁹/L, respectively, and there is no grade ≥3 non-hematological toxicity.

It will be appreciated that treatment for cancer sometimes involves multiple “rounds” or “cycles” of administration of a drug, where each cycle comprises administration of the drug one or more times according to a specified schedule (e.g., every three weeks for three consecutive days; once per week; etc.). For example, anti-cancer drugs can be administered for from 1 to 8 cycles, or for a longer period. When more than one drug (e.g., two drugs) is administered to a subject, each can be administered according to its own schedule (e.g., weekly; once every three weeks; etc.). It will be clear that administration of drugs, even those administered with different periodicity, can be coordinated so that both drugs are administered on the same day at least some of the time or, alternatively, so the drugs are administered on consecutive days at least some of the time.

In certain embodiments, the imetelstat may be administered via a regimen which involves dose reductions. In one embodiment, the patient is initially administered about 9.4 mg/kg every three weeks, the dose is then changed to about 7.5 mg/kg every three weeks, and then the dose is changed to about 6.0 mg/kg every three weeks.

As is understood in the art, treatment with cancer therapeutic drugs can be suspended temporarily if toxicity is observed, or for the convenience of the patient, without departing from the scope of the invention, and then resumed.

Aspects of the subject methods include identifying or selecting a patient most likely to benefit from treatment based on relative telomere length in target cells (e.g. as described herein) of the patient. Target cells can be any convenient cells of the patient, including but not limited to, cells of the bone marrow or peripheral blood of the patient. In some cases, the target cells are isolated from a bone marrow sample of the patient. In some cases, the target cells are isolated from a peripheral blood sample of the patient. The target cells can be granulocytes. In some cases, the patient lacks a mutation in each of Janus kinase 2 (JAK2), calreticulin (CALR), and thrombopoietin receptor (MPL) genes; and has a particular short telomere length in target cells of the patient. As used herein, a short telomere length is one that is less than or equal to the median or mean telomere length, as compared to a suitable control, e.g., one or more known standards as described herein. As such, the subject methods can further include determining relative telomere length by analyzing the relative length of telomeric nucleic acids in target cells present in a biological sample from the individual; selecting an individual who will benefit from treatment with a telomerase inhibitor when the average relative telomere length in the target cells present in a biological sample from the individual is determined to be in the 50th percentile or less of a relative telomere length range determined from one or more known standards, such as determined to be in the 45th percentile or less, 40th percentile or less, 35th percentile or less, 30th percentile or less, 25th percentile or less, 20th percentile or less, or even less, of a relative telomere length range determined from one or more known standards

In some instances of the method, the one or more known standards is a telomere length range established from a plurality of naturally occurring target cells (e.g. as described herein) from a plurality of individuals diagnosed with the disease. In certain instances of the method, the one or more known standards are characterized cell lines. By “characterized cell lines” it is meant that the relative telomeric nucleic acids of the cells in the cell lines are known and relatively constant.

In some embodiments, the telomere length in the cancer cells present in the biological sample is determined to be less than or equal to the median or mean telomere length. In some embodiments, the telomere length in the cancer cells present in the biological sample is determined to be in the 50th percentile or less, 40th percentile or less, 35th percentile or less, 30th percentile or less, 25th percentile or less, 20th percentile or less, 15th percentile or less, 10th percentile or less, or 5th percentile or less, of the relative telomere length range determined from the one or more known standards.

Telomere length in a target cell can be determined using any convenient assays, including but not limited to, qPCR, telo-FISH, or Southern Blot assays, as described by Bassett et al. in U.S. Pat. No. 9,200,327. In one aspect, telomere length can be determined by measuring the mean length of a terminal restriction fragment (TRF). The TRF is defined as the length—in general the average length—of fragments resulting from complete digestion of genomic DNA with a restriction enzyme that does not cleave the nucleic acid within the telomeric sequence. In some cases, the DNA is digested with restriction enzymes that cleaves frequently within genomic DNA but does not cleave within telomere sequences. In some cases, the restriction enzymes have a four base recognition sequence (e.g., AluI, HinfI, RsaI, and Sau3A1) and are used either alone or in combination. The resulting terminal restriction fragment contains both telomeric repeats and subtelomeric DNA. Subtelomeric DNA are DNA sequences adjacent to tandem repeats of telomeric sequences and contain telomere repeat sequences interspersed with variable telomeric-like sequences. The digested DNA is separated by electrophoresis and blotted onto a support, such as a membrane. The fragments containing telomere sequences are detected by hybridizing a probe, i.e., labeled repeat sequences, to the membrane. Upon visualization of the telomere containing fragments, the mean lengths of terminal restriction fragments can be calculated (Harley, C. B. et al., Nature. 345(6274):458-60 (1990), hereby incorporated by reference). TRF estimation by Southern blotting gives a distribution of telomere length in the cells or tissue, and thus the median and mean telomere length of all cells.

In another aspect, telomere lengths can be measured by flow cytometry (Hultdin, M. et al., Nucleic Acids Res. 26: 3651-3656 (1998); Rufer, N. et al., Nat. Biotechnol. 16:743-747 (1998); incorporated herein by reference). Flow cytometry methods are variations of FISH techniques. If the starting material is tissue, a cell suspension is made, generally by mechanical separation and/or treatment with proteases. Cells are fixed with a fixative and hybridized with a telomere sequence specific probe, preferably a PNA probe, labeled with a fluorescent label. Following hybridization, cells are washed and then analyzed by FACS. Fluorescence signal is measured for cells in Go/G1 following appropriate subtraction for background fluorescence. This technique is suitable for rapid estimation of telomere length for large numbers of samples. Similar to TRF, telomere length is the average length of telomeres within the cell.

In other aspects, the median or average length of telomeres from cells within a biological sample is determined via quantitative PCR (qPCR) or telomere fluorescent in situ hybridization (telo-FISH). In qPCR, a DNA binding dye binds to all double-stranded DNA causing fluorescence of the dye. An increase in DNA product during the PCR reaction leads to an increase in the fluorescence intensity and is measured at each cycle of the PCR reaction. This allows the DNA concentration to be quantified. The relative concentration of the DNA present during the exponential phase of the reaction is determined by plotting the level of fluorescence against the PCR cycle number on a semi-logarithmic scale. A threshold for detection of fluorescence above background is determined. The cycle at which the fluorescence from the sample crosses the threshold is called the cycle threshold (Ct). Because the quantity of DNA theoretically doubles every cycle during the exponential phase, the relative amounts of DNA can be calculated. The baseline is the initial cycles of PCR, in which there is little change in fluorescence signal.

In some aspects, telomere length is determined using telo-FISH. In this method, cells are fixed and hybridized with a probe conjugated to a fluorescent label, for example, Cy-3, fluoresceine, rhodamine, etc. Probes for this method are oligonucleotides designed to hybridize specifically to telomere sequences. Generally, the probes are 8 or more nucleotides in length, such as 12-20 or more nucleotides in length. In one aspect, the probes are oligonucleotides comprising naturally occurring nucleotides. In one aspect, the probe is a peptide nucleic acid, which has a higher Tm than analogous natural sequences, and thus permits use of more stringent hybridization conditions. Cells may be treated with an agent, such as colcemid, to induce cell cycle arrest at metaphase provide metaphase chromosomes for hybridization and analysis. In some embodiments, cellular DNA can also be stained with the fluorescent dye 4′,6-diamidino-2-phenylindole (DAPI).

Digital images of intact metaphase chromosomes are acquired and the fluorescence intensity of probes hybridized to telomeres quantitated. This permits measurement of telomere length of individual chromosomes, in addition to average or median telomere length in a cell, and avoids problems associated with the presence of subtelomeric DNA (Zjilmans, J. M. et al., Proc. Natl. Acad Sci. USA 94:7423-7428 (1997); Blasco, M. A. et al., Cell 91:25-34 (1997); incorporated by reference). The intensity of the fluorescent signal correlates with the length of the telomere, with a brighter fluorescent signal indicating a longer telomere.

In certain embodiments, the invention relates to a telomerase inhibitor for use in a method of treating myelofibrosis, the method comprising: identifying a patient most likely to benefit from treatment with a telomerase inhibitor comprising testing a patient for:
(a) triple negative status, based on the absence of any mutation in JAK2, CALR, and MPL genes;
(b) high-molecular risk (HMR), based on the presence of a mutation in at least one of the following genes: ASXL1, EZH2, SRSF2, and IDH1/2; or
(c) both;
wherein the presence of (a), (b) or (c) is indicative of a patient most likely to benefit from treatment with a telomerase inhibitor and administering to the patient an effective amount of a telomerase inhibitor. In certain embodiments, the invention relates to a telomerase inhibitor for use in a method as defined in any of the other embodiments.

Yet another embodiment of the invention is a telomerase inhibitor for use in the treatment of myelofibrosis comprising: (a) screening a patient to determine if such patient is: triple negative status, based on the absence of a mutation in each of JAK2, CALR, and MPL and/or a high-molecular risk (HMR), based on the presence of a mutation in at least one of the following genes: ASXL1, EZH2, SRSF2, and IDH1/2; and; (b) administering the telomerase inhibitor to the patient if such patient is triple negative status, based on the absence of a mutation in each of JAK2, CALR, and MPL, and/or is high-molecular risk (HMR) based on the presence of a mutation in at least one of the following genes: ASXL1, EZH2, SRSF2, and IDH1/2. In one embodiment, the use comprises screening the patient for triple negative status, based on the absence of a mutation in each of JAK2, CALR, and MPL. Yet another embodiment of the invention is a telomerase inhibitor for use in the treatment of myelofibrosis comprising: (a) screening a patient to determine if such patient is: triple negative status, based on the absence of a mutation in each of JAK2, CALR, and MPL; and (b) administering the telomerase inhibitor to the patient if such patient is triple negative status.

Yet another embodiment of the invention is use of a telomerase inhibitor for the treatment of myelofibrosis comprising: (a) screening a patient to determine if such patient is: triple negative status, based on the absence of a mutation in each of JAK2, CALR, and MPL and/or a high-molecular risk (HMR), based on the presence of a mutation in at least one of the following genes: ASXL1, EZH2, SRSF2, and IDH1/2; and; (b) administering the telomerase inhibitor to the patient if such patient is triple negative status, based on the absence of a mutation in each of JAK2, CALR, and MPL, and/or is high-molecular risk (HMR) based on the presence of a mutation in at least one of the following genes: ASXL1, EZH2, SRSF2, and IDH1/2. In one embodiment, the use comprises screening the patient for triple negative status, based on the absence of a mutation in each of JAK2, CALR, and MPL. Yet another embodiment of the invention is use of a telomerase inhibitor for the treatment of myelofibrosis comprising: (a) screening a patient to determine if such patient is: triple negative status, based on the absence of a mutation in each of JAK2, CALR, and MPL; and (b) administering the telomerase inhibitor to the patient if such patient is triple negative status.

In certain embodiments of the invention, triple negative status may be determined based on the absence of a mutation in each of a JAK2 gene having the nucleic acid sequence of SEQ ID NO: 2, a CALR gene having the nucleic acid sequence of SEQ ID NO: 3, and a MPL gene having the nucleic acid sequence of SEQ ID NO: 4. In other embodiments, triple negative status may be determined based on the absence of a mutation in each of SEQ ID NO: 2; CALR, and MPL. In an alternate embodiment, triple negative status may be determined based on the absence of a mutation in each of JAK2, SEQ ID NO: 3, and MPL. In an alternate embodiment, triple negative status may be determined based on the absence of a mutation in each of JAK2, CALR, and SEQ ID NO: 4.

In other embodiments of the invention, high-molecular risk (HMR) may be determined based on the presence of a mutation in at least one of the following genes: a ASXL1 gene having the nucleic acid sequence of SEQ ID NO: 5, a EZH2 gene having the nucleic acid sequence of SEQ ID NO: 6, a SRSF2 gene having the nucleic acid sequence of SEQ ID NO: 7, an IDH1 gene having the nucleic acid sequence of SEQ ID NO: 8, an IDH2 gene having the nucleic acid sequence of SEQ ID NO: 9, and combinations thereof.

In other embodiments of the invention, telomerase activity and level of hTERT expression in biological samples obtained from patients can be determined to evaluate pharmacodynamic effects and/or monitor patients being treated with a telomerase inhibition. The telomerase activity can be measured using the TRAP (Telomeric Repeat Amplification Protocol) telomerase activity assay. The level of hTERT expression can be determined by measuring the level of hTERT RNA expression in the cells in the biological sample using northern blots or serial analysis of gene expression (SAGE) or other methods.

In certain embodiments, the invention relates to a telomerase inhibitor for use in the treatment of myelofibrosis as defined in any of the other embodiments.

In certain embodiments, the invention relates to the use of a telomerase inhibitor for the treatment of myelofibrosis as defined in any of the other embodiments.

### Additional Embodiments

Additional embodiments of interest are set forth in the following clauses:

Clause 1. A method of identifying a patient most likely to benefit from treatment with a telomerase inhibitor comprising:
(a) testing a patient for triple negative status, based on the absence of a mutation in each of Janus kinase 2 (JAK2), calreticulin (CALR), and thrombopoietin receptor (MPL) genes; and
(b) selecting the patient if the patient has triple negative status, based on the absence of a mutation in each of JAK2, CALR, and MPL genes,
   wherein the selected patient is most likely to benefit from treatment with a telomerase inhibitor.

Clause 2. A method of identifying a patient most likely to benefit from treatment with a telomerase inhibitor comprising:
(c) testing a patient for the following:
   i. triple negative status, based on the absence of a mutation in each of JAK2, CALR, and MPL genes, and/or
   ii. a high-molecular risk (HMR), based on the presence of a mutation in at least one of the following genes: ASXL1, EZH2, SRSF2, and IDH1/2,
(d) selecting the patient that has the following:
   i. triple negative status, based on the absence of a mutation in each of JAK2, CALR, and MPL genes, and/or
   ii. a high-molecular risk (HMR), based on the presence of a mutation in at least one of the following genes: ASXL1, EZH2, SRSF2, and IDH1/2,
      wherein the selected patient is most likely to benefit from treatment with a telomerase inhibitor.

Clause 3. A method of identifying a patient most likely to benefit from treatment with a telomerase inhibitor comprising:
(e) obtaining a DNA sample from a patient;
(f) testing the DNA sample from a patient for triple negative status, based on the absence of a mutation in each of JAK2, CALR, and MPL genes; and
(g) selecting the patient if the patient has: triple negative status, based on no mutation in each of JAK2, CALR, and MPL genes;
   wherein the selected patient is most likely to benefit from treatment with a telomerase inhibitor.

Clause 4. A method of identifying a patient most likely to benefit from treatment with a telomerase inhibitor comprising:
(h) obtaining a DNA sample from a patient;
(i) testing the DNA sample from a patient for:
   i. triple negative status, based on the absence of a mutation in each of JAK2, CALR, and MPL genes; and/or
   ii. a high-molecular risk (HMR), based on the presence of a mutation in at least one of the following genes: ASXL1, EZH2, SRSF2, and IDH1/2; and
(j) selecting the patient if the patient has:
i. triple negative status, based no mutation in each of JAK2, CALR, and MPL genes; and/or
ii. a high-molecular risk (HMR), based on the presence of a mutation in at least one of the following genes: ASXL1, EZH2, SRSF2, and IDH1/2,
   wherein the selected patient is most likely to benefit from treatment with a telomerase inhibitor.

Clause 5. Use of a telomerase inhibitor in the treatment of patient that has myelofibrosis wherein the patient is determined to have a triple negative status, wherein the triple negative status comprises an absence of a mutation in each of the Janus kinase 2 (JAK2), calreticulin (CALR), and thrombopoietin receptor (MPL) genes.

Clause 6. Use of a telomerase inhibitor in the treatment of patient that has myelofibrosis wherein the patient is determined to have a high-molecular risk (HMR), wherein having HMR comprises the presence of a mutation in at least one gene selected from the group consisting of additional sex combs like 1 (ASXL1), enhancer of zeste homolog 2 (EZH2), serine and arginine rich splicing factor 2 (SRSF2), and isocitrate dehydrogenase 1/2 (IDH1/2).

Clause 7. Use of a telomerase inhibitor in the treatment of patient that has myelofibrosis wherein cells present in a biological sample from the patient have been determined to have average relative telomere length that is determined to be in the 50th percentile or less of a relative telomere length range determined from one or more known standards.

Clause 8. Use of a telomerase inhibitor in the manufacture of a medicament for the treatment of patient that has myelofibrosis wherein the patient is determined to have a triple negative status, wherein the triple negative status comprises an absence of a mutation in each of the Janus kinase 2 (JAK2), calreticulin (CALR), and thrombopoietin receptor (MPL) genes.

Clause 9. Use of a telomerase inhibitor in the manufacture of a medicament for the treatment of patient that has myelofibrosis wherein the patient is determined to have a high-molecular risk (HMR), wherein having HMR comprises the presence of a mutation in at least one gene selected from the group consisting of additional sex combs like 1 (ASXL1), enhancer of zeste homolog 2 (EZH2), serine and arginine rich splicing factor 2 (SRSF2), and isocitrate dehydrogenase 1/2 (IDH1/2).

Clause 10. Use of a telomerase inhibitor in the manufacture of a medicament for the treatment of patient that has myelofibrosis wherein cells present in a biological sample from the patient have been determined to have average relative telomere length that is determined to be in the 50th percentile or less of a relative telomere length range determined from one or more known standards.

The following examples are offered by way of illustration and not by way of limitation.

### EXAMPLES

### Example 1

### Imetelstat Sodium is an Effective Treatment for Patients with Intermediate-2 (Int-2) or High-Risk Myelofibrosis (MF) that have Relapsed or are Refractory to Janus Kinase (JAK) Inhibitor Therapy

### Introduction

Imetelstat, a 13-mer oligonucleotide that specifically targets the RNA template of human telomerase, is a potent competitive inhibitor of telomerase enzymatic activity (Asai et al., Cancer Res 2003; Herbert, Oncogene 2005). Clinical activity and an acceptable safety profile were reported in a 33-patient pilot study in intermediate-2 (int-2) or high-risk myelofibrosis (MF), where 48% of patients had been previously treated with a Janus Kinase inhibitor (JAKi) (Tefferi, N Engl J Med 2015). This example provides the results of a phase 2 clinical study of imetelstat sodium at two dose levels in patients with myelofibrosis (MF).

### Methods

A randomized, multicenter, phase 2 study of two doses of imetelstat sodium (9.4 mg/kg or 4.7 mg/kg IV, every 3 weeks) was performed in adults with: a Dynamic International Prognostic Scoring System (DIPSS) Score of int-2; or high-risk MF that was relapsed/refractory to prior JAKi therapy (i.e., either no reduction in splenomegaly after 12 weeks or worsening splenomegaly at any time after the start of JAK inhibitor (“JAKi”) therapy). Diagnosis of primary, post-essential thrombocythemia, or post-polycythemia vera MF was required; other eligibility criteria included measurable splenomegaly (by magnetic resonance imaging [MRI]), active MF-related systemic symptoms and platelet count ≥75×10⁹/L. Primary endpoints were: spleen response rate (% achieving ≥35% spleen volume reduction [SVR] by MRI at week 24); and symptom response rate (% achieving ≥50% reduction in total symptom score [TSS] per the Myelofibrosis Symptom Assessment Form (MFSAF) v2 at week 24). Key secondary endpoints included safety, overall survival (OS), treatment response, molecular response, and pharmacokinetic and pharmacodynamic relationships.

### Results

107 patients were enrolled at 55 institutions (48 on 4.7 mg/kg; 59 on 9.4 mg/kg). Baseline characteristics are shown in Table 1 below. Additionally, median time on JAKi was 23 (0.9-89.7) months, and median platelet count was 147×10⁹/L. Triple-negative (TN; i.e., no mutations of JAK2, MPL, or CALR) comprised 24.8% of patients and 67.6% were considered high molecular risk (HMR; i.e., ≥1 mutation ofASXL1, EZH2, SRSF2, or IDH1/2).

TABLE 1
Baseline characteristics

| | 4.7 mg/kg | | 9.4 mg/kg | |
|---|---|---|---|---|
| | (n = 48) | | (n = 59) | |
| Median Age (range; years) | 68.5 | (44, 84) | 67.0 | (31, 86) |
| Male | 32 | (66.7%) | 35 | (59.3%) |
| Myelofibrosis Subtype | | | | |
| Primary | 27 | (56.3%) | 36 | (61.0%) |
| Post-ET | 9 | (18.8%) | 10 | (16.9%) |
| Post-PV | 12 | (25.0%) | 13 | (22.0%) |
| DIPSS Risk Status | | | | |
| Intermediate-1 risk | 1^{a} | (2.1%) | 0 | |
| Intermediate-2 risk | 28 | (58.3%) | 34 | (57.6%) |
| High Risk | 19 | (39.6%) | 25 | (42.4%) |
| Spleen Length (Palpation) - Median (range; cm) | 18.0 | (4.0, 35.0) | 18.0 | (3.0, 32.0) |
| >15 cm | 31 | (64.6%) | 35 | (59.3%) |
| Spleen Volume (MRI) - Median^{b} (range; cm³) | 3352.8 | (726; 7243) | 2997.5 | (890; 7607) |
| Total Symptom Score - Median (range; TSS) | 22.29 | (6.7, 58.4) | 23.86 | (3.1, 57.2) |
| RBC Transfusion dependent^{c} | 14 | (29.2%) | 12 | (20.3%) |
| Platelet count - Median (range; ×10⁹/L) | 153 | (74, 1097) | 146 | (65, 798) |
| Time Since Initial Diagnosis - Median (range; | 48.90 | (2.0; 227.3) | 42.84 | (6.6; 200.7) |
| months) | | | | |
| Time Since Last JAKi Therapy - Median (range; | 1.41 | (0.5, 31.2) | 1.71 | (0.5, 37.8) |
| months) | | | | |
| Duration of prior JAKi Therapy - Median (range; | 22 | (3, 90) | 25 | (1, 73) |
| months) | | | | |

^{a}Protocol variation
^{b}Per Independent Review Committee
^{c}Received 6 units PRBC in 12 weeks prior to enrollment.

At the time of primary clinical cut-off, the median time on study was 22.6 months (range, 0.2-27.4 month); the median time on treatment was 6.2 months (range, 0.0-27.2 months). Six (10.2%) patients in the 9.4 mg/kg arm had a spleen response per MRI confirmed by IRC

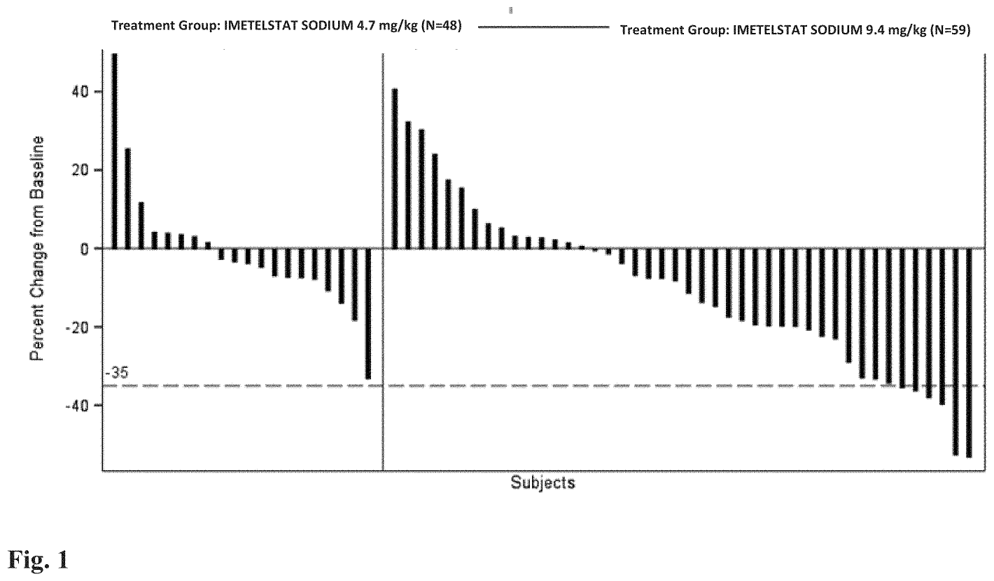

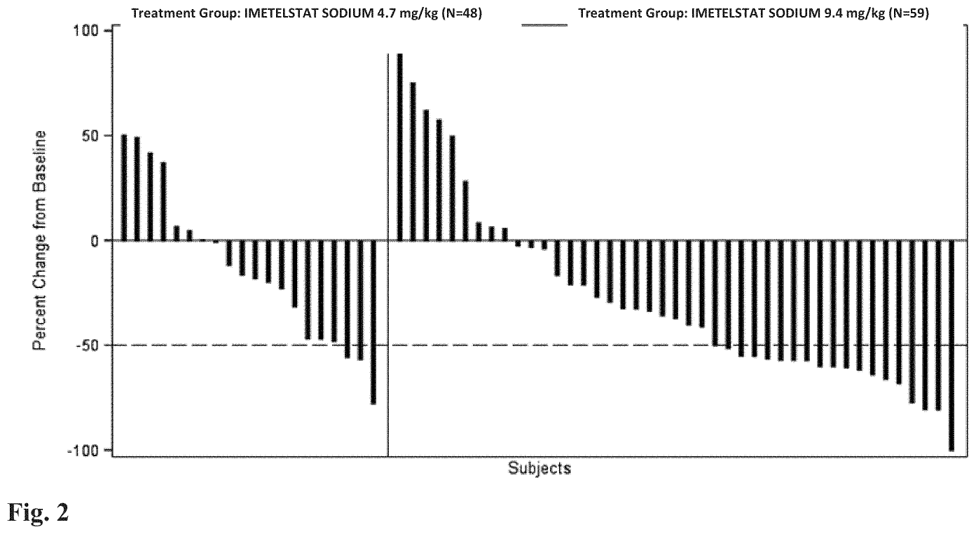

At the time of clinical cutoff, patients were followed for a median 22.6 (0.2-27.4) months, including a median treatment duration of 6.2 (0.0-27.2) months. Median duration on treatment was longer on the 9.4 mg/kg arm (7.7 months) than on the 4.7 mg/kg arm. Six (10.2%) patients in the 9.4 mg/kg arm had a spleen response per MRI, with no responses on the 4.7 mg/kg arm (see FIG. 1). Nineteen (32%) patients in the 9.4 mg/kg arm and 3 (6%) patients in the 4.7 mg/kg arm had a symptom response (TSS reduction ≥50%) (see FIG. 2).

Median OS in the 9.4 mg/kg arm has not been reached at the first clinical cutoff, while median OS was 19.9 months in the 4.7 mg/kg arm. The 18-month survival rates were 76.7% and 62.9% for the 9.4 mg/kg and 4.7 mg/kg arms, respectively. Sensitivity analyses censoring patients at time of dose escalation for subsequent JAKi therapy or stem cell transplant generated similar results. In the 9.4 mg/kg arm, an association was observed between patients who were TN and OS (median OS has not been reached for TN patients and was 23.6 months for non-TN patients). Spleen response rate was higher in patients with 1 HMR mutation (ASXL1, EZH2, SRSF2, or IDH1/2).

The most common adverse events on treatment (all grades) at 9.4 mg/kg were thrombocytopenia (49%), anemia (44%), neutropenia (36%), and nausea (34%); and at 4.7 mg/kg were diarrhea (38%), nausea (31%), anemia (31%), and thrombocytopenia (23%). Grade 3/4 neutropenia and thrombocytopenia were more frequent with 9.4 mg/kg (34% and 42%, respectively) than 4.7 mg/kg (13% and 29%, respectively); most cytopenias resolved within 4 weeks. Grade 3/4 LFT elevations were observed in 7 patients on study. Imetelstat-related hepatic toxicities, confirmed by an independent Hepatic Review Committee, were not observed.

At the time of a second clinical cutoff, patients were followed for a median 27.4 (0.2-33.0) months, including a median treatment duration of 26.9 (0.1-118.1) weeks. Median duration on treatment was longer on the 9.4 mg/kg arm (33.3 weeks) than on the 4.7 mg/kg arm (23.9 weeks). The 4.7 mg/kg arm was closed early, influencing duration of treatment. Median OS with 95% confidence interval in the 9.4 mg/kg arm was 29.9 months (22.8, NE) (NE is not estimable) in the 9.4 mg/kg arm and was reached at the second clinical cutoff.

Triple Negative vs. OS

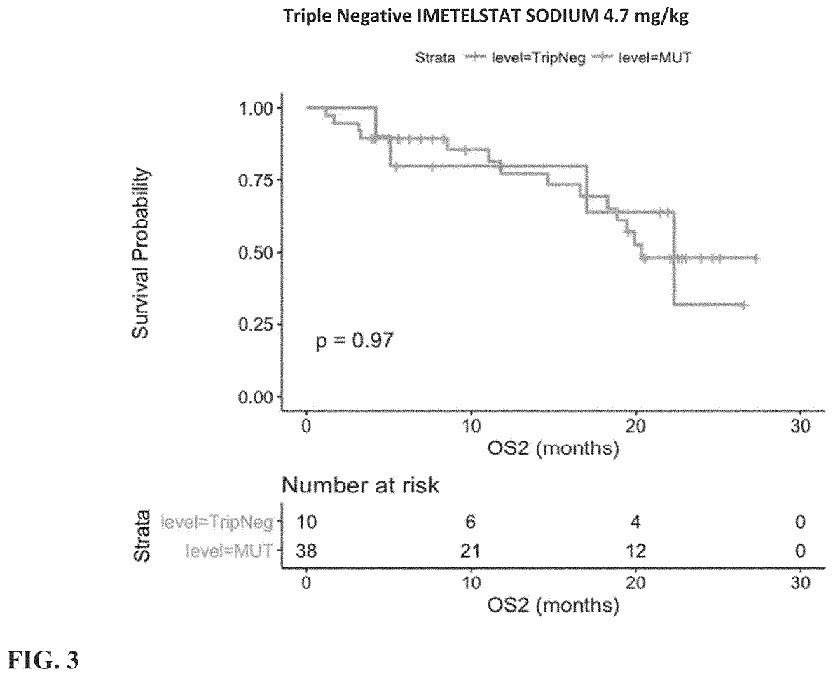

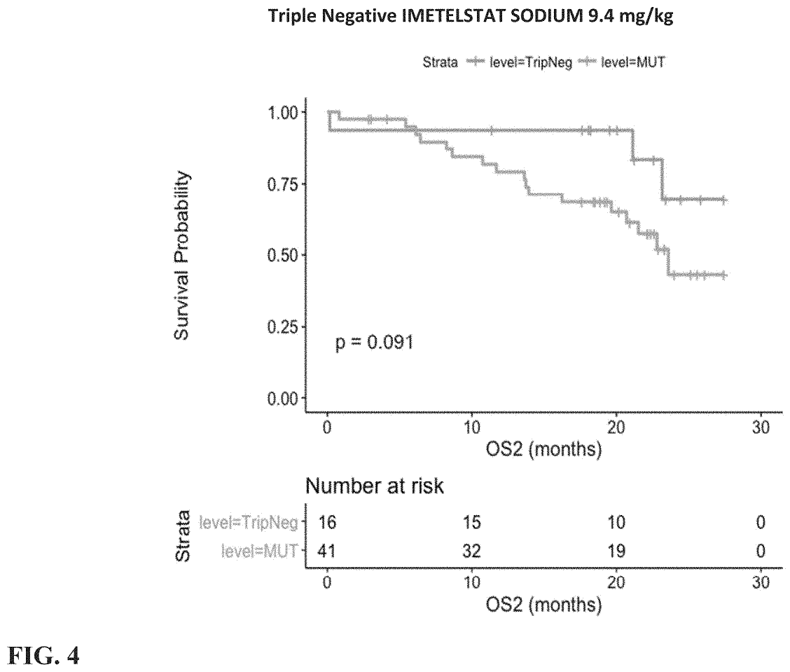

Subjects are grouped by mutation status of JAK2/MPL/CALR genes, triple Negative (TN, the absence of a mutation in each of JAK2/MPL/CALR genes) and Non-TN (with mutation in any of JAK2/MPL/CALR genes). The median OS were not estimable (NE) for TN subjects with 95% confidence interval (23.2, NE) and 23.6 months for Non-TN subjects with 95% confidence interval (20.7, NE) in 9.4 mg/kg arm, while in 4.7 mg/kg, the median OS with 95% confidence were 22.3 (17, NE) and interval 20.3 (18.3, NE) for TN subjects and Non-TN subjects, respectively. In the 9.4 mg/kg arm, a lower death rate was seen in the Triple Negative (TN) group compared to Non-TN group (see Table 2, FIGS. 3 and 4).

TABLE 2
Overall Survival Grouped by mutation status of JAK2/MPL/CALR genes

| Dose | | | Median survival, | | P value |
|---|---|---|---|---|---|
| (mg/kg) | Mut status | Death rate % | months (95% CI) | HR (95% CI) | (log rank) |
| 4.7 | TN | 40 | 22.3 (17, NE) | | |
| | Non-TN | 36.8 | 20.3 (18.3, NE) | 0.98 (0.32, 2.98) | 0.9719 |
| 9.4 | TN | 18.8 | NE (23.2, NE) | | |
| | Non-TN | 41.5 | 23.6 (20.7, NE) | 2.76 (0.81, 9.46) | 0.0910 |

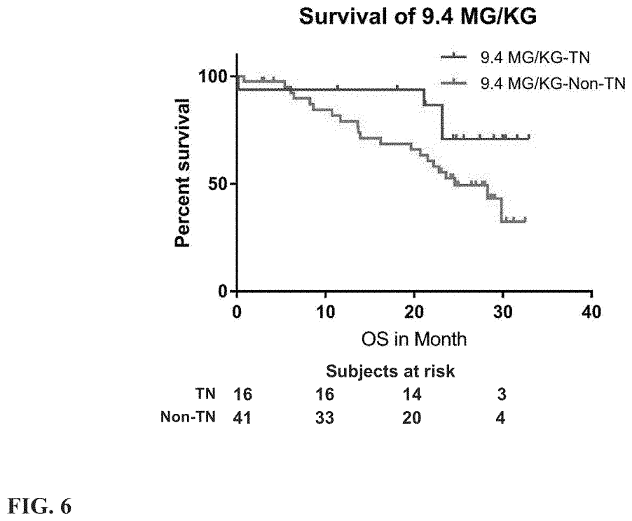

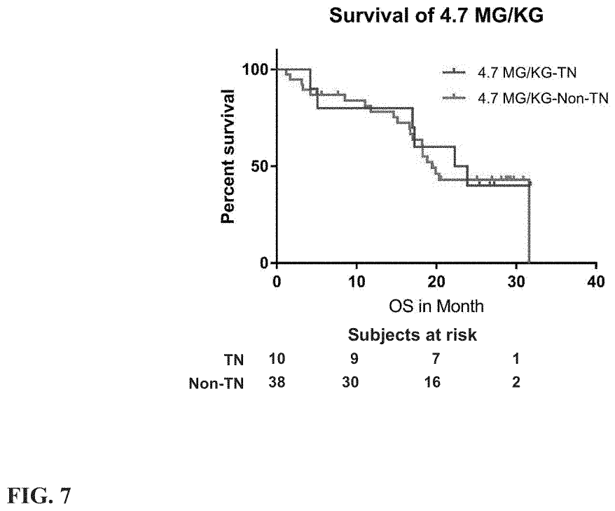

At the second clinical cutoff, a lower death rate was seen in the 9.4 mg/kg arm in the Triple Negative (TN) group compared to Non-TN group (see Table 3, FIGS. 6 and 7).

TABLE 3
Overall Survival Grouped by mutation status of JAK2/MPL/CALR genes

| | | Percentage | Median | | |
|---|---|---|---|---|---|
| Imetelstat | | of | Survival | | |
| Dose | | Subjects | (months) | HR* | P-value |
| (mg/kg) | Mutation Status | Who Died | (95% CI) | (95% CI) | (Log-rank) |
| 4.7 | Triple Negative | 6/10 (60.0%) | 23.1 (4.2, NE) | | |
| | (TN) | | | | |
| | Non-TN (MUT) | 21/38 (55.3%) | 19.5 (16.7, 31.6) | 1.16 (0.46, 2.87) | 0.7562 |
| 9.4 | Triple Negative | 4/16 (25.0%) | NE (23.2, NE) | | |
| | (TN) | | | | |
| | Non-TN (MUT) | 21/41 (51.2%) | 24.6 (19.7, NE) | 2.60 (0.89, 7.59) | 0.0700 |

*HR = Hazard Ratio

Triple-Negative vs. Response at Week 24

In the 9.4 mg/kg arm, a higher rate of response (SVR or TSS) was seen in the TN group compared to Non-TN group (see Table 4 below).

TABLE 4
Baseline JAK2/CALR/MPL mutation status by Clinical Response
SVR >= 20% at Week 24 by IRC

| | | 4.7 mg/kg, N = 48 | | | 9.4 mg/kg, N = 57 | | |
|---|---|---|---|---|---|---|---|
| JAK2/CALR/MPL | Total, | | Yes, | | | Yes, | |
| Mutation Status | N = 105 | Total # | N = 2 (4.2%) | No, N = 46 (95.8%) | Total # | N = 12 (21.1%) | No, N = 45 (78.9%) |
| TN | 26(24.8%) | 10 | 0 (0%) | 10 (100%) | 16 | 5 (31.25%) | 11 (68.75)) |
| Non-TN | 79 (75.2%) | 38 | 2 (5.3%) | 36 (94.7%) | 41 | 7 (17.1%) | 34 (82.9%) |

SVR >= 35% at Week 24 by IRC

| JAK2/CALR/MPL | Total, | 4.7 mg/kg, N = 48 | | | 9.4 mg/kg, N = 57 | | |
|---|---|---|---|---|---|---|---|
| Mutation Status | N = 105 | Total # | Yes, N = 0 (0%) | No, N = 48 (100%) | Total # | Yes, N = 6 (10.5%) | No, N = 51 (89.5%) |
| TN | 26(24.8%) | 10 | 0 (0%) | 10 (100%) | 16 | 3 (18.75%) | 13 (81.25%) |
| Non-TN | 79 (75.2%) | 38 | 0 (0%) | 38 (100%) | 41 | 3 (7.3%) | 38 (92.7%) |

TSS >= 50% decrease at Week 24

| JAK2/CALR/MPL | Total, | 4.7 mg/kg, N = 48 | | | 9.4 mg/kg N = 57 | | |
|---|---|---|---|---|---|---|---|
| Mutation Status | N = 105 | Total # | Yes, N = 3 (6.3%) | No, N = 45 (93.8%) | Total # | Yes, N = 18 (31.6%) | No, N = 39 (68.4%) |
| TN | 26(24.8%) | 10 | 1 (10.0%) | 9 (90.0%) | 16 | 8 (50%) | 8 (50%) |
| Non-TN | 79 (75.2%) | 38 | 2 (5.3%) | 36 (94.7%) | 41 | 10 (24.4%) | 31 (75.6%) |

Molecular Risk vs. Responses at Week 24

In the 9.4 mg/kg arm, a higher rate of response (SVR or TSS) was seen in low molecular risk (LMR) group or high molecular risk (HMR) group with only 1 mutation (mut) group compared to the HMR group with more than 1 mutations (see Table 5).

TABLE 5
Molecular Risk vs. Responses at Week 24
SVR >= 20% at week 24

| | | 4.7 mg/kg, N = 48 | | | 9.4 mg/kg, N = 57 | | |
|---|---|---|---|---|---|---|---|
| | Total, | Total | Yes, N = 1 | No, | Total | Yes, N = 12 | No, N = 45 |
| Molecular Risk | N = 105 | # | (2.1%) | N = 47(97.8%) | # | (21.1%) | (78.9%) |
| LMR, | 34 | 12 | 1 (8.3%) | 11 (91.7%) | 22 | 5 (22.7%) | 17 (77.3%) |
| No Mutation | (32.4%) | | | | | | |
| HMR with 1 Mutation | 48 | 27 | 0 (0%) | 27(100%) | 21 | 6 (28.6%) | 15 (71.4%) |
| | (45.7%) | | | | | | |
| HMR with >1 | 23 | 9 | 0 (0%) | 9 (100%) | 14 | 1 (7.1%) | 13 (92.9%) |
| Mutation | (21.9%) | | | | | | |

SVR >= 35% at wk 24 by IRC

| | | 4.7 mg/kg, N = 48 | | | 9.4 mg/kg, N = 57 | | |
|---|---|---|---|---|---|---|---|
| | Total | Total | Yes, | No, | Total | Yes, | No, N = 51 |
| Molecular Risk | N = 105 | # | N = 0 (0%) | N = 48 (100%) | # | N = 6 (10.5%) | (89.5%) |
| LMR, | 34 | 12 | 0 (0%) | 12 (100%) | 22 | 2 (9.1%) | 20 (90.9%) |
| No Mutation | (32.4%) | | | | | | |
| HMR with 1 Mutation | 48 | 27 | 0 (0%) | 27 (100%) | 21 | 4 (19%) | 17 (81%) |
| | (45.7%) | | | | | | |
| HMR with >1 | 23 | 9 | 0 (0%) | 9 (100%) | 14 | 0 (0%) | 14 (100% |
| Mutation | (21.9%) | | | | | | |

TSS >= 50% decrease at week 24

| | | 4.7 mg/kg, N = 48 | | | 9.4 mg/kg, N = 57 | | |
|---|---|---|---|---|---|---|---|
| | Total, | Total | Yes, N = 3 | No, N = 45 | Total | Yes, N = 18 | No, N = 39 |
| Molecular Risk | N = 105 | # | (6.3%) | (93.8%) | # | (31.6%) | (68.4%) |
| LMR, | 34 | 12 | 3 (25%) | 9 (75%) | 22 | 8 (36.4%) | 14 (63.6%) |
| No Mutation | (32.4%) | | | | | | |
| HMR with 1 Mutation | 48 | 27 | 0 (0%) | 27(100%) | 21 | 7 (33.3%) | 14 (66.7%) |
| | (45.7%) | | | | | | |
| HMR with >1 | 23 | 9 | 0 (0%) | 9 (100%) | 14 | 3 (21.4%) | 11 (78.6%) |
| Mutation | (21.9%) | | | | | | |

For subjects on 9.4 mg/kg, an association was observed between the following factors and clinical responses or OS:
Triple Negative (TN): response (SVR or TSS) was enriched in TN subjects. Median OS not estimable for TN, Non-TN=23.6 months; and
Molecular Risk: response (SVR or TSS) was enriched in subjects with HMR having only 1 mutation, and responses were observed in patients with HMR having greater than one mutation who were treated with 9.4 mg/kg imetelstat.

### Example 2

Baseline Telomere Length (TL) vs. Overall Survival (OS)

Subjects are grouped by the median value of baseline TL. In the 9.4 mg/kg arm, median OS were not estimable (NE) at the first clinical cutoff with 95% confidence interval (23.2, NE) for subjects with shorter baseline TL (<=median value), and 22.8 months (16.2, NE) for the subjects with longer TL (>median value), respectively (Table 6). In 4.7 mg/kg arm, median OS with 95% confidence interval were 20.3 (17.2, NE) months and 22.3 (16.6, NE) months for subjects with shorter baseline TL and the subjects with longer TL, respectively (Table 6).

In 9.4 mg/kg arm, a better OS trend was observed for subjects with shorter baseline TL, i.e., a baseline TL less than or equal to the median TL.

TABLE 6
Overall Survival for Baseline Telomere Length (TL) Grouped by Median Value

| | 4.7 MG/KG | | | 9.4 MG/KG | | |
|---|---|---|---|---|---|---|
| | | <=Median | >Median | | <=Median | >Median |
| | N | Value | Value | N | Value | Value |
| Subjects with | 40 | 18 | 22 | 53 | 29 | 24 |
| baseline | | | | | | |
| Number | 40 | 18(45.0%) | 22(55.0%) | 53 | 29(54.7%) | 24(45.3%) |
| assessed | | | | | | |
| Number | 20 | 8(20.0%) | 12(30.0%) | 35 | 21(39.6%) | 14(26.4%) |
| censored (%) | | | | | | |
| Number of | 20 | 10(25.0%) | 10(25.0%) | 18 | 8(15.1%) | 10(18.9%) |
| events (%) | | | | | | |
| Overall | | | | | | |
| Survival | | | | | | |
| (months) | | | | | | |
| Median (95% CI) | 22.3 (18.2, NE) | 20.3 (17.2, | 22.3 (16.6, | NE (22.8, | NE (23.2, | 22.8 (16.2, |
| | | NE) | NE) | NE) | NE) | NE) |
| 18-months | 68.2 (50.7, | 76.7 (49.2, | 61.0 (36.7, | 75.9 (61.5, | 81.2 (60.5, | 69.8 (46.9, |
| survival rate % | 80.6) | 90.6) | 78.3) | 85.6) | 91.7) | 84.3) |
| (95% CI) | | | | | | |
| 24-months | 39.6 (21.1, | 34.1 (10.9, | 46.6 (21.8, | 54.2 (35.1, | 59.3 (30.9, | 47.6 (22.1, |
| survival rate % | 57.6) | 59.3) | 68.2) | 69.9) | 79.3) | 69.4) |
| (95% CI) | | | | | | |

Baseline TL vs. Responses at Week 24

Baseline telomere length (TL): SVR or TSS response at week 24 was enriched in subjects with shorter baseline TL (<=median value). 17.3% (5/29) subjects with shorter baseline TL and 4.2% (1/24) subjects with longer baseline TL had a spleen response, respectively. 34.5% (10/29) subjects with shorter baseline TL and 25% (6/24) subjects with longer baseline TL had a TSS response, respectively.

In 9.4 mg/kg arm, at week 24, a higher rate of response (SVR or TSS) was enriched in subjects with shorter baseline TL compared to subjects with longer TL (Table 7)

TABLE 7
Baseline Summary of Telomere Length (TL) by Clinical Response

| | SVR >= 35% at week 24 by IRC | | | | | | |
|---|---|---|---|---|---|---|---|
| | | 4.7 mg/kg, N = 40 | | | 9.4 mg/kg N = 53 | | |
| | Total, | | | No, | | Yes, | No, |
| | N = 93 | | Yes, | N = 40 | | N = 6 | N = 47 |
| Baseline TL | With data | Total # | N = 0 (0%) | (100%) | Total # | (10.5%) | (89.5%) |
| Shorter TL | 47 | 18 | 0 (0%) | 10 (100%) | 29 | 5 (17.25%) | 24 (82.75%) |
| (<= Median | | | | | | | |
| value | | | | | | | |
| Longer TL | 46 | 22 | 0 (0%) | 38 (100%) | 24 | 1 (4.2%) | 23 (92.7%) |
| ( >Median | | | | | | | |
| value) | | | | | | | |
| | TSS >= 50% decrease at week 24 | | | | | | |
| | | 4.7 mg/kg, N = 40 | | | 9.4 mg/kg N = 53 | | |
| | Total, | | Yes, | No, | | Yes, | No, |
| | N = 93 | | N = 2 | N = 38 | | N = 16 | N = 37 |
| Baseline TL | With data | Total # | (5%) | (95%) | Total # | (30.2%) | (69.8%) |
| Shorter TL | 47 | 18 | 1 (5.6%) | 17 (94.4%) | 29 | 10 (34.5%) | 19 (65.6%) |
| (<= Median | | | | | | | |
| value | | | | | | | |
| Longer TL | 46 | 22 | 1 (4.5%) | 21 (95.5%) | 24 | 6 (25%) | 18 (75%) |
| ( >Median | | | | | | | |
| value) | | | | | | | |

### Example 3

### Dose-Dependent Pharmacodynamic (PD) Effect

Telomerase activity and hTERT was analyzed to evaluate pharmacodynamic effects for imetelstat. Among subjects with available baseline and post-treatment data, 23 (51.1%) subjects in 9.4 mg/kg arm and 10 (29.4%) subjects in 4.7 mg/kg arm achieved >=50% telomerase activity reduction from baseline, which is the PD effect showed correlation with anti-tumor activity from preclinical xenograft models in vivo. In addition, 35 (61.4%) subjects in 9.4 mg/kg arm and 20 (47.7%) subjects in 4.7 mg/kg arm achieved >=50% hTERT RNA level reduction from baseline, respectively (Table 8). As such, a dose-dependent PD effect was demonstrated, indicating target engagement.

TABLE 8
Subjects Achieved Reduction in TA (30%, 50%) or
hTERT (50%) from Baseline at Any Timepoints.

| | 4.7 MG/KG | 9.4 MG/KG |
|---|---|---|
| Subjects with Baseline (hTERT) | 48 | 57 |
| hTERT >=50% decrease | 20 (41.7%) | 35 (61.4%) |
| Subjects with Baseline (TA) | 34 | 45 |
| TA >=30% decrease | 14 (41.2%) | 28 (62.2%) |
| TA >=50% decrease | 10 (29.4%) | 23 (51.1%) |

### Example 4

Association Between PD Effect and Response at week 24

Higher % of subjects in spleen responders (83.3%) achieved at least >=50% decrease in hTERT RNA expression levels than in non-spleen responders (55.6%); higher % of subjects who had TSS response achieved at least >=50% decrease in hTERT RNA expression levels than in non-TSS responders (Table 9). hTERT RNA expression levels were measured from whole blood samples collected from patients pre- and post-treatment.

TABLE 9
Association between reduction in hTERT (50%) from
baseline and SVR or TSS response at week 24.

| | hTERT>50% reduction |
|---|---|
| Spleen Response at wk 24 | |
| by IRC | |
| Yes | 5/6 (83.3%) |
| No | 55/99 (55.6%) |
| TSS Response at wk 24 | |
| Yes | 15/24 (62.5%) |
| No | 45/81 (55.6%) |

Higher % of subjects who had spleen response or TSS response achieved at least >=30% or >=50% decrease in telomerase activity than in subjects who had no spleen response or TSS response (Table 10).

TABLE 10
Association between reduction in telomerase activity (30%, 50%)
from baseline and SVR or TSS response at week 24.

| | Telomerase Activity | |
|---|---|---|
| | >50% reduction | >30% reduction |
| Spleen Response at wk 24 | | |
| by IRC | | |
| Yes | 2/4 (50%) | 3/4 (75%) |
| No | 34/70 (48.6%) | 41/70 (58.6%) |
| TSS Response at wk 24 | | |
| Yes | 12/18 (66.7%) | 15/18 (83.3%) |
| No | 24/56 (42.9%) | 29/56 (51.8%) |

Aspects, including embodiments, of the subject matter described herein may be beneficial alone or in combination, with one or more other aspects or embodiments. Without limiting the description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. Use of a telomerase inhibitor in the treatment of patient that has myelofibrosis wherein the patient is determined to have a triple negative status,

wherein the triple negative status comprises an absence of a mutation in each of the Janus kinase 2 (JAK2), calreticulin (CALR), and thrombopoietin receptor (MPL) genes.
2. The use of 1, wherein the myelofibrosis is primary myelofibrosis.
3. The use of 2, wherein the myelofibrosis is myelofibrosis that develops post-polycythemia vera (post-PV MF).
4. The use of 2, wherein the myelofibrosis is myelofibrosis that develops post essential thrombocythemia (post-ET MF).
5. The use of any one of 1-4, wherein the patient has not previously received JAK-inhibitor therapy.
6. The use of any one of 1-4, wherein the patient has received JAK-inhibitor therapy and the patient was refractory to JAK-inhibitor therapy.
7. The use of any one of 1-4, wherein the patient has received JAK-inhibitor therapy and is relapsed.
8. The use of any one of 1-4, wherein the patient has received JAK-inhibitor therapy and discontinued JAK-inhibitor therapy due to treatment-related toxicities or intolerance.
9. The use of any one of 1-8, wherein the telomerase inhibitor is imetelstat.
10. The use of 9, wherein the imetelstat is imetelstat sodium.
11. The use of 10, wherein the telomerase inhibitor is imetelstat and is administered for 1, 2, 3, 4, 5, 6, 7, 8 or more than 8 dosage cycles, each cycle comprising:

intravenous administration of about 7-10 mg/kg imetelstat once every three weeks;

intravenous administration of about 7-10 mg/kg imetelstat once weekly for three weeks;

intravenous administration of about 2.5-10 mg/kg imetelstat once every three weeks; or

intravenous administration of about 0.5-9.4 mg/kg imetelstat once every three weeks.
12. The use of 11, wherein each dosage cycle comprises intravenous administration of about 7-10 mg/kg imetelstat once every three weeks.
13. The use of 12, wherein each dosage cycle comprises intravenous administration of about 9.4 mg/kg imetelstat once every three weeks.
14. The use of any one of 1-13, wherein the average relative telomere length is determined by analyzing the relative length of telomeric nucleic acids in target cells present in a biological sample from the patient.
15. The use of any one of 1-14, further comprising selecting a patient identified as having an average relative telomere length in target cells present in a biological sample from the patient determined to be in the 50th percentile or less of a relative telomere length range determined from one or more known standards.
16. The use of any one of 1-15, further comprising screening a patient to determine if the patient has a high-molecular risk (HMR), wherein having HMR comprises the presence of a mutation in at least one gene selected from the group consisting of ASXL1, EZH2, SRSF2 and IDH1/2.
17. The use of any one of 1-16, further comprising assessing hTERT expression level in a biological sample obtained from the patient after administration of the telomerase inhibitor.
18. The use of 17, wherein the hTERT expression level is reduced by 50% or more relative to a baseline hTERT expression level prior to administration of the telomerase inhibitor.
19. The use of any one of 17-18, further comprising altering the dosage of the telomerase inhibitor, the frequency of dosing, or the course of therapy administered to the subject.
20. Use of a telomerase inhibitor in the treatment of patient that has myelofibrosis wherein the patient is determined to have a high-molecular risk (HMR),

wherein having HMR comprises the presence of a mutation in at least one gene selected from the group consisting of additional sex combs like 1 (ASXL1), enhancer of zeste homolog 2 (EZH2), serine and arginine rich splicing factor 2 (SRSF2), and isocitrate dehydrogenase 1/2 (IDH1/2).
21. The use of 20, wherein the myelofibrosis is primary myelofibrosis.
22. The use of 21, wherein the myelofibrosis is myelofibrosis that develops post-polycythemia vera (post-PV MF).
23. The use of 21, wherein the myelofibrosis is myelofibrosis that develops post essential thrombocythemia (post-ET MF).
24. The use of any one of 20-23, wherein the patient has not previously received JAK-inhibitor therapy.
25. The use of any one of 20-23, wherein the patient has received JAK-inhibitor therapy and the patient was refractory to JAK-inhibitor therapy.
26. The use of any one of 20-23, wherein the patient has received JAK-inhibitor therapy and is relapsed.
27. The use of any one of 20-23, wherein the patient has received JAK-inhibitor therapy and discontinued JAK-inhibitor therapy due to treatment-related toxicities or intolerance.
28. The use of any one of 20-27, wherein the telomerase inhibitor is imetelstat.
29. The use of 28, wherein the imetelstat is imetelstat sodium.
30. The use of 28, wherein the telomerase inhibitor is imetelstat and is administered for 1, 2, 3, 4, 5, 6, 7, 8 or more than 8 dosage cycles, each cycle comprising:

intravenous administration of about 7-10 mg/kg imetelstat once every three weeks;

intravenous administration of about 7-10 mg/kg imetelstat once weekly for three weeks;

intravenous administration of about 2.5-10 mg/kg imetelstat once every three weeks; or

intravenous administration of about 0.5-9.4 mg/kg imetelstat once every three weeks.
31. The use of 30, wherein each dosage cycle comprises intravenous administration of about 7-10 mg/kg imetelstat once every three weeks.
32. The use of 31, wherein each dosage cycle comprises intravenous administration of about 9.4 mg/kg imetelstat once every three weeks.
33. The use of any one of 20-32, further comprising determining average relative telomere length by analyzing the relative length of telomeric nucleic acids in target cells present in a biological sample from the patient.
34. The use of any one of 20-33, further comprising selecting a patient identified as having an average relative telomere length in target cells present in a biological sample from the patient determined to be in the 50th percentile or less of a relative telomere length range determined from one or more known standards.
35. The use of any one of 20-34, further comprising screening a patient to determine if the patient is triple negative status, wherein the triple negative status comprises an absence of a mutation in each of the genes selected from the group consisting of JAK2, CALR and MPL.
36. The use of any one of 20-35, further comprising assessing hTERT expression level in a biological sample obtained from the patient after administration of the telomerase inhibitor.
37. The use of 36, wherein the hTERT expression level is reduced by 50% or more relative to a baseline hTERT expression level prior to administration of the telomerase inhibitor.
38. The use of any one of 36-37, further comprising altering the dosage of the telomerase inhibitor, the frequency of dosing, or the course of therapy administered to the subj ect.
39. Use of a telomerase inhibitor in the treatment of patient that has myelofibrosis wherein cells present in a biological sample from the patient have been determined to have average relative telomere length that is determined to be in the 50th percentile or less of a relative telomere length range determined from one or more known standards.
40. The use of 39, wherein the myelofibrosis is primary myelofibrosis.
41. The use of 40, wherein the myelofibrosis is myelofibrosis that develops post-polycythemia vera (post-PV MF).
42. The use of 40, wherein the myelofibrosis is myelofibrosis that develops post essential thrombocythemia (post-ET MF).
43. The use of any one of 39-42, wherein the patient has not previously received JAK-inhibitor therapy.
44. The use of any one of 39-42, wherein the patient has received JAK-inhibitor therapy and the patient was refractory to JAK-inhibitor therapy.
45. The use of any one of 39-42, wherein the patient has received JAK-inhibitor therapy and is relapsed.
46. The use of any one of 39-42, wherein the patient has received JAK-inhibitor therapy and discontinued JAK-inhibitor therapy due to treatment-related toxicities or intolerance.
47. The use of any one of 39-46, wherein the telomerase inhibitor is imetelstat.
48. The use of 47, wherein the imetelstat is imetelstat sodium.
49. The use of 47, wherein the telomerase inhibitor is imetelstat and is administered for 1, 2, 3, 4, 5, 6, 7, 8 or more than 8 dosage cycles, each cycle comprising:

intravenous administration of about 7-10 mg/kg imetelstat once every three weeks;

intravenous administration of about 7-10 mg/kg imetelstat once weekly for three weeks;

intravenous administration of about 2.5-10 mg/kg imetelstat once every three weeks; or

intravenous administration of about 0.5-9.4 mg/kg imetelstat once every three weeks.
50. The use of 49, wherein each dosage cycle comprises intravenous administration of about 7-10 mg/kg imetelstat once every three weeks.
51. The use of 50, wherein each dosage cycle comprises intravenous administration of about 9.4 mg/kg imetelstat once every three weeks.
52. The use of any one of 39-51, further comprising determining average relative telomere length by analyzing the relative length of telomeric nucleic acids in the cells present in the biological sample from the patient.
53. The use of any one of 39-52, further comprising assessing hTERT expression level in a biological sample obtained from the patient after administration of the telomerase inhibitor.
54. The use of 53, wherein the hTERT expression level is reduced by 50% or more relative to a baseline hTERT expression level prior to administration of the telomerase inhibitor.
55. The use of any one of 53-54, further comprising altering the dosage of the telomerase inhibitor, the frequency of dosing, or the course of therapy administered to the subject.
56. A method of selecting a patient most likely to benefit from treatment with a telomerase inhibitor comprising:

testing a patient for triple negative status, wherein the triple negative status comprises an absence of a mutation in each of the JAK2, CALR and MPL genes; and

selecting the patient if the patient has triple negative status,

wherein the selected patient is most likely to benefit from treatment with a telomerase inhibitor.
57. The method of 56, wherein the patient has myelofibrosis.
58. The method of 57, wherein the myelofibrosis is primary myelofibrosis.
59. The method of 57, wherein the myelofibrosis is myelofibrosis that develops post-polycythemia vera (post-PV MF).
60. The method of 57, wherein the myelofibrosis is myelofibrosis that develops post-essential thrombocythemia (post-ET MF).
61. The method of any of 56 to 60, wherein the patient has not previously received JAK-inhibitor therapy.
62. The method of any one of 56 to 60, wherein the patient:

has previously received JAK-inhibitor therapy;

has previously received and has failed JAK-inhibitor therapy; or

has previously received JAK-inhibitor therapy and has discontinued JAK-inhibitor therapy due to treatment-related to toxicities or intolerance.
63. The method of any one of 56 to 60, wherein the patient has received JAK-inhibitor therapy and the patient was refractory to JAK-inhibitor therapy.
64. The method of any one of 56 to 60, wherein the patient has received JAK-inhibitor therapy and is relapsed.
65. The method of any one of 56 to 60, wherein the patient has received JAK-inhibitor therapy and discontinued JAK-inhibitor therapy due to treatment-related toxicities or intolerance.
66. The method of any one of 56 to 65, further comprising administering the telomerase inhibitor to the patient.
67. The method of 66, wherein the telomerase inhibitor is imetelstat.
68. The method of 67, wherein the imetelstat is imetelstat sodium.
69. The method of any one of 56-68, further comprising obtaining a sample that comprises DNA from the patient.
70. The method of 69, wherein the sample comprises bone marrow, peripheral blood or a combination thereof.
71. The method of 70, wherein the step of obtaining a sample from a patient comprises:

obtaining a bone marrow sample, a peripheral blood sample or a combination thereof; and

isolating DNA from the bone marrow sample, the peripheral blood sample or combination thereof.
72. The method of 70, wherein the step of obtaining a sample from a patient comprises:

obtaining a bone marrow sample from the patient;

isolating cells from the bone marrow sample; and

extracting DNA from the isolated cells.
73. The method of 70, wherein the step of obtaining a sample from a patient comprises:

obtaining a peripheral blood sample from the patient;

isolating cells from the peripheral blood sample; and

extracting DNA from the isolated cells.
74. A method of selecting a patient most likely to benefit from treatment with a telomerase inhibitor comprising:

testing a patient to determine if the patient has an HMR, wherein having HMR comprises the presence of a mutation in at least one gene selected from the group consisting of ASXL1, EZH2, SRSF2, and IDH1/2; and

selecting the patient if the patient has an HMR,

wherein the selected patient is most likely to benefit from treatment with a telomerase inhibitor.
75. The method of 74, wherein the patient has myelofibrosis.
76. The method of 75, wherein the myelofibrosis is primary myelofibrosis.
77. The method of 75, wherein the myelofibrosis is myelofibrosis that develops post-polycythemia vera (post-PV MF).
78. The method of 75, wherein the myelofibrosis is myelofibrosis that develops post-essential thrombocythemia (post-ET MF).
79. The method of any of 74 to 78, wherein the patient has not previously received JAK-inhibitor therapy.
80. The method of any one of 74 to 78, wherein the patient:

has previously received JAK-inhibitor therapy;

has previously received and has failed JAK-inhibitor therapy; or

has previously received JAK-inhibitor therapy and has discontinued JAK-inhibitor therapy due to treatment-related to toxicities or intolerance.
81. The method of any one of 74 to 78, wherein the patient has received JAK-inhibitor therapy and the patient was refractory to JAK-inhibitor therapy.
82. The method of any one of 74 to 78, wherein the patient has received JAK-inhibitor therapy and is relapsed.
83. The method of any one of 74 to 78, wherein the patient has received JAK-inhibitor therapy and discontinued JAK-inhibitor therapy due to treatment-related toxicities or intolerance.
84. The method of any one of 74 to 83, further comprising administering the telomerase inhibitor to the patient.
85. The method of 84, wherein the telomerase inhibitor is imetelstat.
86. The method of 85, wherein the imetelstat is imetelstat sodium.
87. The method of any one of 74-86, further comprising obtaining a sample that comprises DNA from the patient.
88. The method of 87, wherein the sample comprises bone marrow, peripheral blood or a combination thereof.
89. The method of 88, wherein the step of obtaining a sample from a patient comprises:

obtaining a bone marrow sample, a peripheral blood sample or a combination thereof and

isolating DNA from the bone marrow sample, the peripheral blood sample or combination thereof.
90. The method of 88, wherein the step of obtaining a sample from a patient comprises:

obtaining a bone marrow sample from the patient;

isolating cells from the bone marrow sample; and

extracting DNA from the isolated cells.
91. The method of 88, wherein the step of obtaining a sample from a patient comprises:

obtaining a peripheral blood sample from the patient;

isolating cells from the peripheral blood sample; and

extracting DNA from the isolated cells.
92. A method of selecting a patient most likely to benefit from treatment with a telomerase inhibitor, the method comprising:

testing a patient for average relative telomere length, by analyzing the relative length of telomeric nucleic acids in target cells present in a biological sample from the patient; and

selecting the patient if the patient has average relative telomere length in target cells present in a biological sample from the patient that is determined to be in the 50th percentile or less of a relative telomere length range determined from one or more known standards, wherein the selected patient is most likely to benefit from treatment with a telomerase inhibitor.
93. The method of 92, wherein the patient has myelofibrosis.
94. The method of 93, wherein the myelofibrosis is primary myelofibrosis.
95. The method of 93, wherein the myelofibrosis is myelofibrosis that develops post-polycythemia vera (post-PV MF).
96. The method of 93, wherein the myelofibrosis is myelofibrosis that develops post-essential thrombocythemia (post-ET MF).
97. The method of any of 92 to 96, wherein the patient has not previously received JAK-inhibitor therapy.
98. The method of any one of 92 to 96, wherein the patient:

has previously received JAK-inhibitor therapy;

has previously received and has failed JAK-inhibitor therapy; or

has previously received JAK-inhibitor therapy and has discontinued JAK-inhibitor therapy due to treatment-related to toxicities or intolerance.
99. The method of any one of 92 to 96, wherein the patient has received JAK-inhibitor therapy and the patient was refractory to JAK-inhibitor therapy.
100. The method of any one of 92 to 96, wherein the patient has received JAK-inhibitor therapy and is relapsed.
101. The method of any one of 92 to 96, wherein the patient has received JAK-inhibitor therapy and discontinued JAK-inhibitor therapy due to treatment-related toxicities or intolerance.
102. The method of any one of 92 to 101, further comprising administering the telomerase inhibitor to the patient.
103. The method of 102, wherein the telomerase inhibitor is imetelstat.
104. The method of 103, wherein the imetelstat is imetelstat sodium.
105. The method of any one of 92-104, further comprising obtaining a sample that comprises DNA from the patient.
106. The method of 105, wherein the sample comprises bone marrow, peripheral blood or a combination thereof.
107. The method of 106, wherein the step of obtaining a sample from a patient comprises:

obtaining a bone marrow sample, a peripheral blood sample or a combination thereof and

isolating DNA from the bone marrow sample, the peripheral blood sample or combination thereof.
108. The method of 106, wherein the step of obtaining a sample from a patient comprises:

obtaining a bone marrow sample from the patient;

isolating cells from the bone marrow sample; and

extracting DNA from the isolated cells.
109. The method of 106, wherein the step of obtaining a sample from a patient comprises:

obtaining a peripheral blood sample from the patient;

isolating cells from the peripheral blood sample; and

extracting DNA from the isolated cells.
110. A method of monitoring therapeutic efficacy in a subject with myelofibrosis (MF), the method comprising:

measuring hTERT expression level in a biological sample obtained from the patient after administration of a telomerase inhibitor; and

comparing the hTERT expression level in the biological sample to a baseline hTERT expression level prior to administration of the telomerase inhibitor;

wherein a 50% or greater reduction in hTERT expression level in the biological sample identifies a subject who has an increased likelihood of benefiting from treatment with the telomerase inhibitor.
111. The method of 110, wherein the hTERT expression level measured or assessed is hTERT RNA expression level.
112. A method of identifying a patient with myelofibrosis (MF) for treatment with a telomerase inhibitor, the method comprising:

measuring hTERT expression level in a biological sample obtained from the patient after administration of a telomerase inhibitor; and

comparing the hTERT expression level in the biological sample to a baseline hTERT expression level prior to administration of the telomerase inhibitor;

wherein a reduction in hTERT expression level in the biological sample identifies a patient who has an increased likelihood of benefiting from treatment with the telomerase inhibitor.
113. The method of 112, wherein the reduction in hTERT expression level is 50% or more.

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art,and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treating a patient that has myelofibrosis with a telomerase inhibitor, the method comprising administering the telomerase inhibitor to the patient if the patient is determined to have a triple negative status,
wherein the triple negative status comprises an absence of a mutation in each of the Janus kinase 2 (JAK2), calreticulin (CALR), and thrombopoietin receptor (MPL) genes.

2. The method of claim 1, wherein the myelofibrosis is selected from primary myelofibrosis or myelofibrosis that develops post-polycythemia vera (post-PV MF) or myelofibrosis that develops post essential thrombocythemia (post-ET MF).

3. The method of claim 1, wherein the patient has not previously received JAK-inhibitor therapy.

4. The method of claim 1, wherein the patient:
has previously received JAK-inhibitor therapy and is relapsed;
has previously received JAK-inhibitor therapy and is refractory; or
has previously received JAK-inhibitor therapy and has discontinued JAK-inhibitor therapy due to treatment-related to toxicities or intolerance.

5. The method of claim 1, wherein the telomerase inhibitor is imetelstat.

6. The method of claim 5, wherein the imetelstat is imetelstat sodium.

7. The method of claim 5, wherein the telomerase inhibitor is imetelstat and is administered for 1, 2, 3, 4, 5, 6, 7, 8 or more than 8 dosage cycles, each cycle comprising:
intravenous administration of about 7-10 mg/kg imetelstat once every three weeks;
intravenous administration of about 7-10 mg/kg imetelstat once weekly for three weeks;
intravenous administration of about 2.5-10 mg/kg imetelstat once every three weeks; or
intravenous administration of about 0.5-9.4 mg/kg imetelstat once every three weeks.

8. The method of claim 7, wherein each dosage cycle comprises intravenous administration of about 7-10 mg/kg imetelstat once every three weeks.

9. The method of claim 8, wherein each dosage cycle comprises intravenous administration of about 9.4 mg/kg imetelstat once every three weeks.

10. The method of claim 1, further comprising determining average relative telomere length by analyzing the relative length of telomeric nucleic acids in target cells present in a biological sample from the patient.

11. The method of claim 10, further comprising selecting a patient identified as having an average relative telomere length in target cells present in a biological sample from the patient determined to be in the 50th percentile or less of a relative telomere length range determined from one or more known standards.

12. The method of claim 1, further comprising screening a patient to determine if the patient has a high-molecular risk (HMR), wherein having HMR comprises the presence of a mutation in at least one gene selected from the group consisting of ASXL1, EZH2, SRSF2 and IDH1/2.

13. The method of claim 1, further comprising assessing hTERT expression level in a biological sample obtained from the patient after administration of the telomerase inhibitor.

14. The method of claim 13, wherein the hTERT expression level is reduced by 50% or more relative to a baseline hTERT expression level prior to administration of the telomerase inhibitor.

15. The method of claim 14, further comprising altering the dosage of the telomerase inhibitor, the frequency of dosing, or the course of therapy administered to the subject.

16. A method of treating a patient that has myelofibrosis with a telomerase inhibitor, the method comprising administering the telomerase inhibitor to the patient if the patient is determined to have a high-molecular risk (HMR), wherein having HMR comprises the presence of a mutation in at least one gene selected from the group consisting of additional sex combs like 1 (ASXL1), enhancer of zeste homolog 2 (EZH2), serine and arginine rich splicing factor 2 (SRSF2), and isocitrate dehydrogenase 1/2 (IDH1/2).

17. The method of claim 16, wherein the myelofibrosis is selected from primary myelofibrosis or myelofibrosis that develops post-polycythemia vera (post-PV MF) or myelofibrosis that develops post essential thrombocythemia (post-ET MF).

18. The method of claim 16, wherein the patient has not previously received JAK-inhibitor therapy.

19. The method of claim 16, wherein the patient:
has previously received JAK-inhibitor therapy and is relapsed;
has previously received JAK-inhibitor therapy and is refractory; or has previously received JAK-inhibitor therapy and has discontinued JAK-inhibitor therapy due to treatment-related to toxicities or intolerance.

20. The method of claim 16, wherein the telomerase inhibitor is imetelstat.

21. The method of claim 20, wherein the imetelstat is imetelstat sodium.

22. The method of claim 20, wherein the telomerase inhibitor is imetelstat and is administered for 1, 2, 3, 4, 5, 6, 7, 8 or more than 8 dosage cycles, each cycle comprising:
intravenous administration of about 7-10 mg/kg imetelstat once every three weeks;
intravenous administration of about 7-10 mg/kg imetelstat once weekly for three weeks;
intravenous administration of about 2.5-10 mg/kg imetelstat once every three weeks; or
intravenous administration of about 0.5-9.4 mg/kg imetelstat once every three weeks.

23. The method of claim 22, wherein each dosage cycle comprises intravenous administration of about 7-10 mg/kg imetelstat once every three weeks.

24. The method of claim 23, wherein each dosage cycle comprises intravenous administration of about 9.4 mg/kg imetelstat once every three weeks.

25. The method of claim 16, further comprising determining average relative telomere length by analyzing the relative length of telomeric nucleic acids in target cells present in a biological sample from the patient.

26. The method of claim 25, further comprising selecting a patient identified as having an average relative telomere length in target cells present in a biological sample from the patient determined to be in the 50th percentile or less of a relative telomere length range determined from one or more known standards.

27. The method of claim 16, further comprising screening a patient to determine if the patient is triple negative status, wherein the triple negative status comprises an absence of a mutation in each of the genes selected from the group consisting of JAK2, CALR and MPL.

28. The method of claim 16, further comprising assessing hTERT expression level in a biological sample obtained from the patient after administration of the telomerase inhibitor.

29. The method of claim 28, wherein the hTERT expression level is reduced by 50% or more relative to a baseline hTERT expression level prior to administration of the telomerase inhibitor.

30. The method of claim 29, further comprising altering the dosage of the telomerase inhibitor, the frequency of dosing, or the course of therapy administered to the subject.

31. A method of treating a patient that has myelofibrosis with a telomerase inhibitor, the method comprising administering the telomerase inhibitor to the patient if cells present in a biological sample from the patient are determined to have average relative telomere length that is determined to be in the 50th percentile or less of a relative telomere length range determined from one or more known standards.

32. The method of claim 31, wherein the myelofibrosis is selected from primary myelofibrosis or myelofibrosis that develops post-polycythemia vera (post-PV MF) or myelofibrosis that develops post essential thrombocythemia (post-ET MF).

33. The method of claim 31, wherein the patient has not previously received JAK-inhibitor therapy.

34. The method of claim 31, wherein the patient:
has previously received JAK-inhibitor therapy and is relapsed;
has previously received JAK-inhibitor therapy and is refractory; or
has previously received JAK-inhibitor therapy and has discontinued JAK-inhibitor therapy due to treatment-related to toxicities or intolerance.

35. A method of selecting a patient most likely to benefit from treatment with a telomerase inhibitor comprising:
testing a patient for triple negative status, wherein the triple negative status comprises an absence of a mutation in each of the JAK2, CALR and MPL genes; and
selecting the patient if the patient has triple negative status,
wherein the selected patient is most likely to benefit from treatment with a telomerase inhibitor.

36. The method of claim 35, wherein the myelofibrosis is selected from primary myelofibrosis or myelofibrosis that develops post-polycythemia vera (post-PV MF) or myelofibrosis that develops post essential thrombocythemia (post-ET MF).

37. The method of claim 35, wherein the patient has not previously received JAK-inhibitor therapy.

38. The method of any one of claim 35, wherein the patient:
has previously received JAK-inhibitor therapy and is relapsed;
has previously received JAK-inhibitor therapy and is refractory; or
has previously received JAK-inhibitor therapy and has discontinued JAK-inhibitor therapy due to treatment-related to toxicities or intolerance.

39. The method of claim 35, further comprising screening a patient to determine if the patient has a high-molecular risk (HMR), wherein having HAIR comprises the presence of a mutation in at least one gene selected from the group consisting of ASXL1, EZH2, SRSF2 and IDH1/2.

40. The method of claim 35, further comprising administering the telomerase inhibitor to the patient.

41. The method of a claim 35, further comprising obtaining a sample that comprises DNA from the patient.

42. A method of selecting a patient most likely to benefit from treatment with a telomerase inhibitor comprising:
testing a patient to determine if the patient has a high molecular risk (HMR), wherein having HMR comprises the presence of a mutation in at least one gene selected from the group consisting of ASXL1, EZH2, SRSF2, and IDH1/2; and
selecting the patient if the patient has a high molecular risk (HMR),
wherein the selected patient is most likely to benefit from treatment with a telomerase inhibitor.

43. The method of claim 42, wherein the myelofibrosis is selected from primary myelofibrosis or myelofibrosis that develops post-polycythemia vera (post-PV MF) or myelofibrosis that develops post essential thrombocythemia (post-ET MF).

44. The method of claim 42, wherein the patient has not previously received JAK-inhibitor therapy.

45. The method of any one of claim 42, wherein the patient:
has previously received JAK-inhibitor therapy and is relapsed;
has previously received JAK-inhibitor therapy and is refractory; or
has previously received JAK-inhibitor therapy and has discontinued JAK-inhibitor therapy due to treatment-related to toxicities or intolerance.

46. The method of claim 42, further comprising screening a patient to determine if the patient is triple negative status, wherein the triple negative status comprises an absence of a mutation in each of the genes selected from the group consisting of JAK2, CALR and MPL.

47. The method of claim 42, further comprising administering the telomerase inhibitor to the patient.

48. The method of claim 42, further comprising obtaining a sample that comprises DNA from the patient.

49. A method of monitoring therapeutic efficacy in a subject with myelofibrosis (MF), the method comprising:
measuring hTERT expression level in a biological sample obtained from the patient after administration of a telomerase inhibitor; and
comparing the hTERT expression level in the biological sample to a baseline hTERT expression level prior to administration of the telomerase inhibitor;
wherein a 50% or greater reduction in hTERT expression level in the biological sample identifies a subject who has an increased likelihood of benefiting from treatment with the telomerase inhibitor.

50. The method of claim 49, wherein the hTERT expression level measured or assessed is hTERT RNA expression level.


SEQUENCE LISTING (9 sequences)

SEQ ID NO: 1 - DNA, 13 bp, Artificial sequence
  synthetic sequence
tagggttagacaa

SEQ ID NO: 2 - DNA, 142939 bp, Artificial sequence
  synthetic sequence
ctgcaggaaggagagaggaagaggagcagaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgtcccggggcttcgcagaccttgacccgccgggtaggagccgcccctgcgggctcgagggcgcgctctggtcgcccgatctgtgtagccggtgggtgttatctctgcctggcttctgggtggggggcagcagctgtctctccacccccttcctaccttttggtcccgcctgccccctgctagtggcagcaaacttctgagccagaaaaatcttcctctttaagacgacacctagaacgagtcttgctggtgatatttgcttccatttgtgtcttataattccctcgctcccgcctccttattcctttttaaaattttctagacacttaaacagcctagaaaaagcatttgtttcccctggatttatgtggtagtagttaactgctgcttctgtttttaggtttcagaagcaggcaacaggaacaagatgtgaactgtttctcttctgcagaaaaagaggctcttcctcctcctcccgcgacggtgggtgtgctgtcctttatcgctgcagtaaaggcgaaggtgtctggagctgggagtgcgtgcgtagggaaggttggaagcgtgtgaagagaggcagggctcattgttgcttactcttctgaaaatgtcagtgctaaacttttaatgttgatctgggagagtaattttggggtgttttgttgttcgtcttggatcctttaagaaacatttttttgttggctttacacataacgtgactttgcagagaaacgaaaaacgttgtccattaccctttcttcttaagcagccagagattcttaatcctttaaagaggagtatttccaagcaataagagatagcttttgggagaacacgttagctattcttgctggggtttctgctaccacattgaatgtttcctcatggttttatctgcctaagcttgctgtgtcggttgaatttacccagtttgttttccagtaattactaatctgttaggagaaactccatttctctgaatgaaagggtaggagcggctgagtatggagaaggaaactggaagtgggaataagggaaatgattgcttttggtttcataataagtttgtacagtgagttgttgggctactgcaaataaagattttgtatcccttctccagaatgcttgggaccaaaagtgttttggattttttttaaaaattttggaatgtttgcatatacaaaatcagacatcttgaggatgggacccaagtgtaaatacgaaattcgtttgtttcatatagaccttattcacacagccactaagtcatttgcaatatttttaatacgtctgtgcatgaaacaaagtttgtattaagtacctatgcggggaattttccccttgtagcatcatgttggtgctcaaaatgtttcagattttgcagcttttcagataagggatgatcagcttgtattttgatataacacccagaacatttgaagatgaataatcaatttttggcctcccttcccccatccgcctcaccttataccactttaagtgtgagaattatttgcttgtacatgcatttgtctgttggtgtgtaacgtatgtacagactggatgtatccagcacttgccttaatagtacatgtatttacctgaataggaaagctgcagtacttgctggccagaagagcacaccatgcttgtttttgctctaactgaatatcctgtatttgcaatttatagcagttttagggactcattagggaattttgtgtgtgtgtgcctttggtgatttgagacaggtgtctcagatatgggaagggaatatgacattgtttctctttcacctggctattactgaaaggtgggatgcttttgaggcagaataggaaagtgcattggctttggagtgagatctggatttgaatcctggtgttcttgccacttaagagttgcattatttacggctggtcatggtggctcacgcctgtaatcccagcactttgggaggctgaggaaggaggatcacttgaggtgaggagcttgagaccagcctggccaacatggtgaaaccccatctctactaaaaatacaaaaattagccgggcgtggtggtgcacacctgtaatcccagctactcgggagggtgaggcaggagaattgcttgaacctgggaagtggtggttgcagtgagccgagatctcaccactgcactccaggctgggcaacacagcaagactctgtctaaaaaaaaaaaaaaaaagaaagaaaaaaagagttatattatttacgactcttttagcttcactttcttataagattgttgtgagaagtgagtaagacacatgcaaagtggtaaactgcctggctgtcataaaagtcttggctactatcacggctgggaggcaaaggagattcagagaagggcagaatgactgcataaggtgatctgcctgtaactcgtgtttcctcctttctagtataaagagataaaggacttcaaggtcttaaactggtgagggagtattacagcctttgtatggaaaggtttgactttgtgtctctgcttaaacccccattgtgttgcccctaccaattattgtgctgcccccttgtggttaactgtgctaagacctttcttttgctcttctcccattacatttcccacccttccccactcccttttagacccctcctcttagctcccagtttcccaaactgtgtgccaaggtgcccagagcacctcaggcttgaacttgtgttttgagttggttcatggtttcaatattagatacctacacccgtttctacaatgtcatgtctttgtaaagatgtattttcagaggttgctccgataaaaagcaactactacatgaaaatgaatgtagaacaggaaatgacagtgtggtattgtccaatttgattctaaggtttgataagttctacagtgctctacactaagttgtaaggacataactactaagtaagttgttcaaacctatttcttttggcctgggggtgtcgtgaaaaatttgctaagactcagagctccctgaatgggtttggaaacctctgccttagcttttagtttgcttttctgataaaattattgatataaagctagtaccagctgatcatggcttaatatatttcctctcctatgtaatatgtttataacttctgcccagttctttcattttaaaactaatcctattgctaacttgttgtactagtcacttcctggtttttcctgtactggtcttctgcttctacttctgttcatctccttaaactccgaatgccccaagtgaaagttaccattttctaccccttaattttcttctaatcatgtaggctttaaattaggcatcatctttaaactacttctttcatttacctcttattcccattcaaaatgtccatatttcctttctttctcatatctctgctttccctcctttactgtgtgaatcccaggacacatttcctgagcctagaatattgaaagtcatttgaacttctgagaattcagtgtcccttccttctgtttatcctcaatagagattccactctaatactccttattatagcattcccttgccccaaaaccttcagtggcttaccattttctacttattaaattttcctgacttttaagatagtctattatcaatccctgccctatttatttagtcttttgtctaaggatctctagtccttaagcttgtaagctgatttccacactttctaataataagctatgcttcttctatcctttaatttttgcccatgttctccatccctcttttttttcctctatttctgtagctctttagtcttagaacattttcacatggtttaattatatgccatgctttagttgtttgatagttatttccctagtgttagccatatcttttcaagttgattgtgtattctttgaggagcagtaccttagattaaaaaaaaaccacaacacacagtggagagcacatgggcagcattcaagaaatacatgttgattgtgtgataaggaactgtagtcaccaaacaaatatttgttaatacttgcaatgtaaaaggtacttggctgtattcaaaagagaaataaagaaatataaaataggctcctttccctaaggacttacagtataattgggcagataagctaaataataaaagttacagatatttaagaccacttgagttgagcagagattttgcataataggaaggaactggtagagggggtaagcaaaggcaaaatatgtatatatgtatgatatgttcagagagcagtacataaataacttgactgaaatgaagatttgtgtgaaagagtagtaagaaattgtgttaaaaatgtagttggaatttgattatggatgtctttaaatgctaggatgagaagaatgtactttgacctgtagatatggagaagctttaaaagttttgaattgagaaagagtttgctgaaataagtattttaggaaaatcaaagcagcaccagtgctaggtctcatatgaattggaaagagaatggtagaaagaggtaggaaaataattgagccctgaagtaatgaggacttagaaataagtcagattgcagagatgtcacaagacattattatggattggataagaagggagaatggaggggagaagagtgattaaagattaccttaccttgaaggtttgcttttcagtgacggaggaaatcaaagagtgttgactctccaagacatcttagaacatctaagtctatatgttttgtatattaggactcagaccagagagatttaaaaatgtgtccaaagttactcagctagttaatgacaaagaaactgccgtttgattaatggcagttactaacaaaaatagttatggcagtgttagtagctggtagaggggatgtagacagttgagcgtatattgtggatatgttgagtttgcggtggccgtgggatgggcaagtgaaaattgatagcatacagtgggacatatgactagaatttgttagcgaagttaaggctggtgctgtggtacttaagagaaaagagtgggattgcccataattagcgggtgggtgaaaggagaggaatgggtaaaggagacaaagaaaaaaacagtatgagaggcaaattagtggataggttaggaggcggaatgatcaacatgttgatcattggcaagaaatgagaagaatcagatccaaaaaaaggtcttaaaaagatgtattcgtgtttttcctaataagaaatctataaaaacagaagaaaattgcttatactttccctacgtggaaataaccactgttaatattttgttctctccggtttaacaaaagacctttggatttgacagttgggtgatcagtggagacttttccaaagctctccatcactaggactaaggatgagaattttaggcataataaaacctaggagatatttttcaacagattgtcattcgtctgtatcgtcttatatatcacactgtccaggtactattcttcctgtgtgcattggttctgtgtttttttctgaatgatagaaggttagacaagccatctctgggtgcttttgtgctggtaaaatttgaataaagtgataaaatatttgtttctaaaaggaaggagaaattcttggtcgataacttataaatctgagggttgaatgattgatgaaattaggggtactggaaagaacaaagagaagcattatttattttgaagcttttctgtatagttacaaagggttaactaggttagaatggtccatgggctgtttactttgcttgcatgtttattttgatcttcccaagaacattcttctgttcatctttttagtatgttattggaaacagagtactcattagttcttactaagtggttttagtttaataaattatgattatgaaataaaatgctattaggtaatatttttataatataaagggccatgtgatgtgagtaagtgcactgttttggaaatcaggggataagggactgaccctgctctcagcatggtatatgatttcaggcaactcacgtaatcagtgagtcttgtctattgagggaatggactaaatcttttctagaaaatttaaaaaggtgggatcacatggtaaaatgtgtataaagtttaaaaaggaaatggtttaccaatgtttatgacaaaaaccagcaaactctttcccacccccccaccccacatctccttttctagtctctaattcctgtacaccaggcaaccacctgtcctctgtactttgccgttttcacctttataatgaatgaattgtttagcatcccctctaaagcctctccattcagttgtgtagaagagctcatcccctttgccttcaagggctttaccattatatttctctcctctttttcttgccttctcaattcttccctctactggattatttccatcagcattactctgattgctattgctgtgtaacagaaactacccaaaacttagtgatttaaaacaactactttattaggctcacagataccatgggtcagtaattgagacagggcacaccagggatgactttgtttttgctccacaatggctactaggacatgagttgggaagaattgccagccaggggtgacttgaaggcaaaggactggaatcatctggaggtgtcttcactcacatgatgggtagttgatgctggctcttgactgggatgttagctgggctgttaactggagcatgtacatgtggcctggatttcctcacaatatggtggcttcagggtggtcacctttcatagtggtgagtagagaattaaaagtgagtgtgtctcagcataaatcttttatgatttagccttggtagctgcacagtataacttctgccatgtcctactggttgtaagggtcagaagcctgtccagatttgatgggggagtggcaagttcacactgcagaagaaaaacatgtgagatgggaaatagtgttatggccatctttggacaattattaatactattgccatagtttgccattttgccaaacttcacatccctcctgtatctgaaatacacgcaaaatacactgagcttctcctcaaaggtccctaattcacatcccatcatgacagtaactcagagtccagtatctcatcatataaatcaggtccaggtacagaatagttttcttgggtatggttaattggatatggttcttcttgaactgaggacccatgaagagacagtttatatgtcccctgcatactttacaaaatggaaaaggcacaggttatctgcagtagacattcctcctcaagaagcatgcaaggtaggcacagtctgtagcaattctgcaacctcactgggcacatgttgccagttccttgattaggtctcagtcctgcttcctggaagtcattcttaataggacataggcttcttctgtttttgggtttaccttctgaattatttatctcccccacccctgcacccaacccatataaattgctttgtatttgtaagagtagctttctgagcttgcttcctgcctgtagtgctccagagtccaaaggcccccttttatatagcaactgtccctttcattccaagctaatacaactcctttaaaaactttgtggacttcctgtgtgtaagtttataatctgtactgccatacccacaaatctcttttgagaggaactctttcctcccctaagctctgtgtgagtctgctgtgggacaacacccttaagatccttagataatatttcgtctgaaaggatctatgaggtactgatttagatcttaagaaaggatcttataatcacactctagattggatctttgctctgaggccctttttatttgacactttgctggctgaaaagattgttctgagtccattacatctccttgaaattctactcaaaaaatgaatagcttattctttagttcatctctctgttctctcaattataagggcagctagaagaagccaaattgctttaacttcctgctgagaatctccttaattcattaggtcaggtatgtattttattttccatgttaatgtaggtgacagttttgctaaactttgccctagtatgtaacaagggtgtcctcttattcagctttcactatcatttctctcactttgcttgaagtcctcactgacagtctccttgagggctcttaggctcccatgaagtttcctcaagaccctttacatgtgtgaacacaaagggtccttatctgttcctggctatatagcaggtccttgaataactgtcatttcttcataacgttgataagaaaaaaatggattcctggctggggccactgtttgtgtggagtttggacattctccccacgtctgtgtgggtttttccgggtgctccaatttcctcccacattccaaagatgcatatgttagtggatcggtgtgtctaaatggtctcagactgagtgatcatgtatggatgagtgtgccttgcagtgggtgatgtcctgtctagggttggttcccaccttgtgccttgagttgctaggataggctctagccactcgtgactgaactgaattgggttaataattatcctacttgtttttattagtctttcttaaatgtgtgtatagctcacatttatttcagtgtttaataatcagagtgtttggaatctttacttagaagtttgatgatgtttttgtggccataaatatgccataggaacttccttatctcaattagcctatggaaaaatggattttgttacacattgtttcacttaaagtcacagtttccaagaacctattgaagacagtgaggattaactgtactagaactgtttcctaacccactggttcttatcagaattacctaaaggacttattgaaacacaaattgccgctgggccccacccccagagtttgtttcattagctctggagtgggacttgaggatttgcatctgtgagttcccaggtgatctgataatgttgatctggggcctagattgtgagaattactgatttaaaccagggattggcaagatttttctattaaggatcataaataaaatattttagactttgtgggcaagattgtttctcttgcacctactcaattccgcctttgtggtgcaaaagcagttatagacaatacgtaaatgaacagacgtggctcatggctgtgttctcataaagctttatttacataacaggcagctggctaaatttggtatgtgtgaactgtgctctgccaattcccaatttaaatcattacttgactccacatctcttcaagctatggccccatttttcactcccctttggaacaaaactctctgagagttgtctgtatttgtctctttttattctttcataaaaaaattatctattttttgaacacatttaaatgtcttagaattcaaaaagtagtaaatatctttgtacatttgtcagggcgtgtgtgtgtgtgtgactataactaaagattaagtagaagaggagttgctgattcaaaagtggtatatgttactaagttcaacaaatattgccaaattgacatagaaataccaagttatgcttttctacacctttactaacattgtgttttcagatgacttgattgttgccaacttgagacgtgaaaaatagaatctcattataattttaatttgctttttattctccttttttagggaactcttcactcatatcttggcccactttctattggatttgtggtccttttactttgctttgtgggagctcttatactaaggaagatagctttgagtttgcgaaatgagttgttttccctaggttgtctctttatctttttgattctgtagatagtgtgattttgttgttgtgcatctaggtaggttccttctaaccctgaaattatattttgagattctcccttgttttcttgttccatctggaatttatactgatgcaagatataagatgggacgcaacttttttcccctagatggttgtccagtttcaatactatttgtcagataatccttcttttctctgctgctgtgaaatgctggtgttttaaattgtgtatgtagttggatctatttctggactttgtttcattctaaaaatggctcctctgaaaccatattgtgagttcttgggtagtgaacatttctatttatttgtttccaagtcctgtataatatttattagcatatgggaggcattcaagtacttgaaaataaaatgaataaatcaagcatttaggaatagaagcagatctttttgccaattattcattttagttattctatttaattctctatttctgattttaaacttgaatttaaccttggttttccattttagttttaccttttttttaaactttattgtgaagtattaagttattttttgtacttactgaatgggcatttgctaacttatgtgtctgctcttattagtctatttttggcctcaaattgttaaaaattcacagtatagttttatttgtatcaaatttgctactgagggaagcttgtcatttcataatggcaatccataggataggtcaagtcatttgagattggaaccattaaggataagttcatttaacattgaattattagatctgtggttaattggccaggttagagaagacagaattacagggaagtctggattttacaacttattattttagagggtcaaatttagaataatatttttaagctttttaagtagaagtatatggatcacctgaggtcaggagttcaccaccagcctgactaacatggtgaaaccctgtctctactaaatacaaaaaaatagccaggtgtggtggtgcatgcctgtaatctgagctacttgggaggctgagacaggagaatcgcttgtacctgggaggcagaggttgcagtgagctgagatcgtgccattgcactctagcctgggcaacaagagtgaaactctgtctcaaaaaataaataaataaataaataaaaataaatacataaatagaagtatactattctgattctcttatttatatgtataatttatttttttaaatacatgaggtattgtagattttttggtttttttgaggaggggcgggggtgatgtgaaatatagttttcattgcatcagatcttcagttgtaatgatattgctggaatccaccatttaggaccctgtcttgttaaaaaaaaattatccttgccttcacccctacccctccctgccacagacttttaaaggtaagtattctgcgataattggattatatttactattatgaaaatttaaaattattagaagtaaaatgaaatatacctccattgtagtaagtttgattcacagtgcctctggttatggtggtcagtaaaagtaacactgtattaggaacagtcttcatggtgggagttctctccttttactcttctgttagtttgttctttttttttttttttttttttgagatggtttcactgtcactcaggctggaggacagtggctcgatcacggctcactgcagccttgccttccctggggtccagtgatcctcctacctcagcctcctgagtagctggcactgcaggcgagtgccactaggcctggctaatttttatatttttttttgtagagatggggtttcaccgtgtcactcagtctggtcttgaactcctgggctccagcaatctttctgcatcagcctcccaaagtgctgggattatggttctggagccaccgcgcatggcctgttagtctgttcttgcattgctataaagaaatacctgaggctgagtaatttataaagaaaagagatttaattggctcatggttctgcaggcttacaagcatggggctgacatttgctcagcttctggggagtcctccgggagcttttactcatggtcaaaagctaagagggagcaggaatatcacatttcaagagctggagcaaaagagaatgtggggaaggtgcacacacttttaaacagccaaatcttgcaagaactcagtatcttcaggacagcaccaagggggatgttgctaaaccattcatgagaaatccacccccatgatcaagtcacctcccaccaggcctcacctccaacattggggattacagtgcaacacatgaaatttaaaggggacaacatccaaactgtatcaaccttgctgtacccctcagcctcattttataggcaacaattgaatatattggttttataaaggtttgatatggaaagattcattaggagtggattttaggacttggggaatgactcagagaagaaaatagtgcatgatagtataagtctatctgattgatgagtaactggaaatccttccaaaacataatgcttcagtgtctgcagaattgtttcatttccatgtttatgtatttcttattgtttccttgtttatccataggtgcttaatttttttttaatctgaatgcattatgataatctttaaataattatttcagaggaatatctgaaatattcagtgttcttctagcatggcagtattttaaaaatcattatgagcttaaattcatactgatttaaacaacttctagatttttttgaatatcaatagcagagggtagttttaaatatttcacgagcagaattaattaaatttcctgtgcaattttattttttatagacaaactcgtcacttcttaaaatcttttaaaaatgtcttggtaatacaggcttggaatatgatggtattgaaatgagatcttaagggaaaaaaaactccaacgggtttttttgttgttgttgttgagatggagtcttgctctgtcacccaggctggagtgcagtggcattatctcagctcactgcaacttctgcctcccagattcaagcaattctcctacctcagcctcccgagtaactgggattataggcgcgcaccaccacgcccagctaatttttgtatttttagtagagacggggtttcaccatgttggtcaggctggtctcgaactcctgacctcgtgatccgcccgccttggcctcccaaagtgctgggattacaggcatgagccaccatgcccggccaaaaactccaacttttagaggcctatttacagcacttgtcccatatttgttcacactttttctgtatgtgtgttgtgaattataaatctggcttttcctacaaatgtggcttttcctgtgtagtcatcattaagcatgattctaagaagtagctcaaaatctcctaagaccaaaaaccacaaaaaacaaaaaaaaaaacaacaaaaaaccaaaaccagaaaagggatacattttgcagaataatttatattttcttctcttcatggcatcagttttctcaggagttctttttttgagatggagtctccctctgtcacccaggctgtagtgcagtggtgcgatctgcaagctccgcctcccgggttcacgccattctcctgcctcagcctccggagtagctgggactacaggtgcccgccaccacgcccggctatttttttttttgtattttcagtagagacggggtttcaccgtgttagccaggatggtctcgatctcctgaccttgtgatccacctgcctcggcctcccaaagtgctgggattacaggcgtgagccaccgcacccgggctcaggaggtcttttaagcttttgcctgtcatgtgtgtacttctgacgctggcccaaggtaaaatgattacatttgacttcggtagcccttaaatgtatggctgttcttaatcttttctaaagcctaaaagttgaattagtattcatcttagatttcttgtttcttaagtgaaagaatcatgacattgtattaaaatatcttatttgcatgtattttatatactgttttaataggcagtagtagtatacaagctttgaccaaatgtctcttcatataaaaaattcaagtgaataaaaacagtcatttaaaaatgacattagtcaaaggggcaaaaaagattacaaatgcttattgtgattgacagaacttgcttaacatattagggttctctagaaggacagaactaataggatatatatatgagtttattaaatattaacttaaatgatcacaaggtcccacaataggctgtctgcaagcttgaggagcaaggagagccagttcaagtctcaaaactgaagaacttggagtctgatgttcaagggcaggaagcgtcttggctaatccacccatcttggcctcccaaagtgctaggattacaggcgtgagcccccgtgcctggtgtacatcatttttaatatctttatagtattctgttggctaagttgccacaatttatcagacccctttaatgtcattaattttttcccaatatttagacttgtttcatagaattattagatatgcatgctactactcctgtcctttttcaaaggacaattattagatattcttgttttcctcatatctttgccaacactgggtattggcattcttcttgtttaccagtcagctaaggaagctaaggaagaaacaatatctcatttttatgtacttttctctgattcttaattatgttgaccatgttttcatttcttttgtgaaatgcctatttatgttcattttcatattaaattgtaagagttacttgtataaatatattaaacctttgtagtatatattttttctgtttttttttaaagttttgattatagtgtttataatatgcagtcaaatttacccatcttttatttcctctatcatttttcacttagatgtggggtttagaaaggaggtacataatgtgacagagtttttggtatagaaataactgacatagttagatataaaacataccaaggaaatagtcggaaatgtccaatttcgaatgtgtaaggagaccagtaagaaaatcatgaagaacagctaacagcagtaatttttaaaagggtatttacaaacacttgaaaatgtttatgtagtttaaaggaatgattcaaatataaagagaagcgtaacataaaaatgaaataaaggcatatatatagtcattgtgtagatggttttctagtaacagttataccaagtttgaaatgatttttaggctttgttatgccaacttttaggagttttctttttaaactttttgagcttatgtttcatcatggccaatttttataaattttccctatatgctcaaaacatttttttagtggaaggttttaatacgtctgtaatttctccggtgtcttaaaaataattagtttgtggagctctttgtgtattggagataccatcttcttgtttgatatatttgtatatgtattaagtaagaactggataaagaaggtggttgggatatgtataaatatcaggtaagaacttgataacgtattggttggtttgcatatgaaaagttgccctcactgtcaggttctttcctgtttctaggaattggctagccctggaaggactgccccttccagggtcagcaaattcccaagatagcaaagcatcaaaaatacagaataaaaagacatgcttagtagagtatatagaaatacaattggttttgtttattttgtgatgtatcctgtgatactggtaaattcatttataagttctagtaattttttggtagaatcctcagcatttttgataaatacaatcatgtctgcaaataaaaagttttatttctttcatttcaatctctatgtcttttatttcattttcttaccagattgcattaggtagaacctccagtgttgaatagaaatggtggtggtgagcatcctttccttgttcctgattttatggagaaagcattcaatatgtaatcattaagtaatagaaggttgtttgtagatgatctttattttattgaggaaattcccttctttttctaatttatttccagaaattttatcatgaattggtgttgacttttgtcaaatgcttttcctgcaacaattgggacgatcttatggttttgctccattattctgttaatgaggtaaattgcattgattcattttcaaatgtcaaagcaaccagtatgactggaataaaacccacttggtgatggtgttctgacctttttatatgctcctggttctatgttctattattttgttatgttcatgagaaacatttgcctgtaatttttttttttttttatgatgtgtttttgccaggttttggtattaggataattctggccacataaaatgatttggaaattatttctgcccctctgttttctgtaagagtttatacagaattggtactattttttccttaaatagttgacagactttcccagggaagtcactttggccaagagttttctttgtgggtatgtttttaatgaagaattcaatttattgaagagatatgatactattcaaggtttctgtttcttcctatgtcagttttggtaatttttgtctttcaaggaatttgttcatcaaatctaacttgttaaagtcattggcatgaagttgctcctatctttttatatccacttactgtttataaaatctgtagcgatatcctgctttttttacatcatatggggcacatattggtaatatgtgttcttttctgtttgtaaattgatcatcttagaggtttatcaactttttaaatcttttctaaaaacaaagttttggtttgattgattttctttattgttttatatattattaatttttgttctttattctttcttcactctctggttttgttttctagctacctaggaagcttacataattaatttcatatctttcttcttttccatcataagcatttatagctatacattctctctaaccactattttagctgtattcccccaaattttattgtttattatcattcaggtgaaaataatttctgatttcccttttaatttcttcttcgaacctgggattatttagaactatgttaattttgcaatactgggagattttctagatagttttttgatatttatttctaattcaattttgctatggccaagaatataattcatattaattcagtcttttcaaatgtttgagaattggtttatggaccaacctatggtataacttagtgaatgttctgtgagcacttgaaaaggatgtatgttctgcagttgttgggcattgtgttctgtaaatgtcagttatatttagttggttgatgtttaggtcttcattatttttactgattaaaaaaattttttttgttaattcctgagaaatgagtattgaaatttttagtgtaatagttgttttatttccctctttacatttttgcttcatgtaattggaaactattattgtgtgcatatatatttaagaagagtaatttataaatcttctatgattagtgtttttatgttctgcttacaagaggtcacattggtctgttttccatcttaaattaatatttgtgtaccgtatgaagtagatagaagttaaggttattttttgccccatatgaataaccagttgactaagccacatgttttgaaatgaccaatatttcttcactgaaatgcaatggcacatctgttccaaattaggtaactgtatgtgagtatgtttttttggactctattctgttccatttgtctatttgttcatttttgtgctaataccaatctgcttgttctatctttatgataaatcttgatgtctgataatataaatctttagtttgctcttcaagattgttttggctattgtaaatcctttgtttttcacagactttaggatcaacttgtaaatttccacaaaaactgctaggattttaattggaagtgggttgaatctatagattaatttggaaaagaaccgacatctgaaccatactgagtttttctgtattcttatatcagcatggtctatccctctcttatttagctagcatttattttttgtagaaatgttttacagtttttattgcacagtttttacagatatttccatagatgtattcctaggtatttgattttttgttactattgtaaatgatactatttttaaatgttcatttaaaaaattgtttgcttctgacatacagaaatgcagttgatttttatatattggtcttgcatcttgtgactttgctaaattcactcagtgattggtagattcttttaaatttcctacatatacagacatgtcctctatgaatactgacaattttacttatcctttctaatcatcataacttttatttagttttcttgccttattgcactagccacaatgtccactatggtattgaatagaaatcatgattttagacatgattgttacgtttttgaactctagaagaacaagatcaatattttaccattaggtatattgtttttttactgttaccctttatcagagtgaggaagtttgcttttattcctagtttgagattttaaaatcatgaacattggttaaattttattaggagtagtttctgcatctattgaaatgatcatatggtgtgttttctcattgtgctaataatgtaaaatatattgactatttaaaaaatttttttctaaattacataaaaattgtatatatggtgtacaacatgacattttgttatatgtatacattgcagaatggcaaaatcagtctaattaacatatgtatcacctcacatacttatcttttgtggtgagaacacttaaaatctactcttagcaattttcaagtatacgatatattgttattggtaatcaccatgatgtacaatagatctcttcagtttactcctcctatctaacagaaatttcgtatcctttgaccaacgtctcccattcctcctaatgggaccctcagcctctggtaactgctgttctacttactctctgcttctatgatttctacttttttagattctatggatcagtgagatcatacagttttgtctttctgtgcctagctttatttcacttaatataatgtcttccaggttcatccatgttgttgtaaatgacaagattttcttctttttttaaggctgaattatattctattgtgtacatatgctgtattttctttatcagttcatctgttgatggacacttaggttgattctttatcttggctattaagaataatgctgcaatgagcatgggagtgtagatattgctttgacatattatttttagttccaatacccagaagtgggattgctaaatcatatggtagttccattttaattttttgaggaacctccattcattttccatagtgactgtaaaaatttacattttcccaccaagagtatacaagagtttctttttctccacacactcaccaatgcttgttatccttatatctttttgataaggctcattgtaacaggcatgaggtgagatctcactatggttttaatttgcatttagtgatgttgaacgttttttcatgaacatgttggccatttacatattgtcttttttttttttttttgagacagagtcttgctctgttgcctaggctgaagtgcagtggtgtgatcatagctcactgcagccttgacctcccaagcgcaagtgatcctcctccttcagcctcttgagtagttgggactgcaggcatgtgccagcatgcctggctaatttttttttttttttttttttttttttttttttttttttttttttttttaggtacagggtctcactgtgttgcccaggctggtcttgaactcctggtctcaagtaatcctcccaccttggcctcccaaagttttgggattacagggttgagccactgcacccagtctgtattgtcctttgaggaatatctgttccggtcctttgcccatttttaaatcaggttatttgttttcttgcttttgagttgtttgcatttcttatctattttggatattaactccttatcatatggatggcttgaaaatattttctcctgttttataggttgtcccttcactctgttaattgtttcccttgctgtgtacaagtttttaaatctggtataatgattgatttctaaatgttaactttgcactcctgggggtaaattcatttggtcaagatgtattcttttaaagtgttattggatttaatttgctagtattttgtttagcatttttttccaaatatgtttatgagagagattccagtgtaatttttttccttttcatgtccttacatattttggtatcatgtttatactctcattttaagaatgtgggtaagtgttttttattttctatttcctgagagagtttgtataagctcaatgtttcttcctcagacatttggaaaaatttaccagcaaagtcactgggcattacattttctatgtggaaaagttttaaatggtatattctgagggctctgttctgttccattgatctatatctctgttttgctaccagtaccatgctgttttggttactgtagccttgtagtatagtttgaagtcaggtagcgtgatgcctccagctttgttcttttggcttaggattgacctggcgatgcaggctcttttttggttccatatgaactttaaagtagttttttccaattctgtgaagaaagtcattggtagcttgatggggatggcattgaatctataaatgaccttgggcagtatggccattttcacgatattgattcttcctacccatgagcatggaatgttcttccatttgtttgtatcctcttttatttcattgagcagtggtttgtagttctccttgaagaggtccttcacatcccttgtaagttggattcctaggtattttattctctttgaagcaattgtgaacgggagttcactcatgatttggctctctgtgtgttattggtgtataagaatgcttgtgatttttgtacattgattttgtatgctgagactttgctgaagttgcttatcagcttaaggagattttgggctgagacaatggggttttctagatatacaatcatggtatattcaatttttaaattactgtatttgtttgttctcatattgctataaagagctatctgagactgggtagtttttaaagaaaagttgtttaattgactcatggttccataggctgtacacaaggcatggctgggaaggcctcaggaaatttacaatcatggtagaaggtgaaggggaaaaaagtacatcttcatacagcaacaggaaagagacagcaaaggacgacatgctgcaaaaatttaaataaccagatctcatgcaaactcactcactatcatgagactagcaaagcgtaaatccatcccatgacccagtcacctctcaccaggccactcctctaacactgaagatcataattcaacatgagatttgggtggggacacagaaccaaaccatatcaattatatatagaattattcaaattttatatttcttcttgtgctagtttaggtaaattgttttaaaaggaacttcatttgtttttaaattggagaatattcctgttgtcttttacatgtttcaatattgtaagctctggtaataatgtctactttttaattcccgatattgataatttatgttttatcttctttttttcttgatcagtctttctagtgttttatcaattttaatgcttttaaaaagccaacgttttactttgcttttctttattttatttttatttttctctgatttctgctcttaagtactttcatttttatggtttttggcttaatttgttgcaacttcttgaaatgaaaatttatttaataacattgttcagcctttcttctcgtcttgtgtatgaatttaagattatgaatatcttagtatacacttttactgcgttttactcattttcattattgattcaaagtatttttaaagttattctttgtaccatgggttctatgaaaatatattgattaatttacaaatatatgcgggtttctagttatttttctgtatatgctgtggtacttttgtcagtggttatattttccataagagatttttaatttaggtgccattggcaaatttccattaattcaaatgcagaacttcacatttttcaatgcatacgtgagttttttttgttgaggtattaagaatagcataaatttataaaatattaagtaatcattgaatgcaaaatatgtcctattttgcaacttaatatacttttattaggttgacgctaaagtaattgcgttttttactcatactttaatggcaaaaaccacaattacttttgcaccagtctaataatgagaattattattattattatattgtcttttttttttttggagtggagtctcactctgtcacccaggctgaactgcagtggtgtgatatctgagccttggctcactgcaatctctgcctcccaggttcaagcaattctcctatctcagcctcccaagtagctgggattacaggcacgtgccaccacgcctggctaatttttgtgttttcactggagaacagggtctcactacattggcctggctggcctggaattcctgacctcaaatgatccccccgcctcggcctcccaaagtgctgggattacaggcatgagccaccgcgcctggccctaataatgagaattcttatcaaactgtttacacagaataacatgagtgggtaatggtgaagctgagtttcacatttttatgcgatattcttcagtgttgctatttgttcagtacttttaagaattatataaattttttatagtctatacataaagtcaatattatgggtcccaaatatataaaatgtttgcttgttccattgtttatgttttgaatacaaattatattttgtatgatttcattaataatttgttgagaattttatggcctaacatgtggtctactttctaaaatattccagcttcatttgaaaaggatatgcattcttcagttgttaggtgtttgcttttgattgcttgtatttctcagtcaaatgtaggaagtggagattaagaaaagggctggcacttttggtgattgaggaggagaaagtatgagatagttgtcttggatagtggaagagtaaatgttctaaagaaatataaaatgatagttgagcagcactaagggcccatttgagtatgaatctaaagagggaccaatctgaatgcatgtgttttcatcagtgatttttatatgtgtgggtaaaagctctgagtagacagagataaactgaactcttttagagtttctttgttatctttggtgtttggatcctttgctttgatttgtatcggtacagtttttaaaaaattaaatgttatctcctctcagcagatctttttaacatgaacccagcatctttaacaccaataaaatttcttctattatttttcttccctgtgttttcattcgtcctccatctagagcttctctttagaattgtactccctggattgatcctcttatttcttacttttatgctcataatatttttttctttctgtctgtctctttctctttctgagtctaggacctgggagtttgtcttgactgtcttccaaattatgagtttgatcctttgagtcgtattattcaactctttcactgaattgtcatctatctctttttctttaatgtatagagaatttattatgcacagccacttgcaaactttaacaagtatgattagagcagcattttctataacagcaacacataagttggaacaacacaaatcttaaaataactggaatggatgagcaaattgggtggtccgaggctcagacatcagcaggtgttaaacacctgtgggaaaggagagaacgatccatttatttatttctataggaaatgcaaaagaggcaaaattaagaaaaatactgcttcatgataaagaataaagccgtgaagaatatccacaaaattgtgtatagtggctacatctgatacagaatggaaacagccaggatagagcagcacaggaacactatgaaatgcccaggctgtcttcattactagtcaggatttttttttttaattttgtaaactttatttattctctgctcctttttcataatctagtgatgctttatgttttcaaaatattcttaaatctctctgagaataccgagaaagattttaaaccaagttatcttccctgcattatctgtttcttctggggtcagttgttttctggtccattttggtcttactcttttattcctacaacccctgccccatccccatcctaactgtaataatccttgataatcattgcatatttatgctttttatgtgtgtgcagacaactgtttttatgcttatagatgccttcctctgtgtattttttttcttcagttaaaattttttttttttgcagtcacagtcttgctctgttgcccaggctggagtgaagtggtgccaacctagctcactgcaggctcaaactcataagcttaagccgtcctcccatttcagcctcccaagtagctagtactacaggtgcacactacatgctggctaatttttagatttcttttttgtagagacagggtctcgcaatgttgccggggctagtcttgagctccaggctcaaacgatcctctcgccttggacttccaaattgctgggattggaggcatgagccatcgcacctagcctatgtatgttttaaatagagtctatttcctctactctgtcattctagtcctatctacttttgatcttcctggaattactgtaaatttctgatttgctgatagcaccttttcttatttgtcggagccgccatgaattcgtttatataaacttttaattttactccttttagctactgttgttacctgttttacttttatatctattttaagccctaccatacaccgttattattcatgctttaaacaattgtcagttgtcttttttttttttagacggagtctcgctctgtcgcccaggctggagtgcagtaaacatgacctccgttgcaacctctgccttcggggttcaagcaattctcctgcctcagcctcccgtgtagctgggattacaggcgtgtaccactatgcccggctaatttttatatttttagtacagacagggtttcaccatgttggccaggttggtctcgaactcctgacctcaggtgattcacccgcctcggcctcccaaatgctgggattacaggcgtgggccaccacgcctggctgtcagttgccttttaatacatttaataaatggaagaagaataatattataattaatcacatatttactatttttgagctcttcattcctttctgtaaacctgagtttacatgtaatatttcctttaatccacatgattttctttatcagttcttgagtgtgagtttgttggtgatgtactctctcagcatccatttatctgaaaacatatttgttttgccttgaattttgaaaattcttattttttataatacaacagatccagtagtatgagaattatttttcaggattgtagaattctgggttgacagctttccccctctccctaccagtactttgtcttctggcctccattacttctgaagcgatgtcagttctatttcttgtcttggttctcctgtatgtatgtgactgcttttaagattatctttttgtctttggtttttagcagtttggctataatgtgacttaagtatggatttgtatagattggattcagatgaactttttggatcggtggtttgatttttttaaacaaacttggaaaacatttagccagtattttttcaagtatttcctctgttccacttatatgtatcagactttgttactcttgttgttcagtggtgtgatcacggctttctgcagcctcaaactcctgagcgcaagtaatcctcctacctcagcctcccaagtagctgggactacgggcacagagcaccatgcccagcaaattaaaaaaaaatttttttgtagagatggggtcttgctatgttgccagggctagtcttgaactcctggactcaagcaatcctcctgccttggcctcccaaaatgttgggattaaaggcatgagccatgactcctggcctagatttcttgatattgtagagtttacatatggcttctttatcttcatgcattatattaattttttactgtaaattctattacatatgtatcatagtaaacaaagtctttaaaataaattctaatatttgggtctgctttgggtccatttctattatatatattcttcttttcatgtttaataagtttcagctgactattgtacattgtgggtgatacagtgtagaaactccggatgatgtggatttcaatctttctctgaagtaatgagttttgttctagcaggcagttaaatttactagcagaccaacttgatcctgcggaggcttggttttaggctttgttgggacaggtctatttcagttttgccattacttctggtaagtggctcttattcttcaagtgtggcttttctggcgtttcagtggaaagcctgagatatttaccagttccctctaacactaaatcctgactctccaggaccagatagctgttgaaatctcttcccagttctttcagatttctagctattgcctgtccctgggcttcgattttcatcctatggatgggaattcaggagtcaggccccactgactcccattttcctgagatttctgccctcaatttttggcttttctagcagctccaaacttcattctctgatactttcacccagtaagactgcagctttttacaagagctctgtcccctcctctggcccctgcctgtacacaacagactgtgtatgtagttgtctcaggatggaagaagagtaagtatgggcctcattcagtgtattccccttctttcaagggtcatatccccccgatttgtatctgcttttgtttctttgacaatgccttcaaatggtgtttttataaatatatatttttaaaaagtttataattgtcgtcatcaggaacattagtgtagtataaactactctctcattatcaaaaatctgtatgttaaaaattacttaaaattgttgttatttatcatttccttggtgtttggggcagaagggaatgcacatgatttttctgaacttctcattaagggaggcaacccagccttggtctggagatcccgaaattataccgtatgtataataaaataattattttgttatttttatgagcatagcattaataaattaaatatttagtaagatttgtatctcaggatttttctacctgctaagaatagatcgtttgacaaggcagatgaggttctttgttgaattataaggtattgaagcctataatttagtagtggaaagagcacatagcaagagttgtatgatgaggttggagaggtaagcagcaaacagataatggaattttagtctaaacattcgtaaaatatgagggtggctcctgtgggagattaggttattgggaggcaaagtagacttagggaaaatagttaggaggacattgcaatagttcaggagagaaataatagctgtttagtgtatgatggtagttgtggaaaaaaaagccaatggatggatttgaaatatagtttggaagtaaaactatcagtacatattgatggactggatataggggtgaaaaaagagaagaataaaggttgaccaaattctttattatctcaaagtatgaaagcttgttaagacctcttttctgtttgtattttctttcttgctcaataagaaaacaacccaaagcactttttttctaataacttatatcttcttcttttgtgataaaaaaaaataggagtggtctttagagtacaatacaaaattcttttacatttcacctcaccatatttaggtttaccaaagtagtataggaataattgtaggaaaaacatagtatatctacttggactataaaattttggtatactaaagaaagtaaatcaaaatggttatatcttgcaatagaatgcaaaatcacatggatattaaaggtaaacatttaaatgtattttatattcttggcttcttcttcagaccactccattgccttttgggacatttctgtggaaaagttgagaagcactgacctatctgaaccttccaatccagccaaacttgtctctactttgttccaggaacatgctgtgtacattcctctttccatgttttgcatgcttttctccctgccaataatgccctgtttttcattacttgtcaaaattctattttccttttaaggtccagcctatgttttaattgttgtgtgaaacttctaaaacctttctttgttttaaataattatgatcattttcttcttgtcattttgtagcacttaatttttaaaatttattttgtacttagatgatgtgtaatttactataaatcatagatttttaagaattgggaggaacagcggaaatttaattaagtaattttattatacaagtgaggaagctgagattcatttgtcatgtaagtagtttgcaataagaaaggatgaaacttatttttaggtctcttggctcccaggttagtaatttttctaatctactgcattacttcttcatgtatatttattgatctgaaaagttgtgtctcttagaattatgtagtatatgagtaaagtttgaaaatatgaacttttaaaatgtgatatttgaggtactggtttttgagtgtatgtgtacagttgattaaaatttattataacttttatttactctttctttttttttttttttttttcgtagagacaggttctcactatgttgcccaggctggacttgaactcctgagctaaagtgatcctctcaccttggcttcccagagtgcagggattacaggtgtgagccactgtacccagcctcttatttactctttattcaacagatttgttaatcaacacatgttgaccagacttaattacagcttttatatactcttcatttattcaacaactttcatgtgtcagagagtatcctaggcactgagtgcagtagtaaataatacattaaggttcttaccttcaagaaactttcattctagtggtacaagacaatgagcaaaatcataatctaatctatggtgggaaaatatcagaacagctgtggccacttgagtggttggagcagggactgatggagaaggagcatggaagaactttctgggatgctgataatttcttagggctttgggttacataggtatatgcagtgtccaaacttattgactggtacacttaagatttgtgcatttgattgtatttaggagggaaaatcctgtaaaactagaatagatgtaacttatatgagagttataaatgacaatatgttaaacacccatgaaactaccacccaccccaggaactagaatgttgccagcaatttgcacctacatatgtgtttctccctagctattctcctgtctatcttgcagaagtaaccattgtgtattgtattttgggattaacatttccttgattttttattttttttttgcgggggcggggggagatttgtttaacacatgtatatgcctaaacaatgtactattcagttttgcttgtttttgaaatttagaaaaacggtattttacagcttacagttttctataatttactgcttttgcaacattgtatttttaagatttatccatgttgcataaagctatagttcatttattttaacaactgaggaacagattctgtagtataaatgcacaaaagtatttacctattttcctgatagtttgggttgtttgcagtttttgccattggaagctgaggcgcaatgaatattcttgtaatgtgtctcttggtgtaaatatgcatgaagtttgtaactagacgtagaattgtagcagttctcagaacgtggtctctagaggatgcctgggtatcctaaaaacctattcaagggtttaatgaaattaaaatgattttcataataatgccaagatgttaaatgtctttttcattgagttgacatatgcacaaatggtacagaagtaatagtgagttaaacggctggcaccttagcacaaatcaaagcagtaatgctggttgtagttgtgatcattgtattctttttctttttcttttcttttttttttttttgagacaaggtctcactttgttactcaggttggagtgcagtggcatgagcacaatcactgctcactgcggcctcggtctcctgggctcaagtgatcctattgttttctttctcgtcatgtacttccaggaaaagaaaaaagccaattcgagtcacttattcttttgattctttttcgtagcaaagcatcaatgcattttctaaatgattgaattcatttaaattcgggaaacatttactgagtgcctatggaattctgggatatgagtgtatgtttaaggcagtgtgatttccagttaccatcctttctgttctaaacacgattgtgtcatattaaaatataaaaagagtaaaaaatatcctgaataaaaatagatcaagctgtacatatatatagattataagtgcaaaatactgagtgtggttaaagcctatgctatgcttgctgggtttcctgtacagaagtaggcattggagacagttttgctcttgtggtacaacatggatgactagaggcaggtccaggatgttacttttactgccagccgtcgcttccctccaaatagactattggaaggggaacctcccgccaaacgctgcctactgctatagtgttggataagttgcaagcttaggaagatgagatgacatgatttaggaacttcaggatgaagtgaagtatctcctgccttgtgactgactggatctgcatcgtatcagatacctcctcaaggcaggaattttaacctgctgttataagacctggttctatatatggatagcccaggtaggctaagttgtggcagccacagattgttagatgaggagggacaccacagactgtgtatgtgtgtgtgttgagggcaaggtgaggtgaggaatggtgcaatgagcctgcaaaccaaattgcaaaatatatgaagaaatgtagtgctaagaaaaatagtcaacaaaattaaaaattgaaaaatgaattttttcctgataaaatttaaaaatgtaagtaaacctctaattgaagtgtaacacacaaacaggaaagtatgctactcataaatgcacagcttaacaaattttcataaagtgaacacacctttgttaaccactacttagatcaagaaatagagtatcacctgaattactggcaggaggcaccccagaggtctccctgtacctcctgccagttattctccatgccctcccactatcccaaccctgccaagctaaccattatcctaactttttttcaccatgagaaagaaatggaaggatatggaagtgagcttgagaggcctccatggtcagtgaacagttctgctaaaataatttagagggaataacaaagaagtagcatttgacggcatagtagctgagaatttcctagaatagaaaaaagacatgaatctttagattaaaagtatgtagtcatttttgatcaagataaagaagaatctacatcaaaacctttccaaaaatattgcagaacattgctgctaatgcaaacaagttttaaactacttgagggaaagccatgacatgtaggagtgatagcagacttcatatttatgttaatagataccacaaaacaatgcagtaatatctttaaagttctggggaaagaaaattataaataggattttgagagtgagggcaacatatttttttcaaagactacaacttaaaaacccacagagccccactgaaaggtctactaaaagatacatttcagcaggaaaaagtgagtcctgaaggaagacccaaaacaatgatgagcacaaaacaatccaaagtcagtggtgggcacaaactgataaaattctagcaaatttactccttaatgctaagaacttcgtttaaattaactttctatcttttcagaaaaccaactcttagatttgttaatcttttccatcatttttatagtttctatgtcatttatttctgctgtgaacttcattttttccttccttctgttagctttgggctttgttcttctttttctagttccttgaggtgtaatgtaatgttgtttgacatctttcttcctttttgatgtaggtatttattgctataaacttccctcttataactgcttttgctgcatttaatactgactataataagatacgatgtaatagatttcaaggaattatgtatttttgaataaattaattctttaaagttgcatatccagttgcagatgaacttcaaaaatcttgcagttttatatctgttacagtaattgccaggttttgttgttgttgttttgatacattagaagttctagaattgttatatcctcttgatgaattaatccctttatcattctagaattaccttgtctctttactgtttgtgacttaaagtctgttgtatctgatatacctttgcatggaatatctttttctatccctttactttcagtctatgtgtatctttaaaggtgagatgaggttttgtaagtggcatgtagttgggtcatgttttttagtccatttagccattctctatcttttaagtggaaagtttaatctatttacattcaagtttattcttgatatgtgaaggcttattcctgtcattttattaattgatttctggttgttctgtaggtcctttgttcttttctttctctcatattgtttagcattgtggtttgttggttttctatagtgataacatttgaatcctttcttgtctgtgtgtgtttgctttaccagtgggtttgatactttcgtcatctgtttttcataatggtagtaattgtcctttttgtttgtttgtttgtttcttttttgagacagggttttgctcttgttctgtcctccaggctggagtgcagtggtgtgatcatggctcactgcagcctcgacctccatggtctcaggtgatccttctgcctcagcctctcaggtagctgggactacagaaacctgccaccatgcctggctaattcttttgtatttttcgtagacatggggttttgccatgttgtccaggctgctcttgaactcctgggctcaagcagtctgcctgcctcagccacccaaagtgctaggattacaggcttgagccactgtgcctggcctgacattgttctttgacttccatatgtagaactccctcaagcatttcttgtaggtctggtctagtagtgttgaattcctcagcttttgcttgcctcagaaaaactatttttcctttgcttaatgaaggataattttgctgggtatagtatccttgacttgcaggtttttttctttcagcacttttcatatatcgttccattctcttcctggcctgtaatgattctgctgagaaatctgctgttagtctgatggagcttcccttagaagtgactagactcttttttcttgctgtttttagaattctctctttgtctttgacaagctgttgtctctgacaacagttctctctttgtctttgacaaactgttgacagtttgactctaatgtgttgtggagaacctgttggaattttgtctttttggggatctctgagcttctgtatctgaatgtctaaatctcttgatatacttgggtagttttcagctattatttcattaaccaggttttctattccttttgtattttcattgtcttctagaatactgaaaattctaatattagtttgctttatggtatcccatatgtcatgcaggctttgttcattcttttttctttatttttgtctaatggggttatttcagaagacctgtcttcaagttcagaaattctttcttcgtagatgctctagaatgtattttttatttcattaaatgaattcttcagtttcagggtttcttgttttctttttaaatgatatctctctctttggtaaatttctcattgatatcctgagttgtttttctggtttctttgtattgtttatctgtatgcgtttgtatctccctgagcttctttaatatcattatttttaattctttttctggcatttcatgaatttcttttgcattggaatcttttggtagaaaattattttgatcctttggagatgtcatatttccctatgttcccatgtttcttgtgaccttacttctttgatatccacacatctggtgtaatcatcacttccatttttttgaatttgctttcatagggtaggactttttcctgaagatttgactggggtgtttgttggccagggcactttgggtttgaatctgggtgcatgcagtagtgtagtctctgtaagattttttttcctttgtaaacagcatcagtggtgtctgtgatttcctcagtggcatagtgtgtggttgtggaggctgtggtgaacttttgctggggatggtgacaccagctggactgatcctcagtcctcagttgtggcagcagttggacaaccatgcctgtacattagccccagggtggcttacattagtaatggtgttagtgggtccaggcagtccaatttttgggtctccaggtgacttgtttgggtaccaggagtggcagtgatgggctgggcagctgagtgggtccacaggcccctgggcagtgagcatggcatgggttatgtcagtagcagtggtaggagaacctctggctgtccagttgtctgtgcttatgtcggcagtgacggcgattggctgggtaggcaagtcctaaaacctgcaggtggcaagtgtgagtgggaaccagctgtggtggtagtggcaggttgggtgggccacatcctcagacccccaggtggaatgctcagttgacactggacgtggacaaactggtgcgatcccaaggcccccagataacatgcttggatacgtgggagtggggtgctgagctgggcagggtgagagtatcctcaggccctccagtggtgttagcaggtgctgtttgtggtgggcaggagcaggatgatttccaatttcctggtggaatgttcaggtgggggcagcagtggctgtgctgtgccctgatgctggggagggtgcagttgctgtcagtgggagcagttgtagggagttggctaaggagtgtgcactgcagctgcaggtggaggctgtagatgtgatgaagctgtactcagggtgcatgcaaatttgcattttgacacctagcggcagcagcctgcaatggtggcagctgtaggtggtagagcttgtcctcagggcacataccaatatatggcagcccttctgctgggagcagtggggttattgccaatggcttgtgctttggtcccagaggcggcagccagcaatggaggtgactgtcggtggaggatgtcagtggggctctaggggtgtggatatgcaggggctgttgggctccagggtaggaggcattctggtgtgggttgggctttaaaaatggcaccgtgctgtagctgcttaggactcaggggtgtgttggaccagcataagctccctctctaaagcaatgtcattgtgcagtctccaggcagctccctatgttactcccagggcccatgaaagttgacgggctctcttgtgtctgggattgcaggagtttgcagtgaaaatgtgggccactgggagtctctcacttactctttccccacattgtgcaggctctctaggcttctggctgatcctggctgagcaggctgccccacttccctctccttccttgcattaggtgttttctatcacttctctgttgaatttccgtgttctctcttagatgacctattcaaagtgtgattatctactcgctattttggttcttctttgtggagcaggtgagtaccagataactctagtcaaccttctggacccctcttcccccaatttgagatctcttcttctgttgtctgtaactgagtttaatgcttgtttgttcatgttaggattttatatcatcgtcctcaattaggttgttaactggaattttataatctttgtccacaggaagtttaaaatgtatgatttcttgcattgtgctttgtatgtagtaatacacgatatttatccagttaatggatttgacagccattgctgtcaaggagcagtccttctttgtgtatgaagggtgccttatcaatattatttccatttgtaactttatttatttatgtattcatttttgagacagggtcttgctgtgtcacccagactggagtgcggtggagtgcggaggtttgctgcagcctcatcctcccaggttcaagcaattcttccgctccactcccagagtagctaggactacaagtgcgtgctgccacgcccagctaatttttttcttttgtatgtttttgtagagatgaggtttcaccatgttgctgaggcttgtctccaacttctgggctcaagctatctgcccgcctcggccccgcaaagtgctaggattacaggtgtgagacactgcgcccagcccatttgtaactttattgttttctcttacaggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtaagtattaaaaaacagcattttcctttttatgcatggattgttttaattatgctatgctaatactaggtacatgcataatatatatttttatttttttaatgcttgtatggctggcgtgtgtgttttcacatgcatagaaaatgaaagtgttactggagtacaatttatggtgaatctgccttgggctaggtatcaaaacaaattgatgcccataaatgtttgctgactattctttataattacatataaggtggttaaaaaatcctgtcatgggctatctcatgacataaggtaatattaaagctagatcctgagagcaacatgaaagcaaaaaattaatgttttgaaatatgtatttgatttaaaatcttgattaaatggctgacaaaccatcattttcttctagttagcttctgaagacctctgccaatctttcctcatatatatttcttatgtctttggtgtatcttgaaatcatttttactcttgagacctgattatattatctatggaccaaaactgtgaaccagagaactctttctatagagctaacacattttactagaccactgaggttactatagagcctactatcatggtatctcatttttaaatgttcttttaatagaaagactagttgacactattttgaagaatagtcgtatgattttatttgtagaggcaaattaaatgtctaatatagtatttataagaatgtatgattacatcaatttattctcttatttaagatcagtataggtcagaataaccattaactgtaagttggaggaagctaggtacatttcaagttccttctagctattttgaaaaatacaatacattgttgttaactatagtcaccctactcttctatcaaacatttgaacttgttccttctgtctgtatttttgtgcccattaaccaacatctcttcatttccctcttcccacccatataaccttgccagtctctgctaccaatccactctaccttcatgtgatcaactttttagctcccacatatgaatgagaacttgttatatttgtctttcagttcctggcttatttcactcgacataatgaccttcagttccatccatgttgctgtgaatgacatgattacattcttttttacgatgccactgggccccaggggagtacgcattctgttgtgagctgggcttcaaaatatcaccttgctgtagctgcttaggactcggggggagcgtgggacccagtgtgaacttcctcactggaataggaaatgcttttgtgctgtctctgggcagccctctatgtgaattttttgtcctgcaaggggctagggtctctcctgtgactaaaattccaggggttcacagtaggaatgtggaccattgggcatctctaacttaacctttccctgtgtggagtctctcctagcttcttgccaatcccggctgggcaggctgtcttgtttcgtctccttccttgcttttctgttgaataccagtgttcctcttggataatctactcaaagtgtgattatatactcatggttttagtttttagtggggaagtggatatgagagacctctggtcagttatcttgactatattgtaaagtctgtattttttgttgtgtgtagtctctgaaatctctgatcctttagcttatagtaagattttagcttgtaagatttttttagcttttaagattttttagcttgtaagatattttttagctagtaagattttagcttgtaagatttttagcttgggccgggcacggtggcttacacctgtaatcccagcactttgggaggctgaggcaggcagatcacaagatcaggagatcgagatcatcctggctaatatggtgaaacctcatctctgctaaaaatacaaaaaattagccaggtgtggtggtgggcacctgtagtcccagctacttgggaggctgaggtgggagaatggcatgaacccaggaggcggagcttgcagtgagccgagattgcgccactgcactccagcctgggcgacagagcgagactccatctcaaaaaaaaatttttttttggcttgtaagattcatttttagcatgtaagattttgctttagcttgtaagatttttttttgacagattttattgaattcctagggccaaaagaaaataatagaaaagaacaaaaaactctttcagtctttgcagattggctctgtgttggggctctcattcagtgtttacccagtccatttacaactctgccttagccttcccttcctgcttgtgctgactctaaagaacagcttgaggtgaaagcttagggtcttttcttctaagttctgttccttgggcatgcatatggcttttctactctccaatacatagaaatgcttttcagtgctgtcatttcccaaataaactctcctctgtcatctctgttttggttttcagtgtgtttattgtttgcctcaactttagacctctgccccagatagcaacagcttgttcatttgcaatgttttcaatgaatatcttctgtgaagctgcttttctgccctgagaaagtcctgaattaagtggaacataggtgtgctctttgcatcatttcttcagtcagtccccagtcagggcaaaagagagaaaaacagttttttgagattaaggttggctctgttcactccagagccagggaccagagtttcacactgggagcaccaattgccatcttcaagactttgcttaactagggggagtggagccggggctggtgggcaggacatgggcgtagtgggtggttaagggaaagtaaaaatgctgcacaactttcctgccatttttaagtggctgttttcttgattcagcatttatctgattgctgcatatctttgcctattttctgtagtttatatgaagttcattctgacagtttttactcattttttttttcctgtttctgggagaaggataggtccttggaactacctactccaccattttcactgatgttactcttttacttttctaatcatggaagtaaagtttaagatttttctatctatattggtgagattacccttggtatcgtagtcaaactaaattcacaaaatgtgtcaatttctctgttcgctcttggtctattctctgtgatagtatacttaagatggaattgtctttcatgtttggttgaactcaaatataaatcttgtctgggcctgatggtggttttttctttcttcattgttattttgttattcttcttccttcccttcttctgtctctttcttgctccttctttcttttcttcttcttttgtttaagtggatagagtttgtttcctttttttttttttgagactgagtcttgctctgttgcccaggctggagtgcagcagcatgattatggcacactgccacctccgccttccaagttcaagtgattcatttccctcagcctcctgactagctggactacaggcacccaccaccacacccagctaatttttgtatttttggtagagacggggtttgccatgttggccaagcttatctcaaactcctgacttcaggtgatccacctgcctcggcctctcaaagtgtgtgagccactgcacccagccagaatctatttctttaatggttatagttctgttgaagttttaaattaatgaatctttttaaagttacattttcacaaaaattgtccatctgtctatattttcaagtttattggcataatgttgttctagcactctttgtcttcataacttataattatgctttttaaaagttcttagtgtttatctgtgcctctttttttcttattatccttgacaaagatctattttgcaaagaatcatcttttattttgttacttctccctattggatttttatcatgtccatctctaacctttctttgggattactctattgtcttctactatattgagttagatacttagatcatttgctcttattttctaatataagcagataaagctacaatttttcctcttgagtttcaccttagttgtattccacaatttttagtacatgatattggtaatcattattcattataaatgcctcccaatgtatttttcctttgacttctgagttatttggaagttatttgtaaataaaagaatgaatgcaagtatactgtttaaaagttgctgctaaataatgttattataaataacttaccgaaattattacacatctgtaagtagacggtcgtgttaataatttaaattaagaatttaagtctttcttgtcagtttgtcatttgtgccgtttctatctacagtggaaaatagtgaaaacataactagaataaaatacttttgtgttctgtgagccttatgttttaaatatataggattaaaaaaattttacatattcatttctgatttaattgaagtgtggttagagaacatggtatgaatggttattctgtggaacttctaaagacttgttttgagttcacgtgattgttgtgtttgtgcttgtgtgtgtacgtgtatgcgtatgtgtgaatgttctatatatgcttgaaaatagcttgtattatttacttgtgtggtttaggtattctcttggttttttagttcagtggctttcaaccatttgttgatgtaccttctaaaatatcttttaagaaattacatatgcactcacattttaagttgatatttaaaatttttcattgtaaattaaaatagcccaaaggacataattattaatgtattgtgtaaaacttgacacattgttcttttaagagtattcatgacttctaaatatttttgtgatttgatacctattaattaaaacaactccttctgtgcaaccacatgcaagcatggatttttattatacctccagttcagggaaacttgaaaatttgaaccaacctttctatagaggacaactgaagaaaccagatgaaatgttttaaaaatatctgcttgaaagagtctcagcagttatattttaaaatactgacacacctcggagatattgtgggtttggttccagactatcgcagtgaagcaaatattgcaataaagcaaatcacatgaagttttaggtttcccagtgcatataaaagttatgtttaaattattctgtagtctgttaagtatgcaataatattatgtccaaaaaagtacataccttaatttaaaaatactttattgtcaaaaagcactcataatcatctgagccttcagtgagtcataatctttttgttagtggagagtcttgccttgatgttgatggctactgactgaccagggtggtggctgctgaaggttgggtggctatggcaatatcttaaaataagacaatgaaggttgctgcatcagtggactcttcctttcgtgaaagatgtctctgtagtatgcaatgctatttgatagcattttacccgcagtagaacttctttgaaaataggaagttgattcactcaaaccctgccactgctttgattaagttgatgtagtattataaatcctttgttgtcatttcaacaatattcacagcatcttcaccaggaatagattccatctcaagaaaccactttctttgctcatccataaaaagcaattccttatatgtccaagttttatcgtgagattacagcaattcagtcacgtcttcaagctccacttctaattttagttctcttgttacttctaccacatctgcagtgacttccttcactgaagattaaatccctcaaagatacccatgagagccagaatcagcttcttccaaactcctgttaatgttgacattttgacctcctctcatgaactacaaatgtttttcatgacgtctagaatggtgaactctttccataagtttttttatttactttgcttagatctgtgagaggaaacactgtgacagccataaccttactaaatgtgtttcccaaataataagatttgaaagtcaaattgctccttatccatgggctgcagaatggatgttgtgttagcaggcatgaaaacaacatgaaattcaatgtacatcttcatcagagctcttgggtgaccaggtgcattgtcaatgagtaataatacttggacaggaatctttttttctgagctttaggtctcaacggtggcttaaaatattcagtaaaccacgctatatacagatgtgctatcttctaggctttgttgtcccatttatagagcacaggcagggtaattgagcatagttcttaatggccatagattttcagaatggtcaataagcattggcttcaactaaaagtcacttgttgcattagctcctcagaagagagtcattcagttctttgaaactttgaaggcaagcactgacttctttctagctatgaaactactcctagatggtatcttcttccaatagaaggctgttttgtctacactgaaaatctattgtttatcagtgatctttgttagatctttggataacttgctgcatcctctccatcaacacttgctgcttcaccttgcacttttatgttagggacatggtttcttttcttaaacctcatgaaccagcctctactagcttcttgcttttcttctgcagcttcctcacctctctcagccttcacaggtttgaagacaattagggccttgctctggtttagggtttggcttgagggaatattgtggctggtttgatcttctatctagaccactcaaacttttgtcacatcatcaataaggctgttttgctctcttatcatttatgtatttactgcagtcgcatttttaatttcattcaaaaactttttctttgcattcagaacttgattgactggtgcaggaggcctggcttttagcctctcttggcttttaataataccttcctcacttagtttaattatttctagcttttgatttaaagtgagagatatgaaactctttaatatatgaaactcttcctttcccttgaatgcctagaggccattgtaggattattaattggcctaatttccatattgttgtgtctcagggaatagagaggcctaaggagagggagagacacaggaatggctgttgctggagcggtcagaacacacacagcatttgttgcttatgtttgtcatcttatatgggcacggttcatagcggccccaaatgattacaattgtaatgtcaaagatcactgatcacagatcatcataactgatataataataaaaaagtttgaagtattgcaagaattaccaaaatgtgacatagggacacaaagtgagcacgtgctgttggaaaaatggtgctgacagacttactagattcacacttgctctaaatccttattttgtaaagaaaaaaaccccacaatatctgcaaagtacaataatgcagggtgcactaaaatgaagtatgtttgtataaaaaaagattgtgattaataattcagttgtaggggttggtatatcaaaagatttcgactgctattacattttgttccgattttaagagttgttactttagcttcatttcaaattataggtgctatgtaaaaatattctgttgtgtacctttaataattcctttctctgcttcttttctaggtatcacacctgtgtatcataatatgtttgctttaatgagtgaaacagaaaggatctggtatccacccaaccatgtcttccatatagatgagtcaaccaggcataatgtactctacagaataaggtactttcttcagtaaagtaactcacttaatgctaaaaggcaaaatgggagaaattatcaaatattttttaattgcaaggtacttaataccagatacctgaaccaattagttagcactcatttaagatttcatttaagattctattctgtttctccatcagagaaactgtttatacatgttgtgtaagtagaatactctgagaataaagctgttttggtgggatgccacttaactccttatgattataattaaataagtaaacaaaatcacagtggcctttattaacaaaatcctaaatgtttgggatattgctggtttgtgcagcgttttgtgcaatagaatgcaatttcacatcttatctttataaagaactttttaaaaagttgacgtgaagaaaagagacttttcccagtgtgtatttgcagcctcaaattgtaagttaatttatataatttatggtaatgtgtaatgcctatttagagaaatctagaagtatttttttgctcgaaaggatgaaacaaagatacgtttgatttgtgtctcacaaatacctgtgatttttctaagcatttatatacatatatatataattcatcataacttcatgaggtgttaagatttccacattaaagataaagaatttggagcactgagaagtaatattactcaaatttattcatggtcacacaggtaggaattgggagtcaggttcaaatccaagcctggtttgcttcataagcttgcctcttaatcacctcatgtgtacttcattaaagagataatgtatatataatatttattattgtgcctatcacttggtgctctctcaataaattttagtattgttttgcttctttctcttcccctattctatataaaaatatgcagttggatgtgtttgcatttactgatgtctaatgtataatttgcactgagttcaaaaaaatattttttatataattattgaaaagacaattggtgaaaaaaataaatgttcaaagaaataatagatgtacaaaaatcaacagcattcttatatgcaagcaaccagttatataatagataaagaagatcctatgcagtgtagctacaaaaacccttaactaaccaggaatggtcttaataagaaaaaacagaagacagtataggaaaattagaaagttatttgaagaatatgaagaattaggacataacattggaatggggagatggaatattacaaagatcaatttctcttatgctatatccttagctctaagccaatcaaaatcctaacaaaggcattttacggaatttgacacattactatgtaaattgagaattattaaataagatagccatgagaaagaagacttacctggcaattgtaaaatattttgtcccaggagatattttcacattatatgaaactacaaaatggtatctattattaaagccacataattaaattcagaatcataaactaaataatgagaatgatggggccagtatatcttgaaagtactccttcatacatatagaaacataataatatgaagaaatcttttcatattaacgtggaagggagagattagtcaacaaatggtggtagaatagttgattagctgtttgaataaaattttagcttcttactttataatgtatatcagataaattatagattgattaaagatcagattgatatgtaaatagaaatgtaaagaaaatcaaactataaaagtctgaattggaactgaattcaagtccttgggaaaataccatttaagttgcaaaacaatagaaaaaatcatatgggaatgactgcaattgtcaactataaaaattttcaaatgtctgaaggtttcctcttaaaatgaaaaggcaggagggtggagccaagatgactgaataggaacaggtccagtctacagctcccagcatgggcgatgcagaagacgggtgatttctgcatttccaactgaggtgtaccgggttcgtctcactggggagtgtaggaaagtgagtgtaggacagtgggtgcagcacactgagcatgagccgaagcagggcgaggcatcgccacacccgggaagcgcaaagggtcagggaattccctttcctagtcaaagaaaggggtgacagacggcacctggaaaatcgggtcactcccactctaatactgcgctattctaacggtattagcaaatggcacatatcccgcgcctggctcagagggtcctacgcccacggagcctcgctcattgctagcacagcagtctgagatcaaactgcaaggtggcagcgaggctgggggaggggcgcccgccattgccgagggttgagtaggtaaacaaagcagccaggaagctcaaactgggtggagcccaccacagctcaaggaggcctgccggcctctgtagactccacctctgggggcagggcatagccaaacaaaaggcagcagaatcctttgcagacttaaatgtccctgtctgacagctttgaagagtagtggttctcccagaatgcagctggagatctgagaacggacagactgcctcatcaagtgggtccctgaaccccgagtagcctaactgggaggcacccccaagtagggggagactgacacctcacatggccgggtactcctctgagacaaaacttccagaggaacagtcaggcagcaacatttgctgctcaccaatatccgctgttctgcagccactgctgctggtacccaggcaaacagggtctggagtggacctccagcaaactccaacagacctgcagcagagggtcctgactgtcagaaggaaaactaacaaacagaaatgacatccacaccaaaaccccatctgtatgtcaccatcatcaaagacaaaaggtagataaaaccacaaagatggggaaaaaacagagcagaaaaactggaaactctaaaaatgagagcacctctcctcctccaaaggaacgcagctccttaccagcaacggaacaaagccggatggagaatgactttgacaagttgagagaagaaggcttcagacgatcaaactactccaagctaaaggaggaagtttgaactcatggcaaagaagttaaaaaccttgaaaaaaaattagacaaatggctaactagaataaccaatgcagagaagtccttaaaggacctgatggagctgaaaaccaaggcacgagaactacgtgatgaatgcacaagcctcagtagccgattcaatcaataggaagaaagggtatcagagatggaagatgaaatgaatgaaatgaagtgagaagagaagtttagagaaaaaggaataaaaggaaacaaacaaagccttcaagaaatatgggactatgtgaaaagaccaaatctacgtctcattggtgtacctgatggggagaatggaaccaagttggaaaacactctgcaggatattatccaggagaacttccctaatctagcgaggcaggcaaacattcaaattcaggaaatagagagaacgccacaaagatactccttgagaagagcaactccaacacacataattgtcagattcaccaaagttgaaatgaaggaaaaaatgttaagggcagccagagagaaaggttgggtaacccacaaagggaagcccatcagactaacagcagatctcttggcagaaactctacaagccagaagagagtgggggccaatattcaacattcttaaagaaaagaattttcaacccagaatgtcatatccagccaaactaagcttcatatgtgaaggagaaataaaatcctttacagacaagcaaatgctgagagattttgtcaccaccaggcctgccctaaaagagctcctgaaggaagcactaaacatagaaaggaacaactggtaccagccactgcaaaaacacgccaaattgtaaagaccatcgaggctaggaagaaactgcatcaactaatgagcaaaataaccaggtaacgtcataaagaccggatcaaattcacccataacaatattaaccttaaacgtaaatgggctaaatgctccaattaaaagacacagactggtaaactggataaagagtcaagacccatcagtgtgctgtattcaggaaacccatctcacgtgcagagacacacataggctcaaaataaagggatggaggaacatctaccaagcaaacggaaaacaaaaaaaggcagggattgcaatcctagtctcggataaaacagactttaaaccaacaaagatcaaaagaaacaaagaaggccattacataatggtaaagggatcaactcaacaaaaagagctaactgtcctaaatatatatgcacccaatacaggagcacccagattcataaagcaagtccttagagacctacaaagagacttagacttccacacaataataatgggagactttaacaccccactgtcaacattagacagatcaacaagacagaaagttaacaaggatatccaggaattgaattcagctctggaccaagcagacctaatagacatctacagaactctccaccccaaatcaacagaatatatattttcagcaccacaccacacctattccaaaattgaccacatagttggaagtaaagcactcctcagcaagtgtaaaagaacagaaattataacaaactgtctctcagaccacagtgcaatcaaactagaactcaggattaagaaactcactcaaaaccactcaactacatggaaactgaacaacctgctcctgaatgactactgggtacataacgaaatgacggcagaaataaagatgttctttgaaaccaacgagaacaaagacacaacataccagaatctctgggacacattcaaagcagtgtgtagagggaaatttatagcactaaaggcccacaagagaaagcaggaaagatccaattgacaccctaacatcacaattaaaagaactagaaaaccaagagcaaacacattcaaaagctagcagaaggcaagaaataactgagatcagagcagaactgaaggacatagagagacaaaaaacccttcaaaaaaatcaatgaatgctggagctggtgttttgaaaagatcaacaaaattgatagactgctagcaagaataataagaaaagagagaagaatcaaatacatgcaataaaaaatgataaaggggatatcaacccgatcccacagaaatacaaactaccatcagagaatactataaacacctctacatgaataaactagaaaatctagaataaatggataaattcctggacacatacaccctcccaagactaaaccagaaagaagttgaatctctgcatagaccaataacaggctctgaaattgaggcaataattaatagtttaccaaccaaaaaaagtccaggaccagatggattcacagctgaattctaccagaggtgcaaagaggagctggtaccattccttctgaaactattccaatcaatagaataagagggaatcctccctaactcattttatgaggccagcatcatcctgataccaaagcctggcagagacacaaccaaaaaggagaattttataccaatatccttgatgaacattgatgcaaaaatcctcaacaaaatactggcaaaccgaatccagcagcacatcaaaaagcttatccaccatgatcaagtgggcttcatgcctgggatgcaaggctggttcaacatatgcaaatcaataaacgtaatccagcatataaacagaactaacggcaaaaaccatatgattatctcagaggcagaaaaggtctttggcaaaattcaacaacccttcatgctaaaaactctcaataaattaggtattgatgggacgtgtctcaaaataataagagctatctatgacataccgacagccaatatcatactgaatggacagaaactggaagcattccctttgaaaactggcacaagacagggatgccctctctcaccactcctattcaacatagtgttggaagttctggccagggcagtcaggctggagaaggaaataaaggttatttgattaggaaaagaggaagtcaaattgtccctgtttgcagatgacatgattgtatatctggaaaaccccatcgtctcagcccaaaatctccttaagctgataagcaacttcagcaaagtctcagcatacaaaatcaatgtgcaaaaatcacaagcgttcttacccaccaataacagacaaacagagagccaaatcatgagtgaactcccattcacaattgcttcaaagagaatacctaggaatccaacttacaagggatgtgaaggacctcttcaaggagaactacaaaccactgctcaatgaagtaaaagaggatacaaacaaatggaagaacattccatgctcatgggtaggaagaatcaatattgtgaaaatggccatactgcccaaagtcctttatggattcaatgccatccccatcaagctacagatgactttcttcacagaattggaaaaaactactttaaagttcatatggaaccaaaaaagagcccacattgccaagtcaatcctaagccaaaagaacaaagctggaggcatcacgctacctgacttcaaactatactacaaggctacagtaaccaaaacagcatggtactggtatcaaaacagagatatagaccaatggaaagaaataatgctacctatctacaaccatctgatctttgacaaacctgacaaaaacaagaaatggggaaaggattccctatttaataaatggtgctgggaaaactggctagccatgtgtagaaagctgaaactggatcccttccttacaccttatacaaaaattaattcaagatggattaaagacttaaatgttagacctaaaaccataaaaaccctagaagaaaacctaggcaataccattcaggacataagcatgggcaaggacttcatgtctaaaacaccaaaagcaatggcaacaaaagccagaattgacaaatgggatctcattaaaccaaagagcttctgcacagcaaaagaaactaccatcagagtgaacaggcaacctacagaatgggagaacatttttgtaatctactcatctgataaagggctaatatccagaatatacaatgaactctaacaaatttacaagaaaacaacaaccccatcaaaaagtgggcgaaggatacgaacagacacttcttgaaagaagacatttatgcagccaaaagacatgaaaaaatgctcatcaccactggccatcagagaaatgcaaatcaaaaccacaaagagataccatctcacaccagttagaatggcgatcattaaaaagtcaggaaacaacaggtgctggagaggatgtggagaaataggaacatttttacactgttggtgggactgtaaactagttcaaccattgtggaagtcagtgtggcgattcctcagggatctagaactagaaataccatttgacccagccatcccattactgggtatatacccaaaggattataaatcatgctgctataaagacacatgcacacgtatgtttattgcggcactattcacaataacaaagacttggaaccaagctagatgtccaacaatgatagactggattaagaaaatgtggcacatatacaccatggaatactgtgcagccatgaaaaatgatgagttcatgtcctttgtagggacatggatgaagctggaaaccatcattctcagcaaactatcgcaaggacaaaaaaccaaacactgcatgttctcactcttaggtgggaattgaacaatgagaacacatggacacaggaatggaacatcacataccggggcctgttctggggtggggggatggaggagggatagcattaggaaatatacctaatgttaaatgacgagttactgggtgcagcacaccaacatggcacatgtatacatatgtaactaacctgcatgttgtgcacatgtaccctaaaacttaaagtataatagaaaaaaatgaaaaggcaaattgtaaagatagtttcaacaaatcaaataaacataaggttagtattcttgtaagtatgaaatttaaaacagtgtatccatttaacaattacttaatgtacatgaatatatagcgtcactagttatgagaataatgaacgagattgcttgctttagcacatagttacaactattcattattcttataactagtgatgctgagaattttttgaaaaggccattaatgatttgttcttcttttgttaaatatttcttcatgtacattaaataattgtttaatggatacagtgtttttaatgccaacaaattagaggatcaagatgaaatggacaaattcctagaaagacacaagttataggaactgctaaaagaaagaacagaaagtttgaataggtctatgacaaataaagaaatcaaattagcaatttttataaaagcccaggcccaagtggcttcacttgtgaattctatcacacattaaaaaaattattactagttattcacaaaaaatagaaaaggaaggaggatttctgagctctttctgtgagttcagtattaccctgataacaaattcagacaaagatgtctagaagaaaagaaaactacagaccaatgttgtaggtagaaaatcaacaaaatacttgcaaaccgaatccagcaatacagcttgaatatgtcttatctgaaatgcttgggaccagaagtgttttggattttagatttttttttttttttttggattttagaatatttgcagagtatacgctggttgaacatacctaatataaaaatctaaagtctgaaatgctctgatgaatatttcttttgcacatcaggttagcgctctaaaggttttgaattttggagcatttcggatttcagattttcaggttaggaatgctcagtttgtttataaaaagatgaaatactatgaccaggtgggatttatcccaggaaatacaaggctagtgtaacttccaaaaatcaatcaatgtaatacaccacattaatagaataaatgaccaaaaccacatgatcatcccaagagacacagaaaaagtatttgataaaatccagcatcccatcattataaaaaaactcaacacaataggaatggcaaggaacttcctcaatttgttaaagggcatctgtaaaaaattcaaaagtaatgtcatacttattagtgaaagactgaatgccttttccctatgatcaggaatgagatgaggatagctacttttaccactttgatttaacactatactgtagattcagggtagggaaattaggcaagaaaaagaaataaaaggcatttatattggaaaataagaagtaaaattgtagtcatcccgctgtatccatgggtttcacatccatggattaaaccaactgcagattgaaaatacttggaaaaaaagttgtgcttacactgaacatgtacagactttttcttgtcattactccctaaagaatacggtataacaactatttacataacatttacattgtattaggtattataaatagtctagagattttaaagtatactgaaggatgtgcacaggttatatgcaaatactatgcctttgtatatcaggaacatgagtatttgcagattttggtatgtgtgggaggttctggaactagtactgcatagataccaaaggatgattgtgtattcacagattacatgatcttgcatattaaaaatcctaaggaattaataaaaaaactattagaactaataaatgagctcaggtcagttgcagaatagaagatcaatatataaaaatcagttgtatttctatacagagacaaagaacaatccaaaaatgaaattaaaaaaatttcattaacaatagcataacaaatttttaggaataaacttaaagaagtgtaaaatgtatacactgagaaatataaaacatcattgaaagaaattaaggaaacacctaaataaatggaaagatatctcatagaggacttcatatttttaagatggcaatactccttaattgatctgtatgttcaatgcaattcctactgaattcctaaatgtctttattgcagaaattgacaggttaatcataaatttatataaaaagcaaagggacttagcatagctaaaacaatcttgaaacaatacaaagtttgaagacttatacttcctaattacaaaatttattacaaaaggactgtaatcaagacaatgtgatactaaaggatgaatatacagattaatggaaaggaattgacagtctcaatataatttctcacattgatggtgattgatttttgatcagggtgccaagacaattcatgggcatagaatagtcttttaaactaacagtgcggggacaactggatatccacatgtaaactaatgaagtaagacaatctacttcacactatatacaaaaaaaaaaagcttaaagtggatcataggcctatatatgagagctaaacttattaaaactccaagaagaaacacagaagtctttgtgtacttagattaggcagtggtttcttagatatgacaccaaaagctcaagtgacaaaagaaaaatagttaagttaggctttatcaaagttaaaaacatctgtgtgtcaaagcacactatcaaaaatgcgaaaagaccacccacaaatgggttccacaaaatggaagcaaatgtttgggaatcatatatctgataattgtcttatctccagaatataaaagaattcttacagctcaacaagacaatgacaacttaatttaaaaagtaggcaagggacttggatagacatttttccaaataagatatacaaatggccaaaaagcatgtaaaaagatgcccaacaggtgggcccctgagcgggcgtccgggaccgtggtgtgccaggcgccttccgcccttaacatgcggtggctggcccaggcggtgcaggagctggagcggggcccgagtggggcgccagagccccagcgcgagcaggagaggggccggagcgcggatccgcgccgcgctgctgaagcctggccggccacccagacgctgccggcagcctggccatggcggagccaaggaaagaatctctatacaaacaaatatgtggctatcaaattggagctgatcaagttccggaccccgcagctgcacctgggtaccggttctacaagcagctcagcgccatagagggcgtccctcaggtctactacctactacagcctggaggacctgttcgaccttcacgcccaagacggtgctcctgatcgccatccggctgatcacgcgcatggattatgtgcacaccaagaacctcatttaccaggacgtgaagcccgagaacttcctggtggggcgcccggggaccaagcggcagcacgccatccacatcatcgacctcgggctggccaaggggtacactggtctcaggaccaagaagcacatcccgtgcagccagcacaagagcttgacaggtacggcgtgctacatgagcatcaacatgcacctgggcaaggagcagagccactgcaacaacctggaggtgctgggccacatgttcatgtacttcctgtgcagcagcctcccctggcaggggctcaaggctgacacgatcatgagcggtacagaagatcggggacacatagcgcgccacgcccatcgaagtgctctgcgagaacttcccagaggagatggctacgtacctgcactacgtgcggcgcctggactttgagaagcccgactgtgactacatgcggcgcctggactttgagaagctcgactacgactacctgcggaagctctccagctacctcttcgaccgaagcggcttcgtgttcgactctgagtacgaggggctcgggaagcccttggcgacccccatcagcagtgtccacaccgacctgccctcccagcctcagcttcgggacaaagatcagccgcacagcaaaaaccaggcgctgaactccaccaatggggagctgaacgcggacgaccccatggccggccactccagcgcccatcagggcgcctgcagagatggaggtgggcgatgaaacgaaatgctgctgtttcttccccctgaatcttctccgtgcggccccttggggagcgagcttgtgtgagggccttggggcccacccacagcggcccagggccagacgctggctggaagccaggacacagactgcagggtcttggccggcggccccatccccgggacgaggggtcacttccttcacgtaagactggccaaaatttcttttttttttttttttttttttttgagacggagtctcgctttgtcgcccaggccggactgcggactgcagtggcgcaatctcggctcactgcaagctccgcctcccgggttcccgccattctcctgcctcagcctcccaagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggccaagactggccaaaatttctacacctgtgtctagtcctcccctccaagagcattaactatttaaaacaaggaataaaggaaaaaaaagaaaggccccctccacccccacccctccattactttgctgaagtgagtagtgggatcctggaggcccccagggctgaggcccagccagctgcccccgttagcgtcataaagtccagcttgtctccctcatccaaaggccgttttctcagtgggagggcaggcctggcctggaggggtgctgtggggccatcttgcccaggcccacctgggagggacacaggcattgctgccaggggtgaggctgtgccccaggcctccccaaaactaaaggggaacggaggggtggggccgcggctgaagccagccccgcaaccaaaatgctgcaccaaagctcggtcgccacaggcacggccaccgcagcctttcacagcctggccccggcgaggggcaggtgggccctgctaggagggtgcttctcgaggcacctgttccccgaggctgtgctccgaccctcagaagctgaggggggtgggccggctggcgtcgcgcggctcggcccctgggccctgctgtgtggaggcgcgcgggctcgtggcttcctgtctctgtgctccgccccctggcaagcagccgcagacaaaatgccttaaagcccccgacccagccccgcaggtgtatgtgcaggggggtctgcgggcggccctggactggctggcggactcccagcgggtcagcttgaggcagtgcccagggcggtggccgtgagtctggtttttgctttaccaagtgtacggaaatggcgtttacgtttctctgatgctcttttgaagccatacaacttaggggctttaaaaaaaaaaaggaaaaatgaaaccctccgaaaaaaagatgcccaacatcattggtcattagagaaatacaaatcaaaaccacgatgggataataataggtctacaaggatggcaaaaatcagaaagacaaacaataacaagtgttagagaatttggactaatttggatgcagtatattgctgttgggaatgtaaagtggtgctgctactttggaaaacagtttggccattccttaaaagggtaaacaaagagttaccatatgactcagcaattctacttctagatatatatgcaatagaaatgaaaacatatccacaaaaacttgtacatgactcttcatactagcgttattcacaatagaaaaataatagaaacaaacccaaatgtctgtcaggcgatgaatggatacagaaaaagtggtatattcatacaatgaaatattaatttggcaataaaaggaaatgaagtaatgatacatgctctaatgtggatgaaccttgaaaacattatgctgagtgaaagaaggcagacagaaaggccacatgttctaaaattagattgtgatatttgtacaatgctatgaataaactaaaaacattgaattttacactttaagtgggtggactttatggtatgtgaattatatctcaataaaggtgttactaaaagacaatatagagtgttctttgtttctctgaaggacaagtttttaacttccatttctccaaataagcaattatttgtttttataccaaattagcattttcagattccctactgatgttactggattaagctatgtttttaactttttgttaatttctacattttttgtttcctactattgcttctacattttgtgcactgaaggaggtagtatattgattttacacttacatttcttgtactttctgcctatattagtgttatccacatatccttttgtctcacatttatatttaatactgttagctgtgttttctaagtatgggataatacctttcagtatgctgtaggtgactatatatagatagtacgtttgtatttgaactatttggaagctgaccaaatgtttttattatcttgtagattttactttcctcgttggtattgcagtggcagcaacagagcctatcggcatggaatatctcgaggtgctgaagctcctcttcttgatgactttgtcatgtcttacctctttgctcaggtatgattatattatcttacttgtacatgagttaaatgataaatatcttgctgtttaataagtcacttaatcaggaaaaactttacatatgggaaaattgcagttctgtcttgcacagcaggtgcagagaagtaacaaaattgagtcttttagcactagtttttaatcccatggtttatggataatagaatctattttataaagtttttttggactgatccttttgaatttctttcttttttaggactttagatcattttaaggatttcgcaaagtatagactgaggtttacaacatgattcatgtagacatacgtggctaaaatattagtaacatttctttatggtttttaaaaaaatttttggacaatttcaggcttacacaatggacaatgaactgtagagaaagtaggttacataaaaaaatgagctctgatagtttgtttagctgtttataggaaggtgactggatgttgatacttagtctaagagctatattcaagttatttgttcactaatacagttatgtcaaatttttgtattgtgttggtatttcataatgctttcttctctattaccttgatttaaacagtaattgaaatattttgtcattaaataacttgtttttatgtgctgctagactagtaggaatagaaattaaaggaagaaaatatattagtgctcttgggtaaaaataggcacttggtatccccaaggtatcaggagttctaaatcagatttgctaatattgtaggaaacaagcccacagatcctcatggctcgcatttctgagaggaagcctagtaagcctagttcagtttatgaacaacaaaaggaaaaacatttctctattggggtatgagaccaacaaactttatttttattgtggtaagagatatataataaaaatgaccattttaaccattttaagtgtacagttcagttgtattaagtacaaatacattgctgtacaaccatcaccaccatccatctccagaacatttttcatcttcccaaatggaaacttcatacccattaaataataactctgtatttcccactcccaatggccttttgcaatcaccattctactttctgaatataaatctgccttttctatgtacctcatacaaatggagttatatagcatttgtccttttttgactggcttatttcacttagcataatgtctgtcaggttcatccatgttgtagcatgtgccagaatttctttcctttttaaggctgaataatattccattgcatatgtatactgcattttatttgttcattcatctgttgatggatacttgggttgctgctgtgaacatgggtaaacaaatatttccttgagtccctgttttcagttcttttcaatatatacccagaaatggaatcgctggatcatatggtaaatttataattttttttgaggaactgctacattgttttctgtagtggctgcactatttttcagtcctaccagcagtgcacaagtgttccaatttctcaacatcctcaacaatgctttgctttgtttgataatagccatctctatgggtttgaggtggtatcattgtgattttgatgcatacttctctaacaaccagtgatcatgagcatcttttcatatgcttgttggccatctgtttatcttctttgcagagatgttgcttgttggctatttgtttatcttctttgcagagatgtctactgaagtcctttgcccatttttaaatcaggttgtttgtttgttgttatattatagaaattattattctggatcttaactccttataagatacatgatttgtagatagtttttcccattctctaggttgccttttcattctgttgcttgtcttttgatggacaaaagttttaaatttggctgtattctaatttatctattttttgtttcctatgtttttggtgtcatatccaagaaatcattgccaaatccaatgttatgaacctttctttctgttttcttgtaagagttttatcattttagctcttacatttaggcattgggtccattttgagttaatttttgtctatcgtgaaggtaagggcccaacttcttttgcttgtgaatatccagttttcctagcaccatttgttgaaagactgtcttttctccattgaatggtcttggcacctttgttgaaaatcatttgaccaccaacctacttttttatacagctcctttatctgtatagccattatgaaaagagaggggaatggagagaggcttctaagaatagccttttgaaaacaggatattatgaggttacaactttctgttgcttttacgggtatattatttcacctgtgctgcagagctagtatattttgggggtaggctagttaagtcccatttataagccaagttttcccaaactattcattcagctactttagcctaatattaatactgcaatgtctagtatttttgatctagaaaaattttgggtgatttcagaatactcaattttatccttctaggaaggattgtcttatctagcatggcaaggtaattccaccatataattctaaattatgtctagatagtaccacaagcaattttctattttttaatgtttttacttctctaaatttgtgcccagaaaatatgatagtatttagattactctaagattgtactgttatcattttatattatatgcatgttgtagactatatatagcaaatgtgaccagtggcattaccagagtgtaagaatatcttgacatctttaaataaccaaaaaatagataagccactgaactgtctttaaagttcttaagacagtacctgtaggaagacagatttggaaaaggtaagcaaataaaaatcaaaagtaagatggcccttttaaattcagtttcctctgggaattaattgcctttatatcatcaaatatggctgctaagcaaataaactgctgacatgtgaacaacttttaattaattcatatctattcaacaacaattggatgtttacctcatgttcttggcatcatttctgattttgtgactgtgactgttttttatttccctggctaatgaagatgtaaatttcttaagttcaaattttgtcagctactcaaatgagaaaaggttttctttcttattttatttttttatagagattgaactcctgggctccagtgattgtcttgcttcagcctgtcagcctcctgagtagctaggtacaggtgtgtgccactatgcctggctaatttttttagttttggttttgtggagatggggtctcgctctattgtccaggctggtttcaaacacctggcttcaaatgatcctcctacctcagcctcccaaagtgctgggagtacgagtgtgagccaccacacctggcctgaaatttttcagtagttctaattaaatgaaatttctgagggtacatactataatatttaaggaatagtatttattatgttttggcatttgagactcttccatttgtaacttttctctagtgaatagtttattatgtttttagtattgccatcactcatttttcatatatttattgtcttctgatcatgacttccatagaatttccacatgagcagaatttgttatctaccagttcttttcttttctttctttttttttgagatggagtctcccactgttgcctgggctggagtgcaatggcgtgatctcggctcactgcaacctctgcctcccaggttcaagtgattctcctgactcagcctccctagtagctgggattacaggtgcctgccactacgcccggctaattttttgtatttttagtagagatggggtttcactatgttggccaggctggtctcgaactcctgaccttgtcatctgcctgccttggccttctaaagtgctgggattacaggtgtgagccactgcacccagcccatctactagttctatagggaaccaaggcaggctattaaaaagaatctaccaagtcattgatcatcttctacttaaacagtataacttacttgcctgttctcaaaagtgtggtgaaataagtgtttaaaattgagcaaaaatctgtctcctaccctgaattaattttgtattagcaacgtttaggcctttgttttgggtgatactgattatttgatggtgtggttcttaatagtttattttcttatatgaaaatatcttctatattttattaatagaaaagcaaatatacttttaaatatgtttcttttaataaaattagtttaaaaaggttccagtaaagctttaatttcatgagacaagttattacatcaagagaagagaaatacagatgtagagtatagagggcccttgtccggtttaaaaccttgattcgcattgttattttaggtaatatggctcttacctttttttctacaaagaaaattggtattatgtttagaatatttgtacttctgctttctttttctttctcccattacatgttcagtcagatttatctgtattcctaaatcctccaggtcaaaatcgtagaacctctgtgtttctctcttcttcattctttagacaatatatagttaataaagttatcagccttttttcagaatcttttagtatcttctgtttcctggatcagatccttattatttcgtatctgagccatacaccagcctcttaaagagcctccgtaaatacactttattttcttcaagcacttctatcattgctactactctgccaatggaagagacatgatgaaaacagatctccatagtttctaggatcaggtcattcaagggtacctgcagtcggaccccaacttacttttctaagttttaatccacattatttcttcacataaacttcaggccctaatcaaattagtttatgcatctgaacatatctggtgcttttgtgtgtgtgtcattccttctatttctttaccctgttttgtgtccatcatacatttctttatttttaaacacactgtgaagtttcaaatcttccatgaaaccctccttgattactccagctgaatataatcttaactttctccaagttcctgaaggagcttttgtgtggtgcttcctatatcatgtctggtgttataattatttgcttctgtttcgcttctaagctaaattttggtccccttgatgactgagaccacatcttatacagtcatgtgtcacttacttatggggatatattctgagaaatatgtcgttaggcaattttattgttctgtgaatatcatgtattgtacttatacaaacctatatgttatagcctagtatatgcctagtctgtatggtatagcctattattgctctaggctacaaacttatatagcatgttgctgtactgaatattgtaggcacttgtaacacaacggtaagtatttgtgtagttaaacatatctaaacataaaaaagatacagtaaaaatgcagtattacaatcttatggaaccatcgttatatatgtggtctgccattgaccaaaacattgttatggggcacatggctatttctttgtatccaacaccacttgtagtatgatacttccaacataattggagttcagtatatatttaactcagtaatatctactttaatttaggtaatttttatgtagaagatataatttgaagatatatttaatgaaataggttgatgttatatgtactctgtaattgggaacccagtgtaaatcagtttccatttgcaaatgaacttttattaaaattgtatcgcaaaatgaatgaaaattaaaggaggaatttatgccatttatttatttattaatcattttagagactgggtcttggtctgttacccagactagggtgcagtggtgtgatcatagctgaagagatcctcctacctcagccccccaagtagctgggactgcaggtatgtgccaccatgcctggataatttttaaattttttgtggagatgagatcttgccatgttgcccaggctggtctcaaactcctgggctcaagtgatcttcatgccttggcctcccaaagttctgggattacaggcatgagccactgagcctggccaatttgtatcttgtaaatgcatatgttctgaaaattatgattaaaatataatcatagattaaaacatgataatgaaacttacgatgagatatttccttcaaatttttggttttagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcaggtaattttcttttgcaaatccttacacataagtgtgagtagagattttatataattcgtatatattttctgtgtttacccatgccttttgattttgtaatactagttaagtactccttatctaaaatgcttggaatcagaaatgcttcagattttggatatttccagattttggaatattggcattgaacttaccagttgagcacccctaatccaaaatgctctaatgagcatttcctttgaacatcatgttgacgttcaaaaagttctggattttggaacatttcgtatttcagatatttgcaagagggatagtcaacctataataaaaaatcatatgggtaagttttatatcttgggagtaaaacacactttgtaaggtgaaattatttaaagagttgtactacaggaaatatcataggtattaaatatctattatttgatttcatttattctgagaaggctagggaagttcagaggaacagaagaaacacctggaggcatactttttgatagaagatcctccagttggctatataaatgacctgaataagtatttcccaaatggtggtgtatattttatttgcattagactcacgtgggggagcctgttaaaatcaaagatttctgggtcctaatccaagagatttggattcagtaaatctgagttgaagcccacaaatatgaatttttacagatgctctagggtattcttatacaaatggtccaaactctgcattcttcaaattattcttctgcatattgaacttgacaatcatggctaatataaaactgctttttaaaaaggtatatataagaatggtttaagtttgaaggcaaatatgtattttatatattacatacttcccagattaaaaatttgagtacatcatgagtgtggttaaagcttccttttttgaaattgttctcccaataactggcttcagtcatttctttgccatgtcatcaaaacagttacatacccaatttgcagagtatcaatttctgtatttctctctttggttttaaatattcttgttttactttcagatgtctgttaggtcttaacctaagggtgtaggccatagggtataattttaggaactcagaaatctagaagattccaaagcccaaaggaaaaaaaaaacacattcattcattcaacagctacttattgaatacttgctgtatgccaggctatagtagaacatagttggaggaagagtatcttagatgtcgttacatagaaactgaaggaattcaggggaacacagttgaagaacagaagctttgacccattgaagatgaatgttataaatatgaagccatctctagacttaagctatcgtggaatatttctgctttggaaaccatgctgtcatccaaagtcttggcacaatctttctatgtagattgtagtttcttccagtttgagaatcatgtcatctattgctcttatccctacattatttagtctagtgtcctaaacctaatgactatttagtaactatttatctgattcttttaactcatgctcttagccaggatataaacacagatgcaaaatgctgcatcatttttttttactgctttactttaaaaatagtgtagatattaaacattcatataagattccatgatattttaaaagagtttattgagataatttacaaatcataaatctcacccatttaaaatatacaatttaatattttgtattatattcacagggttgtataaagatgaccagaatctaattttagaacatttgcatcctttctgaaagaaaccccatgcccattagcattcagtcctcatcccactttcccccagcctctggaaaccacttatctttttgtttgcgtggattttcctgtctcaaacatttcatataaatagaatcatgcaaaatgtggtgtggcattttttttgtctggcttctttcacttaatatgatgtttccaaggttcattcatgttgtagaatgtatcagtacttcatttctttttattgctgaatcatttcattatatagatataccacattttgcttatttgttcttcacttgatgaaaatgtgggctgcttccatgttttggctattgtgactaatgtggctatgaacagttgtatatgagtaattttgtggatgtatttttttcattttttcttgtgcatatatttaggagtggaattcctggaacatatggtaactgtttaactttttgaggaactgcttaacttttcaaaagcagctgtgccacattacattcacactagcagtacatgaagagttccaactctttccgcacttgtttctgtctgtattttatccatcctagtgggtggaagtgctgtctcattctagttttgatgttcatttctctaatgactactgatgtttggcatcttttcatgtactttttggccatttgtatgccttctgtagagaagtgtctatttgaattcattgctcattttttaattgggttatttgtcttttaattattgagtttagaaaattctttttatattctggatacaagtctccacatataatcatatttatgatttgccattatttcctcccattctgtgtattgttattttactttcctgaaggtatcatttgtagcacaaaagttttaattttgaagtagtctaatttgccccatttttctttgtgtcttgtgccatgtggaaatgtggttttagcaacatggatgtctttcttggttttagcaagagcagtttctcttgaaataatggaacagaagccaaactggaatgagttgagggataaatggaaggcaagaaaatggaaatggctatgtacatgctttcaaaaagaggccagataggctgtggccagaaggaaatgtgggattaagagattttacattttatacgggacggacattaatttagcaagtcctttgtgcctgtggattctacatctaatatgccacagaatgaggtccagaaaggcttcctggattagatggtacttgagccttgtcgaatgagtaactagataggagatggatgattggaagaaagattagcatatattacggcatggaaaattatttaatggtacttcagaaataaaatgttccttagaagcacagaaataaattaccttctttagaatattgtacatagagatgaatgtacagaaaagtcagaatatttctaaatggaattgttttgatcacaaaggaattattaggagttgaagctggcctaacaaaataatatacagaagtatgtttgcataatgaaaatggcttccaacaaaatgtaacaatttccaaaattcaggtaaatgttggagcacagcatcataggaaatatttgtagcacagttcaatatatgccaaaatattatcttataaaccattaactctggtatgtgaaatagagaaaaggaatttttagagattcttattaactagactgaggattcatttcattaggggaagaagattaacatcttcttttgtaaacattgatgataacttagagtgtgtggatatatttatatcatcaaacaaaatcttaaagttttatactgtatggatgggggttatgtcaacttacgccacttggccactgtgttgtaaggcctacttaatcatggaaaaaggtggtaacttctttttcaatttttagatttatcttccaatttttgttttgttttgtttttctgtatgtgcttttttatccctagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggtaatccttaatgatatgttcttgttctttgttattttaagtacaatggaaataaaaacaaagtaattttaatcatttgcaacatggtattgcacttctcccatttgatagaagtggaagtttttaatagcgtgaacctatcaaggtcatataattctttgcctactattcttaaatggttttttcatttaattttttatgacaaaataattaccaatgactatattatagtttcagaaagatgtcaaattctaaaagtgattttaaaggagttttgcctttcactactagtgataacagaaggcataattataaaacttgttaactagaaaagattgatattaattgattttatgataatatatacctgctaaataatgaatttattttaggtttgcattgcaagttttgaatttgttaatatccgtgttgttcatagattgttactagagatttagagggatcattttttaatttttcatctttggtagtcacatttccagttttctggcagtggtttctactaatgttaattgaattgaagagtgccatagcacattttagattcctggcatcatttaatgccctcatttttaagtcaagcaagatacagcatagtcttagggttagattgatagagtaaatcacgtttaatgcttatctattgttatcaataccttttttattttaaagaatttggaaagaatgttgtcttcttaagtcttgttttaaaatggctctgtaaattctacccgtttttaattttacatgcttttaattataggatttacagttatattgcgattttcctaatattattgatgtcagtattaagcaagcaaaccaagagggttcaaatgaaagccgagttgtaactatccataagcaagatggtaaaaatctggtaagtttgctttatgattgaataatggtttcattttatagttctcagaaatgtgtattttagaatcttagtaccaaaattattttctggtaggaattttgattgtagttttaaatataactctaaacatcagttcagtaaactttttgataacatctagctttttgtattactatcaacaccatcttcctacagagtttcttgtacattactgtactttttttactcatttttatgattagcaaagcaccagttactttctcccccatccctactctagattatactagttctctgatgtaatgctcacccctgactaaaatattttatcaacagggatgtatatgtgccagttatactgcctaattaaaggagaaaaaataaatcaatgatgtctggaatttatataacaaaatgtaaatcaatcccaggtcagccccacccaagctgtaggacatggttgattctaaaagattaacgctttctctttttttaaatagcttcagaaatctaatttattttaatgtaaataggagaaatgaattattttatactttgctcttaagtttctcatttatcttctctgtcttctttttaactcagatgcttcttgacccttcctggcttaaactagtcctattaatgttatttgtctatttatcttaagtatttatatggcttacctgtctggctgttccagggttatattaccaaaattgtaactaacttgcaactcacttaattcagcatagttttctgttcttagataaaaatatgatagtacacctactgcctagtagactgtgagccttttgaaggtaaggatcatatttttatgtgtgtttgtaccccctacaccttgcacagtactcagggcctagtttagaaaaagcttgataaatattttatggatgcataaaaggatgattaacaattatcaacctcaatgtggatatataataaatgtttctgggagagtttattgatcttaatgtgaacaaactgtagtaacccaaagaacttctccatgggactgtatttggacttggcttaattttaagtctgcaatacagagaagttaagtaactttccaagggcacagtgttagttaagtggtagggctatttaaactcaaatgcatgctttgaaaacctaagctcttaagtgtgacactaacttctttgctaagtatccgtaaagctccttaaaaatattatactgacattttgttggaattgtgcaacaaactgggaagagttgacatttctttctcatattaaattttttattttagggatacattgtatttctccttgtatacaagtattattttatgtttttcagtaattcttgatcattttattcatattgatacatcatgtttcttgctgaattttattcatgatatgttatgctttttctgttgtttattaaaattaatacttcttctctgtgtattttcctaccaaattatttatggatataggaaagtaatttttgcttattattgcattttcccagtgtattatgtgttagtaacattttgtttttactgtcattttcctcttcattctggcatttatttctctttataattttggtaaaatatatataaaataaaatttagcatcttaaccatttttaagtgtacagtttagtagtgttaagtatattcacttttgcatccaatttccagaactcttttatcttgcaaaattgaagtctacacccattaaataaaaactcaccatttccccctcttcccaatccttgcaaccagcattctactttctttttttttttttttttttttgagatgagtcttgccctgtcatccaggctggagcacaatggcgcgatcttggctcactgtaacctgtgcctcctgggttcaagcaattttcctgcctcagcctcccgatagctgggattacaggtgcacgccaccatgcttggctaattttttgtatctttagtagagacggggtttcaccatgttggccaggttggtcttgaagtcctgacctcatgatccgcctgcctcatcctcccaaagtgctgggattacaggtgtgagccactgtgcccagcccctctactttcttattctatgaatttgactactctagatacctcataaaagcaggctgaaacaatactgtctttttgtgacaggcttatttcagttagtataatatactcaaggttcatttgtattacagcatgtatcaatttccttcatttttaatatatgtatgtattctgtgtcatgtatataccacatttgttcattcatccatcaatgaatattcaggtcacttttttactttttgacaattgtgagtaatgctgctgtgaacatgggtgtacaaatatctcttgaaaactctgctttcaattcctttggatatataccttctattagtccgttctcacactgctataaagatactacctgggactggataatttatttaaaaagagggtttaattgactcgcagttcggcatggctggcccaggaaacttaaaatcatggcagaaggtgaagggcaaggaagccatgtcttacatggcagcagaagagagacagtgtccaggggaaagtgccacttttaaaccatcagatctcatgagaactccctcactatcatgagaacagcatgggggaaacaacccccatgattcagtcacctcccaccatgtccctccctcgacacatggggattacaattaaagatgagagttgggtggggacgcagagccaaaccatgtcatacctagaagtagaattgctggatcatatggtaattctacttgtaattttttagggagccaccatgctgttttccatagcagcggcaccattttacattcctaccaggagtacattctccacatgctcatcaaaacttgttattttctgttttctgatagtagccatctgatgggtgtgaagtagtatctcattgtgattttgattcacatttccctaatgattagtgttgctgagcgtcttttcatatatgtattgccatttgtatatcttctttgaagaaatatatattcaagtcctttgctcattttttaatcaggttttttttgctgaattatagaaattatttatgtattctggatattaatcccttttcaaatatatgatttgcaaatattttctcccattccatagattgcttttcactatgttgcttgtgtcctttgatgcgcagatgttttaaagttttagctagttcaacttttgttgcctatttttctttttactttgttgagatataacatgaacccagtaaagcacactaatcttacgtgtatagctcaatgaatttttacatatttgtacatctttgtagccacacccacctcaaaaaacagaatagttctcttactcctagaaggtttccacatgcatcttcccacatattcacctccacccctaacttaagatacctatcatctaccatcacttagttttatctattaataaaggttgtataaatggaatgatactgtatgtattcttttaaaatctagattcttttagttagtataatgtttggagattcatttatgttgttacatgtgtcagttcattcctttttattgctgggtagtattgaattgtttgactgtacctcaaattttaaatgtattctgttgatggacatttgggttgctttcactttggggattttagggataaatttaccatgaacattcttatacatattttttggtggacttatgcactctttttttgtatatatatatcctagagataggattattgagtcatagtgatagctttagtatactgccaaacaattttttgtagttctatcagttcactcttctttcagtaatgcatgaaaattacagttgtctcatatccccatcaactcttaattttgtcagtctaaaaatgtcttagtttgtctggtggatgtgtactggtagctattgtagtttttattttcattgccctggtgagtaatgatgttcaatactttttcacttacttattgttcatctgaatatccttcttatgaagtgcctgtgacagtcttttgcacatttttcattgacttgtccctttctgatcaatctgtagagtctctatagattctaagtcttttgtaggatgtttgtaaaacgtatatagacatacgtcagagatatttctagttcagttctggaccactgcaataaagcaaattttgcaatgaagtgagtcacataaattttttggtttctcagtacatataaaagttatgtttacactataatcaagtctactaagtgtgcaacagcattatgtcttaaaaattaatttaaaacactttattgctaaaaaatcctaatgataatctgagcctccagagagttataatctttctgcttatgagggtattattttgatgttgatggctgctgaccgatcagtgtggtggctgcttaaggttgggttggccaaagcaatttcttaagataacaatgacatttactgcatcagtagactcttcatttcgttaaagttgtctctgtagcatgtgacattgtttggtaacattttacccacagtagaatttctttagaaatgagtcagtcctctcaaaccctgccactgctttatgtactaaactttattctaaatcctttgctgtcttttcaacaacattcacagcatcttcacccaggactagattccatctcaagaaaccatttttttatgcataagaagcaacttatctgtccaaattttatcatgagattgtagcaattcagtcacatcttcagctctacttctaattctagttctcttgctatttctaccacatcctagtgacttcctccattgaagtcgtgaacccctcaaagtcatccatgagggttggaatcaacttcttccaaattcctgataatgttaacactttgaccttcttccatgaatcactgatgttcttcctggcatcaagaatggtgaatcctttccataagtttttctatttacccagctccatcagatgaatcactatctatggcagcttagccttacaaaatgtatttcttagataataagacttgaaagttgaaattactccttgacccatgggctacagaatggatgttgtgtcaccaggtctgaaaataacatgaatatccttgtacatctctgtcagagcgcttgggtgaccaggtgcgttgtcaatgagagtaatactttgaaagcaatctttttttctgagcagatcttaacaatgggcttaaaatagtcagtaaaccatgctataaacaatagatgtgctgtcatccaagccttgttgttccatttatagagcacaggcagagtagacttagcataattcgcagagccctgggatttttggaatggtaaatgagcgctggtttgaatttcaagtcaccaatggcataggcccttagcaagagggtcagcctatcctttgaagtttgaaggcaagcactgacttctcctctctagctgtggaagtcctagatggcatattcttccaacagaaggctgttttgtttacatttgaaaatctgtttgttagtgtcaccatcttcatcaatggtccttactagatcttctggataacttgctgtagcttccacatcagcatttgctgcttcaccttgcacttatgctgtgcacatggcttcttttcttaaacctcatgaaccaacctctgctagcttccaacttttcttctgcagcttcctcacctctctcagccttcatagaatttaagagcattagggtcttgctctagattaggctttggcttaagggaatgttatggctggtttgatcttctatctagaactttctctgtatcatcaataaggctgttcactttcttataatttatgtgctcacaggagtagcactttcagtttccctcaaggacttttcctttggattcataacttgcctttttagtgcaagaggcctagctttcagcgtgtgttggctttcagcatgccttcctcactaagcttaatcatttctagcttttgatttaaagtgagagatgggtgaatcttcctttcacttgaatactcagtggtcattgtaggattattaattggcctaatttcattattgttgtgtctgagggaatagggaggcctgaggagagggaaaaagaaagggaaatagctattcggtggagcagtcagaacatatacaaatttatcacttaagttcaccatcttatataggcccagtgtgtggtgccccaaaacaattacagttgacccttggacatcacaggtttgaattgtgtgggtccacttatatgcaattttttttcagccaaacatggatggaaaatatagtattcacaggatgcaaaacctgcatatgtgtagggctgacattttgcatatatgggttctgcagggctgatttcagaacttgaatatgttcagattttggtatatgcaggggtcctgtaactaatcccccttgtattctgagggacaactgtacaatagttacatcaaaggtcattgatcacaaatcaccgtaacaggtataatagtaataaagtttaaattttgtgaaaattaccaaaacatgacacataggaagtaagcatatgctattggaaaaattgcactgatagacttgtacttgctccacttaagtttgtcagaaaccttccatttgtaaaaaaaaaaaaaaaaaaaaaaaaaaaaaaaggtcatatctgcaaggagcaataaagggaagtgcagtaaaacaaaatgtgtctgtatatcaataagagggatactagttagtgaactgctagttcaaagggtatgaatatttacaactttgatagattattaaaatacttctcaaaaggtgtttccaatgtatctgtacacctgttttcttgtatcctttgccaacactggatgttatcaatcttttaatttttctgccaaatcaatgggcaaaataatgctgtcctgttatatttcatttccttaattagaacctgagattgagcaatacctcatgtgtttataggtcagctttaatatttatacttctgtggattgccttttcatgtcctttgactagttttctttttttatttttaattgttttatacgttttattcccacaatgtatgttaggtatattcctttgttatacatgtttgtaatctccaagtcttttaattttgtttaggataccttttatagtacttgtttttttggctatttgtgttagctaaataccttttatagttcaaacatgtcacatgcttgtctctttattataatttttggttgaggactctattctgtgttctctttatgattatgtaaatattgttcattgctgggccaagaaatatgctgtaatcacattttcgttcttatataactttttccaggagttaattattgctttatgatttttcctttgtatctagctttattttcattttctacaagaactctgtaaagtagtggacttctcaatacaaaatttcacataatcaaacctgtcatatatgatagcatttttacttcttttctggagaagtacctcttcgatcacagcatcttactttgtagtgtggactggaagttttctgggtacagctattattttgtcagctgtaatttttggtcattcctttcttatcctggtaactatgttttcatctctcttatattggatctgattttctaaatctcatgtttgccctgtttttctaggtacagcctctggtatttgttggtatctttaatgtgattgagacaaattagaaaattaatttagagaggatttttacatttttatgatattgaattttcctaccatggttaatcttctatatatttaaataaaaagttgcataagagtgttttgaagtttttgttaattattgatattgcatcctctttgttacaaatggggcttttttcattatataactgactttttgtatgtaaaaatataatacgtctatatattttcccatgtaagtaacatgctgcatttttggctgactacatgttcacatatgtattaagatacaatcaggccgggcatggtgactcacgcgtgtaatcccagcactttgggaggccaaggcgggcggatcattaggtcaggaatttgagaccagcctggccaacatgttgaaactctgtctctactaaaaatacaaaaattagccgggcgtcttggcgtgtgcctgtaatcccagctttttgggagactgagacaggagaatcgcttgaacccaggaggcagaagttgcagtgagccaagatcgcgccattgcactccagcctgggcgacagagcaagactctgactgggcgggggccacaaaaaaatcatagcatagctacatatcctaacattttagtttgtagcatgaatactttagcagtatgtaattggtatgtagggctgatgccgtggctcacgcctgtaaccccaacactttgggagactgaggtgggtggatcacttgaggtcaggagttggagaccagcctggccaacatggcgagaacctgtctttactaaaaatacaaaaattagctgggcatggcggcatatgcctgtaatcccagctactcatgagggtgaggtgggtggatcgcttgagcctgggaggtggaggttgcagtgagccaagattgtgccagtgcacttcagcctgggcgatggagcaagactctcaaaaaaaaaaaaaaagaaaaaaggaaatgctcatgatttctaatgtcaacttttatatcatattaaatagatttctagctgagataactggtatattagtctaagaaggacattggaaaataaagtaaaatgatggctatagagtgaactataactgggaaggtatgataatctgtatttaggaggatcttaaatatcagacatgagaatttgtaattcatattgagtactgagccataaaagatatgagcaatttagaagaaaattgtatttaacatggaatgaagaaaattttcaatatttaacatggagttgactttctaaaaggtgctatttctttttcttttctctgcttaggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgtgagtaatacagacttaaaagtaaatttttagaaaagtaaatgctgtatttacaaagaagatttaacagagtgatacatgtatgtttagaaaaaaataatttgacaagttttttttaaacaaaaggctatttgcaatcagattacataataattagaattatttagtgtataatttaccatctgtatcccaatctataatatttataagttgtacaagtttaacatttgttaatcatgcctatgtgtgattcactcctgcatgttactaaaaaagaaagatctccaatttatgttttattaattttaaaatactgcaaggaagccacagtgcaaaatttgaaaattctttatgtttggatagtccatctccttctactgaaaggttcaaacacaagatatatatgatgtccacatttctagactagtgctatgtagaacttcctgcagcgatgaaaatattctatatctgtgctgtgtaacacagcagccactagctacatgtggctgtttagcatttgagatgtggctaatgcaaccgaggaactgaattttaatttaattttaatttctttacatttaaatagctacatgtggctagtaactatcacattggctcacacagttgtagatccttactttaatactgggtaaagctagctatcatgcataacatctatttagcttaagtcctaatgatctcttagctaggatgtggtttatgttgacaacagatttgaataactcgagcaaagggaaaatctgggatcatgagcatattatgttctttgcatttaacctacccctaaatatttgagaaaatacatcttgggtaatataaagagttgatttagtctatgcttaatatttatttagtaatgaatattgttgctatgaaaaagttgtactacttgaactttatgtctgtaatttacatgtttaatatttttaaaactccttgcctttattcatgtatccataaattcattcaaacattcttttatttatatttactgagtaactactatgtgcaagcattggtttgtatattagaaatatggtgataagtgaaacagaatgattctttcttttgtatagattatattttgcttttctttcagggtgtgcagcttcagaaaacataaaaaattttaaaaaccagtagtctttgtcagttgccatggttcttctcttagactttgttatcgtactgaataataaaatcatgcattcagatttttatgtcagtaactgctatggtagtagagatataagtattttctgatatataatctttatagttaaatgaaggtttctgagagtatggttttaaaaatattaactttgtaatcagcaggcagaaaaatcaaatgaacaaattgttctttgtgattttgtcttataaataccttttattggtttttaaagctatataattgtcccttgaagtggtttgaacttactgcaaaatagcttggtactttgattttttcctttgaaagattattgctaaacatataaagtagaggagacaattctgacagttttagatttgacttttgattgttttagatgacacttggtcataatattatggtgcttgatatattattcaaattgcttcttctttacctttaggatggattttgccattagtaaactgaagaaagcaggtaatcagactggactgtatgtacttcgatgcagtcctaaggactttaataaatattttttgacttttgctgtcgaggttagtatgtcacacttattagtggtaacactttatttagttcatttaatttcctctccatatttatttaccatatttcctttttaatactgagaattatagctttggatatgataactagtataatgcttaatttcctcagagattctgtacaaataaacaagttataattcccagggtttatataaaaatataaaataatttataatgtctatctttttattttgcatttattttattttctgatgttttggtgttaccagtgtgttaaaaggtatttcatcaagtgtcttgatgtttctttgtttgcaaaactggaaatctttgccacatcatgtagaacaaaataaatcggaagcaaattttctgtttattaacagtggttgcagaattagaggaaacagaattagagaaaactaggtcaataacaaacaaatagaaacattaaagcacttttaactgatgattttctaatatttttaagaatagtggtctacaatgcatgtttcaacggtaaaactgcaatgaaaccccactgttaatacagatcattttggatctcatctatgattatatactatcacttatgaggcaatttcatggtataaatcctaagaaactatagaagcttgcaaatttatcttgcttttttaaaacagagaaaaataagaactttgttattcaattattttaatttttataaaatgcaacttatatttggcatatgtagagggtagattacttacagaagggaggttatgccatcactttacctttgtgtaaaactggtaatctatcctaatttcaaagtatcctgttactataacagataatagtacattatattcaagtatgaattcaaaaaaatatatatatatattttatgttcactgtatgtgccaagcctaatatgagagctatgtattatagagtttatgctacagccctaccttcaggaaacttatctactggacaaacaaaaattttcaaatatacaaaaaattctaaatcgaacattgtaattatctagcataggcaaatatagacagtaacagacaggtttacaattattaagaaagggcagccaggtgtggtggctcacacctgtaataccagcactttaggaggccaaggcgggtggatcacgaggtcaagagattgagaccatcctggccaatgtggtgaaatggtgaaaccctgtctctaccaaaattacaaaaattagctgggcatggtggtgcgcacctgtagtcccagctactcgggaggctgaggtaggagaagcgcttgaacccgggaggtggaggttgcagtgagtcaagattgtgccactgcactccagcctggcgacagagcgagacacggtctcaaaaaaaaacaacaacaaaaaaaaaaaaaaacaagaaaggttatttggcataggaagaacctgattgaccttgaggtattggtagattttatataaatgtttctgagtagaatgaatatgtttataaaatgcaccttaaaaataggtgaacaagttcataatatagtgcataagacaaatatttaaaaactgcaaaatattaaaaatgtaatattaaagataattatacaaatacaatggtggtgcagtggaatggctgtatgttcacctttgcttgtgcaaagaggtgagcattagagaggacctcttagaaacagtggtacttgaactgagttttgatgaatgaataaaggttcattgctcagaagaagtgggtatacattcttaggtagaaagatagcatgaagaattatcaaggcatgaagcagcatggcatttgcttgaaatgatttaagtcatttaatatggctggaacgtaggttgtatgttggcagtggcaggagctgagattggagatggaggcagaaaccagctcatggaaggctctatatgctacatttgttttaggctttatcttataaatgatggaaaactattaaggatggtattgttgctattaattatcattgccttcttacatgctttttcataaaatcaacttttaaaccacttttgtaggctgagaaattgtaaattgcccttttgaggaactaagtacttctatcactatactggcactacatcggattcatggttcaaatgtttattctgttgcttgttcttgtgatatcattttgtcagaataatcactgtgatgtccattgtgactatccctccctttctttataattaaacttatacagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaaggtaagataattttctagttatttttaaattactggtcatggattgtttatgtggcgtgaagtatcttcagtacattgatttcaaaggatatatgaaagaagcagctctaaaagttaattttatcttattctattgtacaagcatcatcaaataagattgtttactttggttttggtggtctagaagtgacttgaagttcaattattctaaaatgagattttaaacataatgtgtggtatgatgtcatttattgaactggaagatctcactactaaattcttatgttgttttaagtaaaaattttcatatacttcgaggattttaatttctgtattttaaaaataaagacagtaaagttggaaattccttgaaataatgagccttactattaaaaattattaaaattcctgaaaaattgtatgagttaaggaagttactataattttgaattaatgaaataactggcaggaatacatcaaaatccaaattacattagttaaggaagtgattataattttgaatgtatgaagtaactaagaagaaatatcaaagttcaatgagttgacccctaaaataattttctattataaaaaaagaacaattaggagttattaagcatttcttatacgtagaacacatttcattttactcctctttggagcaattcatactttcagtgtattttgaagtgatatatatgtattttattttttcagataaatcaaaccttctagtcttcagaacgaatggtgtttctgatgtaccaacctcaccaacattacagaggcctactcatatgaaccaaatggtgtttcacaaaatcagaaatgaagatttgatatttgtaagtcattagatactcattactgtcttttttgtccttttaaaacaacatctgttttcttgatttacattcatgtgacattggaattattttgttatatacaaatttagttgtgatttaaatatttttcttatgaaaaatatgccaaccttgtgttagatgttagcaaaattaattatcttaattatcctaagaatggaatcttaattttctttttggaaatttaaaaatgattcttcacaaatctcaaattaaaaattaaaaatctacaaagacctatatcgcaactcccaagttctcaagaaagtaagggaaattcaaggggttaaaaataaagaagaaactgagaaaaaaatccagccatctctcagtgtccatgagggattggttccaggactacccatggataccaaaatctgaggatacttaagtggaacctgtggatatgaaaagttggccctctatatacatgggttttgcatcctatgaacactgtatttctttttttaaataactaattattttttaaaattatttatttatttattagagcaggacttgctgtgtcacccaggctggaatgcagaaatactatattttccaactgagtttccttgcagatgcagaacccgccctgccagtacagagggcccactggatttattgaaaaaaacccacatataaatggacctgtgcagtccaaacccatgctgttcaagggtcaactgtagtacataagatctgataatctggcagtcaggtggtgagggttgatgatcagccacatttatcaagggggttaagatttaagagcaatttaaaagggccaggtctcagacctgtgcaaggccacgagttaaaagtgagactatccctgagtaaaaccaggacacttaagtaaatacatcctcagtaaaacaatatatagaatgtgaaaaattcacccactagcaagcagagaagaaaaggaatattgtcatttttaactgcgcttctggtgaggaaaaggagtctctcctaagaatttgtcatcataagctttggaatttgggcttcattatactacctacatagactgagaaatcctaaattaaaagtggtactctcctaatattgtgtggtgcttggaagaaacaaaaaaatcttttcggaaccacatactcttaacctaggcaacccataattcccacagataaaagcattgcaaacacaagcttttaatagaaaatttagaaacatacaaggaaataatgcaggattaattaagagtcagtagaccaacaaacatcaaaattagaactccaggaacttcctatcacagaattttcagatacagactataaaatacatatacatgtttaaagatagaaaaggaattgaaaccacggagaaataacaagacattgttttaacaaacaaaaaagatgtcaggaaaaaggcatagaaatgataaatatcctcattgacattaaaacagaatatacaggatgaaaagtaaattacttatagctgaagagagatgtagtgaacctcaagacagatctgaagaaattaagaatgcagcagttagggagaaagaaatggattatatgaaagagggattaagtggcatggaaggcaaaatgagaaagacccaactcatatctgagctctataaggagacaatagatagaattagccagaatggtaaagacaagcatcttcatgtgagtaacaggaaacagagtggccaggtaacttgactaacattacaaatctaataaatagtagaccttggatttgaacacaagcagccttactgcagagtgcaggctttcaacaattactttgtaaagatatgcaagaaatactagtattagtagtaatagctgatacttaatgcacatttactatgtgtcatggactgtgctaagtgcttttatgtatttattcacttattgatcataacaatcctatgggaaaatagttattattctcatttttcagatgaagaaaccacgacagctgcagcacagagattaaatagcctgtacaaggtcatacaagtggtaataagagaaagtcagatattctgcctctgcagaccacattctctagtgtgctagtaggaaaggcaaagaaaatgattcccatgtaagcaaatgtaggtaaacactgactctatttttaaaaagtgacaattatagcccattcaggagatttcaaaaccgagtagagccaaaatacacttaattcattaatccttgttttctgtctcttattactatttaaggaagcagaataaaagcattaatttggagtcttaaatctggatttagattctaaggaaaatacttgcttatggatttaaaaaaattcttcctcattgaatgtattttcttgttcctacttcgttctccatctttactcattcttttcttttacctttttctcttgaagaatgaaagccttggccaaggcacttttacaaagatttttaaaggcgtacgaagagaagtaggagactacggtcaactgcatgaaacagaagttcttttaaaagttctggataaagcacacagaaactattcagaggtgtgtatgttctttatattgttcatgtagtttatgctgtttaaagatgtgctctcatatgcatacaacgtactcatgtgtgcagcttttcaaaattgtaatttttaaatgtgtcaaggacttttctgaggatacattcttgtggggctatagaattacagggtttgaaaattaccggattaaaaaaattaattccaaactaattatgtttagcattatgttaggagtgttattacatttctgcatatactgtggccttgattttcaaacttggctccccagagctttatgggttacacagaggctcctctacaattacatttatttgaaaaaaaaacaaaaaaccaagtttcaaaggcactgtactaaggaacatgcttagtgaaatctagatgcctctgggcatcatccagagatgctagctggcagaccccgtggccagttgccagagctctgaacatagcaagatatgatacttacaatatcttaaagggtgttggtgttagaagatgatgtgaaaagttcaagccaaagagttgttctaatccagaataccacagtattggtgattgtgattcactaatcatacccaggggttctagtcacagtttagttgaattagagtgatgttaaaactatgctagtatcctgacacagatgtcgtgatattttatctgcacattcttaattctttagcaagtgttatttaaaggctacatccatctacctcagtttcctatatctatctctgacatctacctctagttgtacttctgtcctctatttcaggtgttatgggtcaagcctgtttgactggcattattcatgattcctgtaccactcttgctctctctcactttgatctccatattccaggcttacacaggggtttcctcagaacgttgatggcagttgcaggtccatataaagggaccaaagcacattgtatcctcatctatagtcatgctgaaagtaggagaaagtgcatctttattatggcagagagaattttctgaactatttatggacaacagtcaaacaacaattctttgtacttttttttttccttagtctttctttgaagcagcaagtatgatgagcaagctttctcacaagcatttggttttaaattatggagtatgtgtctgtggagacgagagtaagtaaaactacaggctttctaatgcctttctcagagcatctgtttttgtttatatagaaaattcagtttcaggatcacagctaggtgtcagtgtaaactataatttaacaggagttaagtatttttgaaactgaaaacactgtaggactattcagttatatcttgtgaaaaaggaaagcaatgaagttaaaagtagaaggttacaatgcccaaacaatagagtattatagtaaacaaatgtctataaaacattttgtgttcatgatagcaaaagagattatggcaggttcaacataacattggaataactggccttttcagtacaaacttatctggaattatgaagacaaagcatataaatgatacacttaatttttaatggaactgacagaaatgattatgttgatatgatactagatatattttttggctaaatttaggtgttcacagaaactactaaaagtataaatcgtaccccatgctttaatactatacaggcatgcctcattttattgcaccttgctttattgtgcttcttagatattgtattttttacatattgaaggtttacggcaacccagtgtctagcaactctgtcagcaacattttcccaacagcatgtgctcatttcatgtctctgtgtcatattttggtaattctagcaacatttcaaactttaaaaaaatcatatggtgatctgtgatcagtaatctttaatgctagtattgtaattattctggggtgtcccaaacagagagaatataagaaggcaaacttcatagatgaatgcagtgtgtgctctgactgctccatcaatgagccattcctctgtctctctccctctcctggggcctccctattccctgagacaacaatattaaaattaggccatttaataatcacacaatggtctttaaatgttcaagtgaaaggaagattcacccgtctctcactttaaatggaaagctagaaatgaagcagcaaacgctgatgtggaagctgcagcagttatccagaagatctagcaaagaccattgatgaagatggcttagagaggaaggcatgctgaaagccaagacaagctgaaagctaggcctcttgcactaaaaaggcaagttatgaatccaaaggaaaagtccttgaaggaaactgaaagtgctactcctgtgagcacataaattacaagaaagtgaaacagtcttattgatgatacagagaaagttctagatagaagatcaaaccagtcacaacattcccttgagccaaagcccaatctagagcaagatcttaatgctcttaaattctatgaaggctgagagaggtgaggaagctgcagaagaaaagttggaagctagcagaggttggttcatgaggtttaagaaaagaagccatgtgcacagcataagtgcaaggtgaagcagcaaatgctgatgtggaagctacagcaagttatccagaagatctagcaaggaccattgatgaagatggtgacactaacaaacagattttcaaatgtaaacaaaacagccttctgttggaagaatatgccatctaggacttccacagctagagaggagaagtcagtgcttgccttcaaagcttcgaaggacaggctgaccctcttgctaagggccaatgccattggtgatttgaaattcaaaccagcgctcatttaccattccaaaaatcccagggctctgcgaattatgctaagtctactctgcctgtgctctatacatggaacaagaaagcctagatgacagcacatctgtttatagcatggtttattgactattttaagcccattgttgagacctgctcagaaaaaaagattgctttcaaagtattactgatcattgacaatgcacctggtcacccaagagctctgatggagatgtacaaggatattcatgttattttcaggcctggtgacacaacatccattctgtagcccatggatcaaggagttatttcaactttcaagtcttattatctaagaaatacattttgtaaggctaagctgccatagatagtgattcctctggtggatctgggtaaagtaagtagaaaaacttatggaaaggattcaccattctagatgccaggaatagcattcatggttcatggaaggagatcaaaatggcagcattaataggaatttggaaaaagttcattctaaccctcctggatgactttgagggatatggtactttaatgaaggaagtcactgcagagatggtggaaacagagaactaagaagtggagcttgaagatgtgactcaattgctacaatctcacgataagccctgaaagaaggaggagttgcttcttatggataagcagagaaagtggtttcttaagatggaatctactcctagtgatgatgctgtgaacattgtgaaatgacaacaaagggtttggaattttacataaacttgattgatcaagcagttgcagggtttgagaggactgaccctgtaaagttctactgtgggtagaaggctatcaaacagcattgcatgctacagagaaatctttcatgaaaggaagagtctgcagtgaatttcattgttgtcttattttaagaaactgccacagctacctgaactttaagcagccaccaccatgatcagtcagcagcgattaacatcaagcaaaaagattacaactttctgaaggctcagattatcattagcaccttttttggcaataaagtatttttaaaaagatatattctttgttttttttaagatataacgctgttgcacactaaatagactccattatagtgtatatacaagttttatatgcattgggataccaagaaattcatgacttgctttattatggtagtctaaaaccaaactggtaatatcttcgaggtatgcctttattttagtgcctcacaaagtttacattcaagaaaattgtttatataattccctattttccactagtaatgcagattaaatacacatctacacttgtatgacattttttcatatctttgaaaattgataatcttctcaaaagcagtggtttggaacctgttatatggaggtatctgagaggctcctgtggaccatgaaggaatccaggattgtttttggaagccaacacttgtttttctggcaaaatatgttttatatatttttttaaaaaagttttaaagataaagcagcgcaaaacatttctgtattttttgcagaattaaaagattaaatggagctagaactcaaattctcctttatgtctatagtactatagacaattgttagtaatttttgtgtgtagaagaacttcaggaaggtttgaatgtctaaaaagttatgtggaagagaaattgagaaattttcatttatttttaaaaaccagccataattcagtactaatatataaaggtgtgctggtatattatatattatacatttatgttatatataataatatataattgtttagactcctactcttgctgttgtaagtaaactaaaaaccaaaaatataatgctgtgtatccattaagttttctttaaagttgtgagttttgccaatttaatttctttacctataatggtcacatgtaagtataaagatttaaattacatatatttaattatatttatacttaagccttattattattacttatattttaatgcagatattctggttcaggagtttgtaaaatttggatcactagatacatatctgaaaaagaataaaaattgtataaatatattatggaaacttgaagttgctaaacagttggcatgggccatgcattttctagtaagtagtacaacctttttatcaaaagatactattttattttataaaacaatatacaaattatctttacctggaaacaaaaaataaatctgtaattggatgccaattcatgtaaaatagtctgtgttaggtgataaaaagagattactttaggcatatgtttatgtatgtaaaatcacttttgaaaaaaaagttttgtcatcccatcaatatcttagaactactaatagacaatctgtatgttgtgtcttttggatttagaaaattcaaccaaaaattatgcagaagtatgtgcttctgtatttttgggccattgtacattatggtccatgagaaagcggtgttgacagagaagaataccagggggactgccagtcatcagacccctcagggtgcacctatgtcctgctgcagaactgaagtagtatttcttaatgtcagctcccatccagaaacacaaaccatgtcagccttagaactcatgtgaaatggcattggtagttttaatttattcatttcatcacaagcatagattattgtgttatttaaaatgtaaaacaggaagcataggagtcataaatttccttttttctcaatgcatgcctccaaattattatactatcatgtatttttcttctttaaatctgttttgggggcttgaacatactaaatgctccagtacttgtggactgatatttgaatatatgtgcgtttaactctaataggaagaaaacacccttattcatgggaatgtatgtgccaaaaatattctgcttatcagagaagaagacaggaagacaggaaatcctcctttcatcaaacttagtgatcctggcattagtattacagttttgccaaaggacagtaagttctagaaggattatatataatgttactaagctttacttgggcagtggtgtaaagggcatgttgttaattttccttgaattccatttaatttgtatgttcctgattaataataaaattatcacttttagcaatttaaattgttagttaaatcttagtctctatttttctttcttataagcatataaccatggactgtgaggtgattttttaaaaacagttttaatctctaattatgtatgaaggataggattttatccatttttagtatgaagttttgtaatattccttgagccacaagtaaattttctaaatattcttttcttctcgtcactaaataaaaatttagcctgagattttaggatttttttctatcctctgtgaagaaattagtttaattatccagctaattctgactttttaaaaccttaaaaaaactttgatttgtgtttgttcatctttactacttaacacagtgggttaaaaatacaaatttcagtcagatagttgagaggggttttttgggtctagataattataactgaattttgttatcttagataatttctctaattggatgttctaaatttagattctaaattgatcaatttttgtgcactgatgtgtcctatttaagctaatagtacttaaacttaatgacttatctgacagtgaagacagtgataactgggcagtagtacaggagtagggagtatggcagaaaattcttacagggtcttacatctggtatcctggaaaattttatcctttggaatcggaagtgaagatggaaaaaaatgtattcatatggtgaaggtatttgtcaatcaatctatttgtctgtctgtctatgtcagaataccagaaagtagggttatattgcttcttcctttcttgaatacagtccttctggtggaatcaaagaccacagaaactttacttgagaaattccgtgatttgagcagagcccacctatatcaattccatatatctagaatattttggtgcaatttagccttccatctgccataaatgcttgttggtccttgggtttttttttttatctgcaagtgggaattgccatggctattaaatcattagaacatctctgaatgatgtaacattacctttactacttacgcattatgaagaaataccaaaaagtatcatggggcaattttaggataaactatataaaaaaaaaagatttggccaggtgcagtggctcatgcctgtaattccaatgctttgggaggctgattgcttcctaaagcgcttgggtggatcacttaagcccaagaggtcaaagcatagtgagctgcaatcgtgccattgcactctagcctgagtgacacagtgacaccctgtctcaaaaaaaaaattttttttttttgaaggtaaaactaatggaaagaggttgagaaaatggtatgccagttgagacactatggtttaatggaaagattagttgattcagaactcactgtacaggattctaattctgtctctgctgctaacaaattgtgtggctttgggcagattactcaatcatatgtctccatgttgtcattgttaaagatgaaatattgaattagatgatcttgaaggtcccttctggttcttaagcttatgcagtcatgtgcatgtgcacatccaacccctccaaaataaagaaaataggcctgattattcaaatgatttgaactttaaagctatttacatataaaagattggtttacttgtgaattatttaaccctactctgttcgtatcatttaaaagttcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagagtaagtttatatagactaagttagaattactctatctctgaactttcatatttctttcacatgatttgtattttttagcccatctaattttaaaaagaaggttggtgtggcattacacaatttattctcagtttgtggttctttaattatagaagctacaattttatgaagataggcatcagcttcctgcaccaaagtgggcagaattagcaaaccttataaataattgtatggattatgaaccagatttcaggccttctttcagagccatcatacgagatcttaacagtttgtttactccaggtatgtatttgaatgatcttattgattttccagctttctatctttattgtatttaatgaatcattactaatttttaattcctttaaaacattttcttgatgtcatttgggccctcttaatttcttatgttcatatggcttttcatgctttatacatattctaatctccttgactgagaattattttcatttttataaaaagaccttttctttttttttttttttttttttgagatggagtcttactctgtcgcccaggctggagtgcagtggcgcgatctcggctcactgcaagctccacctccaaggttcacgccattctcctgcctcagcctcctgagtagctgggactacaggcgcccgccaccacacccggctaattttttgtatttttagtagagacggggtttcaccgtgttagccaggatggtctcgatctcctgatcttgtgatccgcccgcctcggcctcccaaagtgctgggattacaggcttgagccaccgctcccagccaaaaagaccttttctttcagtaacctaattttagttttccatttagtgagttgtaaattatagtattttttttttgcttaaattgacttttaaaaaacaattctctttaacatttctcatattttataattgattgattctctaaacatgagctgtatttgttttgctttgttctctcctttttcaaaatactgagctgaaatgcatgtgtttgacctataggtgctttctaccccagtactactcacctttctcacaataagtatcagtatattaccttactttatgaatactaaaacttcttattttggaaaacaaaattttatcctttgcaaagtctctcttgaaggcctgtcagattatgggtaatgattaaaggctcccattaatataattgaaactatttgagtttccctgtatcatttagtatttttaactcacgattattttggtcaacttgaatgtatatcagtttagtccagagaatgttatttgctaatttaaggtgataatattctttatttctccagattatgaactattaacagaaaatgacatgttaccaaatatgaggataggtgccctggggttttctggtgcctttgaagaccgggatcctacacagtttgaagagagacatttgaaatttctacagcaacttggcaaggtaaattgtcagaattttttcaaatagagtataatcatttcatttaggaaaaacttaagagcgtttctaaagttcactttctacaacattttaaggagtgcttgtagaaaaaaaaggtttggttgtcagatgttggagaaatgctgtgttaaataaacagctatctttactgaatgaagggggccagcccctccacacctgtgggtaattctcgtcaggtgggaccagagactgagaaaagaaagaagacacagagacaaaagtatagagaaagaaaagtgggcccaggggaccagtgctcagcatatggaggacccgcaccaacactggtctctgagtttcctcagtatttattactgttttcactatctcagcaagaggaatgcggcaggagagcagggtgatagtggggagaaggtcagcaagaaaacatgtgaggaaaggaatctgtgtcacaaataagttcaagggaaggtactatgcctggatgtgcacataggccagatttatgcttctctccacccaaacatctcagtggagtaaagaataataaagcagcattgctgccaacatgtctcgcctcccgccacagggcagtttttctcctatctcagaaatgaacaaatgtataattgggttttataccgaggcattcagttcccaggggcaggcaggagacagtggccttcctcttttactaatcctccccagcacagacccttcacggatgttgggctgggggacggtcaggtctttcccatcccacgaggccatatttcagactatcacatggggagaaaccttggacattacccggctttccagggcagaggtccctgcggctttccgcagtgcattgtgtccctggtttattgagactggagaatggcgatgacttttaccaagcatactgcctgtacacattttgttaacaaggcacatcctacacagccctagccttaaaccttgattccatacaacacatatttttgtgagcttcaggttggggcaaagttacagattaacagcatctcagggcaaagcaattgttcagggtacagatcaaaatggaatttcttatgtcttccttttctacatagacacagtaacacactgatctctctttcttttccctataactgaagaacttttcagaggctttaacatgtaatgtgcatgaacatcttcaagaggaagatgaattaggcagtattattcaaattgacttgaccaagagactgttccttggtagcaggagttcattgttaagtgattcagtgaaatacagaaaggaatacataagcttttctgcctgcctttctgcctttagttatctcagggagaattaatcttattgcttagggtctaggattttgactgtgccatattcactagcagagactctgtctattaaaacgtcacacagtcatgtataacctacatattataagtaagaccagggaatgctcagttcttagtaggtccaaccatagtggtaagctctttctcgcataagcttgctaggataaggttctcactagtaagtaccatgttagtgttgtctgttattatcagctattcctaaattcatattttcctgaaatatctccagatgtcattgattttcctcttttctgggaacttatagcatttattgtttcttttatacaacttaatcacagtcttctgttgtttattagatcttggtttttgtttgcttgccctttttttccttgacttaaaaaaagttcctttagatcccaatagattactaggcacacaattttgtcatatattgtcaaaacaatggaatatatactggtatttaagaagtagacataacaaagaatacatcaaaagatcactaatttagattaacaaaagggaaacagcagactaaattataaagttatcttttcaagtaatttttctagttatgaaagtgatatactgcttatagagaaattggaaaatattacaatgaagatacgttccatttgttgtagtctctgcacaactacacaatttctttcaatagtgaaaccattctatatatacaattctatatcttgctatttttcatatatcaagattttcccattattaatattcttttgagacataattttaagtgaatataccataatttatttacaacattcccctttattgtaattttgagttaatgttcttttgctttttaacatttttagtgttttccatagttccgtgagcttaaaattcttttaaacataagtaaattgaagagactctaaggaaggcgttttcaaaatacatgtacaatgaaatatatttcttagtaatttagtttgtaggaaatgaaaggtaaatggtgtaatcattagtcagtcatagatattttaatgtggatatttcctatctgtatttaacaccataattttactttcatctttgtttgtttgaagtaattgatgtgttcagtggtttgtctagatatttgtactgccttactgaaacaatcaaaatgccattcctacaaaaatgattgcatgtcacctttccattttataaaaattagctactgtatccctttgttatccttttcaattagtggtatggtgaaaatattccaaagagggatatatatttaaaattattttcatatgttttaagccaagagaaataatggtatatttttttattttttaaaaaatattttcatactgttgtaaaaggagccaagaaactattgtgaaatcttttcttttgctatttgaaatctagagtgttttttgctttcttcctttatcctcatgcccacatatacttatattcttttttaaattaattttaacatggattatttgtgtaatagccttacatgtggatattaacagtgtaaacagtaccacatataaccatcaagacacctcaattagaatttataggactagaatgacagattcagatggtttaattacatttaaaaagttttctgaaaaaacttttgcaataaaacaaattgcccatcaataagtttgatatcttttaaaatgatttttggttatctgcacatgaagcaaacaaagtgtattccacaaaattcttaggcacagtctgagatagctgccagaaagaacagaaagagttgtcatttatgtcttgtgagtacaatttctgaaagttgaatgagggcgtctctttctggggatgagaagtttactgctctctcttattgcttcatggcagtactgctgggcaaggtaggttgtttgttttacaccatcactctgtaatacatactgtggagctctgtctacatctcccggcaaactgaaccatctcatgatcatagcattcatttctgggaactgctgacaggcaaataggacaggaccagtggctaacctgagattcacatcagctgatgttggtttgcctatgttagaacatgaggtgaagtcgaatacatcatctattagctgaaaagctattcctacattttttccgtactgataggtgatctcatgcaccactgggtcggggcatcctagaacagagactgctttacaactgttggctatcaggctcgcagtattcttgaaggtcttttcaaggtattgtgcaaatctctcatgctcattttcttttgacccgagctgaagaaattcaccacacaccaaatcttcaataactcgggttaaaatagatataacagctgtatttccagtttgtgccccacctatagatactacagaaagaattaaatctccagcaaggacagccttcttttcaccccagatcttgtgtgttttccttttcgagaacgtgcattatcaataacgtcatcgtgaacaagactagcagtgtggatcatttctgcaattaaggctgtggcgcgctggctagcttgcacatgtcgggagttattatgatgaatattacatgctcgggccattagtaccacaataattgatcgaaaggcttttccttttacatcaaagtagtactcagagatttccttaagttctgttgttgatatgagcggttcctttctaataccctcatacagacctttcaagtctctccaaccgagtttgaaaggatcggtgtatttctcaccactgtgtgttttgctgtacggggttgtgtgatgaaactgtgatatactatatacatttggacaggcaggtgttaaatgcttcacaagattaaaatagggtatctgagacaagtcaagtcccttctgcctatgagcctgcgcgggcatcggcagcggcgcgcggccccggcggccccgcaaggctcggggagcggtctccacgccgccggtctccagcaacagccgcgccgcccccgccaccagcgcgaggccacggtcggagtctgaaagtcgcggtaggatggtggctccgcctctggcccgcgatcctcctgccaggacctggagcctgaaacctcttatattctttatatcctttaccttctcgcttcactcctagcggttttgaaatcctctttattcctttttcctccatggtgcagatatgggattcagagatcaggatcttagtgcgactactttgaaattatcagcatcattctcaatcttctctgaatatgattcttattttcctctcttcccatcattagctaacatcttggtccacattccttgaatacttttagtactcattgttttctcatcaccttacctcctgccattattttggatgcccttccaatacttaaaaaaaaaattccttggcttctctaccccaaagttatgattatgttaaatgagttcctttgaactctataatcccatgtaataaccttacttagaaaatttcctaccctgaaatctccctgtctgactataactttatagcctttggcttttttcaacataaatgccttctttttgatatctttggtattgctagtgtctttcacactttcttcttattggaacgtaaattggtgaccctttctagagagcttctcatagagcagtttgtatcaagcgcgtttaaatgttcatatcttttaaactactttcgctggtatccacaattatatcacccccccttcccttgattatctgcttcactttccctattatttcctagtacgggaacaatacaacttagcatttttaaattgtgcttccatccctcaaacactaaaagaaaaaaatcagatataaatctttattgttttcattgaaaattggtttccaacattggttaggcacttatgactcttactggttgtattagtctgttctcacgctgcaaataaagacatacccgagactgggtaatttataaaggaaagaggtttaattgactgacagttacccatggctggggaggcctcaggaaacttacagtcatggcagaaggcacctcttcacagggcagcaggagagagaatgagtgccagcaggagaaatgccagatgcttacaaaaccatcagatctcatgagaactatcacgagaacagcatggaggaaaccacccgcatgattcagttgcctcccaccaggtccttccaatgacatgtggggattatggggtttacaattcaagatgagatttgggtgaggacaatgccaaaccatatcactcccttacttgatttctttcctggtcctatctcaaacccttctggttttctatcttaaatctttcttttcgctgtatttttcttcatctccttcttccataagaaaaatagaggcattccgaattaaaagtgagactgatttttgcctatcaaagtgacaaggatttagaaaaagtataatgatcattgttggcagggatgtgataaaatatacattttcccactgcttattggaacatcaaatggtaaccactgccagacagcttctaatgcagtgtgtatcaagtccctttgaaatgttcttatcttttgaacagctaattaagtatccttaatagagatgtacacaaatatttatttataagaatattattgcaatgacatgactgtatttaaaaatttttcttagtgtacatttatgtttccaatgttaaaattagtgcattcgtgtgacaatatacaataattaaaaattattaatttcaaggaatatatagtggcactagaaaataaattttatgaacacattgttaagtagaggggaagaagataaaacttatttcctacttttgttttctaaaatgtgttttggaataatttctcacaacagcctcatgagatagacattttattattctccactttaaggatgaaaaacctcattttagggagttctgaattgtccgcggtcacagagagagcaagttgtggatcaatattctagttccaatctgacttgagagtcctgggtcttgagagtcctgggtctatccaccttattgtgtggccttctaaactgtgtaattaagagatttaactttgagcctacatggtctaggttttttaaaaaactgacttcaagtatataactgaaaatatagtcacttagaatcttgtaactcaagaactacattgttttatcctgtaagaatgcaataatatccaaaatacactaatttgattaatatcaaaaacctatactttatttaaatgttatagcaacttaccaattctatgtacttaatgagttttaggtaaaaagagtggatttctttaaagacatcttcaaaaaaaaaaatgtagagtcatcccataggctaaaagtttcttacttcttaatttgctttggctgctataacaaaataccatagactgggtagcttaaacatcagacatttatttctttgaattccagggcctggaaagtctgagatcaaagttctggcagggattggtttctagtgaggtttcttcttggcttgcagatggccatcttcttgctgtgtcctcacatgggagagagagattgctctaatgtctcttcctcttataagggcactagccttgtcacattagagttccacccttataacctaatttaacttttatcacctcctcacaaggccctatctccaaatacagtcatattgggggttaaggcttcattatataaattttgggtaaggggtcactgacatttagcccataacagacacattccatccagatcactgacagtgatagcaaaaggtattttgtaaaaaaggaaaattcttcctggaagttagtgacaaaagtttaggaatcctcagcatgtcccttggcttatcttgacaattaatgctcatcatttatttaaatgcttttttgaagtaatttatatttcagcctataattccttttgaagaaattaaatttacagatttactacccacctgttgagatagtatttccttttcttgttctaagagttccttaatataagcttaaagatagtctgctgctgggcatggtggctcatgcctgtaatcccagcactttgggaggccgagttgggtggatcaccaggtcaggagatcaagacaatcctggctaacacagcgaaaccctgtctctactaaatatacaaaaaattagctgggcgtattggcgggcgcctgtattcccagctactggggaggctgaggcaggagaatggtgtgaacccagggggcggagcttgcagtgagccgagatcgtgccactgcactccaacctgggtgacagagcgagacttcgtctcaaaaaaaaaaaaaaaaaaaaaaaaaaaagacagtctgctaattccagctactagaattttctatataattataaaatttatttgtaatttgccttgaaaacttggtatttccatcctaatgtgatgtgtcatttagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtaagctgcccattgaaacctattttaaattcaaggtatgtgtttggcatcctgtgtaatataaatgtacaatgtcttaacgatctggacttatgccaatgcccagagggagaggcattctataatgactagagattgtgtttggtgatcatagactataactataggaagtatttggctagttggttaaacattcttttcacctcttgcttaagcttaccaagaaagtatcatttttaaaggaattgtttttgtttacattttctataaaaatatttttgaattcaatgttttttgttcgatatcaatgaggagaaaggaagttttctcatcagtttattttggtttgcctgaagagttatagaaaactgaaacgcaaatagtttcaaagcttttattcattcaaaagtttttgattcaaaaagactatgttcttaacaactatatcacctttaatgtataataaagttttgtcatcttagatttcatatatgtttaagtcatttatgtatgatagttttctaatatttaatcagtataatatggcagagtaaaacattatttccacctttatgttaaaaggtcggcgtaatctaaaattaattatggaatatttaccatatggaagtttacgagactatcttcaaaaacataaagaacggatagatcacataaaacttctgcagtacacatctcagatatgcaaggtaactaatatcctgattatttgctgtagatgaagcaaccgtgttgaagtagacattaggaaatcatctagacgttttcatagataataaagggaatatatagggttaagaccatttaaattgtttatatttataaaactagctgaaagaaaaatgttttatccatagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtgagtatatttcagtatgataaatgaaattttagagcacagacttcaaactttattgttgttatttaatagtttgccatttctatatttacagtaacagtgaacatttaagtcttttaagtcttacatttaactttttttttttaggtcttatagtcatggttatagtccacgtgggaaaatggcatatgctttatttctattaagtgttgtcttatttatagggattgtaggtttttcttcaaaactgtctcctatttattgaggcttaggggtaattattagtggtgctgtgggttgtggtattgtgttgaattttggtggggctttcatggggttaatagtcttttttatttacttgggtggtataatggttatttttggttatactccagtgatagctattgaggagtatcctgaaacatgagggccaagtactgatatttgaggggccttattattaggattattaatagagttgatactggtttggtgaatagttgaatatgatggggtggtgatcacggttgattttaatagcatagggagttgatttaatttttgagggggaggggttgattcatgaggattttgtaggtgcagctgccttgtataattatgggtgttggttggtggttgctggttgatcattgttagtatttttgtcgcaattgaaattactcagggtaatagattagataattaatagtagagtttaggcctttttggtcagatacagtaatggaggctgaagtttgggttcgtgaaatggtttttggtatagacttttctagtcaaactgggtctagtagaagtgaagctagattttggcttatgaataggcttaagtcggggggctgaatggtgaattgtagctgaataaaaacctagtatactggagaagttaaatgtctataatgggtactttaaggttgttagttatgaaattaagctctattgctagtaagagacctagggtggttacacctagggctgtgagctttaggtggagtggtatagttgtttggggggatgaagtagggataatactgttagtgatgaggaatccggcgaagatactgccgattgctaggtgctgaactgagttaattcggagaggattatcattaataacaatcagaattgtaaaacaaggttgtcctattacagcacctaaggttataaagtaagcacaagtaaatacataaaaacgttaggtcaacgtgtagctcatgaggtggcaagaaatgggccacattttctaccccagaaaatctcacgacaacctttatgaaatctaagggctcaaggaggatttagcagtaaaccaagagtagagtgcttggttgaggttgaataaggccacgaagcacgcacacactgcctgtcaccctccgcaaatatcattctagaaatattatttaaattctctatacatgaatagaggagataagtcgtaatatggtaagtgtactggaaagtgctcttggacaaacaaagtgtcgcttaacccaaagcatccagcttacacccggaagatttcatcacaacctggtcactttgagccaaccctagccccaaacctcactaaaaatactattaaaccatcgtaatcaaaccatttaccttagacaaaagaataggcaatagaaattcttatcttggcacaatagatacagtaccgtaagggaaagatgaaagaactgagcaaagacaagcccttataccttctgcataaagtattaagtagaaataactttacacagagatagccaagtcccccgaaaccaaatgagctatgcaagaatggctgaaagagcgtactcacctacgtggcaaaatagtgggaagattcatgagtagtggtgataagcctatcaagcctgctgatagctggttgtccaagatagaatcttagttcagctttaaacttacccacagaattacttaatgtccctgtaagtttaactgttaatctaaagagggacagctctttagaccctaggaaccaaccttcctacagagagtacaaaataacatcgccatagttggcccaaaagcagccaacaattaagaaagtgttcaagctcaacatccaagtatcttaaattctaatcactctacggaactcctaacattacactggtctaatctattatttaatagaagcaataatgttaatataaataacatgaaaatattctccaccgcataagcttacataagactggaataacccactgactgacagttaacagctagatattttacaataagcatcctattatttatactgttaatccaacacaggcatgctctaaggaaatattacaaaaagtaaaaggaactcggcaaatcttaccccacctgtttaccaaaaacatcacctctagcattaccagtattagaggcactgcctgcccagtgacatatgttcaacgaccaccatatcctgaccttgcaaaggtaacataatcacttgttccctaaatagggacttagaagaatggccacatgagggttcagctgtcatgagggttcagctgtctcttacgtttaatcagtagaatctgacctattggtgaagaggagcttataagcaaataagatgagaagaccccatggcactttaattcattaatgcaaataaaaactcaaaaagcctataggccttaacctactatctctgcattaaaaattttggttggggtgacctcatagcatattcaacctccgaacaacctaaactaagaccacagtagtctaagcgagttaatacacattgacccaataatttgatcaacagaacaagttaccctagggataacagtgcaatcctattctatcatatcaataatagggtttacgacctcgatgttggatcaggacatcctaatggtgtagccgctattaagggttcatttattcaatgattaaagtcctatgtgatctgatttcagaccagagtaatccaggttggtttctatctgtttaacatttctcctagtatgaaaggacaagagaaatacggcccacttcataaagtgccctccccccataaatgatgctatctcaatctaggaaatcaccacacacccaagaacagggtttcttaagatggcggagcctggcaatagcataaaacgtaaaactttacaatcaaaggttcaactccccttcttaacaatatgcctataattaaccttttcctacttatattcccactctaattgctatagcattccttacacttaacagttacgcaaaggacctgataccgtaggcccctgtggactgcttcaaccatttgctgacccaataaaacttttcaccagagaacccttacgaccctcaacgtctactgttaccctttatatcattgcctccaaccctggccctttctatcaccctcctcttctggactccccttcctataccccatcccctaattaattttaatataggcctcttatttattctagccacatcaagcctagcagtctattccattctatgatcaggataagcatccaattcaaattatgtactgatcggcacactacgagctgtggcccagacaatttcatatgaagtcaccctagccattatcctgctatcactactactgctaagtggctcaacttatatgcatgcatcacaatgccagaattactctgaccgctcctaccatcatgacccttagccatattatgatttatttccacactagcaaaaactaaccgagccccttttgacttaacagaaagagaatcagaactagtttcaggcttcaatgtcgaatatgctgcaagttcatttgccctcttctttatagcagaatatatgaatattattgtataaatgccctgactactactatttttctaggagtactacacactatgtattcaccagaactctgtaccataaatttcattaccaagaccctccttttaaccaccgtttttatgaatccgaacagcatacctccaattccactacaaccaactcatataccttttatgaaaaaatttcctaccacttacattagcattctgcatatatctcaatgcctttcctaatttccagcattccaccccaaacataggaaatatgtctgacaaaagaattactttgatagagtaaacaagagaggttaaaatcctcttatttctggaactataggaattgaacctatccctgagactccaaaattctccatgctacctattaaaccatatcctaaagtaagttcagctaaataagctattgggcccattctcctgaaaatgttggttatacccttcccatactaattaatccattagctcaacttattatctctcttactattttcacaggaaccctcatcacaatgctacgctcacactgatttcttatctgaacaggcctaggaataaatatagtagttcttacccccatcttgattaaaaaaacaaatctccactctacagaagcagccaccaaatattttcttacacaagcaactgcatctataattctcatgatgggtatccttttcaacaacccgtcctccgggcaatggacaataataaacactactaatcaattttcatccttaataataacagccctagtaataaagctagaaatagccccctttcacttccgagtcccagaagtaacccagggaacttctctaatgtctggcatacttctcctcacatgacaaaaactagcccctatctcaattatatttcaaattttcccatcaataaacacgaccagtctcctatctatcacaatcctatccattaacgtaagcggttgaggaggacttaatcaaacacaattctgcaaaatcttaacctactcctcaattactcatataggttgaatgatcccagtactaacttataatccaaacgttaccattataaatctgattgtttaccttattttaacaactaccacatttctagcacttagcctaagtataagcactacaaccctgtcactgtctcacacttgaaacaaattaacatgattaacacctatagtttcactaattctactatccctaggagctttacctccattaacaggatttctgcctgaatgagtcatcattcaagaattttcaaaaaacaatagtcttattaccccaaccattatagctatcataacactactcaacctgtagttttatatatgcctaatttactccatctcaatgacaatattccccacatccaataatacgaaaataaaatgacaattcaaaaacacaaaacccatattactcctccctccacttatttcttctaccttcctcttacccatctctccactgattctagttataacttagaaatttaggttaaataagaccagggaccttcaaaacccttagtaaatgaattatacttaatttctgtaacaaacccaaggactgcaaaactctattctgcatcagttgaacgcaaatcaaccactttaattaagctaagtccttgctagatcggtggaattcaaacccacaaaaatttagttaacagctaaataccctaatcaactggcttcaatctacttctccctccatgtggggggagggtggtaggccggagaagccctggcagcattgaagctgctcctttgaatttgcaattcaacataaaaaattacctcagggctggtaaaaagaggtcttgacctctgtctttagatttacagtccaatgcttactcagccattttacccttttttccacttatgttcatcaattgttgattgttttcaaccaatcacaaagctatcggaacactatacctgctgttcggcgcatgagtggggatagtgggtacctccttaagccttctaatttgagcagaattaggtcaaccaggaactctgctaggagatgaccagatctataatgttattgttaccgcccacgaatttgtcataatcttctttatggtcatatcaattataattgggggtttcggcaactaactagtccctctgataattgctgcccctgatatggcatttccacggataaataataatgagctttttacttctcccccactctttcctactcctacttgcatcctcaatagaagccggtgctggaactggctgaacagtttatccccctttagctgaaaacctaacacatgcaggatcctttgtgggtcttaccatcttctcacttcacttggcaggtattctattttaggagccattaactttattactacaattattaacaaaaagcccccagccatatcccaatatcaaacaccccttttcgtccaaccattcctcattacagcaatcctactcctttctctctcagtcctagccgctggcattaccacaatattaactgaccgtaacctcaacactactttttgaccctgctggtgggggtgatcctatcttatatcaacatttattctgattctttggtcaccctgaagtttatatccttattctatcaggctttgggatgatctcccatgtcgtgacgtattactctggaaaaaaggagccatttgggtatatgggcatagtatgagccataatatctattggcttcttagggtttattgtatgagcacaccatatatttacagtaggaacagacatagacacagacacatgagcatacttcacctccgctaccataattactgctatgcctactggcatcaaggtctttagctgattagctacactgcgtggtggtaacatcaaatgatctcccgcaatgttctgagccctaggattcatctttcttttcacagtaggaggtctaattggcattgtactagctaattcatcactagatattattttacatgatacatactatgttgtagctcatttccactacgtcctatcaccaggagcggtgttcgccatcataggaggctttaccactggttcccccattctcaggttatatgcttaatcagacctacgctaaaattcacttcaccattatattcataggtgttaatttaacctttttcccacagcacttccttggcctatccggtatgccgcaacgttattccgattatcctgatgcatacaccgcatgaaatattatctcatccttaggctcatttatctcattagcagcagttatgctaacaatttttataatctgagaagcctttgtttcgaaaagaaaaactataacaattgaacaaccatctagtaatttagagtgactttatggctgtccaccaccttaccacacatttgaagagccaacctacattaaacccttaaatgaaaaagaaaggaattaacttccagaaactggtttcaagccaatcaaaaacctctgtgactttctcgataagatattagcaaattcattacgtaactttgtcaaagttaagttataggctaaatcctatatgtcttaatggctcatccagttcaattaggccttcaagacgctacatcccctatcatagaagaaccgctcactttccgtgaccacgttcttataattattttcctaattagttcctggttctatacattatttccgtaatactcacaacaaaattaactcatattagcaccacagatgcccaagaaattgagactgtttgaactatcttacctgccattatcttaattttaaggcctctcatccctacgtattctgtataaaacagatgaagttaataatccttcccttactgtcaaagcaattggtcatcaatgatattgaagctatgaatatatagactatgaagaattaagcttcaattccttttttttttttttgagacagagtctcgctctgtcgcccaggctgcagtgcagtggtgtgatctcagctcactgcaagctctgcctcccgggttcacgccattctcctgcctcagcctcccgagtagctgggactacaggcgaccgccaccatgcctgggtaatttttttgtatttttagtagagacggggtttcaccatgttagccaggatggtcttcatctcctgacgttgtgttccacccacttcagcctcccaaagtgctgggattacaggcgtgtgagccactgcgcccggcttaagcttcaattcttatataaccccaacaccagacttaaaaccaggagaacttcgactccttgaagttgataaccgaacaattctcccaacagaaatacccatccatatattaatctcatctgaagacatcctgcactcatgaactatccccttattaggcctcaaaacagatgcaatccccagatgcttaaatcaaactaccttaaccactgggcgaccaggtctttactatggacagtgctcagaaatttgtgaatctaaccagttttatacctattgtcctagaactaatcctcttaaaatatttcaaaacctgatccacatctacactaaaatatcactgtaaagctattcagcatttaccttttaagttaaagattgacggagtctacacctctctgcagtgaatgcctcaactagatacttccacatgatccattattatcctgtcaataatcataactttattctccattattcagttaaaattattaaatttcatttatattatccctacaccaaaaataatcaaaacacaaaaacacaaggcttcctgagaattaaaatgaacgaaatctattcatctcttttgctacccctacaattctaggcctaccagcagtagtattaatcattctgtttcccactgtttccaacctctagtcatcaaattagtaaccgattattttctattcaacgatgattaatccaacctgcactaaaacagataataattacccataacattaaaggacaaacctgatcccttataataatatccctaattatcttcactgcctcaaccaacctccttgggcttctaccccattcatttacaccaactatccaattatcaataaacctgggtatagcaatccccctgtgagcaggcgcagtaattacagtcttctgctttaaaacgaaaaactccttggctggctttttaccacaaggcacacctatgccacttatccctatactagtaatcattgaaaccattagcctatttattcaaccaatagcattagctgtgcaattaacagccaacattatagtcagtcatctactaatacatttaattgcaggagacacactagtactattagctattagttttcccacagcttcagttgcttttattattctaatcctactgactattctcaaattcgccatagaccttattcaggcttatgtcttcacactactagtaagcctttatctatatgacaacacataatgacccaccaaacacatgcttgccatatagtcaaacccagcccctgaccactaacagaagctctctcagctctactaataacatctggcctggccatgtgatttcactttaattctactactcttttaaccctaggcctattaatcaacacactaactgtataccaatgatgatgtgatattatctgagaaagtaaatttcaaggccaccatacaacagttgtccaaaaaggcctccaatatggaataattctatttattatctgagaagtattcttctttgctggtttcttctgggcattctatcactctagtctagccccaatgccagaattaggaggacactgaccccccaacaggcatttttcccctcaaacgcttagaagtacctttctgaatacatctgtattatttgcatcaggagtttcagttacttcggctcatcacagcctgatagaaggaaatcgaaagcaaataattctagcactatccatcacgattaccttaggtatttacttcaccctcctacaaatctcagtactttgaggccccttttactatctctgatgggatctatggctcaacattctttacagccacaggctttcatggatttcatgttattactggatcaacatttctcactatctgcttcctccgccaattaaaatatcactttacaagatgtctgaataggaatagctccagtctgcagctcccagcatgatcgacgcagaagatggtgatttccgcatttccaactgaggtacctggttcatctcatcgggactggttggacagggggtgcagcccacagagggcaagttgaagcagggcggggcatcacctcacccaggaagcacaaggggtcaggggatttccctttcccagccaagggaagccatgacagactgtacctggaaaatcaggacacttttgcccaaatactgtgcttttccaacagtcttaccaaatggcacaccaggagattatatcccatgtctggcttggcaggtcccacacccacggagccctgctcactgctagcacagcagcctaagatccacccgtgaggcagcagcctggcaggcggaggggcgtccgccattgctgaggcttgagtaggtaaacaaagtggccagggaagctcgaactgggtggagcccaccacagctctgcaaggcctgctgcctttgtagaccacacctctgggggcagggcatagctgaaaaggcagcagaaacttctgcagacttaaacgtccctgtctgacagctctgaagagagcagtgtttctaccagcatggtgtttgagctctgacaatggacagactgcctcaggtggatccctgatgcttctgtagcctaagtgggagacacctcccagtagaggccaactgacacctcatacaggtgggtgcctgtctgggacgaagcttccagaggaaggatcaggcagcaacattggctgttctgcaatatttgctgttctgcagcctccactgcagataccgaggcaaacagagtctggagtggacctccagcaaactccaagagacctgcagctgagggacctgactgttagaaagaaaactaacaaacagaaaggaatagcatcaacatcaacaaaaagggcatccacaccaaaaccccatctgtaggttaccagcaccaaagaccaaaagtagataaaaccacaaagatggggagaaaccagagcagaaaagctgaaaattctaaaaaccagagggcctcttctcctccaaaggattgcagctccttgccagctggatggagaatgactttgacgagctgacagaagtaggcttcagaaggttggtaataacaaacttctccaagctaaaggaggatgttcaaacccatcgcaaggaagctaaaaaccttgaaaaaagattagaccaatggctaactacaataaacagtgtagagaagaccttaaatgacctgatggagctgaaaaccatggcatgagaactacaggacgcatgcacaagcttcactagccgacttgatcaagtggaagaaagggtatcagtgattgaagatcaaattaatgaaataaagcgagaggagaagtttagagaaaaaagagtaaaaagcaacaagataaagcctacaagaaatacaggactatgtgaaaagaccaaatctacatttgattgttgtacctgaaagtgacggggagaatggaaccaagctggaaaacactcttcaggatattatccaggagaacttccccgacctaccaaggcaggccaacattcaaattcaggaaatacagagaactccacaaagatactcctcgagaagagcaaccccaagacacgtaattgtcatattcaccaaggttgaaatgaaggaaaaaaatgttaagggcagccagagagaaatgtcgggttgcccacaaagggaagcccatcagactaacagcggatctctcagcagaaactctacaagccagaagagagtggaggccaatattcaacattcttagagaaaaagacttttcaacccagaatttcatatccagccacactaagcttcataagtgaaggagaaataaaatcctttacagacaagcaaatgctgagagattttgtcattaccaggcctgccttacaagatctcctgaaggaagcactaaacatggaaaggaacaaccagtagcagccactgcaaaaacataccaaattgtaaagaccatcgatgataggaagaaacagcatcaactaacgggcaaaataaccagctaacatcataataacaggttcaaattcacacataacaatattaaccttaaatgtaaatgggatgaatgccccaattaaaaaacacaaactggcaaattggataaagattgaagacccatcactgtgctgtattcaggagaccaatctcatgtgcagagaaacacatgggctcaaaataaagggatggaggaagatctaccaagcaaagggaaagcaaaaaaaaaaaaaaaaaaaaaaaaaaaaaaaaaagcaggagttgcaatcccggtctctgaaaaaaaagactttaagccaacaaagatcaaaagagacaaaaaaggccattacataatggtaaaaggatcaactgggcaagaagagctaactatactaaatatataggtacccaatacagcagcaccaagattcataaagcaagtccttagagacctataaagagacttagactcccacacaataataatgggagactttaacacctctctgtcaataatagacagatcaatgagacagaaggttaacaaggatatccaggacatgaactcagctctgcaccatgtgaacctaatagacatctacagaaccctccaccccaaatcaacagaatatacattcttctcagcacgacatcacacttattccaaaatagaccacatagaagtaaagcacttctcaccaaacgtaaaagaacagatatcacaacaaactggctctcagagcacagtgcaatcaaattagaactcaggactaagaaactcactcaaaaccgcacaacttcatggaaattgaacaacctgctcctgaatgactactgggtaaataatgaaatgaagacagatataaagatgttctttgaaaccaacgagaacaaagacacagcataccagaatctctgggacatatttaaggcagtgtgtagagggaaatttatagcactaaatgcccacaagagaaagcaggaaagagctaaaatcgacaccttaacatcacaattaaaagaactacagaagcaagagcaaacaaattcaaaagctagcagaaggcaagaaataactaagatcagagcagaactgaaggagatagagacacaactcttcaaaaaatcaatgaatccaggagctggtttttcaaaaagatcaacaaaatagatacactgctagcaagactaataaagaagaaaagagagaagaatcaaatagagggaataaaaaatggtaaaggggatatcaccaccgatcccacagaaatacaaactaccatcagagaatactattaacacctctacacaaataaactagaaaatctagaagaaatggataaattcctggacacatacaccctcccaagactaaaccaggaagaagttgaatctctgaatagaccaataacaggctctgaaattgaggcaataatagcctaccaaccaaaaaaagtccaggaccagatggactcacagccgaattctcctagaggtacaaagaggagctgataccattccttctgaaactattccaatcaatggaaaaagagggaatcctccctaactcattttatgaggccagcatcagcccgataccaaagcctagcagagacaccacaaaaaaataattttagaccaatatccctgatgaacgtcgatgtgaaaatcctcaataaaatactggcaaacccaatccagcagcacatccaaaagcttatcaaccacgatcaagtcggcttcatccctaggatgcaaggctggttcaacatttgcaaatcaataaacgtaatccataacataaacagaaccaatgacaaaaaccacatgattatttcaatagatgcacaaaaggtctttgacaaaattctacagcccttcatgccaaaaactctcaataaactaggtattgatggaatgtatctcaaaataataagagctgtttatgacaaactcacagccaatatcatactgaatgggcaaaaactggaagcattccctttgaaaactggcaaaagacaaggatgccctctgtcaccattcctattcaacactgtgttagaagttctggccagggcaatcaggcaagagaaagaaataaagggtattcaattaggaaaagaggaagtcaaattgtccctgtttgcagatgacacgactgtatatttagaaaacttggtcatctcagccccaaatctccttaggctggtaagcaacttcagcaaagtctcaggatacaaaatcaatgtgcaaaaatcacaagcattcctatacacccataacagacaaacatacagccaaatcatgagtgaactattcacaattgctacaaagagaataaaatacctgggaatccaacttacaagagatgcgaaggacctctggacctcttcaaggaaaactacaaaccactgctcaatgaaatacaagaggacacaaacaaatggaagaacattccatgctcatggataggaagaatcaatatcgtgaaaatggcgatactgcccaaagtaatttatagattcaatgccatccccatcaagctaccaatgacttttttcatagaactggaaacaactactttcaagttcatatggaaccaaaaaagagcctgcattgccaagaccatcctaagccaaaagaacaaagctggaggcatcacactacgtaacttcaaactatactacaaggctaccgtaaccaaaacaacatggtactgctacccaaacagagatagaccaatggaacagaacagaggcctcagaaataacaccacacatctacaaccatctgatctttgacaaacctgacaaaaacaagaaatgggaaaaggattccttatttaataaatggtgctgggaaaactggctagccatatgtagaaagctgaaactggatcccttccttacaccttatagaaaaattaattcaagatggattaaagacttaaatgttagacttaaaaccataaaagccctagaagaaagcctaggcaataccattcaggacataggtgtgggcaaggacttcatgatgaaaacgccagaaacaaaggcaaccaaagccaaaacagacagatgggatctaattcaactaaacagctgcacagcaaaacaaactaccattagagtggacaggcaacctacagaatgggagaaaatttttgcaatctacccatcagacaaagggctaatatccaaaatctacaaagaaatttacaagaaaaaaacaaccccatcaaaaagtgggcaaaggatatgaacagacacttctcaaaagaaggcatttatgcagccaacagacatataaaaaatgcttatcatcactggtcatcagagaaatggaaatcaaaaccacaatgagataccatctcatgccagttagaatggtgatcattaaaaagacaggaaacaacagatgctggagaagatgtggcgaaataggaacactttcacactgttggtgggagtagtgtaaactagttcaaccactgcagaagacagtgtggcgactcctcaaggatctagaactagaaataccatttgacccagccatcccattactgggtatacacccaaaggattataaatcatgctgctataaagacacatgcccatgtatgtttactgtggcactattcacaatagcaaagacttggaaccaacccaaatgcccatcaatgatagactggataagaaaatatgccacaaatatcccaaggaatactatgcagccataaacaagggtgagttcatgtcctttgcagggacatggatgaagctggaaaccatcattctcagcaaactagcgcaaggacagaaaagcaaacactgcatgttctcactcataggtgggagttgaacaatgagaacacttggacacatggcagggaacatcacacaaaggggcccgggggagggatagcattaggagaaatacctaatgtaaatgacgagttaatgggtgctgcaaatcaacatggcacatgtatatctatgtaacaaacctgcacattgtgcacatgtaccctagaacttaaagtataataaaaatatatatacatcactttacatccaaccagcactttggctttgaagccactgcctgatgttgacactttgtagatgtagtatgactattcttatatgtctctatctactgatgaggatcctactcttttagtataaagagtaccattgacttccaatcaactagtttcgataatatctgaaagatattaaataacctgacactagccttagtaaccgacaccttactggccctactactactactaattacatttttacttccacaaattaatatttatatagataaatccagcccctatgaaggcagatttgacccaataacctccaactgcctccccttttccataaaattcttcctagtagccaacacatttctcctcttagacttagaaatcgctctaatactacccctgccatgagctattcaaacaaattacctaacaacaataatcaccacagcccttatactagttatcattttaatcctaggcttaatttacaaaagaactcaaaaagggttagattgaattgaattggtaaatagtttaagtcaaaagaaatgatttcgattcattagattataataaaccatatttaccaaatgccctctatttacatcaacattatattaccatataccatatcactactggtaaaattaatctatcaaacccacctaatatcatccctatgtgcctagaaggcataatactgtcaatatttatcataattacccttataactttaaatatacattttactctagcatctataatacccattatcctcctagtatttgctgcctgtgaagccgcagtgggccttgccttattagtttcaatctccaacacatacggcctagattacacacaaaatctaaatttacttcagtgctaaaaattattccgacaattatgctgttaccaataatatgattctctaaaaattctataatctgaattaacatgaccatccacagcctacttatcagcctcatcaccctattgttttttaaccaattcaacagtaactcatccaacttctcactagttttctcttctgatctgctgatgtcacccctcctaattttaacagcctattaccttttataatcctagcaagccaatattaactgttcaatgaatcacccccacaaaaaagctctatatttctatattgatttccctgcagattttttaaaattatagcattcacagccacagaactaattatattttatattttctttgaagccacactaattcctaccctaattattattatcacccgctgagggaaccaaccagaacgcctcaatgcaagctcatatttcctattgtacacactagtagggtcccttcctctacttgttacacttgtctacactttaaatacttcagattctctaaatatgctagtaatgatatttactgaccaagaactgttagcctccttatccaataatcttatatgactagcatgtattatggcctttatagtaaaaataactctattcggacttcacctgtgactcccaaaagcccatgtagaagcccctattgccggctcaacagtacttgcagcggtactcctaaaactaggcggctgcggtataatacggcttacccttatcctcagccccctaacagaatatatagcctaccccttcctcacactatccctatgagggatagttatgacaagctccatttgtctacaaaccgatgtaaaatcagttattgcctactcctctgtaagccatataccacttgtcatcatagctctcctaatccaaaccccttgaagctttacaggtgccgtcacccttataattacccatggagtcacttcatccctgttattctgcctagcaaattccaactacgagtgagtccaaaacggaatcatatacttacctgaggccttcaaatactcctcccactgatatagcctcatgatgacttctagcaaatctcactaatcttgccttacccctaccattaatctagtaagggaactctctgtgatcatggcttcattctcctgatcaaacatcactattatgcttacaggacttaatatactaattacaaccctctactccctacatgtaccaatcacaacacaatgagggacacttacatattacattaacagttaaaccttcctttacacgagaaaatacattaatatttatacatgttacacctattcttctattatcctaaatcctaaaattattatgggctttacatgctgtagctatagtttaaccaaaacattaaattgtggatctaataatagaagcctgcaacttcttatctaccaagaaagtatgcaagaactgctaactcatgcccccatgcctaacaatatggctttctcaacttttaaaggattggagtcatccgttggccttaggaaccaaaaacattggtgcaactccaaagaaaagtaacaaatatgtatttttccactactataatagccctaatcgccttaatcacaccaattattacgttagtcaacccctgcaaaagaagattcatacccaaattacataaaaatagccatcacatgcgccttcaccattagcctcatcccaacgtttatatatacagaccaagaagtcattatctcaaactgacattgaatgacaatcccaactcttaaactctcactaagctttagactacttctccataatatttattctagtagcactatttgttacctgtagaattctcaatatgatatataaactcagaccctaacattaatcaatttttcaaatattttcctcatcacagtattcttccaatattccaccaacaaactctttcaactctttatcggatgagaaggtataggaatcatgtctttcttactaactggctgacaatatggccgagcagatgctaatacagcagccctccaagcagtcctgcacaatcgcatcggcgacattggttttattttagttatagcatggttcctcttttcctccaacacatgagagattcaacaagcattcattctaaaccctacctctaatccccttccattaattcgccttctcttagcagcagcaggaaagtcagctaaattcggcctccatccctgacttccatccgccatagaaggcccaaccccagcctcagccctgctccactccagcactatagttgtagcaggagttttcctgctcatccgcttctaccctttaatagaaaataacctctgaacccaaacctttacattatctccaggggctgttaccaccttatttacagcaatctgcgctctaacacaaaatgatatttaaaaaatcatagcattctccacctcaagtcagctgggccttacgatagtcacaattggcattaatcagccacatctagcattccttcacatgtgcacccacaccttttttaaagctatattatttatatgctcaggctccatcatccacaatctcaatgatgaacaagatatttgaaaaataggactattcaagactttacccttcacttcctcttcccttattattggtagccttgcacttacagatatgcctttcttcacaggcttttactctaaagaccttattatcgaaaccacaaatactgtcatataccaacgcctgagccctttctattactcttactgccacctccttaactgctggctatagtacccgtgttattttctttgctctgcggactggccatgcaggagtggtaagggcccgggccacgcgcgacgcctgggggccctgctgcatcgcccgtttgttcggggaaggtgggggggacgcttcatgccgccgcgcccagcagggtttccatgtctgagatgcccgctctggccccaagagaatgaaccagccgcagaggatggcatccgtggacacggacaaggagctcagtaacctggacttcagcatgatgttcccgctgcccgttgccaacgggaagggccggcccgcctccctggccggggcgcaattcagaggttcaggtcttgagaactggcccagctcaggctcctggggcagcggcgaccagaacagctcctcctttgaccccagctggacgttcagcgaaggcgctcaacttgctgagtcgcacggcagccactctccattcacattcctgggaccgggactcggaggcaagagcagctagcgcgggcctattcctcctttggtagagacgcaggcgtgcgcagcctgactcaggctggcttcctgccgggcaagctggccctcagcagccccggacccctgtccccctcaggcatgaaggggacctcccggtactacccctcctacgccagcagctctggcggacagaggcggacagcggcctggacacgcagcccacgaaggtcgggaaggtcccgcctggtcttccatcctcggcgtacctgcccagctcaggtgaggagtacggtagggatgccgccgcctatccatccgccaagacgcccagcagcgcctgtcccgcccccttctacatggcagatggcagcctgcacccctaagccgagctctggagttccctgggccaggtgggctttggccccatgctgggcgggggctcatgcccctcgtccctcccgcccagcagcggccctgtgggcagtggcggaggcagcagcacgtttggcggcctgcaccagcacgagcgcgtgggctaccggctgcacggaggagaagtgaacggtgggcttccggctgcatccaccttctccttggcccccggagccacgtaggcggcgtctccagccacacgcctgtcggcggggccgacaacctcctgggctcccgagggaccacagccagcagctctggggacgccctcgggaaggcctggcctcaatctactctccggatcactcaagcaataacttgtggtccagcccctccaccgccgtgggctccccccagggcctgggagggacgtcgcactagcctggagcaggagtccctggtgccttatcacccagctacgacgggggtctccacggcctggagagtaagatagaagaccacctacctgaacgagggcatccacgtgctctgcagccacgccatgggcacggctagcaacgtgcacacgctgctacccggccaccgggcgctggccttgggcttcgccggccccatgctgctggtgggggcagcacgcaggcctggttggaggcgaacaccctgaggacggccctgcgggcagcgccagcctcatgcacatccatgttgccctctccagctagccaggcgccctccccgacctctcttggccttccgactcctgcagtgggctagggcgagcaggccactggggaaatcaagcgggaggaggaggatgaggagaacacgtcggcggctgaccactcaagaggagaagaaggagctgaaggacccccgggaccggaccagcccagacaaggacgaggaggaagatgaccttttccccccagagcagaaggccgagtgggagaagcaggtggccaataacgccagggagcggctgcgggtcccttaagagggctcttaaggagctggggcgcatgtggcaactgcacctcaacagcgagaagccccagaccaaactccttatcctgcaccaggccgtctcggtcatcctgaatttggagcagcaagtgcgagagcggaacctgaatcccaaaacagcctgtttgaaacggcgagaagagaaggtgtcgcgcgtgttcggaggcccccagatggtgctttcagctgcccacctgggcttgagtgaagcccacaaccccgctgggcacgtgtgaaaggtacgcctccgtgggacgagccacccgccctcagccccgtgggctgggcccagaacgcccacttgaggccctgggcttcatccacatccacacctcacacaactgttgtcagcattgagccaacaccgacctgatgaggctcagagtgatgggggcaaggaaggtgagtgatgggggtgaggaaggtgacgctgggtccaggagctccctgggaccccggctcacccctcactgccctcgctccccctgcccccgtatctcagccaccatgtcaccctgtgacctgccccatggaccctgaaactgcatcttggccctgttgtctgggctggcaggagcttttttttttttttttttttttttttttccagtaaacaaaacctgaatgcaagcaacaaaacatacactttgtcagaaaagaaaaaaatgccttaactataaaatgtggagaaatcgtaacatatcacttgagggagatgctgtggaaacttggcttattcttcaaaagccagcagcaaattgtgcctaagcgtaattttttttaaggaaaataaaaagaacattagttatttaaaaaaaaaaaaaaaaaaaaaaaaacccggactgaccttggccaggctggatcagactggcctagagtagacttcagagggtgactcccctggtgggctggtctcagctgatcttgactgtcccgcctccacacagggcccatccatcttccccttgatcccctgctgcaaagacattgcctctgatgccacctccatgaacctgggctgcctggccacaggctacttcctgaagtcagtgactgtgacctgggacacaggctccctcaacaggagcgctgggaccttcccagccaccaccctcacgccctctggccattacgccatcaccagccagctgactgcctcgggtgcgtgggccaaacgctcacctgcagcgtggcacacactctgtggtccgcagaccaggtcagtaccttcagcatctactccagggacttcaccctccccaccgtgaagatcttacagtcctcctgtgatggcagtggacacttacccccgaccatccagttcctgtgcctcatctctgggtacacccaggtgccatcagcatcacctggctggaggatgggcaggtcgtggatgtgaactggtccatcgcctctcccatactggaggatgagctggcctccacacaaagcaagctcaccctcacccagaagcgctggctgcccgaccacacctacacctgccaggtcacctatcaaggtaacacctttgaggacagtgccaagaagtgtgcagattctaacccgcaaggggtgagcacctacctgagccggtccagccccttctacctgttcatccgcaagttgcccacaatcacctgtctggtggtggacctggcacccagcaaggagaacgtgaagctgacttggtcccaggccagtgggaagtctgtggctcaggtcatcctaaggcaagagaagcagtgcaatggcacgttcaccatcacgtccaccctgctggtgggcaccagagactggatcaagggggagacctaccagtgcagggtgacccacacccacctgcagtccacgaccaagatcagcggcccatgtgctcccccacaggtctacgtgtttgcaacgctagaagagccgaggaaccaggacaagcgcaccctcacctgcctgatccagaacttctggcccaaggacatcttggtgcagtggctgtacaacgaggtgcagctcccggacacttggcacagcatgacgcagccccgcaaaaccaagggctctggcgtcttagtcttcagctgcctggaggttaccagggctgaatgggaacagaaaaacgagttcatctgctctgtggtccatgagacagcgactggctcacagaccgtcaagtaactgttgtctgtaaatcccattaaatgtcctcctgccttcctccccactagggctctgtccagctgtgtggtgggaagggctggccagaccttctgtccactgttgcaatgaccccaggaagccacccccaataaacagtgcctgctcagaaaacaaacaaacaaacaaacaaacataaaaaccctagaggaaaacctaggcaataccattcaggacataggcatgggcaaggacttcatgactaaaacaccaaaagcaatggcaacaaaagccaaaatagacagatgggatctaattaaactaaacagctgcatagcaaaagaaactgatcagagtgaacaggctacctacagaatgggaaaaattttttgcaatctagccacctgacaaaaggctaatatcaagaatctacaaagaacttaaacaagttcacaagaaaaaaacaaccccatcaaaaagtgggtaaaggatatgaacagacacttctcaaaagaagacatttatgtggccaacaaacatatgaaaaaaagctcatcatcactggtcatcagagaaatgcaaatcaaaaccagagtgaggtaccatctcatgccagttagaatggcgatcattaaaaagtcaggcaacaacagatgctgaagaggatgtggaaaaacaggaatgcttttacattgttgatgggagtgtaaattagttcaaccattgtggaagacagtgtggcaattcctccaggatccagagttagaaataccatttcactgagcaattccattactgggtatatacccaaaggattataaatcattctactataaagacacatgcacatgtatgtttattgtggcacttttcacaatagcaaagacttggaaccaacccaaatgcccatcaatgatagactggataaagaaaatgtgacacatatacaccatggaatactatgcagccataaacaaggatgagttcatgtcctttgcagggacaaagatgaagctagaaaccataattctcagcaaacacaggaacagaaaaccatacaccacttgttctcgctgataagtgggagttgaacaatgacaacatatggacacagggaggggaacatcacacaccagggcctgttgttgggtggagggctaggggagggatagctttaagagaaatacctaatgtagttgatgggttgatgggtgcagcaaaccaccatgacacgtgtatacctacgttaacaaacctgcaccttctgcacatgtatcccaaaacttaaagtataataaaatttaaaaaaaaaacagtgaacatttattgtaggcttacattgggctaaatgcattttatggataatctcatttaatcctcataacaacccttggagaacatgtatgattattatctctattttaagcatggagaactgaatagcttacccaaggtggcatgactagtaaatcactggaccaggatttaaacccaaaaagtctgatcccagagttcatgcttataagccttcctaaccatgataatgctttaatctggcattaattgttctctaatttgggtaggtttgaaatttttcataataaatattaaaaatactttttgaggaaatgtcgtcatgataaacattagaactgcgttcatctaattacttttagggtatgagtgaaactaaaaagtaagactcatctaaaaataatcatctgttttggccatatccccagtgatacatgaatttaattgtttttattgttttcagaatgaaaaattaaagaggttaaataaaattagctggcaatcaaaaaatattagttatatgaagattcagagatttggttatacttaatgaatacaagctaataaatatttattaagactctactgtgctccaaatactatacagggaggtagggatatgaagataggcttagcatcagaaaaggactgtcccttagtagaaaggctgaactaaaaggaaaagtttgacgtgataggcaaattcaactgacaggtccactacccatagtcccttcaagagctcataattaggaaaagtcatatgtataatatggcactttcaataattaagtgcaataagtgtaagtgcaataattaaggtactgggaaaacataacagtacaaataattcagctgaagatttatgtaagataactaagatgacatttcaacgattggttgaattgacctctccctcatgaatgggattaaggcccttataagaggcttcatgcagcatttggctcctcttgcctttccactttcatccatgtgaggacacggcattcctctcctctaaaagatgtggcaacaaggctccccttggaagcagagagcagccctcaccacacacctgaacctgccagcacctttatcatggactcccaacctccagaagtatgagaaataaatttctgttctttataaattatccattgtcaggtactctgttatagcagcatacatggacgcccagtacatcatgttaatgagttcagaccttctttagtagacagtgcgtaaggtgggaaattagaggctttatccaggggaataacatggtgagattcatattttagaaagatagctctacttttgttatagaacagcagttctgaaagtgtggggactttagggtgtctgtgaagtaaaaaatattttcataataatggatgttatttgctcttttcactcattttttcataagtatagttttccagaggcaataagatatgttttctatttaaccaaacattgaagagacttgcaaaaatgtaaaacaatgtgactcttatcaaagtttattttggaaatagttgttttccataaaatacattacttatgttaacaggtaatggttttatcttaaataaattaataaatatttttatattttattagctttaatatttaatatggtaaatactggtagatataactcacagaaacaaaagttttttggcaacctcagtaatttttaagagtatacgaggaagcctgagatcaaaagctttgagaactactgacagaggggacgttgtattaatgagaggctaatagtagagaggcagctaattagattttatgaggatctaggccactggttttcaaaccttttaagtatatatgctcactaaaagaatttagaaaaaccacatcctcttacacatttttaaattgatagctaatttttttctttgtaagtagttataatgtattttctaaagtactgtaaatattgacatttaaataacagctatgtcaatcctttaaatgtgtccaactgaatctaaatagtgcttgcagtgagcggagacctcgccactgcactccagcctgggcgacagagcgagactccatctcaaacaaacaaataaatagtgcagcaatttgatacccacttatcacccacttaaaaagattatatgaatagacttttatttttttgtacaaactttgtatcattcctttttctctttaaaatagtattcccacttcccttctcattccaaattttgcataatatgttacactttgtatttggataatcttatgatcacctattttctttgtaaccaagatatgtatataagtaagttgaaattaattaaaaattttcctgtgaccaacaaacttgtaactattcacatttttcttctgggttatcattgaagttactaggatacagttttccaaagcaatgtaaataacaaatttggataattgctttaaaaattttattggtgaatggaacaatccctcccctccaattggtttgtatatcctcggatgaatattatttatttctataatgggacaagattaagcataaattctgtgttcattttgcactttacagatgtaaacgctgagaaaacttgtttgtgtaagtgagtggacaggaatggaaggagttttgttataatagtatcattcatttctatgacgccttctgagatatgcaggaaaaaagtacagtaatataccattaaaaattatctgttattgggcagtttcatttgatcagatccattatgtttcaattttattaaaagcagagaattaaaaccacttgactttcaaaaatgagtttactaaccagtcatcaggttcatctgttttctcaatttctgaaaagtataccaaaaagcttttaaaattaattccaactatatgttacactttcacgtggatgtaccttgtttaaactaatacattcagaaaagtttgaaaaaacagactattattaatacattattattaacatattttatcatgttttaaatatatgatttaaacacagcaaacctctcagctacctcaaaggttgagtagattttgtgaagaatgtccttcatacaacccatttggtaatgccaggcatcaaacaaatcacttaatttctggcagaaaaactcagacttcatttttcaagtcactaaatatattaataatatgtgatttttttaaaacctgagaaacaaatgacagaacaaatgcatggaggtatgtttattatgaagctaatgaagcatatccagaggtcctccttccaaggttcctttctaattttaaatagaaaattatgcactttcatgtataattttgtattctttgtcataatgaaagcctaaaaaaaaaaggcttcaaattatataagctttgggacctgtatctctgcctatacatgtgaaaaaatcatgagggcagtcaggataagctgaagatgatatatatatctggcataattaatttcatattatgaaaataagataatttctcattacttaatttgcaataaataatacagagtaaaatatggctaagattataacttatgaagtaaggtatatggtacatggcatggtcattccttcagtaaatgtgttagggggaggtatgaagtgaattctgactttggggaataattagaagcaacttgtatgaaaatcagaaataacttatttttcctcatgatcaaaattttcatatgtacttaatgaacacaaaaaatatatactgctaaaagcaaatcaggcagttagcatatagttttatttttatatttgcaagtagaaaataaatattttatacaatgaaatgaagttcattaaattttatgtaaatattttagaatatgaattgataaaataagtttgagctaacttattgaataataaacactgtcttagtccctttatgctgctataacagaatacctgaaaatggataatttataaagaacagaaatttatttctcacacttctagaggttaggagtccatgaaaaaagtgctggcaggttcaggcgtctggtgagagctgctctcttctttccaagatggagccttgctgcttcctccttcagagaggaggaacaccatgtcctcacatggacaaaagtggaagggcgagaggggccaaatgctgcatgaagcctctcataagggccttaatcctattcatgagggagaagccctcatggcgtaatcatctcttaaaggccccacctgttaataccatcacattaacaacacctgaattttggaggagacacatagcaaacactaaatctgaagtctgaaagaactcaatttttattcacaggattttgcccatgatatcatttttttaaaaactcaatttcatttaaaagaaactttgttatctatattgtttttcttgttcgaaaaggctcttttgttttaagctaacaatatggttgacggtaataactgggggaaaaagtgcctctgcttaaatgttgtaaaaaatcggtgaaacaggattttggactcctggaggatcctttactctttttactacagaagtacccattggatgatctttgtacattattcttcaccttgtgtgaagacctctgtgctagtcatgagataaggatgtgatgtaaactgtaacaatgggttaggagactgattggagatatttctaagctatactctaaagggacttagtgggtcattagacactggagtgaaagatgcgtcaaaaataattaagattttcaaattgagcagctaaatagattgtgatgctatttattgcataagaaacaaaataatcagcaagttttggaaagaggatggaattctgtttgcctatagatctttaatgtggaaatgttctaaaagtctggagcactaaacaaaggtcaaagatatggttataaatttgggagactttagtatataggtggatgatactatctaagggaaagtttttaaccaagagaggaataaatctgatgaagagaccctgggaaaccttcaatttagaagaggcagaatagtagtagtagtgttgggaaagtataattgaaaacaaaatcttccaacccagaaaacctttccacaaagatagaaggaaaacaattttatgattgagtaagcattagagcaggatgtgatgtgtatcacaagcagtcccctaagagatttgcaaagacagaaggaaatctcacccttttatatagccaacctgatataacccctttcattcaagttcttaggataaacattaactagtcctcaagtaagaagacttgacagcactatttgccacatacgtagtttattctaaattcacttggtaattggagtgactttgctaattggctttatacaaaggaaaatacacttttcatatcttcatgacaggaggtagttttgcagcttggaaggaggctcctgctgaagttaggcttctactcccacagaaactgggagtaagggtgctatcttccttgatgactgaattcccaagagatagttacacgttcttgagaaagacattcatgggtcacaaaactggcaagaggctttgaagcaagagatttttaaaagatttacatacacttcaaaaagacaaagaatttacaagttttctgaatgctctaagaacaaagaaggaggagtctttcttcttattttcaacaaggggaattaattttgttctttttaaatttgtatttacccttacataagtgatagaacagacagaaaaaaacagctgtggaaatgtgcgggaaattacaatgagtgctatatgaaaaccagaggaagagcatattttgcaaggagtgagggaatagtgctgtcttcaagaactttgcattccccttgtggaataaaactaacatacatttgataataacataactagatatgtaaaatttgtaaagactaaatgtaaaaagaatatagcattaggatgtttaattacgtagtatggactgccagtactataggaattcagtggcctgagggcaataagacctggagtaggcagggaaggatttgtggagattaggaaacttaaatgggattttgaaggagagattaaatctatgtaattcacatttttaatatcagtctaaaacacatctctgtagctgtctctataataaagataaacttagcaatggaaccatgatactcacagtccactaggccaaaacgttcccttggatagtgttaatgcttgagtgaacaatgacaccattgctgtgactttttcctttctgtattccttagtcacccctcttctggttaacaggttctttcttttatagctttaagtcttgccttggaaccaatttttttctccagcatatttatatggattcatttaaagcttaacttgcaaagagcagcttattttcacttcttagaaggtactctgcagttaaccttggtgctgtctacagagacaaaaataaatttctttggattttagttatctttctatgtcagagattcccaagaccatccccaggttgacgatttgctaggaagactcacaggactcagcatatatccatattcatggttatgattcattatagggaaataacataaagcaaaattaggcactctccgcctggcacatactaaagttacatactcccagaagaaaagtagatgtcagcataaaccgtattgtttgtacaaacaatctaggcatagtgagtcactcttatttagggaattgtggaatccctcctgaaatctaagttcacaggtgccaggcaaaaaggcccttttaatcatagaagcctcaggcctgctgtgataactcttttctctacatgtttttttttttaatttatacaatgatttgcaggtaaaatcaagagtccacatatcaagtaactgtcttttaaatgttattcatatatttacaggtatgctccagaatcactgacagagagcaagttttctgtggcctcagatgtttggagctttggagtggttctgtatgaacttttcacatacattgagaagagtaaaagtccaccagcggtcagtgtgctttttatttactttcagttttttgtttgttcgtttgatttttataaatttatggggtacaagtacaattttgctacatgcatacattgcatagtggtgaagtcagagcttttagggtatccaccaccttaataacatacattgtatcccttaaataatttctcagcatctactccaccaatggctcacccttccaagtctctattgtctatcattccacattctacgttcatgtgtatacattatatagctcccgcttataagtgaaaacgtggtatttgtctttctatggctgatttgtttcacttaagataattacctccatccatgttgctacaaaagacataatttcattcgttgttatggctgaatagtattccattttgtatgtataccacattttctttatccagtcatccactgatggacacttaggttgattccatgtctttgcttttgtgaagaatgctgcaataaacatacaggtggaggtatctttttgatacagtgatttcttgtcctttaggtagataccccatagtgggaatgctggatcgaatggtagttctatatttaggtctctgagaaatctttataatgttttccatagaggttgtacaaatgcacattctcaccaacagtgtataagagttcccttttctccacacccttgccaacatctgttattttttctctctctcatttttttttccattctgactggagtcagacatatctcactggggtaattttttctcttttttttttttgtcattctgactggaataaggcatatctcattgtgattttaatttgcatttctctggtgattagtgattagcattttttcatatgcctcttgtcagtttgggtgtcttcttttgaaaatgtctattgatctcctttgtccactttttaacacgattattagggggttttggttgaattgcttgagttccttataaattctggatggtagttccttgtcagatacaaagtttgcaaatattttctccgattatgcaggttttctgttcattctgttgattattgattttgctatgctgaaattttttagcttaataaagtctcatttgtctatttttggttttgttgcctatgcttttgaggtctttgccatgaattctttgtttacactcatttccattaaagttttctctaggttttttttctagtatttttctagtttcagatcttacatttaagtctgtaatcgatcgagttaatttttctatatggtgataaatatgggtccagtttcattcttctgcatatggcaatccaatttaccagcaccatttattgaaaagggtgtcctttcccatactgcgcaaagcaatctgcagattcaatgcagttcctatcaaaataccaatgtcatttgttcacagaattagaaaaaacaatcctaaaattcatagggaaccaaaaagagccctaatagcaaaagcgattctaagcaaaaagaacaaagctggaagcatcacattacttgacctcaaattatattataaggctacagtaaccaaagcagcatggtactcatataaaaaacaaacacatagatcaatggaacagaatagaaaacccagaaataaagccacaaatttatagccaactgatctttgacaaaatcaacaagaacatacattcaagtttttttttaacatgagaaaggatttatccaaagaatagtaagcctaggactcactttagagcttttattgcttatttttaaggcaatatcaaacaaaataatcccaaacctattcctaaactagcttgaatttttaaaaaggggatattgggaagctagttagtacagtctcttaatattacttttattaaatccagtaagctaattcattctaggaattattgcataagttaaaataaaccttccaaagtaatttttctcttggtcatcatagaattttttttaatatggaaaagtatgaaaaaataaaaatcatctctaatgcaattactcagagaagaccacctcaatattgtggtatgttacctttcaggcttcattttgtgtataaaagtatatacaaagaagtatataaaacttttataacttttataaaagttcacttatataaatatatattattttataaaattaaggtcatactgtgcagtttgctctccttctgtttcatgaagtattacagcatgggtaatttactgtattagaaaaatgtactgcaaatatgttttctaattgctgagtagcattccatcataaggctatatatgtctgaacaatcattttccattcttcagtcttttaagtaacacttcagtgaacatagtgaacatcctcatgcataaatatttatatttatctctgataattatcttagaataaaagtataagtaaaagttatgaactattttaaaatttttttaatttcaaaatgctttctagaaaagctgtgccaaattatatctcaacagcaatgcatagggatgtacatttcatcatttgccactattggttattatatttttatgtacttttgccaagttgataggggagaagttatatatattttaatttccatttcttttcttaatgtggttgactatatatttgttgggtgtttagggtgggtatgtatatatgtgaattatctcttctgtgctttgcctatctttatatttgtaattttttttcccaatttataagagctgtctatattgaaaataagtaaatgatgtttaagtattttcctagcttataattcagattttaattttataaactttgacatatgaaagttttctttgttgtatttatgcttattcattcattttttcttatattctcttttatattcaactttttatttcaattgaaatgtaatatgataaaaatatgagttttttaacaacaaaaaactcaaaggaaatatgtttttagttcatgtgttttgagccctcctcaggggatttgtgttgagtttattacagctatggaaatggaaattatagaagttccatatttaacagccttatgtttttgatcctaaaagtagtttgttttgaaaaggtttgaaaacatacaaaagcacacatatactaaattttttcccattgactggaggaaattgagaaagaattttgctacaaattaaatgtacaaaaaatattgaaagtgggtttgttttaggaatttatgcgtatgattggcaatgacaaacaaggacagatgatcgtgttccatttgatagaacttttgaagaataatggaagattaccaagaccagatggatgcccagatgaggtaacaatttttttttaatccagggtagtcatgcattttcttttactttttactcaaggacttcagttcacttcctgaaatttaatgtgcggagcttccagataaacagcataatcagatgactgtggaacaaggcatggttatgacatgtgccctgtattgaaaattaatgtcttccaccaattaaaagatggcccttagtgttcatttaattttggtttattttctcctttacagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtcagttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaattaagtataaggggttgttcgttgttgtcatttgttatagtgctactccactttagacaccatagctaaaataaaatatggtgggttttgtgtgtgtgtgtgtgtgtgtgtgtgtgtgtgtgtgtgtgttatttatacaaaacttaaaatacttgctgttttgattaaaaagaaaatagtttcttacttta

SEQ ID NO: 3 - DNA, 5883 bp, Artificial sequence
  synthetic sequence
ccgtccgtactgcagagccgctgccggagggtcgttttaaagggcccgcgcgttgccgccccctcggcccgccatgctgctatccgtgccgctgctgctcggcctcctcggcctggccgtcgccgagcctgccgtctacttcaaggagcagtttctggacggaggtaacgcctggtcccgcctcgaggccgccccgacgacgcggccggcccccgatcctggatctgcgttgtcgcccgtaattaccgtttagaggtccaacacggtggcctcccgggactagagccgcgggcgatttctcttctgcgtccctggggagcgcggagggcgtagcggcctcccgcggcgggagttagggttagcccgaggatctctgaaggcacccgacgtgtcaaactagaggttggaatggggagtgtcggggatctcctttcctgtccccagcagcttgtggctctcggcagatgtttggtgtggggggggattagcacagccgctctgacctacccctctaatcccccacttagacgggtggacttcccgctggatcgaatccaaacacaagtcagattttggcaaattcgttctcagttccggcaagttctacggtgacgaggagaaagataaaggtaagagcctaggagtgggtgctcagatccgggaggacttcctggcagaagtccttgtctgtacacacacagccgggacagtccccttggaggaggacaggtggaggaagtgggggagtcttctctattctctaagtcgagggtcctcgcgagtcaaggcccaacggtgacctcactaccgtcccgtctcaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtgagggcctgcttcctggtgctgatctctgtcccattagttagagggagacccagaccccattgactttcttaataatgattttttttggaaggggagctaaaagaataagtcccagcaacaatttattgcattatgatcgcagatctaggctgttaatttaatttgcgtgtttgtatatagttatttcccaatcttactaatgaggattttgagttctagagcactgattttttttttttctcctttaaacttaaggctccacccacagcccattcaggacagaatcagggtctgagtttctcttctcagccttgacagacccgagttgaagaaccaggtcttccttttataaagaggggtgagagcctcgagatgatgggtagtctctgactcttaactggatctgcttcacacctaggtcccgacatctgtggccctggcaccaagaaggttcatgtcatcttcaactacaagggcaagaacgtgctgatcaacaaggacatccgttgcaaggtgtgcctgggggtggtggcaaatggctgtcatggggagattcagaggtcagcctcattggggggtggcccccgctcaccttcttccttcttcaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggttggtgtttgggcaggggctctgctctccacattggagggtgtggaagacatctgggccaactctgatctcttcatctaccccccaggactgggacaagcccgagcatatccctgaccctgatgctaagaagcccgaggactgggatgaagagatggacggagagtgggaacccccagtgattcagaaccctgagtacaaggtgagtttggggctctgagcagggctggggctcacagtggggagtgcaccaaccttactcacccttcggtttccttctcccttctgcagggtgagtggaagccccggcagatcgacaacccagattacaagggcacttggatccacccagaaattgacaaccccgagtattctcccgatcccagtatctatgcctatgataactttggcgtgctgggcctggacctctggcaggtgagacttggaggaaaaaggaggatccctggggtacctcaagtgcataagatcacccaagaggaaagggacagggtaggcaccccaggtgagtctgactcaaaaatggtacttcttgtaaacagtacttcctggtctgtccctgtgaagtcctcacagcaacccctttaaggttatacttgctgtgcaccaagtacttccccaagtacttttatgcaaatcaacttctttacccccaaagacctagaaggtggtcaggtaacccagttagttagctggggctgggcacagtggctcacccttacaatcacggtactttgggaggctgagacagaggattgcttgaggccaggagttacacaactcaacctagcttggcaacacagcgaggagaccctatctctacaaaaaaaatttttttttttgagacagagtttcactcttgttgctgaggctggagtgcaatggcacgatctcagctcactgcgccctccgtctcctggtttcaagcgattctcctgcctcagcctccggagtagctgggattacaggcatgtgctactatggatgccaggctaatttttttttttttttttttttttgagaccgtgccttgctctgtcgcccaggctggagtgcagtggtgtgatctctgctcactgcaagctccgcacgaccccccaggttcactccattcttctgcctcagggtcccgagtaactgggactacaggcaccccccaccatgcctggctaatttttttgtatttttttttttagtacagacatggtttcaccgtgttagccaggatggtctccatctcctgacctcatgaaccacccaccttggcctcccaaagtgctgggattacaggcgtgagccacctcacccagcctttttgtagagacagggcttcatgttgcccaggttggtctcgaactcctggcctcaggtcatctgcccgcctcggcctcccaaagtgctgggattacaagggttagccaccatgcctagcctctacaaaaactttaaaaattggcgagatgtcatgcatacctgtagtcccaactaccaaggaagaaggatgatcacttgagcctggggcatcgaggctgcagtgagccatgattatgtcactgcactccagcctcggtgacagagtgagaccctctcaaaaaaagttgggacttggccggacacagtggctcacacctgtaatcccagcactttgggaggccaaggcgggtggatcacaaggtcaggagatggagaccatcctggctaacatggtgaatgaaaccccatctctagtaaaaatacaaaaaatttgccaggtgtggtggtgggcgcctgtagtcccagctactcgggaggctgaggcaaaaggatgacgtgaacccgggaggcggagcttgcagtgagctgagatcatgccattgcactccagcctgggtgatagcgagactctgtcccaaaaaaaaaaaaaaatgctgggactgaatttttgtctgttttggtcactgaaataccttctgtgcccaagacagttctggcatgtagtaggtacctgaaaaatacctgaataagagagtgagaaacaagaaacaggtgcagagaactgaagtcagtggcccaaggtcatgggggtaggaaaccacaaagctggggtttgaacctgggcagtacagcacctgagtctctccatctttttttttttttttttttaagacagagtcttgctctgtcacccaggttggagtgcagtggcttgatctcggctcactgcagcctctgccttccaggttcaagtgattctcatgcctcatcctctcgagcagctggaattacaggcatgcgccacgacgctgggcttttttttttttgagatggaatttcactcttgttgcccaggctggagtgcaatgatgcaatctcggcggctcaccacaacctctgcatcccagattcaagcgattctcctgcctcggcctcctgagtagctgggattacagggatgcgccatcacagaccccgggctaattttttttagtagagacagagtttcactatgttgcccaggttggtctcgaactcctggcctcaagtgatccgttcgccatgacctcccaaagtgctgggattacaggcatgagcccgtcccgtccctggctgtctctccatctttccatcttttttttttttttttttttttttggagatggagtctcactctgtcacccaggctggagtgcagtggcacgatcttggctcactgcaagctccgcctcctgggttcacatcattctcctgtctcagcctcccaaatagctgggactacaggcacttgccaccacgcctggctgattttttgtatttttagtagagacggggtttcaccgtgttagccagggtggtctcgatctcctgacctcgtgatccgcccaccttggcctctgggcgaggattacaggcgtgatccacctcacctggcctctccatctttttaactgcagtgtcagcggtgttccttgtcttctctgcagatgcaggcagcagaatatagtggttataggaacacaggtggaaaccctgtccaaagcaagggctatcgggtatcacctctgaccatccttcccattcatcctccaggtcaagtctggcaccatctttgacaacttcctcatcaccaacgatgaggcatacgctgaggagtttggcaacgagacgtggggcgtaacaaaggtgaggcctggtcctggtcctgatgtcgggggcgggcagggctggcagggggcaaggccctgaggtgtgtgctctgcctgcaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctc

SEQ ID NO: 4 - DNA, 14966 bp, Artificial sequence
  synthetic sequence
gaagggaggatgggctaaggcaggcacacagtggcggagaagatgccctcctgggccctcttcatggtcacctcctgcctcctcctggcccctcaaaacctggcccaagtcagcagccaaggtgaggtgcacagagggtggagatcacctatgcccaggaagagggagccctgggaggtgatgcagggccccgggaggggaggtagagtaagaggctctcctgctggtcccctccccttccacataaacatgcctgggaggacccagggccaactcaccagctgttccttagatgtctccttgctggcatcagactcagagcccctgaagtgtttctcccgaacatttgaggacctcacttgcttctgggatgaggaagaggcagcgcccagtgggacataccagctgctgtatgcctacccgcggtaggtgctggactgtgccccactccccatgtatctgtccctgcacttagctgagtcccactccagcagctttcctgcctgtccgaggaccactctgaatacaagccctaagtacctactttttgaacagatgtgctttggatgtatgtgggccccagctccagccctacataacccctaatcccacctatcccaggcagtgagaagaaaaatggcagtactagagacagaagttgggcatgggcccaggtctgggtcctcagggctccgcatggtggctgtgtaggagggacctcttctatgccaacagggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggtaagagccatcctcctgtcaccctgccccctccacttgctgcccccagtccagctcccggaatcagacttgcctgtgcccttccagctcagcacggatacccctattaccagaccccctgaggcaccccaagactcccttgtcattcctcccagccttggcatctagagctagaaatgcccaacagatcattcacaccctgttcctcccatcgtcaacaaatcatttattcatccattcaagagttacagaatacctactgtgtgccagacactgggctaggtgatggggatatggagagaaaaagggatccctgcttacaagaagcactgagtctagcaagccatcatactgcaaagaagtcactgttctcatgagagaagaacagaatcaggagcctccctagcctgccctccaggagaatgacagcctacaatttttgaatccagaagctgccccaattcagcccccagcacccctctctgcagtccagaggctgagccatagactgtggtactcagagttctgatgtgccctgtcttgccctcaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagtatgctgaccttcttctgccccacctcttatctcctaccttcaatcttgccccaggaaaggacagaccatactttggggattccagacctaggttcgtccttggagagttagtataggctcagatatgagccacgcctacttaggggcttcctactttgggtcattcccactgacaaaagcaaaggctttcaggcctccaaattaatggagatttcgcaacaaaaccctgaacaccacctgaaacccaccaacttagcccctggtctgtgtgcatatctatccagcaaggagctgagctcctctccacagagatgctgtgcaaatataagggttggaggctctctcagctgacaggcagacctagattgtgaagctgggattttcctcccaaggcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcaggtgagtcaacaaaggaatagggagatggggaggagataaaagaatatctctagggaagcctgggctagatctgaagctctgggaaccatggtccttcctgatgatctcgaacttgccactggacaggaactatgttcagggaaagaagagagaataggagtcaatgttctaagttatatgtgtagaaattatctgaaatctgaacaccctatacagtaggggcacacgggccctgatgggacttacttctttgactttagtggcacttggactgcaatgctttaccttggacctgaagaatgttacctgtcaatggcagcaacaggaccatgctagctcccaaggcttcttctaccacagcagggcacggtgctgccccagagacaggtgagagctgaactgctgattgaggttggtgtcatgggagtgagccacaatcttgcagaaaaaaagaagagagtgttcttggtcctcttcactctccttcctttgtctccaaaccatacagctttcaatgctctcttatctattctgtcatcctccaatcgatattttatcttctcaacccctctctgttcccattgggaatgctttggtttagtttagccttaatcatgtcactgggaccattgcaacatcctacagtctaattcacctccaatgtattccccatgctgctaacagagtgatctttgttaagctcaaactgtgcatgacctttgtaaagctctaactttgcatagtctccccttactcaaacatgtactgcaatttctcattatcaaagccataaaaaatttagcctggccttcaaagacttccaccaagcacctcattgattcttcatcctctccatatctaaatctataaccaagttgtattgattttacctattaactatctctagaatctgcccacttgtctatcactgccattatcctactacaacccatcattaattctctcttgggctactgaaaagacatagtaactgatcaccctgagtcttccaagtaattcttcacataggtgctgaagggatcttttcattttgttttgttttgagacagtctggctctgttgcccaggctggaatgcaatagtgcaatcttggctcactgcaacctccacctcccgggatcaagggattctcctgcctcagcctcccaaggagctgggattacaggcatgtgccaccataccccattaattatttttgtatttagtagagagggggcttcaccatgttggtcaggctggtctcgaactcctgacctcaggtgatccacctgccttggcctcccaaagtgctgggattacaggtgtgagccaccgcacctagcctgaagggaccttttaaaagtgcaaaaatgaatgtaacactccctgcttaaaaatcttcaatagcttcccactagtcttaggataaagaaaaaaacctctaccatggacttgaaaagttctgagactcagcccctacgtacttcttcagcttcatcttgtgctatgctgccctttgatccccatgctccagcacagggcttctgtggtcccctcatatctggcatattcccttctgcttctaggtctttacactgctgtttacccagccctactgcctttacttaacttctacttatcctccaaatctcaactcaagcgtcactttcttagggaccctcccctggccttctttattaagtccaatagcactaatatacattcttgttgtaccatgtaccgcttcttcttcttctttttttttttcttttttttttctttttgagacagagtctcactctgtcgcccaggctggagggcagtggtgccatctcggctcaccgcaagccccgcctcccgggttcatgccattctcctgcctcagcctcccaagtagctgggactacaggtgcccgccaccacgcctggctaattttttgtatttttagtagagatggggtttcactgtaagtgttagccaggatagtctcgatctgacctcgtgatccacccacctcggcctcccaaagtgccgggattacaggcgtgagccaccatgcctggccgtacctctccttcttaagtacttatcatagtcatactttcatatttatagtttatatgattatttggttgataataaaaatctggccaggcccagtggctcacacctgtaatcccagcactttgggagactaaggtgagaagatcacttgaggccaggagtttgagaccagcctagacaacatagtgagacttcgtctctacaaaatttctttaattagccaggcatggtggcacctgtattcctagctacttgggaggctaaggtgggaggatggcatgagcctaggagtttgaggttacagtgagctatgattgcaccactgcactccagcctgggcaacagatcaagactttgtctcaaaaaaaacaaatctctcagaactccagttgagaaaggctgatcttgaccacatatgatccatgagggcaagatcaatgggtttttattcaccaatatattctcacctaccaatagtaggtgcctggtaaaaaattgttgaacactattaactatttaaatgtattcttccagtttttttctatgtatttacatacatctctatacaacatttaaattttattatttttgagatagggtcttgctctgttgtccaggctggagtgcagtggcatgattatggctcactgcagcctcgacctcccagactcaaacgatcctcccacctgagcctcctgattagctaggactacaggcattttctttctttctttctttctttctttttttttttttttaagtagagacaaggtctccctatgttgcccagactggtctcaaactcctgagctcaagcaatcctcccatctcagcccctctaccacgcccagcctaacatgtaatattttttttttttttgagatggagtcttgctctgttgcccaggctggagtgcagtggtgtgatctcagctcactgcaacctccgcctccagggttcaatcgattctcctgcctcagccccccgagtagctgggattatagatgtgcgacaccatgcccagctaattttttgtatttttagtagagacgaggtttcatcatgttagccaggctggtctcgaactcttgacctcaagtgatccacccacctcagcctcccaaagtgctgggattacaggtatgagccaccacacccagcctaatatttaatttttaaatataaattggattacattacatatatggtttgttgactcacctttttgcttaaaaacatgtctcagagatcattacaaaaagatgtaactcattctttttaataactgcatacatatccataggattgagatgcctgagtggactaaagggtagcaaaactgaagtcatcatttcctctatcaaaacttgcttttcctgctgggcacagtggctcacacctgtaatcccagcactttgggaggccgaggtgggtggatcacctgaggtcagcagttggaaaccagcctggccaacatggtgaaacctcatcattactaaaaatacaaaaattagtcgggtgtggtggcacgcgcctgtagtcccagcttcttgggaggctgaggcaggagaaatgcttgaacccgggaagcggaggttgcagtgagctgaaatcgtgcccctgcactccagcctgggcaacagagagaagactctgtctcaaaaaaaaagaaaaaaaaaacttgctttttctttattgtctccgatcctaccatgaattcagttttcctggactgaaaaatagaaatcatcctggacttctcccattccaataccctccaggcctaaccagaaatagtcttgtcatttttaccttttcaatatctcttcaatcatctctttccctgtctctcactgctgctaccctattttcacatcctcatcactttgcacatgaaaaactacaagaatgtcataatggatttctctgcttctagtcggccctcacaaatcctttctacagactgtagttagaatgatcttcctaaagtacaaaggtagctatggcacttccctgcttaaaatccttaaatgtctacataactaccacaaggttagtagtaattcaagttcaagctccccagaaaggccataaggccatccatgacatggtctgcagtctcacatttaccacctaccaccaggccctttatgcgaaaaccatctgcaccacttagaatttcccccaaagcaccacactctcttatctatgcacaaccatgtgcctgggatgcctgtttgtcccttttgcctatcctatttttagttcaaaactcagtctaaacatcgccgtatccaggaagcattccctgaccctcccagtctgggttggatgctcccctttgatgctcccaagccagtctatgcacatcacagaaaaatatcatgtttgtttttctgtctttcccactgaaattagagctctttgaaccttattcctctatgcactcatagggcctggcacagtaccaattggcacatagtatcaattatttgttgattaaatgggtaaacatacccattttagtgacggtgagccacttaggggttataatcaagagggagacatgattcaattgttcaataaagaattattgagagtgactttttgcaaggtactggggataaatagtgcaacaaataaaatggaaaaggggccaggctcggtggctcacacctgtaatcccagcactttgggaggccaaggtgggcagatcacttgaggtcaggagttcgagaccagcctggccaacatggcgaaacgccatctctactaaaaatacagaaattagtcaggtgtggtggtgggtgcctgtaatcccagctactcaggaggctgaggcaggagaatcgcttggacccaagaggcagaggctgcagtgagctgagatcgtaccattgcactccagcctgggcaacaaagcaagactgtctcaaaacatttttaaaaataaataaagtggaaagggttccagcattgttggggcttacattagtagaggggaggggaaaaggagggggagaaaagtaagtaaagaaataaaggaacaaagttataatagagactaaaggggggcgtgtggtgggtggggggtgggtgttgctaatttagactgagtggccaggaaaagcctcaccagggaggtgacagataagccgagatctaaatggcaagaaggaatgagtcacatgaagacctacagtcagagcaatccagggcaaaggcaaagtgcaaagatagcacatttggcatatctatggcacagaaagaaggcctgtgcggatgaagggtgataaactggcatgagaatcataagagatgagaatggcaaggcaaggagaaaccaaatcacagagtcctgtaggcctggtaaggggtttgggttttattccaagtgggatgaagagccactgaaggattttaagcagtaaacaatgacaatttgacttacatttttaaaagatatgtttctctgtgaataaataaataagagtagaagcagaatcaccagtcagaaatttgccttaatccaagcaaggaatgatagtggcttcatctaacatagcagtggtaaagatcaaaatcaatagtttgtgacattatattctggacattatcacatcaacatttttggaaacaaattctggtagctgtgtggagaacaacttgaaggtagtggcaaaaacagaagcagaaaagccagttaggagactattgtcataatctaatagcaaaataacacataggggagagtaggaggaatgacgtatgagaacacgttaaaaatagaggatatttttctccagaaagatgagggttctttaatctttgaaacaatcctgtgaggttggtatcattaacccattttacaaatagaaggaaactggagcagagataaaccaagtaatttgcccaaagttacacaaaggtaagccaagacctcaactcttggtgaaaacccatgctgttttcacatatcatgccaggtctttcctcaaagggaagaggccagacaacaaagctgacttcagaaaacatcatggcaaaatagcaaaaacaaacgaacaaatgaacaaaggaaaaaacaaaaagaaaacatcatggcataaagacagagggaaagctctggaggcccctggaaggaaggagttaaatgataatccctgcaggccatcgttcttgtaggatgggaagccttgggattagtctctggggcaggcctgattcaatgactctgtggggctgggtcttaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctggtaagaactttcttcctcattcttcccacatagttcccacccccactgaatctgaccctgtgcccaggatccccaactctgacccttctgaccgatggctctggtggcacaatgccttgtgcacagaaggacttaagctgctccctgctgacatccctgtagtgcgcctccccaccccaaacttgcactggagggagatctccagtgggcatctggaattggagtggcagcacccatcgtcctgggcagcccaagagacctgttatcaactccgatacacaggagaaggccatcaggactggaaggtatggtcaagcaacaaatgcccacagacctcactacgcaggggatccctggggttggccatgcctgttagcaggagtgaaagtgtctatgtatccagtctctgctagtatatctatgtttatcagattcaactggtatcttagtctctcttgggcatctgatatttctggctactcctacttaacatttctcttccctttgtctttgggatatcagtcttccccgattttcctcctacctctctgactgaactttctcactctcctctcctggctcctcctttccagccttccacaaaatgctgtgcttcctagagtctcatcctaggctcttttctcctctgataaaataaatatgttctccccaggtgagtgctttaactccatggtttcaatgtccatacattaacaatcgcaattttaccattctaactcccatatggaaaggaatttacaaaccacaggcaaattaccaaaatgtttgaagtctgtttcctcattggtgaaatgaggatgaagctatctaccttatggagttatagtaaagattaaatactatatctaaatacctagcatactgcctggcataaggaaggtgctcaatatacattacttaaatattagtttgtctacttttattctcatttgccacacatatatacaactatgtacaacatcttatggttctaccaaaacttcctccccctggacatttcagactcagtctatccagaatggaactcctcttcctcctgaagctttacttttttattgtgataaaacttacatacaacaaagtgtacaaattttacatatatagtctgtacttgtgtatacatccacataaccataaccaggatcaagatataaattatttccagcacctcagaagctcccaatgctccgacacatcccccgccaccaaaggtaatcaccatcctgacattaccattgattcatttggactgttgttgaactctataaaatggaaccctaaagtatatcttcctcaaacttacttctcttctgtgtcctgtctctgacaatggcaccgctacttacccagtacccaagcctagaagcacccttgactcttcttgctcttcccttatcccttccccttactcatcaactaagttgatggcaataacgtactaatagtcctcaaattcctaccaggattgccttagactcatctgtgaccatccagccgtgtcatccatgcttttagcacactaaactactctgtggcacaaaaccaagcattaaatgcttctctggacttccgtggtgccttgcactaattaattgttgtgattatttacctgtttggatgtctttctcattagatatgtaagagacggtgttttattgctttctccccagtgcctaacccaatgtttggagtatcacacagttcataaatgtttgctgccttcctgcttgtacacatgatgcttgtaccccagcgcctaacgcaaatctggcatcctctgcagcatgagtattatttgtggcatggtctgtgtggctctgaatatatctgtttctgggggtgtccatcgcccgacctggagttgtgagaacacccggtagggtgtgcgtgtaccgggatccctgccgagggtgtacctgggtgttggtgttaggatacgtagctctctgaggtgaggtctgtgtctcaggaggtgggcctggcacgtttctctcgggcagagcgtgatcccgttagggagaggctctcggttagggcgctctatcctgttgctgggaagcgtgttttctgccggtggggctctttgtgggaatctccgaccgcctggggattcggagctgcaggatttgggtcaaacagacgctgggctatcgaagccccgacgccgggccaccgcacgcttctttgctcaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccggtgaggcaagccccggccgcaccaaagccgcacagcgcctgcgccagggactgggcgccgggtgcgagtggggcggggctcggagaggggcgaggcgcggggcgcggagaggggcggggccctgaccttgcgggccgacggctgcgcaggtgcccgcagtgcccaggggcggcgaggggcggggccagagtaggggctggctggatgagggcggggctccggcccgggtgggccgaagtctgaccctttttgtctcctagcctggatctccttggtgaccgctctgcatctagtgctgggcctcagcgccgtcctgggcctgctgctgctgaggtggcagtttcctgcacactacaggtaccgcccccgccaggcaggagactggcggtggaccaggtggagccgaaggcctgtaaacaggcattcttggttcgctctgtgaccccagatctccgtccaccgcccgtgcgcacctacggcttcgccacttcctgcacgtcacctctgggactcgccgcggctccttacactctaacacgcccactataccgcccacctcgaacagccccgcctcctgctgctcacctcggcgactaggccaccgtccacccttcagccaaactgcccactccacccccatccaatctgccgtcaggtcccacctcctaaacctagtccaaacaatggccccctttctctagccctacagacacgacctgactcactgaaacagaacctcttgcccctccgatctagcctcacacagccaccagaataatctttctaaaatgcgcacatgaacctatcaattccctccttaaatcctttcaatagctcccaggatatagtccaggctccctatgatgacaagcgaggctctgcaaaatcggcccctgccagcaagcacttcagcctgttctaccctgtgcatcagcctttccaaactggttttctccacaagtcaggttttacttctcagtgcctttgcacataacgtccgctctgcctgaaatagcctccctgtctcactaaacccattcatctgtaaataactttcttcaccaactcttaagaaacctttcctgaccctcccccttttggccgctgctctgtgctccaatacagtaccagtagtgttttactgtgtttatttctttatatatcttccctattataccttacttggtcatctttaaatccttagcacagtgcctgccaaaaagcaggcccttaaataaatgttattaatgagtgactcatttatgtgggaagacgaaggctgaggagaaatgccattggaggctataaacatgaagggtagaatgcatcccacctccatccttcatctgctgcttctttaagacttagctcagaagtcatctcctccagatggtgtcttctgatggtcctgggctggttcaggtgccactctcatgggctaccatctccctctgaacatacctctctttgaacttttaatctgcccatacgtatgagcagactgaaaggtagcctccatgcaggtagccagatccaacaaaaaagttggagaagtaggaggaggtggtctgctgagccaggcatactcagtttcttttccccaatttaccaggtaagtcacaccttctcccctggagagatatgagtgaggtcactgaagcggcctgaaatgttctactgacgtccctccacatggaaatatgaagctaaagaatccaggttctggccgggtgcggtggctcacgcctgtaatcccagcactttgggaggccgaggtgggcagatcacaaggtcaggagatcaagaccatcctggctaacacagtgaaaccccatctctactaaaaatacaaaaaaaattagccaggcatggtggtgggcgcctgtagtcccagctactcaggaggctgaggcaggagaatggtgtgaacctgagaggcggagcttgcagtgagccaagaccgcgccactgcactccagcctgggccacagagggagactccatctcaaaaacaaaacaaaacaaaacaaaacaaaacaaaacaaaacaacaaaaaaatggaggcccctccaatacatcttaatccatattcttcccagatagactaccctctccagtaacttactaataagctccactgattttcttcattcattcattcagctaatttactgagcacctactatgggtcagacactattctacaccctagaaatacaggagtgaataaatagacaaactctcttttctcggggaccatatgttctagtggggtgagacagacaaaaagaaatatacataacaatgtaataaatgtacaacaagtcaaatactataaggaaaaatgaagtacagcaggagatagcctgtcatggggaaaggaagcagcactacagtggtcccctcttatccacagtttcactttttgtggtttcagttacacgcagtcaattaaggtctgaaaaaaattacatggaaaattccagaaataaacaattcatgtattttaaattgtaagtagcacgatgaaatcacttgccatgccgctctgtcccacccgggatgtaaatcatccctttgtccagcatctccacgctgtatatgctccctgccccatagtcacttagcagccatcttgcttatcagattgactcgttgaggtattgcagagctgtgttcaagtaaccctcattttagttaataatggccccaaagtacaagagtagtgatgttggcatattgttctaattgttctattttattagtagtcattattaatctcttactgtgcctaatttataaactttattgtaggtatgtacgtatagaaaaaacatctgctgggagtcttgaaacatatcccctgcagataaagagggactactgtgtttcaggcaaggtagtcagggatgacatttgaacagaggcctacaatgaggtgagggaatgaatccagtgttccagacagaacaaacattggagtgtttgaggaaactgcagcaccctttgttgaacctgcccaccactgacttccctcccctacctgaggttttcttcttactctactcccctgcccaactttattgccttgaccatgctcttatatctccaagccttactccccggcctcactgctcccccaccccaactgctgtcctcctccctgccaatccactgccatggctcagtctgcttctcttccttctcccccaggagactgaggcatgccctgtggccctcacttccagacctgcaccgggtcctaggccagtaccttagggacactgcagccctgagcccggtgagtgtgcttccctcccctgtgcccaccaccaaccctgcctggtactggatccttgccccaacaatacaacttgttcaaggtccttgccctaccagtgtgccatccccaggcactaccccagcactaccccagcccttctccttcctgtacagtccagcccctcctcccacaggatctgctttaatccagcgcctctcctcatctctcccagcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttga

SEQ ID NO: 5 - DNA, 80968 bp, Artificial sequence
  synthetic sequence
cggcagacacgcacgcacccgggcgccgaagggaaagccgcgtctcgccctcccgccccgccgtcggtcctgtctcagtccctcagcagagcgggaaagcggaggccggagccgtgacctctgaccccgtggttatgcggagccgccgcattccttagcgatcgcggggcagccgccgctgccgccgtgggcgactgacgcagcgcgggcgcgtggagccgccgccgcccctcccccaccgccgctctcgcgccagccggtccccgcgtgcccgccccttctccccggccgcacccgagacctcgcgcgccgccgctgccacgcgccccccccaccgccgccgccgccccagccccgcgccaccgccccagcccgcccagcccggaggtcccgcgtggagctgccgccgccgccggggagaaggatgaaggacaaacagaagaagaagaaggagcgcacgtgggccgaggccgcgcgcctggtgaggcggacagccgaggggggctccgtggggcccggggtggggggggctcgccgcgcacccccccactggggggggaggggcaaggccctgcccaccgcgggagggggcggggccatcttcctttaagaacgggacagccccgcaaggcgaggggtggggagcgctccgccgggatgggatgtggcgcctttttccgctcgccctcgcgccccccccgccccgcgcagctaaattccggcggaggggcgagctggcaggccggctcctcccactctgggcagcggggtcccgcgtcccctcccccactatttggcagcgtctgggggtctggggcagcttcgttcattcacccgggggagttgggtttccgggaagggtcggaagctcctccctcgcttcctggtgggtaatggggtggtgcctttgactccgggggtggaaaagcgaccccacattcaaggacgccaatggcatgttgagctttcccaatctaaaccaggtgcgtggagggaagcaagtgcttactcccagcttgaaccctgagcagcggttctctaactttagtgtcctgtgggcgacacctgggaggcggttaggaacgctactcctagggcccttcgtaagacctgctgagcctcagggtctgggatagagcctgggaatctgcgctttcaccagcttcccaggtgattcaaacgcagtggctcacggaccccactttgagaaacgcagactgggctgtttgccttcagaagctggactggaaattggtattttgaggagaactgaatccactttgcaaagaatgtatgttggacaaagacaaagtatctctgttgggagacgttgttcactgaggatttagcaagggagagcgtaacttgagtgtttgaagccgtctcgttcaaccgatgggggttgtgaattttgtgttcagagcgtcagaggacttgcaggtgaaatagcttccttttggattactcccacaaagctcagacactttgagacatattcgggtaattatctgctgataccagtagtaacaggttatactaagatgtcagttcctaagatctgtcacacagcgaggttgtggtgcggatagcataagtgtctcttgacactgtagtttatatgtaatggttgtaattcagttttaatgatgagtgaagctcagacttaattttctctcttattttgaagtgagttttctgcttcaaaggtgttgtgcaaaaaaaaaaaaaatagactgtattgggggggttgacacaaacctgaagtacacctttttctttctcgaagtctgtaaaagttacagatttataaaaactgaaggaatttgggctacagaattttcattggtgcttgctatcctcttccctagtcatttgttgcccagggaacaaaaatagcaattgtttgtatgctgaagttttgtgcttcagtttattttggcctggcatgtgaagtttgaagattttgaggttccgaaatattttttttctgattgagtcaaaattggaagcttgggctagttttcaggatcttgtagaaatttcaggatttctctaacggttctgcacctggaaacaaatataggcctgatggggagtgggagagagctctcgttctcctcctcgacaatgagttttgtgtgttcattgacattctccttggcgttcctgttatgcgggtgtttatgaccctgggcacccaaggaaatttgcttgccagagttttacttgttagaggtaggtgaaagtacttttaagtcttgtgttatttatgtcttgacagaattatgaaatctgtttgtctcaaagttctgttggctcttactagttgactttaggaatttggggtccactgttaagtctctttagattctcgaatttaccgtggtctgaacttcaacatcgaggttgctatttctgttttatcattgactcaacttcaagccaggttatcagggtaagttattcctctttttcttctaaggctcttcattaaaaatagtaatatgaataggaattactttgtttgtttgtttttcgtttgtttgccaccacacccggctaattttttatttttagtaaagatggggtttttccatgctggtcaggctggtctcgaactcgtgaccttaggttatccgcccaccttggcctcccaaagtgctgggattacaggcgtgagccaccgcgcccagcctatgagtaggaattactttgtggtgctgttatacatgtgaaaaaggatttgcgagactttgggagtctgaggtggaataatcacttgaggctaggaattcgagatgagcctgggcaacatagacccggtctctacaacaacagcaacaaaatgagctgggcatggtggtacacacctgtggtcctagctactcaggaggctgaggatagcttaggccgaggagttggaggctgcagtgcccaatgattgattgtaccactgcactccagcctgggtgatagagggagaccctgactctaaaaaaataatgaggctgggcgctgtggctcacgcctgtaatcccagcactttgggacgccgaggtgggtggatcacagggtcaggagttctagaccagcttggccaacaaggtaaaacccagtctctactaaaaatacaaaaattagccgggtgtggtggtgcacgcctgtaatcccagctactgagggggctgagacaggagaatcacttgaacttgggaggtggaggttgcagagagccaaggccatgccactgtccagcctgggtgacagagcaagactcgtctcaaaaaaaaaaaaaaaagaaaaaaagaaaaaggacttgttgagaatgtgatatgcaatttgagtatcttgtgtgtgttttttgttttagaattttccatttctttgaatgggactggatggtaggtgagggggaactttttaagagtcccaggccgggcacggtggctcacgcctgtaatcccagctctttgggaggccgaggcagacggatcacctgaggccagaagttcgagaccagcctggccaacatggtgaaaccccgtctctactaaaaatacaaaaattagccaggcatggtgatggtgcatgcctgtaacctcagctactcgggaggctgaggcaggagaatcacttgaactcgggaggtggaggttgcagtgaacccagatcacaccactgcactccagcggcgacagtgagactctgtctcaaaaacaaaacaaaagaaaaaaccagtcctcttaatgtttaaaaaaagttgctgtaacactgtactggagaatttgtgagagcgtacactggactgagagaaacttgactaggagggaacgtaggacagttgattagaacacctgaacacttctctttgggcaggtgaatttggagtgcttgcaatggcaattgtatgcacttccatgggaacgctttcagattgctcctgaccccactcagattggtgactagagtgttactgggaatccttttaaaaattgtttttgtaggctgtgcgcggtggctcacgcctgtaatcccagcactttgggaggccgaggcgggcggatcacgaggtcaggagatcgagaccatcctggctaacacggtgaaaccccatctctactaaaaatacaaaaaattagccaggcgtggtggcgagtgcctgtagtcccagctactcgggaggctgaggccggagaatggcgtgaacgcgggaggcggagcttgcagtgagccgagatcgagctactgcactccatccagcctgggcgacagagcaagactctgtctcaaaaacaaacaaacaaaacaacaacaacaaaaaaacctttcgtagaggaaagaagattcttaagaatttcagtatagtgtaccccaggagattgatttttgcagatgttaattatgctgttgaaaaatagtctttctttcacacattttgtagtattgtcttttctaggtgtaagaagtagaccttatgtggttgagtggatataaatcctccaacttgttataaacagtttgtttgggagaaatagccaaggggatttccctttaaatcgaggacgctaaatggattctccctggcttcgagaaactctttgttgtatgctttatcttttactttgattcatgactgaccagttcttacatcaaacatttttttcccctttgaagtaagagaagagatacttaaaaaaaaaaatgtagtctagaaagagactaggaatagaacactgctgacttgttcttataccaagagctggaggcataaacagttttaataaaaaagaatactttgtatcttttgatgtaacttccctttagagattctgtttcccctgtcttaaattctcaggctatttctgattgctgtttaaataattggtagttactatttgagtcatgcttcttaaaaaaaaaagatataaaaattcgccagctcagctttcatttaattagtatcaaagaaatgttatctgtggcagtgaacattagtagcttgttccagggtttattgtaggtttgttcaagtcagtactgcccatcttggcacttaggttcttggaatacagaggtggataaggtttgcccctcagggcttatggtcttctggggatgaagaaggtccagacgcaggaacagaattttactacagtcatgatacgtgttatcatagttgtataaatcataaacagatatcccagttgctgcctcttgtctagtcatagtttctcctttcactctcaactgtcctggtttggaagataattgtatgtgttatgaaacaatgggaacattgaaaagagacatttacctctgcctggcagagatcagggaaggcttcctggaggaaggtgctttctgagctgaatcttgaagggtaactaggagttaggccaatcacagagggcataagcaggagctggtgacaacacatagcaggagagaaagagtcagagtgggtgagagggagtgtgtgtatttgcaacgtgtgggaattaggtatatctacaaattattggaacataaagtttgtagagtgtcactgtggatgggtagagaggcagagaccagattgtgagggttccatatgccaggccagtgagcacctactctgtgccagacattgtaggcattacttacattgtctctaatctgtccaacaactcgagaggaagacatcagtatccttcaaaagagaacctaggttcaggttgggttgtaattgccccagtttagaatgtaaaaatttgaatctaactctagatcctgttctttaccggtgggccagtggactttggaagcttcagcttttgcagtatgtgattaaggagctgttgaaggattttatttgctttctattattattattactattttttgagacagggtctcactctgtcaccctggctgaagtgcagtggtgccatcacagctcactgcagcttccacttcctgggctcagatgattctcccacctcagcctcctgtgtagctgggattacgggcatgagccaccatgcccatctagttttttgtatttttttgtagagacagggtttcaccatgtcgcccaggctggttttgaactcctaggctcaagcaatctgcccaccttggcctcccaaagtgctgggattacaggtgtgacccactttacccagccatatatattttttaaattctttaaagagatgaggtttcactgtgttacccaggctggagtgcagtggctatacacaggcacgatcatagtgcactagagcctggagctcctgagcgcagcaagtgaccctcctgcctcagtctcagctgagactataggtgtgcaccaccgtgcctggcctgttgaaggattttagagctgaaagtgatgagggcaggtttgctttgctgggttgacctcagtctataacatgtaagttctaccagagcagggatttggtcttgtttactgtgaatccctagaattgtgtctggcacaaggtacgtgttccatgtgtttgttgaatgaaggatgacaacagagtcagaatggatttgaggaaagcaggactggatggagggataaatattaagaggctgttgtgttgggtcaggtaagagactggttttgggacttggcggacagatgagcgagaaagatgacacctgcgtttctgaattaggtgattatacaggctgtcaatcaaggtagggaatacaggaagaagagtgggtctggagaagaggtgaattagagttgccaattagatgaattaagagtagagctgaattagagtagataatgaaggggtatagtcagtacacaactgaatatttgaatctgaaatttgggggagaagtcagagctagaggtagagattaggtagttatcagaatttaggtagtagctgaaactgagatgataatgttacccatggagaatggaaaatctgaaaagagaagcaaagaataggaccatgggaaataggggttttattttagtaccttctccaaaccttgactttggtgctgaggtcttatgttcttccatagagagtaccttccgcagcagacctatcagacgagatcgggcacattcagggtggtatggctgtagacagcagcatcgtatcagacttctttatgttttgcttgtgacagcagtacttggctatggcagttaaatggactgtaagatgttagcttgatttggagtaccctgagtagctgcacaggtagacaaagtacaaagatattgacagcttttttcttcctttagaagaataacatatctaaaacaactcttaaagcaagagagctgcagttgcacactaagaaaatactgcaatactgccatgacttggttataggtgatgctaacataaatgggatattctgtttctcattgcaccagtcatgcagaatcatagcatcatgtgccagatggcagaacgtactgtggcagttaacaccacaaattaacaacacagtggcagaaagcaccgtttaaaaatatgggtacttggccaggcgtggtggctcacttctataatcccagcactttaggaggctgaggcgggcagatcacctgaggtcaggagttcgagactagctctagtctctactaaaatacaaaaattagtgtgtgtgtgtatatatatatactaatcaaacttccttgggaaagaaaatgtgaatgcatttctgtgtcatacatttcttgtgcagcactatatattctgtggtttagcagtattaacagcatcaagctgaacagtaaccagtgttgctaaggtctcgcatactataccatgtatgctgagaagatatgcatccttagaattgagaattagaacatttcagttaattgcagtaacttttcagtaactagatatatgtgtaatttgatttctgaagtaattttaactgtggaaaaactaagttgtcaccagcggtacctcatagcataactttagcccatgatatatggatggatggatataaatggaaattgcacactgaaattaggacgtttatatttcttcaggtattagaaaactactcggatgctccaatgacaccaaaacagattctgcaggtcatagaggcagaaggactaaaggaaatgaggtttgtattgttcttgttgcttaacatgagggtttcataggatatgtgtctttctgtagttgtagtgatatgagtaagtacacctgtgtatgtatttgtgcttctgtgtctggtagtgagatggaactatccatttcccaaattgtgtgtcgtctcagtggagggtggcagtgggtgaacatccatttgttttcatttagaggtgcagtggctttccccatctccttgttcttccttaaatcttggcatagagcttgttttttgtgtaaggagtcagggcacattttctcttgtgctgttttggaagtttcagacctgatagcctctattgtgcctaacgctacccttttctctatgccgtatcactgaagttctttcttttagaataaaagaacagtggtctcatcaacataaaaaaccactgacttttttccccttactaattgcagtcatatatgatgtgaatttactaaattttgaggtgacctttattttccaaatttatggtgtagttttcctggagaagtaacaagttagaaacatagaatttgcaggcttgaaagccaggactatctttcatctttctttttcctcatcctaacttcaaacttaaaaaaaaaaaaaaaaaaagctgggggtagtggcccatgcctgtaatcccagcactttgggaggccaaggcaggcagatcacttaggtcaggagttcgagaccagcctggccaacatggcaaaaccccgtctctactaacaatacaaagattagccggcatggtgacgcatgcctgtagcctgtagttccagcttgggaggcggaggctggagaatcgcttgaactcaggagacggaggttgcagtgagccgagattgcaccactgcactccagcctggacaacagaataagactgcatctcaaaaaacaaaacaaaacaaaaaacaaagaaagtacttaaatgaggaaagaggagagcactacacagtccttgcctgggactcagagcaaacttcatgtttttgctagtcaaacttagctgctttaattgaccttttagggtcaaaaggccttcactttccagttgtcagttcaaacgttctcaagttgatgttttgtggggagggaagattgagattgtctacatcacatttatttgacttgtacttatccactgtgtagtgttataattgtgctttatttttgtttttatgggctatttcccatcatgctgctgtctgcactgctgttggaccactttgcctatagaaggtaaatgagtccctttgggacatatggaattgagaaccttaacttgtttctgcttcttgttcttagaggaattatgaagcagtcatgatggctcatgatgagtgagcagctctgtaatttgtagacagaggtataatatgtaggagtcacagaagagctgtgccagcatgctgttccccttgtagcagagatgctagcacacccacttgatactccagattttgcatagggtttcttttagagattggttcttggtgaaaaattttttagcatccagtgtgttccaaggcaaaataagtaatagttttttaatggtttttttagactcagaatttatttttaacactgcataaattagggcttctagttctgaaaaatgcaaagaggcttacttaagctatttatatattttttaacatgctggggagagggaagagagggtgcacaggtgactcctagcactaatatgctagtgccaaactcagttaatattaataatggtcatttgttgatatttttggtagcttttctatttaaagattcagtaagtagcatcctttgcactgaattgtcacgttttgtaacctgctgttgtggcagggaccatgaaacaacttagtatgccattgtttcagtttattttttaagcattactgggaactaaatccacttgacttactttttgcatttaggtttaaataattgttcctgggtagatacagaataaaactcaatctccccacttccagcagagaaaaaacaaatgaaatactgttctttttgtgtacttactagaaaatgtttaactggaaaaaaagttctgcttgttttattggcacagcaccattgattctgtgattctttgtcatagtattctgtggcagtttctgagtatgatatgtagattgaaatatagaggctaggaaacttttaatagtaaaattttactaaaaatttattaacttattaaaatataacttgaaaaaattgtgaggtttttctaaatttcttggaaatttgatttatattgtcaaaaagcactgaagaagtgttagttatggatttcgggtatcacataatgtttattccagagttttctgagtgtgatttatgtgaatttcatttggttattcttttactattttcacagtgaaattgtcatgagattttttcttctgtatttcttgcttagcttcttctcatttaagaatgagttggagggattaggtctttaagtaggcagatggattgtataaccctcatccattcttttgtggttttacagtgggacttcccctctcgcatgcctcaatgctatgctacattccaattcaagaggaggagaggggttgttttataaactgcctggccgaatcagccttttcacgctcaaggtaagtgatatgaactctcttttggtgcagtgattcttggacttttaatgtgcataaaaatcacctggtaggtctggaataggggccatgaatttgcatttctcatatgcagtcaggtgacactgatgtttttcttttttttagacatagtctcgctgtgtcacccaggctggagtgcaatggggcgagatctcggctcactgcaatctctgcctcccatgttcaagtgattctcccacctcaacctcccaagtagctgggactacaggcacatgccaccatgctcggctaatttttttattttttttaatagagacggggtttcaccatgttggccaggctggtctcgaaatcctgacctcaggtgatccacctacctcggcctcccaaagtgctgggattacaggcatgagccaccgcacctggccagatgctgttggttctaaatggcactttgaggtgttctaatatgaatcagatattagaaattatttagtcatagcagctcttgtttgtgtgttcatcttttcttgtctatatctttgtagattgtacaaaactgaagcataactatggggataattgcagatgtgcctttttttttttttttttttttttattctgagatggagtctcactctgttgcccaggctggagtgcagtggtgcgatctcagctcattgcaccctccacctcccaggttcaagcaattcttctgcctcagcctcctgagtagctaagagtgcactaccatgcccaggtaatttttgtatttttagtagagacggggtttcaccatgttggccaggctcgtcttgaactcctgaccttgtgatccgtccgccttagcctcccaaagtgctgggattacaggcgtgagccactgcacctggccacagatgtgccttaattaggattttgttgtagtaatgatgtgaaggataaattactggtcagctaatgtaagacctactctatggaaaataactgaaatgtgaacatatttgtggaaattaagtgacagtaattataaagtaagagtacttaaatcaggtagagttcataataaagcttaaatagggccgaaatgaacaggctaattatttttccaccatttgtgatatatatggtaatcatttagaccgggggtctgcaaagtttttctgtaaaggaccagaaaatcaatgtttttttgttttgtggatcatactgtctttgtcttaaccactcaggtctgccattgtagtaggaaagcagccatacccaaatgaatgggcatggctgtgttccaaaaaaaaaaaaaatttatttacaaatgtttgttttacttacgaaaacaggctgcaggccagatttgatttggggctgtactttgtcgacccctgatttagacataagatgacttgtcgacattggagaaactgaaaatttgatgctgaccctttcgttcttccatagtgggttataaattttagcagtcagtacaaactttttcctttaagttttgttttcttgatggactgtattcaaaataaatacctgaacaaataccaactatataacatgaagccataaaacttggtctttatatataaataaagtttatcttggggtcagatgtctgtgctgttttctatatttttttatatccaatagtttggtctttttcctttcagttttgtatgcttttcttgtaatcgagccttggaagagacagagccactgagcccaaaacctagctttgtggtcggctgtggtggctcatgcctgtaatcccagcactttgggaggctaaggcgggaggattgcttaaactcaggagtatgagaccttgtctctattaaaaaaaaaaaaaaaaaaaaaaagccttgcgtggtggtgtgtgtgcttatagtcttagctactcaggaggctgaagcaggagggtagcttggagatggaggctgcagtgagctgtgatcacaccactgctctccagcctgggccacatagcaagaccctgtctcaaaacaaaaacttagctttgtaacctgggagggatttaacccttctttttggtggaatatgcatccctggcattgagtcagtccttaataaatgatagcctttatgatgatctgaggtttatcattcttaattgcctgttaatggcatttttctattaatattatctattttttgagatagggtcttgttctgtcacctgggctggagtgcagtatcatagtcacagctcactgcagccttgacctactaggctcaagtgatcctcccacctcagccttctgagtagctaggacttcaggcgcataccactatgcctggctaattttttgtattttttgtggagaaggagttttgccatgttgcccaggctggtctcgagctcctggggtcaagcgatccactggctttggcctcccaaactgccaggattacagacgtgggccactgcacctggcctattattttttaatttaatattaatttatatttttagaaacagggtctcacactgttgtccacagctggagtgcagtagtataatcatagctcaccgtagccttgaactcctgggctcaagcagtagtcccacctcagcctcccgagtagcgtacaccatcaccccagctaatttttaaattttttcctagagatgaagtcttgctatgttggttaaactggtctcaaactcctggcctcaagtgatcttcccaccacagcttcacaaagcactgggatggattacaggtgtgagcaaccatacctggccctctattatctcatatatagcttattgttacatatgctgtaattatatatagttataattatttaatacatatatgtttattacaaatatgcctgttaatggtatattctgtttttaaaaggaaggataattacttttttctatgagtaccttaagggcaggaactgagttttatttgtgtttgtaatttggctctgggcagaattcttggcatagtacgtgctttatgtgtgaactgaattatatttcttcatcttaattttacaggtgtgagccactgcaccaggccccttcatcttaattttaatatatctttgaataaacaccattgtatgaacctgctgtaagcttgggagtggtctgttagtctacagcttgtgtctgagatgtgctaattgaatatttgctcagtacctcatcttaactgcctttggctttatgttgcttatccttcatagtatcttgttcattggccttttacatccataggcatcacttctctgatattcgttgtgctcttttaatggattaatggtttgcttggttggttcctctagttagactgtaaactccttgagagcagagtctgtattttattaattacccacagtactaggtacatagttgccttcaataaatatatatttaatgaacgaatttagtgaataatggctacatttttgtacttcgttattatttatttattttttttttctgagacagagtcttgctctgtaacccacgctggagtgcagtggtgtgatcttggctcactgcaacctccaccgcccaggctgaagtgattctcctgcctcagcctcccgagtagctaggactacaggcgtgcaccaccatacctggccaatttttgtatttttagtagagatgggattttactatgttggccaggctggtcttgaactcttgacctcgtgatccgtctgcctcggcctcccagagtgctgggattacaggtgtgagccaccgcgcccagccttcattattcttttgacatttgatgataaatactgtttatgattttctttttctttttttttttttggtagagacagtctcgctttgttgcccaggctggtcttgaacttttggactcaagcgatccacccacctcagcctcccaaagtgttgggtttacatgcatgagccacctcgtgtgtccttgttcataattttgatgggctgggcgcagtggctcacgcctgtaatcccagcactttgggaggccgaggtgggtgaatcacttgaggtcaggagttcaagaccagcctggccaacatggcgaaaccccgtctctacgaaaaatacaaaaattagccgggcgtggttgtgcacgcctgtaatcctagctacccaagaggctgaggcaggagaatcactttaacccgggagatggaggttgcagtgagctgagatcgtgccattgcactccagcctgggcaacaagagtgaagctccatctcaagaacagttttgatgaaattgtccctagatttgcatctggcatctagctcatatctcagagccagaatgaaggatgcttacctctgggtttggtaaattttagcttggttaattatgtgtgtgccttaatttttttgacctattataaaagcaatatacgtaaatgcaacagagaaaatgaaagtaatccataaaattctacctgtggccagctgcgatggctcatgcctgtaatcccagcactttgggaggttgcggtgggtggatcacttgaggtcaggagttcgagaccagcctggccaacatggtgaaaccctgtctctactgaaaatgcgaaaattagctgggcatggtggtgggtgcctgtaattccaggtacttggggaggctgaggctggagaattgcttgaacccgggaggcagaggttgcagtgagccaagattgcaccactgcactctagcctgggtgacagagactcttaagaaaaagaaaaaaaaattcagatacctgtaacctaaataattaatatttgaaaaaatttcatcattgcttctttcattcagttaatatattttgaaccacttgccatggtgttatatatagacatgtcttatatatgtgtatatatatgtgtgtatttatttattttttgagacagagtctcgctttgtcatccaggctggagttcagtggtacaatcatggctcactgcacccttgacttcctgggctcaagcaatccttccagcttccctagtagctgggactacaggcgcatgccaccacacccagctaatttttgtagtttttgttgaggtgggaattcactagtcttgaattcctgagctcaagcaatcctcccatcttggcttcccaaagtgctgggattataggcatgagccactgtgctcacccagttttttatttttagatacagggtctcgctctgtggcttaaagctagatggtgcagtggcaccatcatagctcactacaaccttgagcgcctgggcttaaggaatcttcctgtcagcctccccagtagctgggaccacaggcatgcaccactgcacctaattaaaaaaaattttttttatagagatggagggggtctcattttgttcctcaggctagtctcaaactcctggcctcaagcaatcctctgcttcagcctcccgaagtgctgggattacaggtgtgagccacggtgcccggccccttattctttttgaaggctgttttaagaattccagatttccagccatgaatgtgtgattactaactagtctcttgttatatgcctcatcaacaccacacttctcccagaagttgtgcaggcatttagtttattgttactcaggaatggatgagaaaggatatcaaagatctgaaccagctgattaccatcttggtcacttgagaactcaggtctgtccaataaacaccctaatccaaggtggtgttaaatacatatatatatatttttactttacgtttatttattttgaaaaatttcaaacctatagaaaaattgaggcagtaccatagtcttagtccattttccattacttagaatatccaaaagtgagtaatttataaagaaaattaatttatttcttacagctatggaggccaaggtcgaggggacatatctggtcagcgttttgccatgttggtcaggctggtctcgaactcctgacctcaaggcctgccttggcctcccaaagtgctgggattacaggcataagccaccgtgcccagccacctctgagcactttagatagtatgtcttaagaacaaagacatagcccggcgcagtggctcacgcctgtaatcccagcagtttgggaggctgagtcgggcggatcaccttaggtcaggagttcgcggcaaaaccccgttactactaaaaatacaaaaaattagctgggtgtggtggcgtgcacctgtaatcccagctactcgggaggctgaggctggagaatcacttgaacccaggaggcagaggctgcagtgagccgagatggcaccactgcactgtagcctgggtgacagagcaagacttggtctcaaaaaaaaaaaagaaaaaaagaacaaagacattttcctatataaccacaatgccgttatcacactcaagaaacttaacattgatatgattgtgtataatacatagtccatattcaaatttttctctcatttcaaaattattctttgtggcctaaaatcctgtatccaatcatgatatttttgaacaatccagactagttgttgtatagaagtaagtagtttgttttttttgttttgttttgttttgttttttgagacagggcctggctccgttgcccaggctggagtgcagtggcatgatcacggcttactgcaacctctgcctcctgtgctcaagcaatcttgtgcctcagcttcctaaataactggaattagaggtgcgtgacaccacgcctagctaatttttgtatttttaatagagatggggttttgtcatgttgcccaggctggtcttgaactcctggactcgaagcaatcctcccacctcagcctcccaaagtgctgggatttataggtgtgagccaccaggcctggcagttaagtacattttttaaggactaataaaaatgtagaaatcttcgttatttccggaatcctttatgttttactgccagtactaatgtagtaatgccagacagtagtcatgtctactgttccaggttgtccaaaggattcagcagcaaaggatgaaagtggtgggaagttttgcatagtggtcgaccaagggctagtttggtgagtgtcttgcctatgataatggacaggaagacaccagttattttattttatttttattttattttattttattttaattttattttgtgttgtgttgtgttatctttgagatggagtctcactgtatcacccaggctggagtgcagtggcgcaatctcggctcactgtaacctctgcctcccgggttgaagtgatcctcctgcctcagcctcttgagtagctgggattatatgcgtgagccaccacacccgactaattttttgtatttttagtagagacagggttttaccatgttggccaggctggtcttgaactccttacctcaggtgatccacccaccgcagccccccaaagtgttgggattacaggcgtgagccaccacacccagctgacaccagttatttaactcacatttctctgttttccttttaaaggtagtagtgacacctttgaaatcttttggcattttctttgcctgttctcccttatgtgttaaggataagcttacgtgcagaatggcttgttaagttttttctcctccgtacttggaaatctcatctggtgggacattagggtcagaattttgtatattatatattatatatctatcatgatatatataatgtattttatatattatatattatataaatatcatgatagatatataatatataattatatgttatatattttatatttataaaataaaaataaaaaattgcaattattttcaaatagtaaaaccacgtagataaaatataattctggccaggcacgtggctcatgcctgtaatcccagcactttgggaggctaaggtgggtggatcacctgaggtcaggtgatataatatacaacagatataatatgtctattatatattatatataatgttatatagatatctatattatatataatatattaatagatatattaatataatatatattatatattatacagatatctataaatatattatatataatatattatagatatatctatgtatattatatcacctgacgtcaggtgatccacccaccttagcctcgcaaagtgctgggattacaggcgtgagccacgtgcctggccagaattatattttatctacgtggttttactatttgaaaataattgcaattttttattttattttttggttctggaaacatttgtgtggacttacctatgattgtcaggtttactgatttgctggagattttttgggcatgacaaagggtttcttctttgataaaagtttttctgggtaatgtgatggtacaggttgagtatcctttatctgaaatgcttgagacatgaagtgttttggatttgagaatttttagcatcttggaatatttaccacatacatagtaaggtaccttgaggatggggcccaagtctaaacacagaattcatttgtgtttcatatttatcttatatgcatgaaacaaagttgtttgttttcttttttttttgagacggagtctcactctgtcgcccaggctggagtgcaatggcatggtcttggctcactgcaacctccgcctcccaggtccaagcgattctcttacctcagcctcctgcgtagctgggattacagacatgaggcaccatgcccggcaaatttttgtatttttagtagagatggggtttcaccatgttggccaggctggtctcaaacccctgacctgaagtgatctgcctgcctcagcctcccaaagtcctgggattacaggtgtgaaccaccgcacctggccccaaaataaagttttgactctgtgtgtgtgtgtgtgtgtgtgtgtgtgtgttttggagacagagtctcactctgtcgcccaggctggagtgcagtggcatgatcacagctcactgcaaccttagcttcctgggctcaaatgatcctcccactttagcctcccaggtagttgggactacaggtgcgtgccactgtggctgagtaatttgtgtgtgtgtgtgtgtgtgtgtgtgtgtgtgtgtgttttaatagagatggggctttgccatgttgcccagcctggctcttgacctcttgggctcaagccatctgcccaccttgacctcccaaagtgctaggattataggcgtgaaccaccatgcccagcctttgactgtgttttgatggcaacccatcacgtgaggtcaggtgtagaaatttccatttttggcgtcaggttggcactccaaaggtttcagattgtggagctccttggagttttggattagggatgctcagcctgtacatttgacagtttatttacagatgcttgcatcagtgcttggagctagtcctcagtggtgggataggtgaaaattatctctactttgagttcttgatttgaattcttttttcttaagcttttcctaaagtcatgtgtcagtgttgattattgcccggttagaggtaggatttgtattgtttttttacacttggaatttcctgttgtcataagatcactgtgaactcccttgtagtgttgtacattcttaggactttcatgctgaaatactatgcttttataaaatggttgttgtgtctttgctgagtttaaatagggtccaggccaggcatggtggctcatgcttaccctgtctcaaaaaaaaaacaccctcaactaaatatagttaaataggccaggctcggtggctcatgcctgtaatcccagcactttgggaggcggaggcgggcggatcacctgaggtcaggagttcgagaccagcctggctaacacggcgaagccctgtctctattaaaaataaaaaaaaaacctagccaggtgtggtggtgaacgcctgtagtcccagctactcgggaggctgaggcaggagaatcacttgaacccagagacagagtgagactctgtctctcacacacacacacacacagataaataaattgggttcataaatggtgattccagcactcactgtcaggtataacttcagtctttttttttttttttttttttttttttttttttttttttttgagacagagtctccttctgtcacccaggctggagagcagtggtgtagtctcagctcattgcaacctccaccttctaggttcaagcagttcttctgccttagcctcccaggtagctgggattacaggtgcagaccaccacgcccaactaatttatttatttattattattattattattattttttagtagagatggagttttgccatgttggtcaggctgactttgaactcctgacttcatgtgatccacctgccttggcctcccaaagtgctgggattacaggtgtgagccactgtgtccagccaacattattcacttttgttgttacataagatgtcacggccaggcatggtgggtcacgcctgtaatcccagcactttgggaggctgaggtgtgtggatcacctgaggtcaggagtttgagaccagcctggccaacctggtgaaaccccgtctgtactaaaaatatgaaaattagctgggattacaggcgggcacctgtaatcccagctactagggaggctgaggcaggagaatcacttgaacccgggaggcagaggttgcagtgagctgagatcgtgccattgcactccagcctgggtgacagagcaagactgcgtctcaaaaaaagaaaaaaataaaaaagatgtcagttatggctatttgtactttattatacttcatgtgatgatatatggttcagcatcagggtcttttttcagttaagttcagttgaacccttttcctgtttagaatattttttaaaagtataaaaaaacttaataaaagaaatggatggctgggcatggtggctcacccctgtaatcccagcactttggtaggctgaggcgggcagatcacctgaggtcaggagttcaagaccagcctggccaacacagtgaaactccatctctactaaaaatacaaaaattagctgggcgtggtggcggccgcctgtaatcccagccacttgggaggctgaggcaggagaatcccttgaacccgggaggcggagattgcaatgagcctattgcgcaactgcactgtagcctgggcgacaagaaggagactccgtccctcccataaaaaaaagaaatagatgactacatttaaaatatcttccatttaaatccatagtttacaaataaaggaaaaagcagtggaggaagagagtgagataattacttcatttttacaatgttaaataattacaattaaaatttttgtgtacaattccacatttaatagcaatttgcaaatgccttattgccaggtgtggtgtcttacgcctgtaatcccagtactttgggagcccaaggtgggagaattgcttgagccctggaattcgagaccagcctgggcaacatagcgaaacttttgtctctacaagaaataaaattagccaggtgtggtagcctgcatctgtagttggaggtactgggggagctgacgtgagaggattgcttgagcccaggaggttgaggctgcagctgacatcaagccactagactctatcctgggcgactaattgagaccctgtctcaacgaacaaagaaacaaaaaaaacccaaatgatttacttaccattaaataccaagaaaagaatgactggagtaagcttagatttagatacaacaaaatcatggagcttatcatttgaaaggcatttttgacttttcataggtgtgtagcaagcaaagtggtccttttgaaaaatgcaagtcacatcatgtcacttttctgcttggcctttgcctacttctcgaatggttctcctagcacattctaactggtcattattcattgtgtttcagccattctggccttcaacctatttctcaaacaccccaatcttgggtatactttagggcccttgcatttgctagctcatttacctggaaaattctgcggtaacttcttgcacataataggtgctaataaatattggctgcatgcaaccaaaaaagctggcttcggtagtagaagtagtagaagagaacattaccaaagtccttttttttgagacaagatttccctctgttatttaggctgagtgcagtggtgccatcacggctcactgcagccttgacttcttggactcaaatgatcctcccacctcagcctcctgagaagctgggaccacaggtgtgtgtcaccatgcctggccaatttttttatttgtagagaagaggtcttactgtgttgcccagaatggtgtcaaactcctgggctcaagtgatccacctgcctcagcctcccaaaatgctgagattataggtgtgagccactggtccagcctcttaccaaaatctttttttttttttttttttgagacggagtctcgctgtgtcgcccaggctggagtgcagtggcgggatctcggctcactgcaagctccgcctcccgggttcacaccattctcctgcctcagcctcctgagtagctgggactacaggcgcctgctaccacgcccggctaattttttatatttttagtagagacggggtttcaccatgttggccaggatggtctcgatcttttgacctcgtgatccacccacctcggcctcccaaagtgctgggattacaggcatgagctaccgcgcctggccgcctcttatcaaaatcttaaacttgttaattcttgtatctcaccttgcttttatatcagtttttgttatagaatgtgctttctttttaggttatttttcttctgctcagcaagttaactttgaagaatcttttttttcttgccttttttttttttttttgagacagaattttggttttgttgcccaggctggagtgcaatggcgcgatctcagctaacggcaacctccacttcccgggttcaagtgattctcctgccccagcctcccgagtagctgggattacaggcatgaggccaccaggcctggctatttttattttttattttttatttttattttttactagagacagtgtttttccatgttggtcaggctggtcttgaattctcgtcctcaggtgatccacccgccttgacctcccgaagtcctgggattacaggtgtgagccagcgcacccggcctcttgccctctttagagcagttctacataaatctgtggaataatctgctgacgttagaaataaacctttctcagggtgtggccataatctggttcgttgtaattgctgaaggaaaaaactgaacccttaggaacagtttaaacattctaggtggtttttagatatattattcctgttagagttttgaactttgatcatcttgttagaaaaaagaggccaggcactgtgcctcatacctgtaatcccagcattttggaggctgagtcgtcaggaagtcacttgagcccaggagtttggtaccagcctgggcaacatatggagacctcgtctctacaaaaaaaaaaaaaaaaaaacaaaacctgttagccaggcgtggtggctcatgcctgtgttcccagctactcaggaggctgagatggaaggattgcttgagcctgggaagttgcgactgcagtgagctgtgattgtgccactgcactccagcctgggcaacagagtaagaccctgtctcaaaaaaaatgaatttttgcattcatttaaaaatgaatggtagcaacattttagataataacttatgtagtctgtgtgaatctcatcaaatatatgtgatttttaatgcttggaatcctagaattttagtgagggaggagctctcaagttatctaatttactgtcattttacagataaaaactaaacttctgatagatgaaaggatttataccatacatctatatgataattgcatatggctgagttgaggttagaacccaggacccaatctttgatttgatttcttttcattatgccaatttagaataaaattttttctcttggttttctgacctttggccacttcatcttttgaaggatcttgtaagttccttgatatttctcaaaatgtcagttttattctcttcaatgaaagttcttgttaggtgaaagtagcctttcatatccttaaatgtgaacctgttccttaggctggagtttttgtactctccattatattggcatttatagttgttggttttcttttttttttttcttttgagacggagtctgtctctgtcgcccaggctagagtgcagtggcgcgatctcggctcactgcaagctctgcctcccgggttcacgccattctcctgcctcagcctcctgagtagctgggactataggcgcccgccaccatgcccggctaatttttttgtatttttaatagagacggggtttcactgtgtttgccaggatggtctccatctcctgaccttgtgatcctcccgcctcggcctcccacaagtgctgggattacaggcgtgagccactgcacccggccagttgttggttttcttttctgtgccagattgagtgatatatggtggtactaagagcactggaataggagtcaaggactgaggtacagcatagcacctcatgtattacttggtatctccacttgtctctaacactgaagaggtataattggttgtttaaaaactgtgggtgtaaaacgctagtatcccatgaaatggaactgcagaatataagtggatgactctgaaactgcttttgaaacaagcatgagggctcagtttctccatctctcgtgggctccaccatcactgtctgacatcctgatctcctaaggcatctttcctccatcccagagctctgttgaaaatctcttgggctaaaaaccagtacagaccttctgatactcacgttctagcctttttagattgttgtgtgtgaagagtaagtcagctgtggatatttttctttcctattttgtgaggcatttggtatttactggaggctgcctgttttgttgcaaaccttgttccactgtgtactataggaggtctgctcagaaagcctagttagtagagcagtaccatttgctttggtcaattgtagcagcagtctgttttttttttttttttttttttttttgagacagagtcttgctgtgttgctcaggctggagtgcagtggtgtgatctttgctcactgcaacctccgcctcctgggttcaagcgattctcctgccttggcctcccaagtgggattgcaggtgtgtgccaccatgcccggctaattctgtatttttaatagagatggggtttcacccccaggctggtcttaaactcctaacctcaagtgatccacttgcttcggcctcccaaagtgctaggattacaggtgtgagccaccgtgcccggccagcaatctggttttaaaggaggctttgacccctcatttggtaaaaggttccttctttgactaattcccaaaggacatctcgatctgtcttcaaaagaaggggtattccgttgtcagtattcctagctagctctgggagaagagagaacttgattgttttcagggccctggtcagagtctggttttatgcccaagggatgaactgccttttgtccttctacctcctgaagttcagagccctgtgaaaatgaagtggtaaaggtcaagtagcagtagtttgaaaagcaccagcactgggacgattttgtggtagcctagcagagtgggcaggatgcttacttcagaggtgctaaagatgtatttttttttggaacaagaaggttataaatgtttctattaagtttgattttaaagcatgagggttctgggaaaatgagaagattgtacatactctaggcttccttgggactgcatttgaaaaaaaaaaaacttttctttgggtcagccagtatgaaaactgcatttgatctcaatccttcagatcaggcttttggggagagttagttgaagaatttgtttagtctaagaaagttgcttctagcttagctaaatctggaagaaaaaatggaacctgatgtttgagtgctaactgtattccagacagacgttatactccttatattggtccttttgaagcataaatatgtttccaagtcctataataatgaggaaattgcctgtgagagtttaagtaaattgtacaaagtaacacttttggttcagagatttgtttttaatacagagaggacaggttaggaactttgaagcttcagctaaatgagtcagttctcaaatgtttgagaagcatgctcaaggatctggaggaatagcttaattaatttgtacatcatgagaggagtagggagcaaaaatatgtataatttcttctgaatgatgctgtcttttttgatgcagtggtgctaaatgggctttgcttgtatgatacttcttaaatgcattagctaattattttgttttagtcttcttttttgaaatacagatgactctttgagctggttttctttggaaagttatctgtcctgacaattctacttctaaaacttcaactttttttttttagtcttcatttccattgccaccaccctatcctaaattatcccgttatgctggatactgaaatagcttctgcctggtctacttactttcacttcgcccccttacgatccagtgctttgctccttttaatccagtggccagagggatttttttcaaaacataatttgattataacagatctatgtttgatacttatccatggtttcccatggcagttgcagaaaaatccaaaccttgtttggccacactgcccttctttcagtaatttgaattccttaagctttttccagtttcaggatttcccattttgaactctttctcctccactttttcctggccacaggtttcaagagctgacaacttacctcagttcttaagaggatccttcatttatctctaaattggattttcctgtgtccaaaccttacttgttatcacaagtttataattatttgggtgattgtttgatgtgtctctcctgactgggccttaggggcagagaatctgtattattttgttcttgtttatatccctaggagctcttatggtaggtattcaataaatgtttgttgaataaaggcagtggaaaaacacatagaagaggtgctgtggcagcagttctcagtgccaacagttgaattggtttacaatagtaatttttagtgtcaacaaggagatgtgtagtatggaatgtttattagcaagatctttttctcatggaatggaatgtttgaaaacctccaaagtgtaagtttttttttttctcaaaagaaaacagtcatatttggggttacttttttttttgagacagagtcttgttctgttgcccaggctggagtgcagtggcatgatcccggctcactgcaacctccacctcccgagtttaagtaattctcatgccttagccactggaatagctgggattacaggtgtgtgccaccatgcccagctgttttttgtataattattagagatggggtttcgccatgttgaccaggctggtctcaagctcctggcctcaagtgatctgccagccttggcctcccaaagtgctgggattacagatgagagccaccgtgcccggcccattgttacttctgaaatcgaaatcaaatgccatattgattcctgatcttttgtgtatgacctctctctctcttttttttttttttttgctacctctccccgaaagtttttaggatctttttttccacgattatgtaccttgatgtgggtcttttctcgccctttgtgctattaggtctagcaactcatgtgtctcaggtctgggaaatgattttacagtttttccagctggtgcgtaactagctgtttgttctgatactgtcagtatttaactttatgttagaaacatttcataatgcaaatgaaaaatttaagccaggtgcagtggctcatgcactttgtgaggctgaggtgggaggaacacttgaggtcaggagttcaaaaccagcctggccaacatggtgaaaccccttctactaaaaaaacaaaaaaacaaaaattaactgggcatggttgtgcacccccataatcccagttactcaggaggctgaggtacgagaatcactggaactgggagacaggtttcagtgagccgagactgagacagagcaagaccctgtctcaaaatataaataaaatacataaaaaatttaatattatgtatataatatccagcccaggcttttctatttgtcatgccctgtttctttcacatcatagccttactcagatgcctgctgatccttgattgaggtgtgatgcttgagagtcaggctcttaaatactactggcgaattcagtgcattgtggttttgtgacttcacttgtctcttacagaagtggggaagtggtttttacttcgatgcatggttctgaagtagagatgtctatctcctgatgcacactttaaataatccccatgttttccactccatactccatccttgtctggtttaagctcttagcctgtctgcagttcaggggatgaatctgcttggttcttttcagcatctccctctgcaacgcttagatctcatcttcctttgctttgctaactattctctttgcttctccatttgtttctctgtcattgatgacttccaagttatatccccatcctggatattccttttgaacgtcagactcatatattcagttacctcctcagtatctctattcggatatctaatggcatctcaaaattaacatgtgcaaaaccaacctgttccacccttgtgttggctgtataaattagtgggaactccattttttaagttgcttaaatcagaaacctgggagtcatcaattttggagtctgtccctttgctcttgtggcacatgtaatcacgaataaatcctgttggctctatcttcagaatatatctcattttatcactctctctgccaccagagtggtttaagccaccttcatctcttaggttattttagtagcttcctaactggtctccttccttccattcttgccaccttcctcaacaccgaagatatattttatttatttatttatttattttgagacagagtcttgctctgtcaccccagctggaatgcggtggcttgatcttggctcactgcaacctctgtcttccaggcttaagcaattctcctgcctcagcctcccgagtagctggggctgcaggcatgcaccaccatgcctggctaatttttgtattttttagtagagatggagtttcaccatgttggccaggctggtctcgaactcctgacctcaggtcatctgcccgcctcagcctcccaaagtgttgggattacaggcgtcagccaccatgcttggccaaattttttattcctttgggtaaagagatgtattttatttcctttaatttggtaatatagtgagttatgttaatagattctttttttccagtgctcaagcatccttgcattcctaggataaatggtacttcaacatgattttccttttaaatatacattgctggatttaggttataaattattgagaatttttgcatctgtatttacaagtgttacattctaatttcaggccttttccagttttattatcaaaacaagctgtgctgtttttcctctcttgattctcagtagtagtctctttaagatagtgattattcattctttttggtagatcatgcctataagacctgggcatgtatgtttttattgtgggaatatattgtttttactttccttgttatgaaatatataatttattttaaaatatgtatatacagagtttgtgttcctttaaagaataatagctaaaaataaattgcacgcccccacatccaaccatcgtcaaacaaaacatcttttacactctattttatttatttattttcccccacccctctagagtatacacccttttggagcctctgagcattttcctttccctcactgctttagaaacctctattcagatttttcatattaatgattcttttgcttttaacccacatgtgttactgaagtttaagtcctaaataataatttttcctgctttaaaatatatttttatgtcaatgaaatcatactggtggatgtattttctctgatttgctgcttttgcctctcatttctgagcttgatctatgttggtgcatttatagaaatacttcatattttctgctgtgtatcatcttatgctatcaacgtgccatatgatttacctgttcttagcatgtatttgggttgtttctggttcttgctgtaacaaacagctgcatttagtgcttgtgtatgtgtctcctgatgcatgtgtgcattcacttctctggggtgtatatgttgctgggaatgggactgagtattgtgagtgtataatagtatcttgtttttgttttttttccctctcagtttatggcataatttacgtacagtaaaatttaccaccgcccccctgccccgcctttttctttttctgaaacagagtctcgctctgttgtccaggctggagtgcagtggcacaatctcggctcactgcaacctttacttactgggttcaagcgattctcctgcctcagcctcacaagtagttgggattacaggtgcctgccaggctaattttgtagagacagggtttcaccatgttggccaggctggtctcaaactccttacctcaggtgatccacccaccttggcctccccaagtgttgggattagaggtatgagccaccacgcccagccaaattcaccctttttagtgtacaagtatacagctttgtgagagtttgatttctttagttcaaatttgttttaaagttatatttggtattttaacagtgctatttaaatatatgccatatatggcttgaatgcaattatcctagtacagatggtccttgacttatgatgtttttgtgtccctgtaagcccaccttaagttgaaaatattaagttgaaaatgcatttaatatgactaacctgctgtaaatcctaacttagcctaccttaaatgcacttagaacacttaaattagtttacagtgggcaaatcatcaaacacaaagcctatttgtaataaagtgttgaatatcttatataatttattgaatactaaagtgaaaaacaatagttgtatgagtagtcagagtatggcttctcttcaatgcatatcactttcatataccatcgtaaactcaaaaaatcataagtcagggtatgctgtatattttattggtgctaacatacttccattttaaaatacaggaaattcacctttttttgtgtatagtcttgtaagttttgacagatctatatacagatatgtaatccccacccatgattcttttttaatttaaaaaaatatatttttgagacagtctcactgtcacccaggctggaatgcagtggcatgatcgtagctcactgcagcctcaaactcctggccttaagtgctcctcctgcctcagcctcccaaagtgctaggattacaggaatgagccagtgtgccctgccaccaccataattaagatccagaacagtttcatcatcccagaaactttccctgtatccttttgtagtcagcttcttttttcacccccagctcctggtgaccactgatttgttctctgtccttgcagtactgtcttttccagaatgccatataaattgaatcctacattatataacactttgagtctggcgtcttttacttagtgtgaagtatttgagatttatttgtcttgtgatgtgtattagcaatttatttcttttttcttttttttctttttggagacaaggtcttgctctgtcacccaggctgaagtgcagcggcacgatcatagttcactgcaggcttaaactcaagcaatcctcccacttcagcctcctgagtagctgggactgcaggcgcacaccaccatgttcagctaattttttttttttcttggtagagacaggatctcactctgttgcccaggctgatttcaaactcctggcctcaaggaatcctcccacctatgcctctcaaagtgctgggattacaggtgtgaaccattacagttggctagcagtttatttctgtatattgcaggatagtatctcattgtatagatgtaccacattttgcgtattcattcttcagttgatagacatttggactacttcctccttttggctgctgtgagtaatgctgctgtgaataaatatttgtgtgtaagtttctatgtggacatacagtttcattattcttgggtagataaaccttggagtaattgctgggttgggtggtaatttctgtttaacttttaaagaaatagctaagctgttttccaaagtggcttcaccatcttaaattcaagtttttgttttcgttgtatttgaggcagagctttactctcttgcccaggctggagtgcaatggcaagattatattatagctcactgcagcctcgatcttccaggctcaagcgatcctctcacctcagcctcccaggtagctgagagtacaggtgtgtgtcaccatgcctggctaagttttgatttttagtagagataaggtctggctatgtttcccaggctggtcttgaacttctgagctcaagcagtcctcctgccttggcctcccaaagtgctgggattgcaggtgtgagccaccaagcccagccaaggtttttgtgttttagtaaaatttctgctcttcctggcaatacttttttctgaaatttactttgatattgatatagtcactccagttcttttcagtgcttagtttttgcatggtatattttttccatccttttgtgttatactgtctgtttcattatatttgtagttggaccttgattttttttttttccctaattcttttacttgggatgcttggacctaatgtgtgtctagtttttcctaatgtgtcttttacttgggatgcttggaccatgtgtatttaattattgatatggttgggttaagtccaccatctagctatttgttatttatttatttttagagccaaggtcttgctgtgccacccaggctggggtgctagtggcatgatcatggctcactgaagcctctaacttctgggctcaagcagtcatctcactgcagtctcccgagaagctgggattacaggcgtgtgccaccaagcctagctgattttttttttttttttccgagatggagatttgctcttgttgcccactctggagtgcagtggcgcaatcttggctcaccgcagcctctgcctcctgggttcaagcaattctcctgcctcagtctcccgagtagctgagattacaggtgcctgccaccatgcccagctaattctgtatttttagtagagatggggtttcactatgttggccaggctgatcttgaactcttgacctcgtgatctgcccgcctcggcctcccaaagtgttggaaggccgaggcgggcagatcacgcctgtgctgggaggctgaggcgggtagccactgctcccagccctgaaatctttcctcttttctaagatagacatttaatgcacacacacattttgatatgctatgttttaattttcattcagcttaaaatatctaatttttcttttgatttattctttaacttactgattatttagaagtgtgatattatattttgggaataaaaatccagagatcttactgttactgatttttgatttaattccattgtggtaagagaacataccttatatgatttgaatccttttaaattgattgagacttgttttatggatcagaataaggtgtcttgataaaggttccacgggcacttgaaaagaatgtgtattctactgttgttaggtagagcattctattaatgttgattggcttgttgattgataatgttcaagtcttctgtattcttaccgatttcctattctatcagttatagacagagggtgttaaaatctcaattctgttttgcttgttgattgataatgtttaagtcttctgtattcttaccgattttctgttctatcaattatagagagaggtgttaaaatctcaattctgtttgtttttggtttgaaatttacttataaggtacataagcatttatgattgtcgtcatcttgatgaattggtctctgtcattttgaaatgacagaacttactttgtctcataatatagctactcgtgcttttcatttgattagtgtttggcatatatttttatattttaaaaaaacttatcacctgtgtctttatgtttaaagtggttttttttcccaggcatcatgtgtttgggatcttgcttttatccagtttgatagtgtttgccttttaattggagtttgtagactatttatgtttaatgtgattactgatttttttttgagatggagtctcgccctgtcgcccaggctgcagtgcagtggcacgatctcagctcactccaacctccgtttcccgggttcaagtcattctcttgcctcagcctcccaagtagctgggactgcaggcacctgacaccacacccggctaatttttgtatttttagtagagatggggtttcaccatattggccaggctggtctcaaactgctgactttgtgatccgcccacctcagcctcccaaagtgctgggattacaggtgtgagccactgcggctggatgtttactgatttttatagagtttaaacaaccatcttttctatttgtctgatgtggtgtttttttcgtttttgtttttttaagatggactgtcactctgtcgtctaggctggagtgcagtggtgcaatctcaactcactgtaacctttgcctcctgggttcaagcagttttcctgtctcagcctcccaagtagctgggattacaggtgcgtgccaccacacctggctatttttttttttttttttttggtttgagacagagtctcgcactgtcacctgggctggagtgcagtggcgcgatctcggctcactgcaatctccgcctcctgggttcaagcgatactcctgcctcagcctcccaagtagctggtattacaggcgcccatcaccacacccagctaatgttttgtatttttagtagagacagggtttcactatattggccaggctggtctcaaattcctgaccttgtgatctgcccgccttggcctcccaaagtgctgggattacaggcgtgagccaccgtgcctggcctaatttttgtatttttagtagagatgggcttttgccatgttggccaagcaggtctcgaattcctgacctcaagtgatccctgcccttggcctcccaaagtgctggcattacaggagtgagctacgccatgcctggcctggattgaggattttttagtggttcatttttaagtcctttcttggctcattagtactttgttattttagtggctgctttagtgtttattgagttcagcttgtgaaactcagtctttaatattataccacttcacctatattacaagaaccttatagcagtacacttcattttcccttcagactttgtgctatgtttgtcatacacttcacttttacatatgttgtaaactccacaatacattattatttttcatttaagtagtcaatttttttaaaacaatttttttttttttgagacggagtctcactcggttgcccaggctacagtgcagtggtgcaatcttggcttactgcagcctctgcctcctgggttcaagtgattcttatgcctcagcctcctgagtagctgggattacaggtgcgccccatcatgcctggctgatttttgtattattagtagagatggggttttgccacgttggcccagcaggtctcgaactcctgagctccagtgatccgcctgtcttggcctcccaaagtgctgggattacaggcatgagccaccatgcctggcctaaaaagattttaaaagtaagaaaaaatgtcttttactttatatgttttccatttctggtgttctttattgcctatgagcggttccagattctcatctgccagattttcttcagttttttctcgtcagttcagattttctttctgccagaggacttcctttaatgtgtttcttgtagtgtaggtctgctggtgatatctttaagctttggtgtgtttgaaaaagtacttcctcttgtttttagaaagatatattcctttgatacagaattctaggttgacaggtttttttcattgtgtactttaaagatgttgctccactgtctctagcttgcattatttctcctgtaaaatctgttgttattttaatctttgttcttcagcgagcaaatttatcttttttgccttttttggttaatttttaagattttctttttatcactgctttttcagcaatgtgtttattatatactttgttgtagtttgcgtcatatttcttgtgtttgaattctcttgagcttcatggatgaaatttacgtattcagctactatttcttgaaatatttttcttttgtacttccccaccgcatctggagtctgtaattacatgtatatttggctgcttgaagtagtcatacaactcactgatgcactgttatgttttttagtcatttgattctctttgttttgggtagtttctactgatcttcatttcactagtctttatctttcccatgttaaatctgttactcttatccagaattataccttttatccttaaaaattcagtttttttcttttttttgagacggagtctcattctgtcacccaggctgaagtgcagtggcatgatcttggctcgctgcaacctccgcttcccaggttcaagcgattatcctgcctcagcctcccaagtagctgtgattacaggcatgcatcaccatgtctggctagtttttgtatttttagtagagacagagcttcaccatgtttgccaggctggtctcaaacccctgacttcaggtgatccacctgcctcagcctcctagagtgctgggattacaggaatgagcgaccacacctggtttgtttcttaaaaaacatcttatatttctctgcttaacgtgctcaatgttgaacatgtgaaatataataacttttcataatctcttctgattctgtctctatatcatctttgggtctgtttttatgtattctatttttcttgtcattatgggtcagatttcccagttctttgcaatcctaataaattctgtttagatgaaagacattgtgaatttcactttgggttgtggatatttttgtattcatgttggtatttttgagatttgttctggagtgcagatgagttagctggaaactaattcttttgaggcttgctttttagctttgttaggtggggcgtatcctttctgtgtttatttttgtaagggtatacagatatacatgttttcttatttccctctttatttattcttatttaatctgtaacaatctttttccttatattcgtaacattcttagttagggagtaattacctcttccctaactgttgcattagcttcttaattggcactctggttcccagactttttctgtctctaatgcagtgtacagactactgtcagaataattctaaagcacaattctgatattgtcactgtattgcttttcagtgcctacagcagtaggattcacttaaaaaattattcccctaatagtaatataaaaaatttttttgagctcaaactctcattctctctcagtatacatatttgtttatacatgaggtcacatactgcttactaagttagcattttttatgtgttcattttgaaataactctgaacagtggtatccaatcttttggcttccctgggccacattagaagaagaattgtcttgggccacacataaaatacactaatgatagctgaatagctaaaaattaaacattgcaaaaaaaaaaaacaaacaaaaaaaactcactgtggctttttttttttttccctgtatttttctttttcccgagatgaagtcatgctctgttgctcaggctggagtgcagtagtgtgatctcggctcactgcaacctccgcctaccaggttcaagcagttctcctgcctcagcctcccgagtagctgggattaaaggggtaagcccccatgcctggcctttttttttttttttttgtggggtgagcgaccatgcctggctttttttttttttttttttttttttttttttggtagagatggggtttcatcatgttggccagtctggtctcgaactcttgaccttgtaattcacctgccttggcctcctaaggtgttgggattacaggtgtgagccaccactcctggccttttttttttttttttttgagatggagtctcactttgtcacccaggctggagtgcagtggcatgatctcggctcgctgcagccactgcctcccgggttcaagcagttctcctgcctcaccctcccgagtagctgggattacaggtgtgtgccaccactcccagctagttttttgtatttttagtagaggcaggtttcaccatattggccaggctggtgaactcctgacctcaagtgatgcacccacctgggcctcccaaatgctgctggaattgcaggcgtgagccaccatgccaagccaattgtgttttaagaaaagtttatgaattagtattgggcagcatcgaaagtttacaaagtttgcgttgggccgcattgaaagctgtcctgggctgtgtgcgacctgctggccgcaggttggacaaacttgctctgggacataagtttaaaagttataggccaagtgccttggctcacgcctgtaatgtctttgcatttcatcagtttttccactgttatctttctgttacagtacacggtcagaataccacattgcatttagttgtcatgtttccttagatgtgaaacaaatttaccaattgtagtagggtatttacgtatagatcggttcgtttgtagccttatagtatctagttaaaacactgttataaagttattgttatgtatccttttgggtgtggtttctttccttagaaaaatacatttaaaattcattcatgttgttaaatgaatcaataggttgtttctcttttaaaaattgagatgaaattcacataacaaaattaaccattttatttttttaattttatatatattttctgagatggagtctcactctgtagcccaggctggagtgcagtgacgcgatctcggctcactgcaacatctgcctcctgggttcaagcgattctcagccttaaccattttaaagtgcacagttaagtggcatttagtacattcacagtgttgggtaaccatctttatctggttccaaaacatttttttttctttgaaagaaaaccccgtatttattaagcatttttctctctgtccttttcccttcactcgcttctggcaaacaccagtctgctctgtttctgtggatttacctgttctggatattttatacaaatggaatcatagcgtatgtagccttttgtggctggttttgttttttttgtttgtttttttttttgttttttttttttgagacggagtcttgctctgtcgcccaggctggagtgcagtggcgggatctcggctcactgcaagctccacctgccgggttcacgccattctcctgcctcagcctcccaagtagctgggactacaggcgcccgccactacgcccggctcattttttgtatttttagtagagacggggtttcaccgttttagctgggatggtctcgatctcctgacctcgtgatccgcccgcctcggcctcccaaagtgctgggattacaggcgtgagccaccgcgcccggcctggttttgttttagtaagatacttttgctggattaagaataaggttgggccaggggtggtggctcatgcctataatcccagcactttgggaggccgagttggcccagcctggccaacacagtgaaaccccatctttactaaaaatacaaaaattagtcaggcatggtggcgggtaccttgtaatcccagctacttggaaggctgaggcaggaggattgcttgaacccgggaagcggagtttgcagttagccaagatcatgccactgcactctagcctgggcgacaagagcaagactcgtttcaaagaaaaaaaaaagaatacgtagttgacaggtttttttttctttcaatactttaaagatgatataccactgtcttgtcactcaccttgtttctaacaagaaatctgctatctttgttctgtattttgcatgctttttttttccttgctgcttttattattttctctttattactggttttgagcattagattatgatatgccagtatggtttttcttgtgtttattgtgcttgtggttcattgatgttcttggatatgtcggtttatagctttcttcaggtttcggattttttttttttttggccatttttttcaaattattttccatttaatttctcttttctttttagtcaggtactctttatccaaacattaggctgcttgaagttgccccatagcttactgatagtttttgttttaacctgtgttttatttgaaagattctatttctgtgcctttgaatttactttcttttgcagtgtgtaatctgaagttagtcacatttagcatttttaaaaatctcacatattttactctttttttttttttttttttttttttttttttaaagactgagtctcactctgttgcctgggctggggtgcagtggcacaatttcagctcactacaacctccgcctcctggtttcaagcgatcctcccacctcagcctcccgagtagctgggactacaggcacccgccactacgcctggctcatttttgtatttgtagtagagacggggtttcgccatgttggccaggctggtcttgaactcctgacctcaagtgatctgcttgcctcagcctcccaaattgatgggattacaggtgtgagccaccatgcccggccatattttacctttttttatcgttagaattttgatttggtttaaaaaaaaatctttcctgtctctacttaagtttttgaatatatgaaatgcagatataactcttataatgtccttgtgtactcattctaacatctgtgtcatttcagggttggttgcaattgactattctctttagtatacttttttttccttgcctcttgcctggtgatctttgattggatttcagacatagagagttttacctcgttgggtgctggggatatttgtatttctacatatgttctttaactttgttattggatgcagttaagtcacttgaaaaccgttgattcctttaggtcttgtttgtaagatttgttagctgggaacggtgtagtagttggtttatggctaattcttcctcacttctgagacaagattcttcccaaatcttgagggttgtcttgagatttccagtctaatagctacttgagtgggtaagggcagagtactgttcataattcttttgggtgttgttttcctctccttgctgcccatggtcttgggtagtttcatcacactatgggccactcagtactcagtgagtacctctctagatctcctcagccctctactgtgtggtattgtcccatgaattctagctgcctaagtcctgcactctgagctgagctcgctgtcttcaattttgagttctgcagcctccatagggcttctgcttctgcatggtgcccaggaaactctcttcaggcactaagtttggccatcataggactcatatctttgtttctcttcctttggggattactgtctctcattgcctgctgcgcagcatcttgaaagctatagtttcatgttttgttagttattttagtggtttaaggtgagagggtaaatctggtccctgttgtgcttgaaaagtttttttcaacttttcttttagaaataattttaggtttactaaaagctgcagaaataatacagagttctgaggtgtctttcacccacttcccccatgataacattttacataaccatagtatagttattaaaactaggaaattgacattggtataatagtgttaactaaacctcatgccttatttcatattttcacatgaactattttgagctggcatggttaatatacagttctatgaagttttatcacgtgtatagatttcgtgtaattatcactagtcaggttatagaagtattccatcattttaatgaaatgcctttgtactactcctgtgtagtcatcatgtcctccttccagcctccaccaccaatcaaccaaccaactttaatcctggcagctaccagttgtctgaattttatcactttcgctataattttatctttctcagaatgtcataatacagtagtccccgttatctgtggtgatatgttctaagacccccagtggatgcctgaaaccagctatagtactgaaccatatatgtgtgtgtgtgtgtgtgtgtgttttttccccccccccctttttttttttgagacggagtctcactctcgtcacccagactagggtgcagtggtgtgatcttggctcactgcaatctgcacctcacaggttcaagcgattcttctgcctcagcctcctgggtaactgggattacaggttcgtgttaccacgcaggctaattttttgtatttttagttgaggtggggtttcaccatgttggccaggctggtctcgaactcctgacctcaagtgatccacccaccttagcctcccaaagtgctgggattacaggcgtgagccactgcacctggcctactatacatatacgcctatgatgaaatttaatttataaattaggcacagtaagagattaacaataataaaaaattataacaatatactataataaaagttatgtgaatgttgtttctctctcaaaatactgtaatattttcaggctgcagttgaccatgcgtagctgaaagcaagggtaagtggtgactactgtaaatagaatcatataatatgttacctttgagattggcttttcttttttaactcggcaaaatgcctttgaaatcgatccaagtcctatgtattaacactgtttctttttattggtgagtagtattctattcttgggcacactacagtttacccattcttccattgagagcatttctcccccctaaattacaaacagagatggagtcttgctttgtggttggtctccaactcctaggctcaatctatcctcctgcctcagcctcccaaagtgctgggattagaggcatgtgccactgtgcctggccccactgaagaacatttgagttgttcccagtttggggctattacaaataaagctgctgtgaacatttgcatgcagattttagtgtgaaaatatttttatttctctaggatgaatacctacctatgagtagaattgctgggtcgtatgatacatgtatatttaactctgtaagaaacgactacactgttttccagagtggtggtagtttttcattcccactaacaatgtataagaaatccaattgttctgcttttttattagcactttagtattgtcagtatttttccttctgttcattgtaatagatgcatagtagtatctcatgccttaaatttgcatttccgtagtggctaatgatgttgaatatcttttcatctccttaggttccatttgaatattctctttggtgtagtttatgttcaagacttttctattttcaaattggattttttcttactgttgagtgttctagacacaagttatttgttggatacgtgatttgcaaatattttctcctaatcttttaatttgcctaagagtgtgcttcatagagcaaaaggtttgaattcttatgaagtctaatttatctatttttaaattttatagttttttttggtgtcatatctaagcactctttggctaacaaaattttctctttttttctaaaagttgtatgctttacatttaagtctgtgattgcatttgaattaatttttaaattaagcataaggttacatcaatgttcattttcctttttgggggaccaattattccaaaaccatttgcttaaaatgattattctttatttattgaattgctttctcacctctagcaaaagtcagttggccatattttgtgaaaagttctattcctacgcacactgttctggctcattgacctacgcatctgttcctctgtcaataccgtactgtcttgattactgtaatgtagctgtatagtaagtcctaaaatcaggtagtgttattcctccaccttcgtttttctttttcagagttgtttcagctattctggtttctttgtctttacatggaaattttatttatttattatttattttcttgagacagcctcagtgttttgcccgggctggggtgtggtggtatgatcatagttcactgctgcctggaattcctgggctcaagggatcctcctgcctcagccttccgggtagctgggactacagatgtacaccaccactcccagctaatttttactttttttgtagtgctggggtgtcaccctgttgcccatgttggtctcaaacacctgggctcaagtgatcttcctgcctcagcctcccaaagtgctgggattaaaggcatgagccaccacagtcagctgtgtatgaattttagaatcatattatctacaataaagttttggtggagtttcaatagtaaatgcattaaacccattggttagtttggggagaagtgacatcttggccatgttgtcttccagttcatggatatagtatgtttttttacatttatttaggttgtcttttttttttaaaggtacataataattgtacatatttatggggtgcatgtgatattttgatacaggtatgcagtgtataatgatcaaagcaaggtatttaggctatccatcacctcaaatatttatcatttctttatgttgggaatttttcaactcttcccttctaggtattttgaaatataaacaaattatagttaactacagtcaccatactgtgctgttgaaccctggaactaatttcttctatctaactgtatgtttgtacccattgaccaacccctctttatctccctgcccccttcccagctctggtaacgatcattttactctctgcctccacgagatcaactcttttagctcccacataggagtgacaacatgtgatatttatctttctgtgcctcttcctttacttaacaatgtatttaggtcatctttggtatttttatcagcatttcataggttttagcatgcatattttgtacagttttgttagatatatacctagttttttttggtaaactattataaatgaaattgtgcttttaatttttgtttctaattgctgattgctaatatatagacattattgatgttagtgtgttgatctgtattctgtgacctttataaactcacttattagttctacaagtttttctcctagaatctttgggattttctctgttagaccatcatatcatctgttaatagggccggtttaatatattcctttctgatatgtgtaccttttatttccttttcttgcttgattgactagctgggacttgtttgctgtgtcgaatatgagtgcttagtgtggacattcttgccttattcccgctttcagatagaaagcctttagtctttcaccactagtatgatgttagttatagacattttatagatggtctttatcaagttgaggacgttttcttctattcctagtttactgagagtttttatcatgaatgtatttgttgaattttgtcaaagtttctgcattgatataaataattatgctttcttttcacatcttattgttttagtttgcatttccttgatgatgaattccttttcatatgcttactgaccattttatcatcttcctgcgtgaattttctattagttttgcccatttaaaaaaagtttttattttgtagcaattttaagctttcataaagttctaagtacagtacaaagaactttattttttcctgacccattttagagccctgttgttccttactatttttatttattttttgagacagggtctgtcactctgttgtacagactggagtgcagtggtactatcatagctcactccagggcataaacgatcctcctgcctcagcctcctgagtagctgggacaagtgtgtgccaccatgcctggctaatttttttaattttaattttttgaacttctgggctcaagcgatcctcttgcctcagcctcccaaagtgttgggattataggcatgcaccaccgcacctggccctacccttaatactttagtattaatgttttagtatatttgccttaaagttaatgacattctccttgctacctgtccacaatacattcataaaattcagaaaattacactgagtcattattaccatctaatcttcagatttcattaaagtttagctaattgtccatgtaatgtcctttttgtccaaagtatctaggtcagaattatgtgttgcatggagttgtcatatgtctttggtctccttcattctagaacagttctttaatctttctttgattttcgtgaccttgactcctttgaagactacagaccagttattttgtggaatcttttttagtttgaattttctcataattggattcaggttttgcatctttgtcaggtatagcagagaagtgatgctaggttcttagtgtgtcctgtcaggtagctctttatttcagttttttcctattattgattatctctttgatcacttggttaaggtagcatctgccatccttcatcactataaagctcctttttacccccacccctttttttttgagatggggttttgctcctcttgcccaggcttggagtgcaatgtctcaaaaaaaaattaatagtaatatatacagtcatagttagaagccaagatctagtttctaaatgtttgctgctgttggagtgttggtgatcccagttcctttcagtgggaaggggctaaggaatatatgggtatatttacatacatacacatatgttcactcaatctatatttatatctgtctttatctaaatatgttgaaaactatgagcttaccccaataatacaagttctaatccagtcattctagttttttccctttccatatgtataagtcctttttccaacaataataaatctgggctgggcgcagtggcttacacctgtaatcccagcactttgggaggctgaggcgggtggattgcttgaacacaggcgttctagatcagcctgggcaacatgtcaaaaccccatctatatggggaaaaaaaaaaaagttagccaggtgtggtggcacatgcctgtagtcccagctactcaggaggctgaggtgggaggatcacttgagcccaggaggtcaaggctacagcgagccgagattgcaccactgcactccagccagagctactgagtgagaccctgtctcaaatctattccctaatatatttacctaattatttaatcacccactcttcactgttgcagccctttcttcatgcagatgtcctctttactttgcttggactccaacaccctgtgttgggttggctccactctcatcccatggatgagttcactcaagctctgaccagtcacatcagggtgttctcccatatacatgcccttctcaacccgcgtgggcttcaatactctgtcctttgtcattccctgtttgggcatttcactcatcttgatctccaactctcacgtcataccacactcagatacacacagataccttctgtgctccactcaggctctgactctctactcttgtctacctagcctcaggcctccacttttgtatggatgcctaccttgctgtgtaccatgtaatggaagggaagactagtttgggggaagagaagggcagctcttcccatttaaaaagattggtttgtctttttgttttaggtttatagaattgaaatatataggtgggcttggtggctcacgcatgtaatcccagcactttgggaggctgaggcaggtggatcactttggtttaggagtttgagattagcctggtcaacatggtgaaaccccatctctactaaaaatacagaaaaattagctgggtgtggtggagggtacctgtaattccagctactcagaaggctgaggcacaagaattgcttgaacctgggaggtggaggttgcagtgagctgagatcgtgccactgctctccaacctgggcaacagagctagactccgtctcggggggaaaaaaaagaattgaaatatatgtatacacacacatatacacactctggatacatactgtatcccgagtatgtgttttgcaaatattttctcccagtctgtggcctgccttttatttcttttcttttttttgagacagggtcttcctctgtcacccacgctagagtgcggtagcacagtcacaactcaccgcagcctcaacttcctgggctcaaagtgatcctcccacctcagtctcttgagtagctgggactacagacacgtgccaccactcctggcccattatgtctgttttttgtagagatgacgtctcactatgttgcctagggtggtctcaaactcctaggctcaagtgatcatcccacctcagcctcccaaagtgctggtgagccaccatgcctggcctggcctggcctgcctttgcatttttattaaagtgtcttttgattagaagtttttttgttttgaggaagtctgttttatccacattaaaacaatatgtttttaggaacttttgcctatctattaatagtgcacaaaaatattttttggctttatatttttggctcttaggtgtgcgatttatgtcaaattaatttttatttttgagagggagtctagctttgtcacccaggctggagtgcagtggcacaatcttggctcactgcaatctccacctcccaggttcaagcaattctcctacctcagcctcctgagtagctgggattataagcacttgccaccatgcctatttttctatttttagtagagatggggttttaccatgtttgccagcctggtcttgaactcctgacctcaggtgacctgcccgcttcagcctcccaaagttgctgggattacaggcttgagtcaccatgcctggcctcaaattaatttctatgtacggttcgaggtagtcattcaggtttgcgtttttacgtgtttatcttgtttcagctttatttgttgaaatgatgttcccctctttattgaatagctttggtgcctttgtcaaaaatcagttgagtccatacataaggatatgtttttgtactctgttctgttccattaatctatctatttttataccaatattttactctcctgattttagtaagtcatgagattggatagtgttagtagtgtgaatcctctgtgttcttccttcttaaaattgcttggactgcttttggttctttgcatttatttttctttcttttttagagacaaggtcttgctctgtcggcaggctgtagtgcagtgacgcagtcctagcttactgcatcctaaaacttccgggctcaagccatcctcctgcctcaagctcccaaggcgctgggattatgtgtgtgagccaccatgcccagccttctttgcattttcatataaattttagaatcaacttgtcaatttttatcaattaaaaaaattgggattttgattaggattatgttgactttgtagggagaattaacttctcaacagcattttttttcaatccatgaacatggtataatctccagttattatcatctttaatttttcttagtgttttatagtttccaatgaggtcttgcatgccttaacatttttttctcaagtatttttatttttttaattttttaaattaatttatttttttgagatggcgttttgctcttgttgcccaggctggagtgcagtggcggaatcttggctcactgcaacctttgcctcccagtttcaagcgattcacctgcctcagcctccctagtagctgggattacaggcgggcaccaccatacctggctaattttttgtatttttagtagagacagggtttcaccatgttggccaggctgttctcaaactcttgacctcgtgatctacctgccttggccttccaaagtgctgggattataggcgtgagccaccatgcccagcctcagctatttttattttcgatgctattatacatggaatttgttttaaaaatttgtttcccagttttttgccctagcaggtatatagctgatttttcaataacttgtaattttgacataatttcagacacgattgtatttgctttataattctagctacatgttatttttttcatgtactaaagtgcagacataatgccccctaacctctaaatagttattaacaacaagtacattctctttcataaccacattgcaattatcaatgtcagaaaattaatgttgatatggtactgcttgctgtctaatgtgcatactttgagctgggcttggtggtatacacctatagtcttagctactcaggaggctgaggtgggaggatcgcttgagcctaggagttgaaggctgcagtatgccatgatcaaccctgtgaatagccactgtactccagcctgggcaacaatagtgagatccctatctttaaaaaaaaaactgcatattttactgaaattttacctattcttttctaatgccaattataaagcaaaattctggttgaggatctcatccttgatcacatggtacatttgtttgtcaaatctcattttctttaatctgaaaaagttctcacaaacattgtcttttatgacttaattttttttaaagtacagaccatttattttgtattaatagtaagttctccaatatggatttgtcttgatgcttttcattattaaagtaatgtttttgacaggaatacctcagcatgtcctcctcagtacattttatcaggagacacgatacctgtttgtctgatttctgataacgataacttcaaacacttggttaaggtggtgtctgccttatttctctcctataaatagtaccttgtagagagatacagtagtcccctttatctgctggggatgtttcaagaccagtgggtgcctgaaattacagatagtcccaagccctatatatactgtgttttttcctatgcatatgtacttttgataaattttattttattattcctttatttagtttttagaaatacggtcttacaccgttgcccagcctggtcctgaatgaacttctggcctcaagtgatcctcctgcactggtctcccaaagtgttgggactacaggtgtgagccactgtgcccagccccccaagtttaatttctaaattaggcacagtaaaagaataacaacaataactaagaataaaatataacaattgcaacaatgtactataatcaaagttatgtggatgtgatccttctttctctctcaaaatactgtaattttttggactgctgttgaccacaggttatttaaactgtggaaggtgaaacttcgggtagagggcacttactgtactttgagactatatgaatagtctcatgtactttcattcactggttttagcatccactgatgaaggattcctacctaaaacagttagtacgctgaggctgggtgcagtagctcacacctgtaatcccaacactttgggagaccgaggtgggtggatcatttgatgtcaggagtttgagaccagccttgccaacatggtgaaaccctgtctctgctaaaaatacaaaaaaattagctggacatgatggcgcatgcctgtaatctcagctactgggggagggtgaggcaggagaatcgcttgaaccctggagatggaggttgcagtgagctgagatcgtgccactgcactccagcctgggcaacagaacaagactctgtctcaaaaaaaaaaaaaaataaaaaaaataaaaaaaaattagtactctgagagttgttgtcaagtggtgattttttttttttttaagttccaccatttatctgacatctattagttgtggtttttcagtaagggggagagctttaatttattcccccatttgtttattcgttgtgaatttatggattcttttttttttttttttttttttttttgagatggagtctcatgcgctctgtcacccaggctggaatgtagtggcacaatctcggctcactgcaacctctgtctcctgggttcaagtgattcatcttccttagcttcacgagtagctggggttacaggtgcctgccaccatgcccggctaatttttgtctttttagtagagacggtttcaccatgttggccaggcttgtctctccaactcctggcctcaagcgatccacccgcctcagcctcccaaagtgctgggattacaggcataagccactgtgcccggccatggattcctttttttttttttttttttttaaaatatgaagactcgctctgttgcccaggctggagtgcagtagtgcgatcttggctcactgcagcctctgccgcctgggttcaagtgattctcctgcctcagcctcacaagtagctgggattacaggtttgtgccaccacacccagcttttatatttttagtagagatggggtttcaccatgttggccaggctggtctcgaactcctgacctcaagttatccacccgctttggcctcccaaagtgctgagatcataggcatgagccattatgcccagccctacccatggattcttattttattattgagttataatcttactatcatttttaatgtacaaattgtcgctgatttggccagtggaattgcatgtactccagcttttgtgtcctttttatgtgttttcctcattcctgaacacttctttacttacctgctttctgacccagcaagatgttctgggcttagcttatattttcccagccccacttctagaatcatccattttcctggggagctctggtttcttttagcttagaatagtatgcatgtatacatctgtacctgtttctctctttgtctgtctataggtaaatatagtattcatactaataccttgaaattagtctcttactgggggtttcattctaactttacctttctatatgtgcatcttgctactccaacattgaacaacctggcttctattatgtactttgtatctacctacttgctcagttttagtataaacacttagtagttttataattgttaacccattccactgtggaagaacaaatctaactagaactcagtatttgtttatagttcatttaggtttttagcctaaggatatatagtcaaaatacttgaagaattacttgggttagatctgtccttgtgccccttcagtgtggttatgttattcactttaagtatatgtcagtaaaaattttaaatgtatcacaaccacttcctctttttagtgctgtgggaaaacaagcactgttgcattacccttaagaaaagaaataaatttttttttttttttttttggaaacaggtctcactctgtctcccaggctggagggcaatggtgggatcgtagctcacgtcagcctcccaagtagctgggactacaggcatatgcatcctggctggctaatttttttttttttttgcagacatgggggtctcactatgttgcctaggcttatcttgaactcctgggctcaagtgatcctcccgccttgccattccaaagtgttaggattataggcgtgagccactgtccccaggtcaagaaaacaaatggatacttttttttgttgggggatttgatactattaacaaaattatatttgcatctaccttttgaccgattgtttccatttgattattcaaggcagtagtatttgtagttgcagaatattaaaagccatctaaatgctggtggtacatgagggagtggttatactttgatacatcaacatcatggagtatcatgcagctgagaagaaaatgaggaagttttcatgaaacgatagagtaatttccagaatatattgttaagtggaaaattgaagagcaaaatactctctgaggcacgcagattttgttgttgttgttgtttaaggatgaaggataaataaaacagatgtgtatattatcatttgtacaaaaagaaacacaagaagaatacaccggaaacaatgaggttggttaggggactaacaggaatggaatggagagttggagggggatggaaatggaagggatggtggggagcagtgtcacttactctgagtataaccttttatatagttttgatttgtaatcatgttatatttcacataattcaatatgtagataaaataacaaagatgggaaataaaaaacaaaatgggttacaaacaaaagatgaaacccagtgctattaacagctgtacaattcttgatgtgttcaacgtatttcattcttctttctgaatgtttgttcctggagccatcttttttcctgctctccgcatggacagtttgtggtgtaaggtggccagccatcctggtttgcccacgattgaggggtttcccggggtgtaggactttcagtgctaaaactggaaactcccaggcaaactggggcctgttggctactcttttcacgtgcatgtggctgttttcctcggactttccttatatgtaccttgggaattctcttcactttctcctggtatgtcctgcctttcctagatctcacacaaatctcatttattgttactcctttagttgtgtgcgcattctctacacaatacatttatgtagagcagattgacgtagagttgcttctagagaacaggtgcctaggaggtaaattgaggccttacatgtctttattctgtgttccacactagattgttggttgggctcagaattccagaagatgttactcttactactacattcttcagagctgcttttgagaaacatgccattgtgatttctggatatcgtgtgttttttttgaccggaagattttaggatctgtttctctcctgtgttttgagatttcatgataatgctgtggaatgggtctttttcattcattatgctgagtgttttgccttattaaactcggaaactggagtcttctcatttggggaaaatttcttgtattatttctgtcttttggctttcttcctctggaccttctatttgttggatgtgggactctcttaatggagcttctttttcttttttttttttaatcagttctctaggagataatctgttgtttttggcctctcatttttaatttctagcagattgttcatgttctttaatgttgctttatttttttaaaaaaatattctggctgagctgtagtacgtgtctgtggtcccgactactggggaggccaaggtgggagaattgcttacgaccaggagtttgagtccagcctgggcaacatagcaacaccccatatcttattttaaaaaaagcactctatgtttgttttgggcctctttgagaacatttactatgaaattttatttatctttgtttcctttcagttcttcttttgtttattgtgattatctttcatataacaatgtgcttaaatagctggtggttcttgataggtcactcaacagtgagcaaactgatggatcctgtgtttgtgagcagtgtttattggctgtgatagagggtggggacctagatgcttttaggggatcctcctgttatccatttctgaaagtgttttctcctaggctggtcactttttccaggaggagaaatcctttcatctcttgccatctacatgacagtaggctgtggttggggtcatagtactactactatttggggagctttgggagccaaggtggggagggaagctagtggtgtcatgattttctggtgttttttgtttgtttgtttgtttgtttgtttgttttgttttgtttgagatggagtttatctcttgccgcccaggctggagtgcagtggtgccatctcaactcactgcaacctctacctcctgggttcaagcaattctccagcctcagcctccctagtagctgggattacaggtgcccgccatcacccctggctaatttttgtatttttagtatagatggggtttcgccatgttggccaggctggtctcgaactcctgacctcaggtgatcttcctgcctcagcctcccaaagtgctgggattacaggtgtgagccaccatgcccagcctgcgtcatgattttctattgttggtcttatttagagtgtggcatctcactccatgccccatcccttgagctgtgcctctagtcttctgttccagaacttccgtggttcagtttaatcagaatataaatctgttttcctctggagtgggagaagggtgtattgccttactgtttagggacaggaaggagacctgagggggtctgactgttttattcagagtttgaaacagttctcctgttctcagcccctgcctctcaactcttgtctgcagtggttcctggtgcctcccacttccctgcgtgctccgggcttttgaagcactggcttgctttttgtggcttttccctctgcagccataaacttcaactttccttctttgctgagtcagttacttcttaccttcctgctcaacctggtctttcagatttctgttgacatccctcatctactgcctttttccttccccttactctttgtctttatgcatgtacacttttactgttcccttatagtcattctagtgaaccctcaggaagatgcaaagaagtatatgttcattttgccatgtttatccacaacccccaaataccattaaacaaaagaaaagaaaaaatatttaaaaaagaaaacaaaaacaaaaacaaaacaaacccttaataaatggagagcagctatgccatgttgttgattaagaagattctatatcgtaaagatgtcagttcttcccaatttgatttgtagattcaatgcagtacactacaaattttgaaaattgagatataatagttatatgtattttgggggtacaggtgatattttgatacctgtatgcaatacatagacatttttctttgtgttgggaacattaaaattcttcttttctagttattttgaaatatatagtaaattattgtttactataattttcctattctactattgaatattagaacttactccttctatttaactgtaattttataccccttaaccaacttctattttgccatgtttaaatatggaagctcaagcgtttgtacagagaaagtggaaaatgcccttggcttttatgccaaaccccaatccctctgtccccacctgccaccctggcctgttgcccctgttcttcccagtagctacaaccactatcattaatttaacgtgcatcctcctattcatatggaggtatgtggtatgtttgtttatataagtgtattttattttctttaatgtttaatagtgtaccatatcaatatagctacattttatttatccagtcccctgctatgaatatgtgcgtttttcactgttttctaatccctgttataaacactgcagcaacacttatccttaatctccatgtacaaacgtgtgagaatttctctaagggactattcccttaccttttcatattgtatgcttgggtaaaatggaagtgacttggaggctctttctgtgggggttagtgaaaagatttcattacattttctttgcattttgtaattatgatgagaaaaatataaaacttataaatcttttttaaaagttttgagaaacatgctttgttacgatcatgacatcaagttttatgcttaaaaagtctgtaattccttgtgtttaatgatgaactcttcaaattcctgatacagatctatagtcccttattaacaattctgaaatccaaaatattatgagaactgaatttttttccccttgactgatttagtagtaaaacctgacctgtgtaaactcattttgtgtcaaaactctgatgtagattgacaggtctaggttagcttatttgtttcacttggtctttgtctacattttatttttttaaaaattaatgtgtttgattatgggttgctggtggtgctgatacacaatatatgcatatattaacattctaaaattcaagaaattctaaataaaaacacatctagacccaaagatttcaggtaagggattgtggacctgtggtcaccattagctagaaagtttcagctgtgagtggcaacttacaagaatttctttctggttgatagaaactcctttcttattattagtaagaactcagacaatttctttacgttatattccaagagtgtgttcattttttaaataacagctcttccttttctttcgttgacatctataatattgaaattgcttttgataatgcaaatggatttcacattcaagagaacctttttatgttaagtattttgtttaaatggagaagccttttctcaagcatgtatggagattgctttccttgttttaattcttctttaggattttcatgagaaaattaaaacatcattaatggaccattttagccacagttttccaaagttgcattttgactttggatctttaaactttctgtatactttaataggtatgtgtaggtttaaaaaaaattttttttttttgatcagaatcttttgttttaggccaggtgtggtggctcacacctgtagtcctagcacttgaggctgaggagggtggattgcttgcgctcaggagttcaagagcagcctgggcaatatggcaagaaccccatctctacaaaaaataaaaaagttggccgggcgcggtgactcatgtcagtaatcccagcactttgggaggctgaggcaggcgaatcacgaggtcaggagttcgagactagcctggccaacatggggaaaccccgtctctactaaaaatacaaaaaattagctgggcatagtggcaggcgcctgtaaccccagctacttgggaggctgaggcaggagaattgcttgaacccgggaggcagagattgcagtgagctgagatggagccactgtactccagcccagatgacagagcaagactctgtatcaaaaaaataataataataaataaatacaataaataaataaataaattagcctggagtggtggtgcatgcctctagttccagctacttgagaggctgaggtgggaagatcacttaagcctgggaggtcaaggctgcaaatgagctgtgatagcaccgtggcactttagcctgggcccgcacagtgagaccctgtctcaaaaaacaaaaacaaaaaatacgttttagaactatcagttaaattttgtaagccattgtttacctactttaattgggatttaattcatagaccatacagttcaccaatttaaagtatacaattcagtggcttctagtatattcatgaagttatgcaaccatcaccatggtctgattgcataatatttccatcaccccaaaaggaaaccccagtaggtcattcctattctcacctcctcctagtccctagcatcaccagtctactcatttaattactaaaactttctaaaaagcaactcctctcatcttgttcttcaagagtatcttggctctttgtgaccctgtgcttttccatgtaaaattctgtaaaagatcgtgttgagagctttattggaactgcattgaatcgacaaatcaaaatgggaagaattgacatctttttttttttttttttttttttttttttttttttttttttgagacggagtcttgctctgtcacccaggctggaatgcagtggcacaatcttggcctactgtaatctccgcctcccagcttcaagcgattctcctgccttagccttgcgagtagctaggattacaggtgcacgccaccatgtctggctaattattgtatttttggtagagatggggtttcatcatgttggtcaggctggtcttgaactcctgacgtcgtgatctgcccgccttagcctctcaaagtgctgggattacaggcttgagccaccatgcccagccaaattgacatctttatgatataaaatcttcttaatcaagaacatggtatagctctccatttatttagggtttgtttgtttttgtttttttgagacagggtcttgttctgtctctcaggctagagtgcagtggtgcgatcatagctcgctgcaacctgaaactcctgggctcaagcaatgctcccacctcagcctcccgagtagctgagactacaggtgcgcgtcaccatgtgcagctaatttttgaatttttttgtagagcgggggtctccttacgttgctcatgttggtcttgaactcttgggctcaagtgatcctccttcctcagcttcccaaagtgctgggattacaagtgtagccaacatgcctggccttaccaaggttttaagcctttcttgataaagttttatgttttcttcataaaatgatgatatttcttttgtggaatttatccctgggtaacttacgttcgtttttgttataaattatatttttgttataaattatatttttgttttcttttttcttcaaccctggcaaatggatatattttctttttttttttttttgagacagagtttcgctcttgttgcccaggctggagtgcagtggcaccatgtcagctcactgcaacctccgtctcccattcaggcaattctcctgcctcagcctcctgagtggccgggattacaggcgtgcgctaccacgccgggctaatttttgtatttttactagagtcagggtttcaccatgttggccaggttggtcttgaactcctgacctcagatgatccacccacctcggcctcccgaaatgctggcattacaggcatgagcccccacacccggctgatggataaattttctaattgatgatgttaattggtgatgttagtatcacttgatttttccttttttttttttttaatgtgatctcaaatgtcacaatgtggccaaaatcttttactaggtctgagtctcttggatctttaatgtaaataattatgctatctggaaattatgacatgcttttctaatcccttcttgtttctattatgcattgaaaggaattctagtatactgttgaatggttggtgttttcttagtttttcacagccttcaagtcagccttctcctaagagatagtacttattttttgctcaggaaagatataaggaaaggcataactgatattttgcttttttttttacttcagaatctaatcttgggcagtttaaactttatcaatttaaatgttagctctcctttgctcagatttgccacattcgtagtgtcttgtattttttaaaacatctgacctgtgggagaacattttattttggtttacttattacagatggtttattttttttcaacattagcttcttttctagttaccttgccaaactagtatacgttatgcattttagaaacacttatttgtattaatagatgaacgtgagtaaaaagtatacagcaagtcttctttgtactgttttactcttgcaaaagtagaatgaaaagcttttgcagctttcctgtaaaattgaatttctttccatataaaaatgttcaaaatgtatttgtaaaaggaatacgtactcatcatcagtattaaaaaggttcagaataaagaatccctacaactacagtttttaaaagacaactcaaaaaaaaaatgggtgaaagacttgaacagatacttaaagaggaagatatgaatatggccaataagcacattaagaagtgctcatgcagccataaaaaagaatgagttcatgccctttgcagggacgtggatgaagctggaaaccatcatcctcagcaaactaacacaggaacagaaaaccaaacaccccatgttctcactcataaatgggagtgaacagtgagaacccatggacacagggagggaaacatcacacaccggggcctgttggggggtggggggaaaggggagggagagcattaggaaaaatacctaatgcatgcggggcttaaaacctagatgatgggttaataggtgcagcaaaccaccatggcacatgtatgcttatgtaacaaacctgcacattctgcacatgtatcccagaacttaaagtaaaatttaaaaaaaaaaaaacttcaaaaaaaaaaaagtgctcaacatctttagacattaggcaaattaaaatgaaaatcattgaaatctagcactctgaattaaaattaaaaccacaatgaggtaccattatactcccactagaatggctaaaataaaactgccagcttcaaataatggtgaggatttggatcaactggaactctcctcaagtgctgttgggagtataattttttttttaataaagttagctttcatccatcttatgactcggcatttctactcctagatagttattcaggagaaatgaaaacgtctccaaaaatttacaagaatgttcataatagctttattcataatagctagaacctggaaataacccaaatgtccatcaagagagaatatacaaatggttgtatatgagaatattagtctgtaataacaaataatgaactactgatgcatgcaacaatgtagatgaatagcataaacaggctgagtgaaagaagccagatacaaaagaatatatatgtatgagtccatttacatgaagaccaagaacaggcaaaagtacgttatggagatagaagtcagaacaatgattgccttcgtggggtgagaggggtggtttcttttcttttttttttttttttttttgagacggagtctggctctgtcacccaggctggagtgcagtggcgtgatctcggctcactgcaagctccgcctcccgggttcatgccattctcctgcctcagcctcccgagtagctgggactacaggcgaccgccaccactcctggctaatttttttttttttttttgtatttttagtagagacggggtttcaccgtgttagccaggatggtctcgatctcctgacctcgtgatccgcctgccttggccttccaaagtgctgggattacaggtgtgagccaccgcgcccggccgagaggggtggtttcaaatgggggttgattaggaaggggcacaaggaaacttttgggggtgttggaaatgttctatatcttgatatgcatttgttagaactcattaaatggtacacctaagatctgtgcatatcactgtgtttaaattaaactttaatttaaaaaaataggggaaggataggaggaggtatacattgatccaagggcattctcctacgtaagcacaatacagttatcacactcagaaaatttaacattaatgcagcattaccatctagtatgcagttcacattcaaatatcccagtgtcttgaaatttgctgagagtagatcttaattgtcttcacactccccatctcacactctccctctcatggtaactgtgtggtgaaggattggttaatttttttttttttttttttttttaaagacagagtcttgcactgtctcccaggctggagtgcaatggcatgatcttggctcactgcaacctttgccttccaggttcaagtgattcttgtgcctcagcctcccgagtagctgggattacaagcgtgcaccaccatgcccggctaatttttgtatttttagtagagacggggtttcaccatgttggccaggctggtctcgaactcctgaccttgtgatccacccgccttggcctcccaaagtgctgggattgcaggcatgagccaccgtgcccggcctatagaattttttatatgtaggatttagtgaaagatcacccattattaatttttttaaaagcttcatgatgaaaattattcttgtaaaatgttaaaatcttgaatcctagggcatgttttctggcacctgttattttcagtaacccatttcgtttttgtcagacatatagggttaatgtatgaaaagtcaattatgtcaaacttatagagtttatatggaaagattaatgatcttttacaaatgcagaattggctggatgtggctaattgtagaattggcctgtaacctagcacttagggaggctgaggtgggaggatcgcttgagtccaggagtttgatttttttttttaattttttattttttgagacagtctcactctgtcgccctggctgaagtgcaatggcatgatctcagctcactgcaacctccacctcccaggttcaagcgattcttgtgcctcagcttcccaagtagctgggattacaggtgtgtgccaccatgcctagctaattttttgtgtgtgtgtgtatatttagtagggggggtttcatcatgttggcccaggctggtctcgaactcccagcttcaggtggtccccccatctcggcctcccaaagtgctggattacaggtgtgggccacaatacccagcattttttatttatttatttatttatgtatttgagatggagtttcaccctgttgcccaggctggagtgcaatggcatgatcttggctcactgcagcctgcatgtcccgggttcaagcgatcctcctgccacaacctcccgagtagctgggaatacaggcatgtacaccacacccaactaatttttgtatttaagtagagtcagggtttcattatgttggtcaggctggtctcgaactctggacctcaagtgatctgcctggcttggcctcccaaaatgctggaattacaggcttgagccactgcacctggccaagcccaggactttgagaccagcctgggcaacagaccaagaccctgtctgtacaataaaaattatgcagtattagaatagcaaatcttactcttaaaagttttgttttccttgtctttttactgaaaatgcatggctttagaaacttctgggtagtttgaattgcagacaaccaggccctattcctgtttgtttaactaatgagatttgtaaacagtacttggtttccccagactctaggacctagaggaaggaaattttgatttagctcttggtaaagcatttaacacctaatttttctttttcattgttattttcttgatcacagaaaacaatacatgttaatagtagaaaatacccacaattgaaaagaatataaagcacttactaaaactctaccacctaggctaggtacggtggctcacacctgtaatcccagtactttgggaggctgaggtgggtggatcacctgaggtcaggatttcaagaccatcctggccaacatggtgaaaccccatatctactaaaactacaaaaattagctgggcgtggtggcagccacctgtaatcccagctgctcgggaggctgaggcaggaaaatcgcttgaacccaggaggaggaggttgcagtgagccgagatcatgccgctgcactccatcctgggtgacagagcgagactcttgtctcaaaaaacaaacaaaaaaactgccacctagagatggtggtggccaacagcttagtaagaatttctcttgcatttttctatgcaaacacattcaaattttcattttttaaaaaagatgatagaatcatactgtatgaatttaattgcctaattaagcttgtgtatcatcgtttctcttgtcattaaatgctgtacagctttcttttaaatggctatctgtggtagtgttccttgaaggaaataccttaaaattttttgtttttaaacttttataattgtgggaatgtaatacagtgaatgcataggacacaaatgttcagcttaaggaattattataaagtgaacaccctgtaactgccacttagatcacgacatcaaatatggccagcagccaggcgcacctcccctaccccccactgccccttcccagtcacagccccattcttccagctgaaggtaacttctgtcttgactttatgaaagttattttcttgctttcctttagattacctacccgtctttgcattcctaatgctgtagtttagttgagcttattttgaacttttcacaaacggaatcatcaactatgcattattttgtatctggctttcattcactattttgtttttaatattcatctatgtcgttagtagctatagttcattcattttttgttgctgtataatcatccatttattgaatgtactacaatttgattctgcctttggcagccactttgattgtttataatccagatttcaaatgctgcatcatgaacatgtgggttgtgatttgtgtagatgtgcatgtacttctctaaggaatccaagcctaggagggacgttgctgggtcagaggatgtttgtatcttcaactgtaccagataaggccaaattgtattctcaaatgcttggatctgttttcagtgtacttgttctactcctaattaacactacttgatattgtcaaactttaattgtagccattctgatggatgtggtagtatctcattgcggttttaatttgcattattttgcattttccttattgttagtgagattaaatacccttttatatgttttggctacttcagtatcctcttgtgcagtgcctaattcacatcttttatttttcttctgagttgccttttgcccattgatttatatttgttccccctctctccccgcccccaagaaggagtctggctctgtcacccaggctggagtacagtggcacaatcttggctcactaacctctgcctccttcgttcaagtgattctcctgcctcagcctcccaagtagctgggactgcaggtgtgcgccaccacgcccagctaatttttgtatttttagtagagacagggtttcaccatgttgaccaggcttgtcttgaacccctgacctcaagtgatccatccgccttggcctcccaaagtactgggattacaggtgcaagccaccatgcccggcctgatttataaatgtagtctaaatacaagttctttattggttatgtaagttgcagatatctttttcagctctgtaggtaataatgttttttgatgaataggaggtcttaattttaatgtagtttaattgatttgttttctcctttgtaagaatcaatatttttcatgtcctgtgtaaatctcgtattgggataaaagatacatatatattttttaggttttggctaatagttttccgttggcagcatgtgaacctttttatgccatttttactttcatcaagggtgtatgtatgattttctcatactcttgtcagaactggagatttggggtaactttgccagtgtaatagactttcttaattcattaaggagtattttatcttagtttgcccatgagagaagagagattaaatttgttttattataccaagaacatggcagtcaataagcataactaaaaatattatttagcttttgattctgaagaccatgtatggccagtaaggaaaagaggtaaagatgtagaaaactgttttcttttttttctttcttttttttttttttttttttttttgagatggagtcttgctctgtcgccaggctggagtgcagtggcgtaatctcagctcactgcatcctccacctcctgggttcaagtgattcccctgccttagcctcccgagtagctggtactacaggcgcgtgccaccacacctggctaattttttgtattttagtagaaacagggtttcaccgtgttggccaggatggtctcaatctcctgacctcgtgatccacctgcctcagcctcccaaagtgctgggattacaggtgtgagtgagccactgcacctggcctcttttttattaatttaagatacatgattttggctaaaactaatgaaaataaattgatgaaacttcaatgtagtggatcaagagactcatgaaattgatgagttagattgttgtgggcacctgttttaaatttatggtgcctttttagcagaatcatgactttatcaactctttttcacatacttttattcaaagaagacattctcctcctctgccccaacttatcttcttcttgtctccaactgccagtattctcatccacactctttgtagttttgaggtgtgagataactcccaagttcttcttgggaatgggtagagtttaattctgagtctacatgtcgtgagtctattggaagaagcttttcttaggaggtgaaatttgggatggagttctcctctacactctagccctgtagccacaatctttggtttagatctttggcattttttgttggacatctttcatagtgtctacctcttcttcctgcctctaggcttcccgcggttgccagatgaaattccgaaaactcaaatcattcctgtagttaatattcttcagtgactccggaacaatcaaattcttgatccctccacatgggtcttaccatctgccctcgtgccctctctagcttcctgcatattgagttccccacagccctagtttccctgagtttggaatattctttcacatttgctcatcccactatctagaaacttcctacaatctctctactgccaacctgtctgtctagttgctaatttattttaaaacccagcttaagcaacacatccttttggatgtgttacttccctgatcgttattcacctccatgccgtctttcgctcaactcattgaagttaggtgcccctcttctgggctgccacggctctccatgttcacctatgtactgtactttgtattgtaatcagtttaattgacccccctacttatttgagctccttgagagcagggccttgattcctgctccttagcactccaccctccccagaactaacatggtatagcttataggaggcaccctataaatgtttaatgaatgtttgaatgcaaaagtgtatctaactttcttaatgatatatttatgaaaagaacttgctgagaaaaaggtcagttagaaagggttaggaacttgtcagtattctaaatccctctttttcaaaagcatacacatttaggaatgactgccttgctttaagaatttgtagggttttgttcacctgagttgtaccttgctgtcacagaaggatgccctgcagtggtctcgccatccagctacagtggagggagaggagccagaggacacggctgatgtggagagctgtgggtctaatgaagccagcactgtgagtggtgaaaacgatggtaaggaccctttaatggatgggtgagggagccacagcaggcactagggactaacctttattttcctctcttccagtatctcttgatgaaacatcttcgaacgcatcctgttctacagaatctcagagtcgacctctttccaatcccagggacagctacagagcttcctcacaggtaaggaagaggtagagcctagcctgggaggatgaatgatgacaggtataaggcaagatccctccttctcctgtagtgagctgtgaacatgttcagagagtgaagaatagaagtctggtctccagctgttagtagcatttaacagggggccgcaggaaaagtgaacagtaggtttgagcagagtaagttttttctctttctttaccttggctgagtgattttgttcattctttttttttttttttttttttttttttgagatggagtcgcgcctgtcgctcaggctggagtgcagtggcatgatctccgcttactgcaacctctgcctctggggttcaagcaattctcctgcctcagcctcccgagtagctgggattacaggcacgtaccaccgtgcctggctaatatttgtatttttagtagagatagggttttgccatgttggccaggctagtctcgaattcctgacctcaagtggtctgtccacctcagcctcccaaagtgctgggattacaggtgtgagtcaccatgcctagcctgattctgttcattcttgtaatatacacgtgaacattcagggagaagtgtggttagtgcagagtagagagaacaggagattgggagtgagcagtttgaatgataaccctgcacttactagccttgagacttggggaaaattccttacctgtaaaatggggataacagtacatttttgtagcttggaatgatgcttggcacagtgaccagcacgtagctcagtaacattaaccagtgctcttgtcagcattatttgacagatctggttgaagacgaacttcattttacaagagcgtgagtagagatagtgtcgccagggaatgcttttgtggctctgcagttgacttgggctctcttttgttctctcttggaacgcaggcgaacaaacaaaagaaaaagactggggtgatgctgcctcgagttgtcctgactcctctgaaggtaaacggggcccacgtggaatctgcatcaggtatgtgtaaactcatggttgtgatgctttttcctcaggtcctggggacctggcctccctctgtcagcagtttctgacctaatagtgcgatactaggagagctgtcgggccacaggcaagagccctgccaatggatgaggcctgccataggttctagtgctgggctctgctgtgtgccttcctctttgtctcccagggcagtcgtgaggatccaacggaggatttgattatgccagtgctttgtaaaaggtgtagtgctatacaaataaagatggcagtttggcacctgtaaggtgatattttaaagccagaccatgaagtggtggtttctctcagcctaaggctgggagatggaagcatcccagtattgctggcagcatctcagggagagctgggagaaatgagcttgtctgagagccatgggcgcggcttggtgatacttttgaccagtggaatgctgtgccttcagggttctcgggctgccacgccgatggcgagagcggcagcccgtccagcagcagcagcggctctctggccctgggcagcgctgctattcgtggccaggccgaggtcacccaggaccctgccccgctcctgagaggcttccggaagccagccacaggtgagtggcgtggcacttatttctctgcctgtaaagggggcccctgcaggcctagtgagaagatgatgtcttctattccaattcctttaccagtttatttttacttcttgggtggccctctattttttgtttatttttactttgtaatttgcttatttcatttgtagctctctgctaaactgtgagaactcctttcaggcagagatgatgccatactgtttctcgtatgtccctgtaggcctagtctatgcctgccttgtactgcatagtaagtgtttgagtgagtgaaaactgcttctgagcttctcagtgaagaattccaacaccccttttctctcttcggtagcatgttgccacatttgttcccaacatctgtagtctgtatcagacgctcccttttttacaacctagtcagtatgttgagtggaagcatgtgttgaattttggggatgcaaacaaattcagtagtgctattatctatacctcccacccctcaccccaccccacccccattagggttatttgcaatattagtatgctctttgctatttcaggatttaattcttaatgacccacatgctggttttttttgtagatgaaaccattaagaagacaagcttttgcttctgagaattagagtaaacctttgcccatgtgagaactaggtttgctgtcttgctgcttttccagctcatcagcagtgctcaatctaaggtctcacaggtctctagtcctagtgagtttttcagataaaagtaacatttgctgaggcttctgtgagaggtattagcatctcagttggctttgaacagtccttagtacatttctgcatggagatcatccctctccagcccattcacagaaggcttctgcagatttttctatagtatactaactagatttacttttagataattctcatgatttctccagttgagctttgtggagcctgttctccagctttggagggaagctcagaacctgccccttgcctgattttctgtgggaagtgtccttttcaggctgtgacacagcacggtacagagttttgtctgaggagatgcaaccccaagtgttctcctgggtttgatggcaatgacagtaacctggaaagaatatacaggttaggatggactggacaattgagcagattgtgtgcagataactcctgggtagcttgggtgctgtcaccaaagttttcccatcagttggcatttgttttttcttttaaaaagctgaaatctataccttgcttcaaaaatcataggtcaaatgaagcgcaacagaggggaagaaatagattttgagacacctgggtccattcttgtcaacaccaacctccgtgccctgatcaactctcggaccttccatgccttaccatcacacttccagcagcagctcctcttcctcctgcctgaagtagacagacaggtgcacatgggcagcctcccctttgcctctctctgggtgggcttctgttctcttttaagtttatttattaggatttttttcccccttgatccttctaggtggggacggatggcctgttgcgtctcagcagcagtgcactaaataacgagttttttacccatgcggctcagagctggcgggagcgcctggctgatggtatgtagacttggtcatctcggacggcttgcgacgcacctgtcgtgtggtgttgcatgtctcctggtatttaaaacccaagctcgctcttctcaaagggttatttagtataagacaggctttcctcattactaataaatgtggtgggtattaatatgccacacagctctgaagaaataacagggtgtttaggtttagactttcactatattgtatgtatgtactcattcatcatctcctttttatcattaaaactataaacacctattccagtaatcatggcatggtctggaagggtggaggaaagtacactgtggagttgagaagctccaccatgctctgtctttttaggcattgtcacttggagtgattgttagcttctattaatttaattttgtggggaaagtgttgagagggctctaagaacacccctaggtttgatgatttgctagcaggactcagcatatagttatatgtacagggctgcggtttattacagcagaagaatacaaagtaaaatcagcagatggagaggcacatggggtggggtccaggggaaaccatgtgttggcttccaaggtcctctctcagtggtgtcacacaggacacatttaattcccccagtaacaagttatgataacatgtatgaaatgtctactagggaaacctgttaaagactcggtgcccagggtttttatcaagggctgatcatgtaggaagccactgcttggcacatacccagcttccagattcccaaaaggaaggcaggtgttcagcataagccgtattgtttgcataaacaatttaggcacagtgaacaactcatatcagttagggtggttgggaaccctcctgaaatctaagttcccagatgtcagccaagggctgttcttttaagtaggcctttctaagagtaaacaatcaggcctacctactatgttaactttttatctacacaggaaatatgaatggttcttttgtttgttgttttgctttttcgatcaaggagttgcttggtctcactttaaggaagagtgaatttcccttatagtagatgtgttagctctgtccctataagagcatgatgtgagagagcctttagaagagacgtgttgttttaaaactgggagattcagctgtccataagacagacattaatatcccgaatgcacttactagaagaggtttatttctccctaggtgaatttactcatgagatgcaagtcaggatacgacaggaaatggagaaggaaaagaaggtggaacaatggaaagaaaagttctttgaagactactatggacagaagtaaggcagttggagctatgagtcctggtctggggttttgaggggatagaggtagatggtctcaaaatagcatatacttatgtgttggacaagaatgtggagattgcagtgacacttgggtcatttatcttaatgccattcatagtgaagctaacagaagtttttccatggttagattgtgcaccacacagatttattttgttctgagatatctgtgtttctgggtccatattattcatagaaataagagacatgtccactctggcctgaaactgatggctgtgattttgatttgcaggctgggtttgaccaaagaagagtcattgcagcagaacgtgggccaggaggaggctgaaatcaaaagtggcttgtgtgtcccaggagaatcagtgcgtatacagcgtggtccagccacccgacagcgagatgggcattttaagaaacgctctcggccagatctccgaaccagagccagaaggaatctgtacaaaaaacaggagtcagaacaagcaggggttgctaaggatgcaaaatctgtggcctcagatgttcccctctacaaggatggggaggctaagactgacccagcagggctgagcagtccccatctgccaggcacatcctctgcagcacccgacctggagggtcccgaattcccagttgagtctgtggcttctcggatccaggctgagccagacaacttggcacgtgcctctgcatctccagacagaattcctagcctgcctcaggaaactgtggatcaggaacccaaggatcagaagaggaaatcctttgagcaggcggcctctgcatcctttcccgaaaagaagccccggcttgaagatcgtcagtcctttcgtaacacaattgaaagtgttcacaccgaaaagccacagcccactaaagaggagcccaaagtcccgcccatccgggtaggagactgtttgattcctggctgccctggagccaggttttctttgagggtcatgagattatagagcccttaactctggggccctgaagttaattatgtgggaatgtagagctttttatgagaggtttgcttgagacagccttcaagtttagtgagataggttcttttcttcctcacttttttgttcactctgttgaagttatcttgagaggtcaaaatcctttggggttttaaaatctttttaagatagcattagattctttgctatagtgactcacacagtcccaccagaaattaaatttagaagtgtggcatataacagcccttgagcagaatcttctctgaatggtgtgttatggcgccattttactatgatgatttcattgttttaaggaaattatttgattctgtatgccatgacccttaagctactagaatcctagttttgctttacagtccctaggtcagatcacccagtcagttaaaactattttctaattctttttttgcagattcaactttcacgtatcaaaccaccctgggtggttaaaggtcagcccacttaccagatatgcccccggatcatccccaccacggagtcctcctgccggggttggactggcgccaggaccctcgcagacattaaagcccgtgctctgcaggtccgaggggcgagaggtcaccactgccatagagaggcggccaccactgccatcggaggggggggtggcccgggtggaggtggcggcggggccaccgatgagggaggtggcagaggcagcagcagtggtgatggtggtgaggcctgtggccaccctgagcccaggggaggcccgagcacccctggaaagtgtacgtcagatctacagcgaacacaactactgccgccttatcctctaaatggggagcatacccaggccggaactgccatgtccagagctaggagagaggacctgccttctctgagaaaggaggaaagctgcctactacagagggctacagttggactcacagatgggctaggagatgcctcccaactccccgttgctcccactggggaccagccatgccaggccttgcccctactgtcctcccaaacctcagtagctgagagattagtggagcagcctcagttgcatccggatgttagaactgaatgtgagtctggcaccacttcctgggaaagtgatgatgaggagcaaggacccaccgttcctgcagacaatggtcccattccgtctctagtgggagatgatacattagagaaaggaactggccaagctcttgacagtcatcccactatgaaggatcctgtaaatgtgacccccagttccacacctgaatcctcaccgactgattgcctgcagaacagagcatttgatgacgaattagggcttggtggctcatgccctcctatgagggaaagtgatactagacaagaaaacttgaaaaccaaggctctcgtttctaacagttctttgcattggatacccatcccatcgaatgatgaggtagtgaaacagcccaaaccagaatccagagaacacataccatctgttgagccccaggttggagaggagtgggagaaagctgctcccacccctcctgcattgcctggggatttgacagctgaggagggtctagatcctcttgacagccttacttcactctggactgtgccatctcgaggaggcagtgacagcaatggcagttactgtcaacaggtggacattgaaaagctgaaaatcaacggagactctgaagcactgagtcctcacggtgagtccacggatacagcctctgactttgaaggtcacctcacggaggacagcagtgaggctgacactagagaagctgcagtgacaaagggatcttcggtggacaaggatgagaaacccaattggaaccaatctgccccactgtccaaggtgaatggtgacatgcgtctggttacaaggacagatgggatggttgctcctcagagctgggtgtctcgagtatgtgcggtccgccaaaagatcccagattccctactgctggccagtactgagtaccagccaagagccgtgtgcctgtccatgcctgggtcctcagtggaggccactaacccacttgtgatgcagttgctgcagggtagcttgcccctagagaaggttcttccaccagcccacgatgacagcatgtcagaatccccacaagtaccacttacaaaagaccagagccatggctcgctacgcatgggatctttacatggtcttggaaaaaacagtggcatggttgatggaagcagccccagttctttaagggctttgaaggagcctcttctgccagatagctgtgaaacaggcactggtcttgccaggattgaggccacccaggctcctggagcaccccaaaagaattgcaaggcagtcccaagttttgactccctccatccagtgacaaatcccattacatcctctaggaaactggaagaaatggattccaaagagcagttctcttcctttagttgtgaagatcagaaggaagtccgtgctatgtcacaggacagtaattcaaatgctgctccaggaaagagcccaggagatcttactacctcgagaacacctcgtttctcatctccaaatgtgatctcctttggtccagagcagacaggtcgggccctgggtgatcagagcaatgttacaggccaagggaagaagctttttggctctgggaatgtggctgcaacccttcagcgccccaggcctgcggacccgatgcctcttcctgctgagatccctccagtttttcccagtgggaagttgggaccaagcacaaactccatgtctggtggggtacagactccaagggaagactgggctccaaagccacatgcctttgttggcagcgtcaagaatgagaagacttttgtggggggtcctcttaaggcaaatgccgagaacaggaaagctactgggcatagtcccctggaactggtgggtcacttggaagggatgccctttgtcatggacttgcccttctggaaattaccccgagagccagggaaggggctcagtgagcctctggagccttcttctctcccctcccaactcagcatcaagcaggcattttatgggaagctttctaaactccaactgagttccaccagctttaattattcctctagctctcccacctttcccaaaggccttgctggaagtgtggtgcagctgagccacaaagcaaactttggtgcgagccacagtgcatcactttccttgcaaatgttcactgacagcagcacggtggaaagcatctcgctccagtgtgcgtgcagcctgaaagccatgatcatgtgccaaggctgcggtgcgttctgtcacgatgactgtattggaccctcaaagctctgtgtattgtgccttgtggtgagataataaattatggccatgggaaacattgtatatttagtgtgtgtattttgataatgattgatcttaaatctgtatacagaatatcattgatataatactctttaggcaggagcactcttgccttcccccaaaatttacactgctaaagccctctgtcacttggcgacccttctggtcttgctggaggggtttcctgggtataacccattgggctgcccaaggccagccagcctgagctctcctgcaagacagagcctgatgtggcacggagtggggttgcggggggtggggggactgcctgactcccagagggacttgaaactgaagcaagaaggttgcattctccaccaagggagttaacctacctgaactaagtagaaatgccagtcttccactaccccctccctgccatcttttcttctgctactttggggagttgatggccaggaaagaagccagcacagggttaaagtaactcctggcattgcccaccagggggctggtgcacctgctgacctcagggtcacagttgagtcatttgccagttgacggagcaagtttgaccttggttctgttgctgaagcaaatttggaacttttctgtctcagtgtgatccactaacccacaggatcatttggaaccttgaatagctctgcttggacaatggggttggggaatagggttgtctttcctatgaaaatgccatctgtagaccttgtgagtcagccgtccagatgtttgcaggtgaattcctctgcttgacatcctccctgtcactttggaccctatgggagtgggcatctccacgcacctgtgtatgtgaaagtcattttacatttcaaagcagtgtgtgtttcttatttttatatttttaactctttattcttggatgtataaagtgaactttttggcttctgtaagtatgctctatgcacctctaatgttttatcatgtatttatatgttgtacacagtactggctgattctgtaaatggatgtattgtacagagaacatgaacgtctcttcctaattttacatcttcagcatcattgcattaaagtggtgtaatctccttctctacatctgttgtcagagccactgagtgctgtgctgctcgacgtgagggtgaaatgattgacttgtgacctgccaggttgcccgatgccctgttgggtcaccggctggacctgctgcagcctgcagagccacagtcagcctgcccacatgccaccgagcaaacgcatcttgcttttcacatctctcctcctacagccttaatggctgcttgctgccatatgtgacaaatcaccaccaccagtgttaagtgcttctggattcatgggtgagttccctgggcagcccccaggaaggccttccagatctggctccagggtcaccacctgtcacagcaatacctgggaccatgctctcctgggactgtgaggctccttttgacgtacttttgacatcaggcaggtttgggaagaaacaaagccatgcctgctcctgcctctctcccaacatgtttccagcaagtagatgcccctgtgtgtgttttcccttgccttgtttcctgccttatatcttgtatttcgacttattacagagttgagggttcttgcttaatttagatcaagtataaaatttgtatgacttcaagtctcattttatctgaaaggtttttttctcatttaatctgatgtggcattttcgtcatctgaagcatgagtgacaagttgggaatgatgtggtgatttagaatgcagtattggccaagtccaagttgtcaacttaagcgtctgtttaccaaagaccgggaacaggggcccaaacatgtccagtcctcttcttccctctgctggaacctttggggacactcaagggtacagtttgacactgatctggtccatgaggctgcccagagaaagcactgcttctgtatgtctcttgtggtattggaacaataaacccgtacaacctgca

SEQ ID NO: 6 - DNA, 76894 bp, Artificial sequence
  synthetic sequence
tccggtcgcgtccgacacccggtgggactcagaaggcagtggagccccggcggcggcggcggcggcgcgcgggggcgacgcgcgggaacaacgcgagtcggcgcgcgggacgaaggtaacgcgccgctgcgggcggcccggccggcggggctccgggagtgcgaaccgggcggcggcggcggcgccaggacctccccgccactgctgtgccggtcccgggtatcgccgagcggggctcaccggggcgccgcgtttgtaggcgtgcggggggtggagggtgaggggagagcccccctccccggaaggagctgtgagcttcgggctggcccgcgggaccctgcgagtgaggcagcgcacacccctggagctgcggggcggggggcgtgtggggggctcctttcccggtgcttgtcctggccggggcgccggggccggggtcggggcgttccggtgcccggtccagggtggcagctcgagcctcaagtctcctttgtgtggcgcggccgcggcgggagcgcccgggtgggacgggacagacacaagttggtgggacccagccgccgagtccgcactcagacaaaggacggactgtgtctccggagggctcggcgcgaggggaaggggcatgacacccgggcccgccggcgggcgggagaggggcgtggggaggcgcgggccgctgccggggtgggcagggctcggccctcgcctcgcgcacgcccccggtcccgggcgcgccgaccccgggctggggccgcggcgctcgcgggacgacgttcgcggcggggaactcggagtagcttcgcctctgacgtttccccacgacgcaccccgaaatccccctgagctccggcggtcgcgggctgccctcgccgcctggtctggctttatgctaagtttgagggaagagtcgagctgctctgctctctattgattgtgtttctggagggcgtcctgttgaattcccacttcattgtgtacatccccttccgttccccccaaaaatctgtgccacagggttactttttgaaagcgggaggaatcgagaagcacgatcttttggaaaacttggtgaacgcctagtgagttgccactttctaaagaatcgtaatccttaaacattacaagctgttttggccaacttgtgtgatacatctgagaatggcgacaggtgctaacgaaatcaactgtcattcagccagattattccccaaatcccgacgggttgaatgaggatgaggtggtagaagtaggtgatcaaaatagaatggctatgacaaatgtgtgtttagattatacggttagcagtttcttattaattggtctcgttttctgtgaacaatagctttggtgattcagaaaagactgcatggagaaatgtttgttgcctaagaaatttaacgtgtgctgtccacagttaaacttcctttatcacacaggatctgtaacatttaaattcatttggaaataaaaatgtgtctttgctatttacttaaaggcttgctttttagggttcaagcaagttattattagtttaattttgtggcgttttgatgtcatcataaaacagcatttaataaacctttgaaaaacacgaatgctgtcacaattgaatgtagtttatacttttaggggttttggagtttattttctattactcttcataagcattagcaactaattcatgtttaggaatactgtacaagtgtgtaatcctaacttgtttatttgtaattttatttgcaggaaaacaagtatttaagatatattttaattttctattcatggtctctaaagttaggaataggtaggattctagtcatagatataaagcgtcttgttctctcttttcggaggtgtgttgggtgatgtaatatttaaggtttcccggaaacaggaacactgcatcttaaattctcgtttttgtggaaagtgaatatattccaagaaaacaagttagaaaaacataggcgttcttctggttgcactgtcctatatgataaagatctaatgtgcatctgctaacttaaggtttcataaaaatggagacaaataaatgcaatataaatttattacatatattttggagagtgttgacaccactgtacgggcattccaggtgctggtctaagtgttgagacattataacaaaaaagtaacgtttatattagttactattaattctgtatttaaaaaatcctattaataatcttgtccattatgagataaggtagttatgcagatttttggcagaaattcggatattttaggatacatatgtgtgtgtgtgtgtgtgtgtgtgtgtgcgcgcgcgtgtgtgtgtgtgtatatgtttttttttttttgagacagagccttgctctgtcgtccaggctggagtgcagtggctggatctgggctcactacaatctctgcctcccgggtttaaggaattcccctgcctcagcctcccgagtagctggaactacaggcacgcaccacgacacccggctaatttttgtatttttagcagagacagggtttcaccatgttggccaggctggtctcgaactcctgacctgaagtgatgtacccgcctccgcctcccaaaatgctgatccagacgtgagccaccacgcccagcctaggataaaaatttctaactttgcccttgagtgttctctgtatttgataattactattctttgtacttgccatgagatattttgaaaaacatctctttggggaattggtttaaaaattcatattaaattattttctggtacaattgagaatgtatttagtcagtttaagtagcagtgacttaaaacacacttgttgatctagtgtagttagaaaaagttctgatactttgatacctagctttgccttgcacaaatacatttaagtgaatatctttatgttggccatttaaaaattttaggcaaatttctccaagtcatactggaattttcccaggataccttccttcttttgaggtagtgtgctcaaggattctgaatttatttcagtattaccctttacatgagcagtgattgagaaagttgatctgctttttaaaaaccaacaaggacaaatcctccaatataatgtattctgtggttaattagcaatgatttatggattagccttgaagtctttatatcagtatagatgacaaattttctgtaaatcatgatagattctatatgagtaggtgaaatcttcgggggagagcataagtgaacagcagcaattcattaaattcagagaaaggttcatggtcagatgatacatactttaccttctggaggtcttccctgaattcccactcttgtgttcccacagggttttatcatattttattgtagcatgactcaattgtattgtaatcacttgtttatgcatccctctcttccctacagtcaaagtgcctcctggtagaggctctgtcttatttttttctttgtatacctagtacctacgttggtatttgctagagaataggggttttaaatatagttgaatgaattaaggaaatatgaaaaatgtagggaaataatttgacaaaggaacttgcaaagtgaacaggatatgttagtattcatgtgaggaactttgaaatgtgtttttctccctctttaatagggaatgattacaaagaaaaatagttgtcaaggtgaacaagttccaagtttttcctagttataatctgtagtactaaacattagatatcctttagatgcaactattacactcggacaactggagagaacttgatgaatcatctggatatttagtaatagtatttgcttaatttgatttaatgttgatattgatgatctctttgcaaaaatataaggaagggaaagggcgagagagggtagtagatcttttaactgaaaagctgttttgttataattcatgagttctttattggagtaaattgaggtaggttttggagctaaaactgaaggatgtagcgacagtgtaattggatcatttattgggatgcttttcatcattcttttaaaatgatttaaatacagattagcaaatttagtgatacctcaaaagttacagctggtaaatttctaagatagttgcaccattccatatcttgccactgttccactacttggtataaaaatatgacaaatttgtgtttagattatgtggttagcagtttcttaataattggtcttgttttccattaacaatagcaagacagaacaagatcatagctcactgcatccttaaactcctgggctcaagggatcctcctgtctcagcctcctgagtaactggtattaccagtgcacaccattgtggtgacttatttaattttttaattttttgtagagatgggatcctgctttgttgcccacgttgatctcaaactcctggctccaggcaatcctcctgccttggcctcttaaagtacagggatgacaggctgagccattgtgtttgacccagatactttttttttttgagacggagtctcgaaagtgattcttgtgcctcagcctcctgagtagctgggactacaagcgtgcgccaccacgctcggctaatttatttatttattatttattatttatttttttgagatgcagtgtcactctgtcacctagactggagtgcagtggcaccatcttggctcactgcaacctccacctcctgggttcaaatgattctcctgcctcacctctcaagtagttgggattacaggcatgcaccaccaggcctggctaatttttgtatttttagtagtgatgggatttcaccatgttggccaggctggtctcgaactcccgacctcaggtgatccacctgccctggcctcccaaagtactaggattacaggcgtcagtcactgtgcccagcctccgataatttttatattttagtagaaactgggtttcactgtgttggccaggctggtctggaactcctgacctcaagtgattgacctgcctcgtcctcccaaagtggtgggattataggtgtgagccaccacaccctgctgtgttgtttttgtttttgttttttgtttttgtttttgttttttgatatggagtctcacacttgttgcccaggctggagtgcagtggcgtgatctcggctcactgcaacctctgcctcctgggttcaagcgattctcctacctcagcctcccgagtagctgggattacgggaacgcaccaccacacctggctaatttttgtatttttagttgagatggggtttcaccacgttggccaagctagtctcgaactgctgacctcagatgatccgcctgcctcggcctcccaaagtgctgggattacaggcgtgagctactgcgcacgcctggcctggatactttttaaacatttttaaattaaaaatttttttttggagacagggtcttgttgtcacccaggctggagtgcagatgcgtgatctcagttcactgcaacctctgcctcctgggctcaagcgatccttccatctcagcctcccaagtagctgggactacaagtgcatgccaccacgcctggctaatttttgtattttttgtagagacagggttttgccatgttgcccaggctggtcttgaactcctgaactcaagagatccacctacctcagcctcccaaagtgctgggattataggtgtgacccactgcacccagcttttgtttttaatatttgtaagaaccaccttgtgtctgccaacaggcgaattaacaaaatgtggtatataaaccaatgaaaacataaaaggagtgaaataaagacgtaaaaggagtgaaatttcaaaataaaagtaaaaagatttcaaaataattcagacataaaaggagtgaaattctgatacatggatggagctggagaatattatgcttagtgaaataagccagatacaaccttttgtacaaaaatacaaaattgtatgatctcatttatatgaggtagttagaagaggcaactctatggagacagaaagtagaatagaggttaccagggctgtgaggggagaggtgaatggggagtttaatgaatacagagtttctgtttggaatggtgaaaaaattctggagatggataatagtgatggttgaacaatattttgaatatatttaatgccacagaattgtacacttaaaaatggttaaaatggtaaattttacacgatatgtatctgtatctatatatatctctctctatatctatatatcttaccagaatacaaaatttaataacacactccgaaaacctttacagatgaggaaactgaagaaaactgtctacaggggaggagttaagaatttgcccaggattattcagctgggaatttgcattcgggatccaaacttagttctgtttcactacatattatctactccatattatctgttctgtgttatctgctggctttctgggtgattaaagatatgtcagctccgagaagaatgagtttatttgaatcattcagaaagttacatttaaaagtaggtaattgtagtttgatggaaggtacagtgtgaaaccctagacagactaaaggttaactttgaggatttctttctcagccagagtggtaatagtatgcatttgagaggggaggagagtagagttctaaggatgtggtctttggagacagtttcttgggttccagtccctgagctaccaatttgtgtctggggtgttatcctcttgatgtcttagcatccctatctgtaaattggtgaggataatgataacatctgataaggtggttgtgaggattaaaggaattgatacatgtgaaatccttagaactgtacctggcaaaaagtgtttgataaatgattttcagttattgtgccaatattattttagagttgatgtactttctcattaatggaaccaaacacttctcaagttaaaattacgtgcttaggactggtaagttacaaaaatggtaccacacgttttatctatttcaatttagaaatgtctgttgattaaatgtgttcgctttaaactactgaaacaatgtagacatttataaaatgaaagcgtattgatccctgttatctcattcgctacctttaacggtttggtgtatattcttccccaaattttcaaatatattcatatatgaatatgtatttttacatacattttataaaaatgggaccaagttatttggttctaacatggcttttttttaaggtcaatacaaagatctgttttattaaaaaataattgatattcctttagggctcactatatgcttggtactcttctaagtcattattttatatagatactataatatcgagagatggagagattaagtaacaactagttagtggtagaggaaggattttaatttgggtacgttagcttcaaagtcctgatctcccagccagggatcatttttggtaaggcctgtgagctggaaacgttaacacttttaaaagagttgtaaaacaataccaccccacttccctgaagaacatataagggagactagataccgcctgccaagcctaaaatacttaccatgtggccctttacagagaaagtttgctgccccttgctctaagccatccagctgtacctctttggtgtaaggggggtgcatagtattccagtttatgaatgtgcattacgcagcaaaccaatctgttgtgattgacattgttttctctcctgaaaagaagtgaacatccttatgtatctttgaacatttgtgtgacaattttctatagagttggctctttcaagattatgaacatttctagttttaataggtgttgtcaagttatattaatttttagttaaaacaacaactgtattgaagtataatttacatacaataaaaagcacacatttgaagggtatgatttgaggagttttgacaaatgtatgcacctgcaccgctgcctggatcaagatctataatggttgccatcatctcagagtcctttcatcctcttttacagtcattctctcaacttttttttttttccctccaagatggagtcttgctctgtcacccaggctggagtgcaatggcatgatctcggctcactgcaacctccgcctcctgggttcaagcaattctcctgcctcagtctcccgagtagctgggattacaggcgtctgccaccacacccagctaatttttgtagttttaggcgagatctcagctcactgcaaccttgacctcctgggctcaatcgaacctctcacctcagcctcccaagtagctaggaccacaggcatgtaccaccatgcccagctaacatttattattaatatttttttgtagagatggggttttcctgtgtcgcccaggatggtttccaactcctgggctcaaatgattctgccttggcctcccaaagtgttgggattacaggcatgagccgcggcacctgacttgtagtaaactctctgaattaatattccattgtaggcatgtgctacagtttttaaattcatttacccatggatggacacataggactgttgtcagctgttgataaagctgctatcaccatttgtatgtctttcctggacatgttttagtggtaaatattgattttactttgtaagaaaccgttaaactcttttccaaaatagttgtaccattttaaattgaaagttacagttgtaactgtgcaggagttacagtttcttcacattttcattgacacttcgtgttgccagtcttttaaattttggccatcaaatgagtattaagtatctcattgtgggtttgtgtttctcagatgatcaatgatgttggaacatcttttcatatgcttattggccatttgtgtactttttttggttcaagccttttgtccctttaaaaaattggattgtttgtctggttgagtggcaagaggtctttatatgttctgggtacatagtcacattatctgtcagattgtgttgcaaatattttattgttcatttttgtttgattttgtgtatttttaatactataaagatcaagttaaaactttaatatgggaagcataatcagataaattatgtgaaacaaattgtccttaattcacgagtcatttaattagtgtaacaaaatgttatgcatttgcagaaacttgtaaactaaaaggatattattcatatgctgttaggtgtatggatgataattttttttaattaaactagttttgaaaattattgtatttagtaattctcttcattttgcataattcaaaccttttcatttattagtgagttaagccttaaattttttcttcaaaggataaatgagaatattaaaagtaaaaagtgaccttgatcttagaatggggtatgtagaaatgatgattgccaaacttagtttccctactttgacaatcaagtaaaattttttttttttttttttgagacggagtcttgctctgtcccccaggctggagtgcggtggcgcgatctcggctcactgcaagctctgcctcctgggttcacgctgttctcctgcctaagcctcccgtaaatttttttattatagaaatggatggcttttcagattatatatacttggtttctatacactattttatttttgtaaagtagcagttcttttgctcaacacctgaattgcccccacaataaatttttagtttttcttcaatattcaagtaatacataacttttccttttcctgtttaacaaagaaaaaaatatataaagcaagctgttggacctccattgggtgttgtttaccaccactgtaggtgatcgtggcattgtccacctcagtcttctcatggctgtggattcaagttaagaaattcctgaaggtagcattccaggtagtctgtagaacagcccaaactctctgaattagtattatctctgataggtgttttttttttctttgcttttttatttgagacggggttttgctctgtcacccagcctggatttcagtggcacaatcttggcttactgcaacctccacctcctgggctgaaacaatcctcccacatcagtctcctgagtagctgggaccacaggcacatgccaccatgcccagctaattttttatatttttgttagagacagagtttcactatgttgcccaggttggtctcaagattcctgagctcaagcgatgttcccatcttggcctcccaaagtgctgggattacaggcatgagccgctgtgcctggccctggtagttttcttcttatatctttctcctgttgatctcacttgattttcttactagtctacaggaaatttcacgtggagattctgtgcctgtcctctttctggaatatcttccttattttagcatctcaacatgctgttcagcttctggcttgaaaccacccctctcccatcccaaccaagttaagcagcttctttcctgcttagaagtaccataacatgtgtattaatctttttttttttctctctccttcccttcattttaagctcctttttattaatctgtagggcagagcttagaaaaaggtttttgtatattgtaagtactaataaaatatttgtgaagttgatttgggacttgggagacctctcttctgtaagcaactcaataaataattatttaagtaccatcctcacccagtgtactgttagctgctgttatgatattgattactgtaaaattttaaagtatatccttggtagactgttgatcaaagtcagaaagcttgagaaactctgaggcaaaaaaaaaaaaaaaaaaaaaaaatgaagagcatgaaatgattctgcaaactgatcttacaatgatctttctttttttttttttttttttgagacagagtctcactctgtcacccaggctggcgtgcagtggcaggatcttggctcactgcagtctctgcctcccaggttccagtgattctcctgcttcagcctcccaggtagctgggattacaggcacacgccactacgcccggctagtttttgtatttttagtagagatggggtttcactatgttggccagactggtcttgaactcctgacctcaggtgatctgcctgccttggccttgcaaagtgctaggattacaggcatgagccaccatgcccggcctctttttttcttttttcttttcttttttgtacacagtttcactctgttgcccaggctggagtatagtggcgtgatcatagctcactacatcctcagactcctgggttcaggggatcttcccatctcagcctcccaagtagctaggactatagacgtgagacacgatgtctggctaattttttacattttttattttttgtagagttggggtcttatatgttgcccagggtggcctgccaccttgtcctcccaaagctgggattataggcttgaatcagatcccagtgatctttatccctcagctgcatttatatgggctaaattcatacatagtatatcaagatgtcttcaggaagtttaaaaaaaaaaaggtatcaaaaagacagtatgtcatgactgctttatctattttagtctcttagtttttgatactaaagactagaccggagaaatgctttaacaaatcacaaaaatctaagaaaatagacagttaattatttttaaacagtggaagaatttcaatcataattaaagcagcttttgtattttttgatgtcatagcctatagtgaaatttcatatattctgtttgtagatacatgagttcctacgatatgcttgcacataatagaactaattactatttgttgagaacctaacctgtacctaatatacatgtagtttctaaatcaaacattttactttttttaaaaattgtaaaaagcccatagtaaattaatcatgtttaccatttaaaaatgtacagttcagtagtgttaaatacattcacattgttgcacaacagatctctagaacttttttgtcttgcagaactgaaattctgtacccattgagcactaattcccacttccccctccccccaaattcttggcagccacctttctactttctgtttccatgatttttgactgttttagatacttacatgcatgggattaaacagtatttgcccctttgtgattagcttattttgcttagcataatgtcattgaggttcttccatgttgtagcatgtgacagaatttttttcttttctgtaaaggctgtatactatttcgttgtatgtatagactgcattttaaaatccatttatctattggtggacatttgggttgcttggtgatttggtgattcactattcatggttagccaggagtaatatgcagtaaatgttggtatacaaatatctctttgagatcctgctttgaattcttttggatatatggttgcccattgaacaacacagctttgaactatgtggatccatctataagtggatttttttcaaccagccatagatagaaaatatactattcggctgggcgtggtggctgatgcctgtaatcccagcacttgggaggctggggtgggaggatcagttgaggccaggagttcgagaccagcctggacaacatagtgagacctcatctctacaaaaaataaaaaagttagctaggtgtggtggcatgcgcctgtagtttcagctacttaggaggctgagacaggaggattgcttgagcctgggaggtcgaggctgcagtgagctgttattcatgccattgcactccagccagggcaacagaacaagaccctgctcaaaaaaaaaaaaaaagaaaatacactatttgtgggatgtgaaacttgtgtatagggtgggtgggctttttgtatatgcaggttccataggtcagatttgggggtttgagtatgagtgaatttggatgtatacaggtggtcctggaaccagtcttattcttcctcttcttttttttttttttttttttttaattattcgtcttttattttttggaaccactcttctgcatatgctgtgggatgactgtatatccagaattgtgattgctggatcatatggtagttctattttaatgttttgagtaacctcagtactgttttccataatggctgcacgatttttacagtcctacccacagtgcacaaaggtttcgatgtctctgcatccttgccaacacttgttattttctgtttcttttggtagtggccaacataatgggtgtgaggtgctatctcattgtggttttgatttgcattaacaatgttgagtatccttcatgtttattcaccccaaatattttacttttgaatttattgaaaatcatgcattttaattatgtaaaactcaataccatgcatcatttgttaaatgggaaaaggagaatttgtccttatgactattataaaatattctaataaggtgaaccttttttttttttttctttgagacaaggtctcgctgtgtcatccaggctggaatgcagtggcgcagtcttggcttactgcagcctctgcctcctgggttcaagtgattctcctgcctcagcctcctgagtagttggtattacaggtatgcaccaccacacctggctaatttttgcatttttatagagtcggggtttcaccatgttggcctggctggtaataagatgaactttatacttttttttttttttttgagacagagtcccactctgtcacctaggtgcagtggcacgatgatggctcactgcagccttgacctcctgggctcaagtgatcctccacctcagcctctcaggtatctgggattacaggtatgcaccaccacccctagctaatttttgtatttttttgtagagatggggtttcgccatgttgccccggctggtctagaactcctaggatcaagtgatctcccaccttggagtccccaggtgctgggattacaggcatgagccactgcattgggccagaactttatacttattaaacagtatgctgatgataggaaaaaactgtgcaatccttattcataaaattctgatctaaaacgtgttatattaaaattaattaataatgcaagcaaaattggcaaatttatttgtaaaccccattttgggctacttcgaaaattatttatggatcagaaaaggttgggaaatacttttctagagtaattgagcaggccctgtagatggcagaagtgttgcttgaactggcgtagaggaagaaagtgttgaattgccccacttaattccttaagtaattttgcctctttggagaatatttgcctgaaatagagaactcaagttatgctatgaagattctttttgaaaaatagagacagggtctcacattgcccaggctggtctcaaactcgtgggctcaagtgaccctttcaccttggcctcccagagtgcagggattacaggcatgagccatcatgccagaccaagaagatattttggagggctacatcccaccagtagcttgatgttgtacaaagagtggtaggctctgggcgaggctctgccattatactagttgtatttccttggcacgtagcttttgtggatctctcctcttcatggtgacatgaatgagatgaataaataatggaacaacttgtgtttactgagtatctaccatgtatgtgcctgcagagaggatgtcttcattttgcagatgaagtaattagtaaataacttgcctaggttccctcaggtagtgggcccttgaagaatgggcttttatggccaagtgcattggctcacgccagtaatcccagcactttgagcggctgaggtgggaggattgcttgagcccaggagttcaagaccagcctggacaacaaagtgaggccccatttctacaaaaaattagccacatgtggtggcatttgcctgtagtcccagctgcatgggagagtgaggtgagagggtcgcttgagcccagaaggtagagactgcagtgagccgtgtttgcacccctgcactccagcctgtgtgacagagtgagactgtgtctccaaaaaaaaaaaaaaaaaaaaaaaaggctattgctgcctatagggtcctttttagtgcaaatactaggattctaagcacttggtttctgttacccaggtcaaactatttctttggagagtttgattaccatcacaggtgatggtaagattctccctagctggcatttaatccagtggttatatgcatctttcccaaattctttatcaagttggtcaattcatagaacatctaatgctgattataagcctattatcttgcttgtgtctctgcactatgggcataatagcacctaaaagcttttgctataatggactatgatgtatctgaataaattacctttataaacaatcaaatctaaattggcaagttcaaatttacccaatttttcttatagaatttacctttgcagttaaagaaataattgttttttaccatttgtctagtatgacttgttatttacaaatagcatgtaactggccagctgtactatacacattttctagaaaagaaaatttcttcaaatttcttattgggtgccttagtcttttacccattagggctattcaaataaagaatgagatattctcttattctctagaaagagacattctatactaatggaaataccaacttttaaatctagactaccaaaaaaaaagtcatgtttttctaccctagaggctatagattcttaatgtcagccactcatcctggccctctcattttcttcttttcccatatggaagttctagggtgatatagtttggctctgtggccccacccaaatcacgcgtcaaattgtaatccccaaagttggaggaggggtctggtgggaagtgattgaatcatgggggcagacttttcccttgctgttctcctgacagtgagttctcgtgacagtgagttctcgtgagatctggttgtttaaaattatgtagcacctgccactttgctctctcttccccctgcatcagccacgtataatgtgctggcttcccctttgctttccactgtgatgataagtttcctgaggcttccccagccatgcttcctgtatagcctgctgaactgtaagcaaattaaacctcttttctttataaattacccaccctcagctctttatagcaatgtgagaatggactaatacagaaaatatgtaccacagaagtggggcatcacaataaagatacctgaaaatgtggatgcagcttttggactgagtaatgggcacaggttggtacagtttggagggctcagaacaagacaggaagataagggaatgtttggaacttcctagagacttgttgaatggttttgaccaaaatgctgatagtgatatggacagagatggctaggctgatgaagtctcagatgaagatgaggaacttattggggaactggagtaaaagtcactcttgctatgtgttagcaaagagactggcagcattgtgctcctgctctagggatctgttgaactttaaacttgagatgtttagggtatctggcggaagaaatttctaaaagcagcaacgtgttcaagaagtggcctggctgcttctaaaagcctgtgctcatttgcataagcaaataaatgacctgaaactggaacttaaatttaaaagggaagcagaacataaaagtttagaaaatttgcagcctgccatgtggtagaaaagaaaatctcactttctggggagaaattcaagctggctgcagaaatttacataagaggacgatacatggggtaaaatgctttttcctcccctaaaagccttggtattaatctggaaatagcaggaaggtaactgtaggaattatataatgggtttccctgtgcttggtcatgtctttgtatcatttccataggtcagtggagactcagaaaaccagtttctctgggcttttgataagtaatgttatagtaatcaacagatggtaactttcggtaaaacaaattgtcttcatctgttttgtgctggtataacggaatatctgagactgggtaatttttaaaattatttatttatttatttttgagatgggagttttgctctttttgcccaggctggactacaatggtgcgatctcggcccactgcaacctccgcctcctgggttcaagtgattctcctgcctcagcctcccgagtagctgggattacagttgcctaccaccatgcctagctaattttgtatttttagtagagacagggtttcaccatgttggccaggctggtgttgaactcctgccctcaggttatctgcctgcctcggcttcccaaagtgctgggattacaggcgtgagccaccacgcctggccaagactgggtaatttataaagaatagaagttggccaggcacagtggctcacgtctgtaatcccagcactttgggaggctgaggcgggtggatcacttgaggttaggagttcgagaccagcctggccaacatggtgaaaccccatctctaccaaaaaataaaaattagccgggtgcgttgatgtgcacctgtagtctcagctacttggaaggctgaggtgggagaatcacttgaacccaggaggtggaggttgcagtgagacagaattacgccactgccctctagcccaggcaacagagtgagaccctgtctcacaaaaggaataaatatatatatatatgcatatatgtatatatatatgcatatacgtacatatatatgcatatacgtatatacgtatatgtatatgtatatatacatatatgcatatatattatatataatatatagtgtatacaatatatgtatatacgtatatatatgtatatatgtatatatatgtatatacgtatatatatgtatatacacatatatatgtatatacgtatatatatttttcacagttttgggggctgggaagtttaagatcaaggtacctgcatctcatgaaggctttcttactgtgtcctcttatagcagaagatagaaggggcaagctgggaataactccctcagtcagactcttgtataagggcattcacaaggcaggaaccgtcgtggtctaatcacctctgaaaggccccatctcttaatactgttacatggacaacacctgagtttttgagggacaccttcagaccatagcacagatactgacaagtctcatcaattattaagtaaatagtacttgaagggtttgaaattttgtagtaccccggcagtgtgtttatatttgtgataaattttaagtataggaaaatcatggaaagacttactgctcataactgccgtacctcgtttacctctatctttccctaaactactactgttcttagcaagatcatgaactgtggggccgacataagccagaggagtttctgaagagtctgcttcagtgaggggttctgttgatagccatagaataaagaaatggcactttgcagacattggtacttccttgaaagaactggctgtggcatatacagaactcaaaaattgaaattactagataatggttacttggtttagattgcctggaaattatgagaggttctgtaaatgctttgggattagtagtttgaaccacctagtccattttatgattctgtcttcagaatatagaaggaattgcatcttatttggggattaggatataaagttagtgtacaaggcaaaatagcatgtaggcaaagttacccatgcagagtttgtgggaagatgccttaccacaaaccagtaccttctttttaggttcaagaccagcccatttttcctctagaatgagaacagatcattgctcttctgttgagcacagagttgttggtttgtacttaagggcaatcctgttagaatcactcctgagtggctaaatatcttcagtctgagagacttgtccagcaggttatgggaacagcagattcatgtcacctgtctcttgctcacttaggcaaacgcttagtgtctggaatggtgggtagaacccttgtatttactaccaaaattgagctttctctgacatttttgctcaatgtatgttagatttgtgtaagttaaattagatgttggcttatgcgtggtataagatttaaaatagacttcttagcaaaaagaacacctggtatcagattgacttaccccttcattttgtttgcttttattcagtgaagtgttgaaataacaggtatctcatgtctcatctttgaattataatgaattttgtatcatagctttagtttctggtacttttttttttttttttttgagacaagtctcgctctgtcacccaggctgaagagcagtggcgcgatctcagcttaacgcatcctctgcctcctaggttcaagcgattctcctgcctcagactcccaaatagctgagatcacaggcacctgccaccatacccggctaatttttgtatttttagtagagatggggttttaccgtgttggccaggctgatctccaactcctgacctcaggtgattcgcccgccttggcctcccaaagtgctaggattacaggcgtgagccacttggccccagcctctgatacctatttttttccgtttttcctccaacagtgtttctgtaaactgagtatatactcgatgaaatgagaagatttaacttataaaatcgatttagtatagttttcaagaaaaacttaaactttttttttctttttttccttttttttttttttttgagacagtctcactgtgattgcccaggctggagtgcagtggcacaatcttggctcactgcagccttggcctcctgggctcaggtgattggttctcccacctcagcctccagagtatcttggactataggcacatgccatcacgcctggctaatttgtatttttagtagagacagggtttcaccatattgtccaggctggtctcaaactcctggactcaggcaatctgcctgccttagcctcccaaagtgctgggattacaggtgtgagccatggcccccagctgaaaactttaacctttagatgtcaattcagttagtgagtctgttttatctgttgttcaagcaaattctttaaaacacttaaaatagttcagttcttcagttggttgaaatttatatggccatttcttttctaagcagtactctcagataaacaccagaatgctggtctatggacttcagtttgaaatcaatagtttactgtcttgctggaaggaggaaagtgatcttactgttgtggaaaatatatcactgcagaatctactcacttttgctgcaacggtcaagttaatgttttctatcattcagctaagattgctaaattagtataactttcctttgcctttgaactttttagcactacagaaaccaggactacaaatatgttctttacttttttctgttattttcccctatcatgagacttcttgaaggcgaatgctgttcattatgctttctcatctgtactttcatgtacatgtaatccactgtagtgaaggcactaaaacctgttgtattttccacatttcgatagatctctcacaggatttaacacttttggaatgctcagtaaattattattgactgcttgataatacctctttgttcctgcctttccccaagtccttcaacagaagtttcttctagcactttgcaggtcagttgagtggactatttgagtccctcacagtaatgagcttgtgtgtttttgtcattcttttttctttttttgaaaaaacaatagagtcatttaaggaagcaaaaaggtccctgggtcaagatttttattttatcttttatggtggtagggtgaggtgtgagtatttagggagatggttggtaggatggcgttgtcttagggttgcaggaggatcagaagggtgactgtaactactgatgatatcttacagtggtgaaggagaagataagtgaaaacagaggcaatttgtttaaacgaattttagtagatattagtctcttaatttggcactgacatatgacagattatttcatttaaattctgtagtttgacaggtataataaagtacccttcattaaatattcttcacatatactcttgtttgcctttttctagttgcaagagtggtgatttctatgtttcattataaattttagaaatatataatttgaatatattgtctgttctaggttctctgtgagtcaagaaaaggcagggtattatgacttcggattcgtatgatgtctggatatatttaaggggagcagttctcaaagtgtggcctgggattatgggatcaaaactattttttctttctttctttcttttttttttttttttgagacagggtctcactctgtcatccaggctggagtgcagtggcgacatcttggcccactgcaacctctgcctcccaggttcaagtgattcttgtgtctcaacctcccagaaactattttcataataatactgagttgttccttgcttttttttaaaaaaacaaaacaaaacaaaaacaaacaaaaaaaccatgttggacatttgtacccacggtgcaaaagcaatgaataaacactgttggcaccttagcacttgcagttaaaaaagccagttttgtttgagaatgtccttgatgaagtactaaaaatgattaattttattaaatcttgatccttgcgtgcatgcagttgtaacattctgtgcaacaagatgggaatgtgtgtaaagtgcttctgctgcatattgaggtatgttgggttttgagcaaaagcacttacgtgattaagttgtgagctccactagccacttttttcatggaaatttcatttttacttaaaagagggtctgacaaaccatgattattcagacttaggtatttggcagactttttttaaatcaatgaagtgagactgtcacttcaaggaaaacaactgaccatatttgttggcagagagaacatttgggctttcaagcaaaaattaaaatttttggaaaacttttgtctgttactgtgagcatgacagcttcccagtacttaaagactttctgatgagattaatgggtttaacaacatgattttttgaaatatgccaacttctggaaaatctgcgtaactcagtggaccagtactttttagatgaggaatgatgatttataacattgtgcctgagttaaagattcatttaaagtgaatagactaatgaatgttaatgtggcagagtaggaaaagttcactgatacaatttcggattccatgttgcatctaatcttgtaagaaagaaattaccactgctaactgaagtatggtggttattttgaggaaaagcacctgtgagattgagttgtgaactgaactagccactttttaatagaactgttttggtgtaatatcaaagcacgagatccacaatgacctcagaaggctattcaaacactctcccttttctaagctaagtatgtttgagactggattttcttcatatacttgaaccaaaacaacatattggaacagattgaatgcagaaacagttacgagaatccgtttcctattagacagatattaaagaaattggcaaaaatgtaaagcaccacttactaattaattttttttttttttttttaaataaaagaaggctatgttgctcaggctggtctgaaactcctgggctcaagtggtcctcctgcctcagccccgcaaagtgctaggattataggcatgagcaaccacgcttgccctcactcactaattttttttaaaggaaaataagtttctttttaaaaatgtgttatttatattaggtacagttggccctccatattttcaggttcagcttctgtgcattcaacaactgcagatcaaaaatacttgtggaaaaaagccaataaaataatagaacagggcgggcgctgtggctcatgcctgtaatcccagcactttgggaggccaatccgggtggatcacctgaggtcaggagttcgagaccagcctggccaacatggtgaaaccctgtctctactaaaaatacaaaaattagctgggcatggtagtgtgtgcctgtaatcccagctactcaggaggctgagacaggagaatcgcttgaaccctggaggcggaggttgcagtgagccaagattgcgccactgcactccagcctgggtgacagagtgagactctgtctccaaaaaaaaaaaaaaagatacaaataatacagtataacaaggatttatataacattttacattgtattagatgatcataagtaatctagagccagatgatttaaagtataaaggagaatgtgcataggttatatgcaaatactgtgccattttatataagagatgagcatctgtggattttggtatccatgaagttcctggaaccaataccccctagacccccatggatactgagggcttgctataacagtttttaataaaaataaaaaacaggccaggtgtagtggctcacacctgtaatcccagcactttgggaggccaaggtgaggtgattgtttgagcccaggaattcaagaccagcctgggcaacatggcgaaaccctgtctctgctaaaaatagaaaacaatagccagacatggtagtgtgcacatgtggtcccagctacctgggaagttgagatgagaggatcacctaagcccaggagggcgaggctgtagtgagccgtgattgcgccagtgcacttcagccagggccagagtgagaccttgtctcaaaaaaataaaataaaataaaagtaaattgtaaaaaactctttagtttttcatttctattatgttaaatgttaatagctataacacatataaactaaacaggtcatgagaccaaaatgctgaagaatggtgttaatggtgttcttacttactttgaattaccgtttttatttttctatgctagctaattactatgtgaataattaacctcaatgaagttctgtattctctgttgtagcagtggctgatttagggctcagttgtcttttgacctgttcttccttctcatatgattgcatctcgatttttaattttagacttagtttttaattctaaaattaaaaatctttttttttttttcttctatgcccactgctgtcaacccagtcagggcccttatcactttattcttgaattacttcagttctccattttttttctcatgtggtgtttttcaaacttgagtgttgattggaatcccagggttaattcaactttaattttatgaatatgatttcatataagttccttatgataaaaccagagcagatttacaaattaaatatagaccatcaaattagctgtacatatttaaggatgagattttgttaaaaccaaatagtcaatgttagagcttatagaatggcatagcctcaggatcagctttataatcagtttttttaaatttggcttgataatactaacagagaatggctatttgcataaatatgttttataaaatggtgttaacttttagtctttgttcaggataatgagacctactagttaaccagaacggggccagcaagcaactcttgaatgctagttttaatgcctgatcaaaattctgtaaagctgtgttgatcactgaggcgttgcggatatactgagcctgcattaagtggatatttaatctaattatttctggaatctttaccagaaaacatgctcttacatagtttgctcctgggtttgctgttggtgaactgccgttggttggtgggatctctgtgtcccatgggctttgccacacagctggggctctgagtacttcagctcacattcctttgtttgccacccttgccttaactcacttacctgtattgttttttctcccatagcactcaacatcatcttacatgtaaacattttactttgttgttgttgttgttgttgttgttgttgtttttgagacagagtcttgctctgttgcccaggctggtgcagtggcacatcttggctcactgaaatccctgcttcctgggctcaagtgattctcatgcctcagcctccttagtagccgggactacaggtgtgtgccaccactcctggctaatttttgtatttttagtagagacggggtttcaccgtcttggccaggctggtcttgaactcctggcctcaggtcatccacctgcttcagcctcccaaagtgctgggatcacaggcatgagctactgcacctgacactttgtttttaactgtttctgtccaactagattttaagctgtataagggcacagatttttaattgttaattgctaagtcttcagtttctattgtagtctctggcttgtattagatgctcaataaatatttgttgacttaatgaatgcatattaacccttcttttctttattcaactgcaaactcaaatactcttttctgagttgtgtgctctcactgtgggtaaagaccagtttttctcttcattttaaacacaactaaactggccctgaattgtatagaatccttttgatttgttgtttgatcagtacaagttcagtcagtggcacagttacttcccttgattcagattctgtagaagcctaaaagtgcattatctcttcaagtagacatgtcatcatataggctgctatggagatctttttttttccttttaaattgtttgtgtaacagtttctgttttaaaacagtggaattaccagcctggacactatagtgagacactttctctacaaaaaataaattaaaaaaaattagctgggtgtggtggagtacacctgtagtccccagctactcgagaggctgaggcagaaagattgcctgagcccaggaatttgaggcttcagtgagttgggattgcatcactgcactccagcctggacgacagagtgagaccttgtcccttccaaaaaaattgaaaaagtagaattatatttgggagtttttttcctcaaattcctttgaagttaagatttataaaagtatgggaggccaagctgggattatcatttgggcccagatgttccaggctgcagtgagctacggtcgggccacggaactccagcctggggaacagagtgagccctgcctcaaaataataataaaaataataataatagtacaagctctcttaattactacctgttttttttttgagacagagtcttgctctgtcacccaggctggagtgcagtggcgctatctaggctcattgcaagctctgcctcccgggttcacgccattctcctgcctcagcctcccgagtagctgggactacaggtgcccgccaccacgcctggctaattttttgtatttttagtagagatggggtttcaccttgttagcaagaatggtctcgatctcctgacctcgtgatctgcctgccttggcctcccaaagtgctgggattataggcgtgagccactgcgcctggccactacctgtttttttttttaatcctatgtttttaaataattctaatcatctattcatattttttgtttctcctatttaacattttatgtaacatttttagattttaaaatttgtttctgttatcacttctaaaagtgaatgttatttttaaaaatgaatgcctttacattgggttgttatgtaacctaatttgctttaacaaataactttattttggagtaattttatttttaattatttatttatttatttatttatttgggacggagtctcgctctgtcgcccaggctggaatgcagaggtgcgatctctgctcactgcagcctccacctcctgggttcaagcaattctcctgccttagcctcccaagtagctgggattacaggtgctcgccatcatgcccagctaatttttgtatttttagtagagactgggtttcaccatattggccaggctgctcttgaactcctgacttacgtgatccgcctgcctcagcctcccaaagtgctgggattaacaagcatgagccactgcacctggcctattttggaatacttttggatttatatagccatggtatatttgttaaaactaagaaatcagtatcagtatgttgtcattaactaaactcaagactttatttggattttgccagttattccactaatgtctttctttggttggttggttggttggtttcaggatccaatccaagataccacagttcatttagttatcatgtctcctaagtttttctgatctgtggttgttctcagcatttccttatttttcatgacctcttaatcgttttaaagtgtattggtcagatgttttgtaagaatgtcctttgatttcggcttatcggatgttttcttatgattaggctgaagttatgggttgaggtctgggggaaggataccacagaggtgaaatccccatctcatttcatcacatcggggagtacataatgacagcagtacttaccattgctcatgttaatggttatggttttttttcacagaaacattactgtttccctttccagactcttctttggaagtgaatcattgaatctagctcacgctcagggggaaggaaattgaattctccctcccagaatggggacatacactcatgtatcagttggaattcttgtcaggagagttgttccttctcctccagttgtttatttaatcacttatttatatcatagactcatgaataattattttattctgttataatactgttatgatttactctgctgctcaaattattccagctttggccactgagagtttcttctgtttgcttccagtctcggacctatccgtctcctttcatctttggagcatttcccttcttctactggcttgccttgtagtttccctgtcccaaccctagaatcagctgtttctgcagaccctgcttccttgtatttattggaaggtggtatttagaaacaaaagtcttgggtgcagggtatgcttattttattgctcctggggtgtcactgcttccaggccctctgttggacagaccctggaaatgcaggtatttcagctaacctgcatgtgcatacatacctacgtttctgtatctatccgcatatattttaaaatacacgtgagttcatattgatatatctgactaattcatcaccacctcttgcttatttgtaacttctttctgtgacattgagaaacttggcacacattatctacattttatttacttatttgtttaaccctagtgtacatgtagagtattttcagaattacagactggtacccttatgagaaacaaatttaccagctagagtacagagtagattgtccaaagttctgtagattagctccccccgacctcctgctccttccatgagattatctcataaaattataatactgttagacttgctgtatagtttgcattctatcctgagatcccctgacatcctgttttgtttttatttgcatacagtttaccatttaaaaaaagtctttatattgagttgttaactttttaatataaatgttgtaattcctgatattactcatctgccatttatgtagttgatagtcactacttttttttttttttttttttttttgagatggagtcttgctctgtcgcctaggctggagtgcagtgacgtgttctcagctcactgcaacctccgcctcctaggttcaagcgattctcctgcctcagcctcctgagtagctgggattacaggcgtgcatcaccacgcccggataatttttgtatttttagtagagacggggtttcaccatgttgggcaggctggtcttgaactcttgacctcatgatccacctgcctcggtctcccaaagtgctgggattacaggcgggagccacagcgcccacctgatagccactacttttttaatgctgatatagtgcttaccatctgtcaggcactgtctatgcaccgtaaataaatttacttctcacaatgactctgaggtggaggctcttgaattagccttttttcatgaggagagtgaggcacaaagatgtgcagtaacttggccaacttcacacccagctgctaagtgggagaacttggatctgaacccagggaatgagctttggagtccaaactcttgctttttgtttaatgctgtgtataggacttagaattaatacaactaactcttctcttattagtttagtttattacttggaatttggtgtgaaatagagagtgctgctaccccaattctttgccccatgtgtgtaacaggcattattaatcatgacagcactctctctgctgagctagaacatggcctggcctaaggccaataatccttatggacacaaccctcaaagtggttgtatgagatgaactctccttgccatctctgatcttgtctgttttgaatcctgattctagtgcccagtgcatggcactgcgtgcctgcttctcctggattcctctctcatggtcttcctttttacagggacaggttttctatattccccctcaggcataggatctgtttctcttaagtaattaatttttatcacttaccaataatttaacatctatatgtctaagttctctttatattgtttttcttatccttacctacaaaatctcatttaccattgatttaaatttcagtatattgaaacaatggattgtaacattaaaaataaatgaaattgccaaatgatactaacagctgccttcaaagcatttaaacatacaaacaggcactattgaagactggagagttatcaaaattggcaattttttggcagtcttgctgtgaagtaaacatttttaaaaggaaggacattgagaatattgtaatcatattacttacatcttgttgggtttggatttgaccttacatagcctggtattcagttaagagtcaaagttgctgctattaatttagttgagaaggataaatagtacatgtagtttattgttttctttagagaaatagaaacagaaacatttatttggccagaccaaataggtggtcattcccttgatttagcaaatccttgaggtagaattttttttttttttttttgagacggagtctcactgtgtcgcccaggctggaatgcagtggcacgatctcggctcactgcaagctctgcctcccgggttcacgccattctcctgcctcagcatcccgagtagctgggactacaggtgcccgccaccacgcctggctaattttttgtatttttagtagagacggggtttcactgtgttagtctcgatctcctgacctcatgatctgcccatcttggcctcccaaagtgctgggattattggcgtgagccaccgcgcctcccggtagaatttttaaagaagcaataagggctctgacaatacctatgggacttttttcaaattattcagttattggcagctgggtaaagagggacaggaatgtctgatttaggtaaatacctctcacaagtggagtaggggcgtatctggtagagaacagtgctgattccttggacttgtgtggcctggccacctgagaggtaggtaggaaggtgtgtcttggccatgtcctggggcctctgtaaacctgagaggatggaggaagctctactttttctgacgatgctgttggtgataatgaagtaggctatgagagcccatcttccattaattcagatgttcctatacagttggccctctgtaaatgggattctacatttgtagattcagccaactgtgggtaaaaatacttgaaaagaaacaaaagtgtgtgtactgaacatgtacatacttttttcttgccattattccctaaacataatagtttaacaactatttatgtaatatttacattgtattaggtatttagtaatctagagatggttttaaagcaggggtccctagtccccaggccgtggactggtacccgtctgtggcccgttaggaactgggctgtacagcaggaggtgagctccgcctcctgtcagattggcagcagcattagattctcacggaagcgtgaaccctattgtaagctgcacacgtgagggatctaggttgcacactccttaaaagaatcttttttttttggagacggagtctcgtctcactgtgttgcctaggctggagtgcagtggcatgatcctggctcacctcaacctctgcctcccaggttcaagtgattctcctgcctcagcttcctgagtagctgggattacaggcacctgcctccacacccagctaatttttgtatttttagtagaaacagggttttaccatgttggccagactggtctcgaattcctgacctcaagtgatctgcctgccttggcctcccaaagtgctgggatatgagccaccatgcctggccatccttatgagaatctaactaatgcctgatgatctgaggtggaacagtttcatcctgaaaccatgtaccctctcccctctttgtggaaaaactgtcttccacaaaaccagtccctggtgcaaaaaagttggagactgctggtttaaagtatatgggagaatgtgtatacgttatgtaatagtgtaccattttatatcagggacttgagtcttcgtggattttggtatctgttgggggtcctggaactaatcctccgtggatatggaggaacaactgtatatatggtctcagtaataaatacgtatctgagataccctctgtattaagaaaaaaaagatgggaatttcttggctcatataactgaatagttccgaaatttgggaaggtctgcctgacttagggcttaggtcatgagactacagttctttcttggctttgacttttgttttttgtagttgttgactctgttctttgaggctcttctccaagatggcttccaacaagtccttgggcaaacatttttccaggtttagacctaaggggagaagagtgagcattcttgtcctagcattgcaggaaacctgagattttcactgtgactgcactgattgtggccgtgtatctaaccctgtactggtcactatgtctagggagatgtaccttggtcaggccagggtcacaagcagcttgagtcagcttcctccagtgtacttggtagttctcagagaagagttggtgtgaagttcccaagagaagggtgaattgaccctgggtggcaaaaacaacatagcatctactctagtgggataaagtaagatggtaaggcatggtgttcagggtatttctgagtttggtttgattcacaaaatagattgtgaacactgtaattgcccttaattttagttaacctatgtaaatgtaaatctgtgaggtgtcactacattctgctaaagctttgatgggctcctgccatattctggtttggcttttgtattaaagagccattgttgttttggtttcactgtatacaggtagacttttgttttaatgaatagaggtataatctgttttgaacaggaaaagctgcttttgtattctggataaagttcatactagtgggatttcacctatgaaactaaacaaatctttgaaaaatttaaaaaccatatgtatttacacagtgtccattcagtagaagtcgtttaaataaaggcttctgccaatatatgaatatttagagctgtattttattattttactttttttgagacagggtctcgctctgtcacctaggctggagtgcagtggtgtgaagacagttcactgcagtctccacctcacaggctcaagagatcctcccacctcagcttcctgagtagttgggaccacaggtgtgcgccactacacctggcttgcttgcttgcttatttattgatttgagatgggagtctcactatattgcctaggctggtcttgaactcctgggctcaatcctccaaccttggcctcccaaaatgctgggtttacaggcttgaaccactgtacgtggccttgaatctgtgttttaatactatgcttacttggctgtggtgttgtgaaaagatcactgaaaatggagtcagaggcctgatttgagccagtcgtttgttgtgggggaaggaggtcaggggagctaacatctaaaggctcactatatgccaggcacagaaccaagtgtgtttgcatgtatatttcgtttttgttgccagactttgaggtaggttttatggataaggtctttaaggcaatatcagcttccttttaaaaaagaaattccggaaactgagttttaggctgaagatctctaactggtagtagggacaactgaaccacagggtcctaactgaccctgcgatttatctccttttgcggggggtttcttgataatagggtgcactttacctcattttttggctcaagcatggataggccacccttccttttcatacctatagctaagctttacaaatgatatgctgataagatacaagctactcgttattcatgtgggttaatagacctgtttgtttgcttgtttttaagtctatagccgccccacccccaatctacaatttcaccttctaaggttttagttactcattcaaactgcagtctgaaaatgttacgatattttgagagagagaagactctagctacgtaacttttgtaacaatatattgttataattgttcattttaccattagttattgctgtcagtctcttactgtgccttatttataaattaaacttcatgggtatgtacgtataggaaaaaacatggtatatttagagtttagtactatctgcagctttaggcatccgctgtggggttggcggggggcggtcttgggagataagtggggactactgtacaattatcaggcacacacaggctctgggattttacaaatgagtaaaagtggttcttgctgttgaagcacttacagtgggaatagagtgaaatacatgaaaatgtgattttaatatgttataaatgctatgatggtgggagtttgttttgtgtaaaacatccttttaattggtactttaaattttaatattctttcacaggtctacctatttagtcttacactttcaaagaactacctggatgctgtagattttcatgatatactttattaggtatgttattaatggtagaaacagcatggaaagtcttccagaatattagacaaggacagttctagtactaaaacataaaatgctaactaatgtcttcatcaagacataaaatatgtatcttaaaaaataaattgtaagccaggcgcagtggctcacacctgtaatcccagcactttgggaggctgaggcgggtggatcacaaggtcaggagattgagaccatcctggctaacacggtgaaaccctgtctctactaaaaatacaaaaaattagcctggcgtggtggcgggcaccggtagtcccacctacttgggaggctgaggcaggagaatggcgtgaacccgggaggcagagcttgcagtgagcggagattgcaccaccgcactccagcttgggggacagagtgagactccatctcaaaaaaaaagaaattgtaataacacccacattatacatcagtgaaaactaaacacgttactaccctaggccttattgcacaggggtgctacctccaaggagaaatttgtctaggcagcagatggactagaggtgattagcctatgagcgaatgaggctacagatcattcctttttatctgattccttttctttctagttcctaggccttggaagcactaagtggtcttaagtaatttgcatagaattagttgagttcatctgttaactaactagcagataggaagaaaactattgtcatgaaattatttaaaaaataataatgctccagtttcttctcatctttgatgtcctttggtcctacctcactgccttcctaacaccattttctgctttacctcaaagctggggtcatcttgagtttagcctgcttaatccgagtgactgtcagctttattccactttagcaactcgcaggcaaggccacacttggaaacttttcacttggaatagttctatcttggtgatttcatcagccttctttatgtcaaatacactcaaattcctgcccattcttatctcttcgttcccttcaggtccttagtccttttaatttgtgactttcattctccaggtccattcttattaaatgtctgccagccagactttagttgccccctgtccagctttctcttgctcagacctaagatttctttaggttctttctttgccttttgaaatccagctcagcttttaagattgagttccttgttacctttcctgccatccttctgcagttcctaatattcttttctttctcccaaagtgcttttgtataaacagtcagccttccatatccgtgagttccaaatccatggatcctgaattcatggatttaaccagctgcagataaaaaatattcagaaaaaaaaagatggttgcatctgtactgaacatgtctgtactgttttgcttgtcattattttctaaacaatacagtataacaactatttacatggcatttacattgtattagggattataagtaatctagaggtgatttaaagtatatgggagtctcttatatcccaggaagccaggtaaaaaaaaaaagtatatgggaggctatgcataggtgatatgcaaatattacaccactttatatcacggacttttgagcatctgtggattttggtatccgaggggtgtcctggaaccagttccccagggatactgaagtatgtctgtctatctcatactatatttttccctttgtcttaggtagactatcagctccataggggcaaggatttaataatatttgtatattcattttattcaagttcgtatacactgcttgggtcataatatttattacatgcttgagaaaatgaatttcttcgcccctttgttacagctctgagtaaacagccatctgccttctctgtcatctgttggtggttgagtatttctgtagaaagttacccattggcctcaggactcttactctaaatcttcttcttaggcagttttctctgtgcatgaagtttttatgtaaacaaatagatgaagcctgccctactcatttatttgctcaagccagaaagtcaccttcttcttcactttccatatttaaatcatcatttggtggaattttggcctaagcaactcttgaattcacgtacttttccctgtcatcgccagtgtggtgtagaagcctctgtcaccccttgtcgggatgctgtgctgcagcatcatctaacctggttgcagttattctttcactccctcaccgcacacccttttacttaaaacactaaaagtggcttctcattgttcttaagataaagcacaaattgttagtgtggcctgtaaagctttgcatagcctgacagagaatgtcctgctaataatttgaaggtacaggatgattttaatactttaggagaaaatgttctaggaaaagacgcttgtttagacttaaggtgaggactctgcagtatgaattagacatctggtgaactataagctgtccccgcatttaaacataattggttctgagagcctgcaactaaagataaggcagaagaatttactttgcatttcctgcattcctcttttcgcttgatagcagaaacccctcatgttaataaaggtggcacaagaggcaaaaatacagactttatcacagtgtttaaggagaggtgcatgattaagtgtgtggggagagagtacctttgtacattttattatatggtgaactgtatgttttctacttttagtactgtttgtaaattttacttcttcttggatttacctttttcagttatattattccattatgccttgctactgtaacagctaatgatgaaaaacaggatctgtctttatattttcttccctccacaaatgtggatctcatagagttgaaaactaggttgtgatatagtatagtatacctaattcctgtaatgggatcatgttcctataatatggccgcaatttagtgtagaatttttgtaaataaaagtgtattttaagtttaacttaaactttcaatgaagtgttttaaggatttaaccatgcagcacaaatgagcacctttctgtaaatgccaacagtgtaatatgtgtcatttcttcactgattgttagtttgctgcggattaaaacacaggtgatcatattcaggctggttagattagtgattttaatatgaaaccattgcttttagaataatcatgggccagactgggaagaaatctgagaagggaccagtttgttggcggaagcgtgtaaaatcagagtacatgcgactgagacagctcaagaggttcagacgagctgatgaagtaaaggtataattttatcttttgtgaaaatgaatatacaattaaggataggttttttcccctcctaagttcaataagtttttatttaaaataattctagccaagtttgtatttacaataagatgagtgaatacttggaagcttaggttcttgatctgtcttaacattataccatgcacattaatcagtctttgtttagtatacttttatttgcatatgcagctgcagaaatgcctccctaaatataaagggatgcatttcatgattttatgtttctaaagtcattgtgattaaaaaaaaaaatacacacacacacacacacacacacacacacacacacacacacacagtcaacaaagaaaggatggcaatcgtttcctgttctctaaccacttgcaataaaataacagatgtggtgtttttgtattatttgaatgtgggaaactatagtgtaaaatctatatgcacctaagaaaatgttcttgatatttttgaggatacactagatttttaacctgctttacaggtgttatacattttctaacaatgaacaatttctcctttcctctccttcatttttttttttaagagtatgtttagttccaatcgtcagaaaattttggaaagaacggaaatcttaaaccaagaatggaaacagcgaaggatacagcctgtgcacatcctgacttctgtgagctcattgcgcgggactagggaggttggttaacataacatgttgtagtaattatcatcatcttttggtttatgctattggaacatttgtttggttattgggaggaaatcattgacctcagggtgtccatctttttattgctatttctttttaatgtgcaactgttaatgactgctgtagatcacactattagctattcctataacagagttctaagtgtttggaatatactttgaactaggtagatgcctagattttataagaatgttgatttaaaaaaaaaagcttttaaatcaaatactctttaatgtcctcatcagatcactatttagagcaaggttttctgactcactgttgtaaccccaatttgaattagccatgtggtaagaaatgttttaatattcatggacaagaaagctttttattatatattttgttcaaaggctaagcctctctataagaatttttaaatttcttgttgagagattggaatttctttttctatagtactttctgaacgagctagctcttaattactccaagagtttcctattttcaaagaaaggcagtaaatctatggtagaatctataattaaatatcccgttaaaacatgattgcagcaagatgagaagaaaatggcaaaagtctaaatagcttaggcatttctgtttttaagtgcctgtgttgtatagaaagacatggcataggatatttaaaggggagttgaagcaaaaggggaaagagcgagttggaattaaaattgcactatgtagtagattgtttatctgaatgtctaaatatgggaagcacctaaacaatggaatttgttttaagataatttttcaacgagtagccatttttaatataacattattgtaacaagcaacaatttgctattgtatgaggccttctggagattgtgtgacatatgagcttcaaagacttgtcagtgatatcccagtagcaacagacataatcctaatgcagattttagtgatattctggtggtcttgatggtgacacttggaatgattcttagtcatttatgcgttttgttaggtgacacctctgtaaagctctgacttataattgtataaatattgattatacagtgacactttcctcctcatctggtacaggccagagcctagatgttagaggtcagtggcatgggggaaagggattctttttgatgcccatcagaacgttcttaaagggaataaggatggggcttttaagtaattgctttgaaagtttttatttggtgaccttttagaaatgttgaattgaaagaaaaacctcattttctctctggaggaataaatcttagaaatttggagaaattaggtagattcacatggaaaacttatttttgccacccattatgaatctcagaatgctatcaaaagcctaacttaggtttataataaaagtcaacagcaataatgaattgtgtcactgtgctaggtactttgtacacattacctcttttaatcttcatgataaggaaggagtattacacttttttttttttaatccaggcaaggaaactgagccttagaagcatttagtcagtttgcctgctctaatgatctgatgtgggcccagaagagcacactttgctcagacacatgaagtattagtgaagtacattattgacatacaacttgaattgctcatagtatatttggcattaaatttaagagtattgtttgttcaagtggatatttactaattgaaattttcttttaattttgtcaaatggagacaaatcgttgaatataaagatacctaatgttcgtgagtttatgtgaaaaattgggagaagaaatcagcattttaataaaacaaaattgttttctaaagttagtacttccacagtaccacacacacacgcagattgagaataaacattttcacagagtatgggggaaaaggaggaaaataatccccagccttaggaatccagtttttgttgcatagaactctgacctgattaaggtcagtcattttttttgttgctggtgttggtagctgatcctgcttcagatgatccatctattatttcccctttccgcctttttaaaccatcaccactgacttttagaagccagagtttatatcgtttgtgataagcttcttggtgttatcacaataaatcgtaaagctttagggctaaaaaggactgtagtggtctcatggtccaacctcctgacttttcagatgagaatatatccctgtttattttttctttcgtttaagcatttccatctacatattatttcttttatatatttgaaagattctagtctgaagtaggtcaggtagggaggagaaaataaaagtgattcttgtttggcaaagactttcctagtggtaggcttggcagaatttcagactgcagtattgattcaaacacgtaaagtctatagcattcaacatttgaagtgccattatagcattgttttaattaactgcttgcttttataagctaattatatgtaagtgctggtgtcttcaatttaaatcctctgcagcatgacaaatcaatgacaatttttagttcttttgtcaggtcttaaacttatactcccatttggccgggcgcggtggctcacgcctgtaatcccagcactttgggaggccgaggcgggcggatcacgaggtcaggagatcgagaccatcctggctaacaaggtgaaaccccgtctctactaaaaatacaaaaaattagccgggcgtggtagcgggcgcctgtagtcccagctactggggaggctgaggcaggagaatggcgtgaacctgggaggcggagcttgcagtgagccgagatcgcgccactgcactccagcctgggcgacagagcgagacgctgtctcaaaaaaaaaaaaaaaaaaaaaaaaaaaaaacttatactcccatttgtagttgtagaagtggatatactcagtttgaaaatgactgattttctccaagttattttggtaaagaatgtcggacaaaagtttactgaagtgagtgctttagatgctgacttcaggtcagggagctctgcatttgaacccaaagaccacatctccagtatgtggttttcttactgtgtgtggttttgttttgtttttgtgacagggtcttgctctgttgcctaggctggagtgcagtggtacaatcacagcagccttgacctcccaggctcaggcgatcctctcacctcagcctcctgagtagctaggactacagtcacacgccaccacatcgggctaattttttttttttttttttttgagacggagtttcgttcttgttgcccaggctggagtgcaatggtgcagtctcggctcactgcaacctctgcctcccaggttcaagcgattatcctgcctcagcctcctgagtagctgggattacaggcatatgccaccacgcccggctagtttttgtatttttagtagagatggggtttcatcattttggtcaggctggtctcgaactcctgacctcaggtgatccacccaccttggcctcccaaagtgctgggattacaagtgtgagccaccgagcacatcgggctaatttttttgtattattttgtagagacagggtcataccatgttttccaggctggtctcgaactcctgggctcaagcaatccatctgccttggcctcccaaagtactgggattacaggtgtgagtcaccatacccgtcccttaatgtgtttttaagatactttagggcaactcttttttttttgtttttgagattatagaacctaagcattattgttcatagaattttaatacagaatgtgtttacttttaaaaaacacttctgactttttgaagacagtgactatatctgttttacgtttatttcttttattcttaagttaatcttttaaagaagcatccagcttcacactgtcattaatcaatacatataggtcaagggatgtacattttattcccataatatgtcactgttagtcaagaaattgttactaggtgactttcataactttgtcttgtcaaccagtaataactatgttctgtaggtcttcatattggtagttatattcttcattgtgtctctttttccatttttaatgaagaaactggtttaagagttactcttgaggttctcattaatgtatttctttttcgtacatctgcccatttcttgctgataatcacaagacacttctccttcactgacttcatatcaaaatgttttctactgtatgtgttatgactgaaagagaagtttgcttagatagtgcctgtcttctgggcaaatggtatgattagtttagtaatttaatccatcttgccaatatacatttctagattcccaggagaatataatgttcttgtaagagttttgtatcttgtttactaatgaaaagagagaatatggtgtatattcttggggcttcagaatgtctaatagaagcagcatcagaaggggatcacggtcctcgtatgaagaatttaaaaacttttggacttttaaatatttacagatttgctcttcatagtaatatttgaaatgattttaagttcattgcttagcatttgatattagcagatttttttggggggagaagcttctatcaaaataatgtgtaaattattttgtatgttggcagtttggacttaacatttttgttgaaggagaagaaaaatacctgttacatgtaaacactcgattgcattttttattgatttagtaatgaagttccttcagttaattgtaaatgaaaatttagtcactgtttacaaagaaaaaataatatcctagtgtatttcatgtataatttagtgtactttactcagatacattctttgagtgtagtaacatggtttctttatacattgcaggattataggccctataggtttagattctcatgaaccttttgagcaaatatggcagaaagctggtcacttttctcatgtctttaaattaagtggatggctcttcaagtgtctgtcttactcttagtctgaacatatacactcattcttattgcctcaaggaagtggtctctgagttgctttctgtagctccttgcctcttttaactattattggtatttacttggagagttgagtcttgttaggttaactgtcagtcattcccacctgtataacttgtaaatccatgcagcctttgctttctttctggctttatttaggctgaaatagatgtaacatttcttaaatgatcatataaggattttttttttcttgtgagcagaaactaatataagcattttatgcttttccaaagggttagcttaaaaaaaaaaggtcatttgtgatatttgagttcatatagcatcatatattaaatacatgtcatttattatgtttatattttaaatttgattatatatcttcagtttaaaaggtgaattttaaaaagtcaaatttaggcaactaataaaactagcagaagttgcttttcatttgtagtttttctcctagaatattgtatatatagtttgtgttttataaattattgttataaagatcatgtatcttccccaaatgcaaaaccgagttctgacaactatttaaaaataaaacaacaaatccaaacaaacaaaagtgcttggttaatctggaatgttagaaattgttttattctctgaagaaacaagaaaattgcattagaccgaggacttttgccataatttagctttataatagggattgacttaaatttactccttaggattctgtcttaccttctcttagcagattttataaaagaatatcacttgagtaacattttggaaaggctgggcagatttcataagctacccaacaaggcagttttatactagctaatgtgactacctgttgggctaatgaaactagtttaaaagttttatctttttctctccactgaacagatcattgtaaaatgattattgtaaaaatttagataacacagaaataggtaaaacagaaagcaaaagtccctgtagtccaactgctctggcttttattctttcagatttcattttctatatatactaggaggttaataatattttatttttaaaaattattggccagatatacatcctttccattttgggccatcactttacaacctctgtccaaattttcattcttttaaaaaatatgtagtgtttgacattatggatataccataatttattgccccattcttgatggacatttagatttttaatttttacttttgcaatcagtgctgtactgaactttatatacatccttgcatcttttgagtaattctgtagaagttcctacaaatagaattattagatcaagatgatatgcatgtttaaaaatttagcagatattaccaaatgcaccctcaaaagttttacattacacccccaccagctgtttatggaatgtctgtttacctatattctcaacacaatattatcaatcttaatcttcagtccaataggtaaaacattgttagaattggttataccgtgtcttaattattcagtgaagttaaatgtcagttactgtttgtttatatttgtgaattggctgttacagtgttggatcttgtgcaattaggttgggcttttcttatgattcataaggactttttgtatattacaaatactagttttggctgggtgcagtggctcacgcctgtaatcccagcactttgggaggccaaggtgggcagaccttttgagatcaggaattctagaccaccctgaccaacatggtgaaagcccatctttactaaaaatacaaaaaaattagctgggcatggtggtgtgcgcctgtagtcccagctacttgggaggctgaggcaggagaatgccttgaacccaggaggcagaggtttcagtgagccgagatcacaagactcaatctcaaaaaaacaaaaaacaaaaaaaaatagttttttttgtcatatgttttgcagattgccccccccccattttgatgttatttctgatatatcttggttatacagttttttcttttaatttttgtctttcttaaaggttccaaaagtctttcttcacttcaagattataaattatctacctatctttcctttagttctctgttttcatttttatattctaatatcatttaacccatcaggaatttcctttggtgtaaagaatgggtaggggatttggtttttaattttgttctttttagtttcctggttgtttcaacactatttactgagtaattaatgaatagtgaggggcagttgtaaacttgtttttgaagatcagcagaatcagccttcttggttggtggtttcctgggcagttctaccactcgagcaagacctgaagtgtttcccttcagaaaccacagcctgtgccatcttgctcactcactttccttccttccttccttccttccttccttccttccttccttccttccttccttatttgacagagttttgctctattacccatgctggagtgcagtggcgtcatcttgtctcacctcaacctctgcctcctgggttcaagctattcttgtgccttggcctccagagcagctgggactacaggcatgcaccaccacacctggctaatttttgtattttagtaaagacagggtttcaccatgttggctaggctggtcttgaactcctgacctcaagtgatccgcccaccttggcctcccaaagtgctgggattacaggcctgagccactgcgcctggtcttgcccactcacttgaacctctactactttgtagtattaaacccagatccactgatttaagcaaggcttcaaaacaattttactgccacagcctatataaactatagatttatataatactctctgttccattttagttgatttttcccccaatgacagaaattgttcttttttctactcacaggactgttattgaattagaaatttgaatttacatgtggtggatgaacatcactcttgttacctgcttcatgtacttttttgagtgcttactacctgtgatgattattctgtttctgatctccagtacttagcaaaactggagtttttccgcaaagctgcagatgagggaatgatttaccttaacagcccttcaggattttaacctaggattaagtaggtttgcatgtttgtgattcctttcattcagggcccggcttctgaaggtaggatgcggggaagagctttaaggatctaaatttcctcccagcctgagcaacatagtgagatcccatctcaataaaaaataaaaaaattagccagatgtggtgacacgtacttgtagtcccagctactcgggaggctgaggtgagataatcatttgagcccaggagtttgaggctgcagtgagccatgattgcaccactgcactctaaccttggcaacaagagctagaccctgtctcaaaaaaaaaaaaaaaaaaaaaaggagtctaaacttcccgatactgtattccaaatgtcctatgttaaatgctgtgttatcattccccgccccttggaatgtagaaaccgttaattacaggataaaagtgaactgagaagcaaatacccttgtgttgtagggtagaggttcttcacccaatgtggaattggtctgtaacaaatactgaacaacacaaactgaaataatttacaatctgatatgaattactctttttcatttcttaactccaacacttgattagttgctttcattaaatacatctgtgtcaatatcttaatggggttttttcttccgtttttgtttctgcctctctcctgttggagcgactcatttcttttcaagtgtcgtcttcaatctcccttctcctattttgccttctcaacctgattatgaattcacttccattattgagcttctctttactttatccaaatttgctatttatctcattgcagtttttcttcagtacttactttgcataaaagtagcctgaacttaaattatatacacggtcattttcaccagaagggctagctaatttaaaacttgggcgtgttatcttttctttcctcaagtcctgcagaatatagggtagtttcagaaatcacccccagcttttctctagacttggtgacagctgatgctttcttctgactcttcctttcctggagtgccgaccttcctgcaccttcctcgctgttgtcacatggaaaggctacaggatgttagacaatgtcaacagtttactatgtctctggcatgaaataaatggaccaaccagtatctgagaatattttttacttggctaattgattgtatgttttcccctactatatggaagttggacttagcctttctcatgttaaagattattcagaggcaggacggccatggctgttgtctttgggatgtccactgtaaaatggggagtcatacctacctcacagggtagttgtgaaatttaaagaagctaactttggtaaagtctctagcacagtgcttggcacatagtagggtgtgttaatttctttcccttccctgcaggccattcacctgctcttccctctacgattttccataacacagtttttacttggaaccagccttctgccaagagtctcagtttggttgtgtactcctacaactactatttttggcttgacttccctctccagctcccagtggtccagccacacagtcgcctattgtaccagctggttgatgtaagtctatccatcctcagaggaacctggttacttaccagtaagctgtgtttctctgaaaatgtagcatccttggtaatgattctagtcccccccatcccctttttttttaaaccattgatgatgaatcatctctaacttccttgtgtgatcctcaagttggaactggaattagagaatctaatttgttgataaaggtctcctagtaagttcctgagtccaggacacatggctctgaccaggtgaaatgtggagacccatgaggtaaaaattcttttcactactctgagtgacatgagacttaattatgtaaactgtcaaggatgatacattttcaaagaaaaataagttgctggtaagtaactgggatttttgagtaatttttctgtaagtcaataaataagaatcccacgtttgttttcaccaacaaatgcttttgcatttgaatatttcataaatgaaaggaatatggttgttagttacggttaatagtaaagaatgggataggtttctagatactagaaatacagtgaggtgggggtagaccatcagttttctaaataggtgctctcttagcttgaaacagaaattgaagtctccattagagaaaatcatcaagtgtagtctgttcattccagaaacattttttgagtgcctactgtttttttgagacactgtgctgagtatcgtttcaagacagaggtgaagccacacttggctcagctgcttttgccctcagagagcttatttcttgtctcttagtgactctcgcatgactcagaactgaggcgatgggatactggggtatgacagaggtatgggactggaagtagtacgtagtgctgcagcctgtgccttaatattttctgtgcttccataagttctttctgttaatgggaaaaatcagctgacttcattcagaatttgcagagtatttgctgtatcactagagcaaccatagatgaagcaaagccaatgctccttctttgtcatagttgaaaactcattctctaacccttatgtgcgactttggagttaggtgttaaagcaggtgaataagtcagtcggagaggcagcccactccccattaagctttcaaagcacttcttccttcagaagccacagaattaatttattcctgccaaatgttaaaaaaatcggaattttttttttttaagaaatgaaaattgttttgtactttaagtcactggatagtatttgatactatcagtatcagtaattatattcagtatattcggtaattataagcacctccacccccaaatttacccagttaagtagaaagaaggaaagaagacttggtagtattaataagttgttgttggaggagatgctcctattacattatgtctctattttctttttttttttttttttgaggcggggtctcccgtcgtcacccaggctggagtgcagtggtgctgatcacaacttactgcagcctttgcctcccaggctcaagtgatcctcccacctcagcccctcaagtagctgggactacaggcacgtgctaccttgctgggctaattttttgtgtgtttttagtagagacagggtttccccatgttgcccaggctggtcttgaacttctgggctcaagcgatctgcccgtcttggcctgccaaagtgctgagattatacgcttgagccactgtgcccagccatgtctctgttttatatagtaaatactgcatggctgtaattgttattttaccccatgagcattgcaagggcaagaactacagtgcagtaattaattaagctcacgggtgttgcagtcagactttaaatccaggttttaatcctggttttgtaatttgctacctttctgactagacaaattgtataacttctctgaacctcagtgtcctcatttgtgaagttttgatgctaaaactacaccataaggttgtaatgaggaataattaagataatatatacaagcacttagcacagtgcctgcaacatagtacgtgctcaataaatgttaactgctgtttttattggattattggatggatggatgaaggattgtatcagaggccaaatagcaggaaggggaaagattgtgggctctggagtgttagtaaactgttcacaccttcttgtttactcgagttaagagccttattagtattattacctttatctttgcctttttggaaagcaaattaatagatgaatcactgtcacagttcttcctaactactccatccttaaacctttacttccactaaggaaggaattatttttgtgctgcagtgactgtgatgattgagagattatcccactggcctgatgcaacccaatgctgagggtcaggtgtggcccatatgccagatttctactctgaatttcaccacaactagttctgctatttttaagacattttaaataaaaaatttaaacatttaatatgtgtgtgtgtgtgtgtgtgtgtgtgtgtgtatatatatatatataatgatttttcagtttcattttactacacttcttttttgctctattttcattgattgtggattatgaatgtatgtctaggatatttttgagaaggctctggaggacttacacctgggcagtagtactctttaaggaatctcggattcgtagacagggtggaaagtgctgtggttatttaggcacagagttataccatatttcagagaatcaaagatgcaaaatttttcacattttaacattggaaacaacatgcatcttagaatcactgtgataagaatggattatacctttgtttagttggcagcattttttcttagtgatacagaagataatgatgtaccttgaaatcgctgtcatcatagattgtatgaaatatggtgtttcaagggactatgtgcactgacacattttagcaatatggtatcatggcaatataaggagcgggaagagcaggggctccagagtcaggcttttggagtttacattctagcccgagaaccatgcgcaactttgggcaagctacttatcctttcttttctatagttttctcacctgtaaaatgggggctaagagtagtacccatcttacagtgttgtaatgaggattaaatagaagacatgtaatgtgtttaaaatagcctagcacatagaaagtacttagtaatcagctaatattattaattcagagattacagttttgcagaatagacttaaatttactttgttgtgattgaaacacaaacccattacccttaatatttatgtatcatctgataatatcagcaattgacacttagattttaaaaaaggaagataaggaagattaactgaacaagttttcttattctttttattttattttattttattttttttttttttgagacggagtttcgctctgtcgcccaggctggagtgcagtggcgcgatctcgactcactgcaagctccgcctcccgggttcacgccattctcctgcctcagcctcccgtgtagctgggactacaggcgcgcgccaccatgcctggctaatttttgtatttttagtagagacggggtttcaccgtgttagccaggatggtctcgatctcctgacctcgtgatccgcccgtctcggcctcccaaagtgctgggattacaggcgtgagccaccgcgcccggcccaagttttcttattcttattaagtaagatttaagtggcttatttggtatacatattactattctgtgtggttggaatgtaagagaaatgaaggatgagctaggaaataaatgtataggaaaagcatggaggagttaatcagatcgctgaatctcatataagtacagtttttgattcttactgatattggtaagtatgggaagggttggaggttttgaaaatttggaaaatttgaagcatttttgcaccctaagtaaaagaaaagagagaatcctatttggtttatatgaccactttactgtcctaggtaaggacttttttttcaaaattaaatatttgggtaggcagcatctctttacaatatgatggctacagcttaaggttgtccttttattcttaagagtcaattttattttttctgttttagtgttcggtgaccagtgacttggattttccaacacaagtcatcccattaaagactctgaatgcagttgcttcagtacccataatgtattcttggtctcccctacagcagaattttatggtatgtaaataagatatcttctgcttatttgataataacttatttgaagtaaaaaagcatagataattttattgtcaaggtgaatcaagacagtataaattctaaaaagctatcaaaataacacttgaagtaatttttaaaaattgattaaggtagtttttttaaggcctgtgcagtattactcttttcctatactctccaaaggaaaagtgttaagcagtaaccatacctaggattaaaaatatttaaaagatgggctgggcattgtggctcatgcctgtaatcccagcactttgggaggctgaggcagatggattacttgagtctgggaattggagatccacctgggcaatatggcaaaacgctgtgtcctcaaaacaaaagttagctgggtgtggcgcatgtctgtagtcccaagctactcaggaggctgaggtggaaggatggcttgagggaggcggaggttgtagtgagctgagattgtgccattgtactctagcttgggtgacagccagaccctgtctgaaaaaaacaaaaggaatgaagcttatagttcttatccttaaaaaagtcagtgtcctgtaatctaagtctgactttgagtttttgttaattgagaacttattatttaaaacaactttaggtagcattttattgtgataatgtacattatggtgtaaaattcatagggcttgtgaaacatgagctgaaacatagttccattgctaactctgacgcagcagcttttgtaataaaatgctaaagagaaactgaagcatgtgttcttgacttgtattgctaaataaaaaatatatagttgtattacttaattgatatataaagtgggaaggctaataaaatgttgatagaagtccttggtgaacctgctttggggaagtttttagtaacttttatactttgtacagtagagtctcaacttcagttactcttctgtagttgtataaatttgagtgtagtttgagaattacattgcatgaccttgagggaagtttcatgttttcacagactgaaattctggaagaatatattatttaaatttgtcattaatttatgaatcacccatggattttgctggtctagcctgggtgacagagccagaccctgtctgattttgctgaccaaagcagattaggcacacaatctttattgtaactaagatacgttttggagagcaggatgatgtggatatgattgaaaagacaaaatatctaagtgtcacttgattttggcttagatagaaagaaatcaacccctagtttaaatgagattagtgcaagtgaaatgagatgaaaacaggcctctagtgtgtacataagcttgaatttgaatatatgaatacattaaaacctgcattttaattatatgctccttttatcccactttttcttttccttgtctgtctacggtacttgttatctttatgaactctccagttgtcctaaatttgttacctagggattatttgagattttttccacattccctggctgctactagtcctgtgtgtggcctttcccagcgtgttcttacctgtgggccatgatcatagtgctgggccgagcctacgccatctcaccctttaaagagcttctgtgtcctcccagtcaggcatagccagtccattctgcttattataattatctttctaaaattctgttttcaaccaatactcttttcaaaggcctcttaagacttagacttgtcttcacttggaattagtctctttaaacatgtacttcagaactgtccagtctttatctataactgctgtcatttttattcttcattgaaaatgctcttcctcctatgaatgtcaactttctttaccttgtatgcttaactattctattctgtcttaacttttgtttgattggctattcttctttcttctctgttgtcttccaaagcttgataaaaactcatctatcatctttactagcactgtcaactttgatattcatctcattcatctaaatattgaaatattttatgtttttgtagaatgttcttattttacatataagtacatatccttttggcataaactttaatggactatcaatactctgtatccctagaattttattatattggatgtcttacatttttttttcattttaccttaaactattacaagttaatggctgttttaataactggatcatcaagtggagtttgggagtcttagggtagttttttggtatgggtctttatagttgatcttgatacaccatatttcatcatcctctattaacatctaaagtgcgcgttaaaactgatgatggctgggcatggtggctcatggtgtcagacacctgttatcccagcactttgggagccaaggcgggtggatcacttgagctcaggagttcgagaccagcctgggcagcatagtgagacactcgtctcaattaaaaaacaaaaacagaaaaacagacgatgtcttctcataatctagagcatctttttctttcttagaggtatgtacaataatggtggtaagttttaatgattgatgacagactaagtgcattattttttctagaattaaattttttttctgtcaacagttttctcagatatgcttattggtgagaggggtcttttattttttgataaattttagagtttgctctctgactagtattttaaatctggagaactgggtaaagacatgtacacatgaaaaaaaggttgtattttagttatttcacatttgatacttagagtaaacctgctttttaaatatatttttttcttttaggtggaagatgaaactgttttacataacattccttatatgggagatgaagttttagatcaggatggtactttcattgaagaactaataaaaaattatgatgggaaagtacacggggatagaggtgagccatatgcttcttctcttggaaaaaggggctaaagaattgagagacacttttgtttatgaagcatatagggcatgagaaaaattttaatattaaaattaagttttgcactgaactataagggactgaacctgggcccaggcctaagattttcagtattttccctaactatttttgggaaaattagttaggaggatcatgtttgccacctgataattatgcatagttggttataacataatggcaataacatgtaaattaaatagaacttgagtgtatgtagattttaagattctttagtagtaaaattaattaggattggggctctgcttctgtggcatataggggctccaacagcccctactactaaaaaataccgctcctctagggactacagtacctgaaattcaatccttagagtcatggtttcagttttttgtgtccaggaggccctgttaaagtgtctgaaaggataatatttgtaatatcatcatccattgtcattatcagcagaaagcagggtctgaaatgatcagttaaaggacagatccattgtttgggaggaggccaaagccacaatcagttcctagaagattcagttttctactagaaagttttggggaggggccattctacagaaagtagcaggtattaaagataagtttgcttcctttgcctaacaccagtcctgaaaatgatcttatcttttcctcccctcatttcaataataatgttttagtgccagttactaggctatgcctgttttgtccaagtagaaaaaggatacataaacttttgacattattgcttctcctgtgtgtttctgacatttgatgcgtttcagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatggtatgcctgattattaggattctgtgtagccattacagctttctttcaaaaacagtagaaagagtagaaatagggcttagtttcttctttctctttttctttctttgttaaatatcacattgatagcagtataatcaaaattaacatattatggccaggtagggtggctaactctgtaatcctagcacttgggaggctaaggtgggcagattgcttgagcccaggagttcaagaccaacctgggcaacatagtgagaccccatctctactaaaaagtaaaaaaatgagacagggtgattgcttgagcccaggaggtgtaggctgcattgagctgtgattgtgccactgcacagtctggctgggtgacagagtgagaccttgccttaaaaaagaaaaaaaaaagcataatacttgcaacattatcttcacttgagtatctttgtttcctttggctagcaaagaacgtaattttttgtgtgcttcactgggcatcattataatatataacattgatattcaccgtgtacaagttgtgtactaatcacttcttatgcaccagctcacttaatcctcataatggcttcatgagatggatgttattatcagatccattttctttttcgagacggagtctcgctctgtcgcccaggctagagtacagtggccccatctcagcttactgcaagcttcgcctcctgggttcacgccattctcctgcctcagcctcccgagtagctgggactataggcacctgccaccacacccagctaattttttgtattttttagtagagacggggtttcaccgtgttagccaggatggtctctatctcctgacctcgtgatccacctgcctcggcctcccaaagtgctaggattacaggcgtgagccactgcgccccacctgtcagatccattttctatttgaggggatagatttagagaggttaagtaacttataagattacactattgggaagaaacagagtcaatagttggtgttctctacttggcaaagctcatgctaaggcagcatagaatagaataatttggcgggtgaaatagcaatttctaagaagcgtatttcaaaaaagagaaaataacaaaaaggtaccttattgaccttattgatcatctagttcagctccttcattttacagatgagtaaacagacccaagaggtaaagtgatcactctaagtcacaaaatcagttagtagcagagctgggagtagaacctaggttttctgcttcccagtgctcttaaagcttttcatatttgaatttgaattttgtttttgactgactggcattccacagacatgtaaagtatgtgagaagtaagtaaaacacaaaatagagatgatttcttagaaaatcagctttgttatagagacataattgggtagagaaaatgaaagatcaaaaacttgtttacttccattctttttttcctttcatattctcctgtttagataaagaaagccgcccacctcggaaatttccttctgataaaatttttgaagccatttcctcaatgtttccagataagggcacagcagaagaactaaaggaaaagtaagaatttgttcctttgaggcaatcttgccctgtatggtatgtaactcaccattttggtttattacaaatggaatcaatgtagcggatgagccactacactttgtttacagtgtttgcaagatacttgaagcaacatgtgtacttgtggtactctgcattacaaaatatttcctgtagatagaataaatacatatgtataatttatatatacacataaacacacacatatacataaaatttacagtagtacatttacaggaaaattatctggatactggtagaaatttgatgtttagttaaaactaatataatatccttttccatgatatatttgaatcatatcgatgaatttgattcttgataacaccatgcacaatatttagttggctctttatctaaatagagccattcctttatgttttaggcaagataaacatcaaaagtaacacatggaaaccttttagaaactgttttcaaaaaagtgatttttgtttcatgtttacttaaatttttcttggttaatgtcagatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattgtacgttttcattttctatctttgttgtggaattatgttttaaagcagcttggagagtgctatcatttattacaactagaatttatcatttaatcttgtagttactttgacaaatatctttagatctcagtattattatttttactttgttgcttgaggattgctctgaaatcttttgcatgtatttcatttggactactattttccactaacatggaattcaggtcagtgatttttttttttttcccccgtaagtgctaactaagcagttgttttacaataaaactattattatgaactggtagcccagagcaacattgctgatattgatgaataaattgtgttaaataagaaattggccaaaaaaatgttcaggtaagtgcttgtcgatttaatgtaattttattttattttattttatttttttgccctctgggtaagttgttttttaaaaatacctgcatttgaatacaagtaaaacgtaaaatgttcttagtgaaaatgtaagtatggtatatacaaaaatacttttttcctctggcatttctagggaatgtggtttcttagtaaattttcctggtaattgaaacttctaagcacagtttaaaagcgtcaactacttttgaggcacatattttgaaaatgtatttaatacttcctaattttcctagagtagtttgcttattatagtttggtttttcctatattgtattttagtacgtctgtctatggcatggtaatttctttaggggctttgacccattgtaaaatcgttttcccctgtactcaacggccccacattgctttgttgttttgagttcctgtgcctaccattgacagatttattcctcctcgttcaccaatgataacgtctttttaagcttgcccaagcagctgtacctcacctcctcctaagtgtttgctctacttgtttttgctgcagctaataatgtgttgagtgatgtaatacattggtctggaaatgttaagttttgtttttgtgtgtgacatattagtggtttagttatcttaaaatagaaaaacttgaagtagatttttagaattatcattatattgttacctccttgctcttgttctaactcctcatctagtttaaattgatttggtagttttaaaaattcgtagtaagtcatccatttgttgcttcagccattggacagttaagcccttgaaatagttgtcttgaatttcattttaatttccctttacacaccatgaatttatgaattacatcgagtttttattaagactattggacattgtatgtagatatcactatagcaaaagaattgagtggaagcactattttggagtcgctgaaaggtccttccaatttgttgtttatagatgtagcatttaataaagtttttttgctgtaaaaatgaaccatagaagtaggacactacttttaaacattacaagttttttattgtgccattcagagatgagtaatttttttttttgttaacttgaatcttgaacctgttacttacctcattctagctggggagaacagtggaatattttactttaataaagttggctttcatggggctaattggctgcctccttgtagcatttggtggccatgggtccaactgtgctaggcccacttttacttctttttacagtagagccagtgtcttcatcattcagtcctattgagttctgtaggaacttcattgttttacgttaacttcgacaaaataccacacatggtatatttttcttaggtaaaataagttactaaataaatcattaaatatatataatcatgcattacttatggacggagatgcattctgagaaatgtgttgttaggtgattttggcattatgtaaacacagagtgtacttacacaaacctagatggtacagcgcatgacacacctaggccacatatccctgcagcatgccactgtactggatactggaggcagttgtgacacattggcaagtatttgtatgtttaaatcataataaacatagaaaatatacagtaaaaatacaatattatcttatggtaccactgttgtatatgcggtccatcattgactcagatggtatgcagcacatgactgtttcttttttgttgttgttgttgtttttttttttttttgagacgaagtctcgttctgttgctcagtctgaagtgcagtggtgtgatttcagctcgctgcaacctccgcctgctggattcaaaggattctcctgtctcagcctcctgagtagctgggattacaggcgcccgccaccacgtccagctaatttttgtatttttagtagagatggggtttcaccatgttggccaggctggtcttgaactcctgacctcaagtgatccacctgccctggcctcccaaagtgttgggactacaggtgtgagctaccacgcctggtcgattttatgttgaatatagcattttgtgctttagttttctggtctcttatcttggggatacggtagtacttcctttacagagctgttgtgaggattagatgaattaatgtggggaagatacttaaaatggtacctggctcttgttagctattgttgtttttaaaataggtcatcattcacatggtttacgtttccaaaaagtattaatatgaagatgtgctgggcgcggtggctcatccttttaatgccagtgctttgggaggcagaggtgggaggactgcttgagcccaaaagtttgaggctgcagtgagctatgatcatgccactgtactccagctgggtgacagagtgagaccccatctcttaaaaaaaaaaaaaaagtatgaaaatgtgtacaataaagtctcccaatgactttctgttccccatccttcgaaattttacctcactcttccacttttattagtagttaaccacttttattggttctcgtattttttttacagtttctttgacaagtataaagatacccttttttctgaccgtcttatttacatggtggcatagcattcaggcttttccctttttttggaataaggataggatttaatattgtagctattagctttatctgtatagctgcatagcctgtgctgtaggaggttccagaacttactggactggtctctaatggtgaccatttggattattttgagccttatgctgtaacgatgccacagcaaatatctgtacccatttcatgcaagtttaagtttacctgataaactctatttaggtattgccaaatgacctgccatatggggatgcagcactttaaagccctaccagcaaagtcagagagccctgcccatagaaggtgttaccaaactaatttttaaaattgattttataagtgaaaaatggcttttagcattatttttaaattcttgtttttctttgttttttaatctctgcttcccacaggtgataagcattgttttaatatctggatttaattcctttgaatgagggtaggatcttcctatatgttgaagagtgaactatttttattctttgtttttttgattttgtgagacgggtctcgctctgtcgcccaggctggagtgcagtggtgcagtcttggctcacggcgacctctgcctcctggattcaagtgattctcctgcctcagccttctgagtagctgggattacaggcgcgcaccacctcacctggctcatatttttatttttagtagaaacagggtttcaccatgttggccaggctggtctcaaactcctgaccttaggtaatccacccacctcagcctctcacagtgctaggattactggtgtgagccactgcgcctggcctatttttattcttggtctcatagatctttttgatgtcttaggagctttttatataatcaggaaattaggttttttgaaatatgagttgcaatttttttcaagtttatttggtaaaagtcccataagcaaagtttaaacttttttttttttttgagacagtcttgctctgtcgctcaggctggagtgcaatggtgtgatcttggctcattgcagcctttgcctcctgggttcaagggattcttgtgcctcagcctctggagtagctgggactacaggcatgtgctaccacatccagctaatttttctatttttagtagagatgtggttttgccatgttggccaggccggtctcaaactcctgacctcaagtgatctgcccgcctcagcctctgaaagtgctgagattacaggtgtgagccactgcacctggtcagtttaaacattttttaaaaagtagttgaatttatcagttagctttcccattccaagattataaagcgtttctcactttttctagtatgttttaggcttcatttttaaaaacataaatctttgatccatttggaatttaatatgatgtggattataaatctgatcctcctcctcacttccaaagttgcctagttgtttcatttactgaaaagttcatctttcactaccttgagatatttccttcattgcctgttgccatatgtatttgggtttctcaacattgtgttcctttagtctattcatgaggcggtattcagagatctcctggcaattcttgtttttctatataaatttatttatttagagagagagatatatatacatacacacatacatacatacacagggtcttgctctgtcacccaggccagagtggtgcagtggcacaatcatggctcactgcaaccttgacttcctgagctcaagctcttctcctatctcagcttcctgcgtagctgggactacaagcatgcaccaccattgctggttagttttctttttctttttttgagatagggtctcactatgttgcccaggctggtcttaaactcctggcctcaagtgatgctcctactttgggctcccaaagtgctggaattacaggcgtgagccactgcacccagcctatatacatttcaaaatctgtttgtctagttctttttaaaaaaaattagtggtatgtttattggaatcccattaaatttataaattacctaatttctttttaaaatataagggttaagtctaactaaccataccatcacctctctttttgtgtcctttaattatctcccaagttttctttatgtgactcttgtacattttttgttgagtatttctaggtattaaacttgctgctgctgttgggctttttccacattttgtcttctgttttgtaccctgctaccttactgcattgtcttcatgttttatcagtttctcagtacattcttaggagttttccagctgtgtaattatactgtctgtagttattaatggttttaccttctatccagtctttataactgaagcttctttctcttttctacctactttatttatttattttttggttgggcgggggaggggagtggggaggctttctgataaaaaccagaaagcctgctagacaaattctaaaagagctgtaactgtcttttctacctactttagcttgtagtttcagtacagtttaaaaattagtagtgctggtagatagagataaatgttctgtctcttctggatgagttttagaaaattattctttacatccaggttttgagatttatttgtatgaagtttatgtgttctctaatgttcgttttatttttgttaatattcttgttctctatatatttcattgtcacttttttgttaagaagtggtttttgtattttaaattatgtatttttttcttcagagaaccagctacaatgttagtttaactcataaacagaaaaacagttgtattagggttccttaacaaacgtaatccagcaccagattagtattggtgttaggctgtctggagcctaattgctcacacgcttccaacagaatctgagccttgaatttttcttctttaaaaacacaactttgtccttttttttttttttccttccttttctcagtctttaaatatggctttttacaaatatttaaaagatgatttgttttgtggaaatgtcacttgtttttctccaaattaattttcatcttgtataattctgaatagtccatttccagcatttcccttttgtatatgaggaagtaagtttttgcaagaaattctgtgaagttagcactgctgtcaatgttcacactcttttcatgtagcagtgtacccgcatttgaatctttctgttacattattttggaattttaggtttcaaacagtgtatctttctaaaatccgtttttttgacattgtaattttctgtggcaagacactataagctctcaccaggttcttggattgggtatgattcacaggaattgaatgttagtaatctggggctgtcctctttatcattaaaaatgagtttttacaactggtgacaagctgattgctgacctttctttagaaatcaagaaattgtatgtcagaattaaaaataggaaatctgatttaattgtagagggtctatcaactgcttagacagagatagtaagattcaatttaagtaaatgtttataaccatagttatgcgcatcagttttacttgcaaggagaaaaatgagaaatcacgactcattccagaatttactgtttttccttcctgcttaatggttggaggaggaggaatggagaatacgttgtgatcattcagtaagagcctgaaggaaagttgtatgaagtaacgtaaaccacatattagcaagattataatccattaattgacttttccagtggaactggaagagtgaaaaagtaagtattttatagttatattagattctttgtttcatttattttgcagcttttcatgcaacacccaacacttataagcggaagaacacagaaacagctctagacaacaaaccttgtggaccacagtgttaccagcatttggtaagacttagtgcctaattattgcagagggtacttgagaggactttgcactttggtggaggtgatcaagtcagcgttaatgttgtctgcacccatgctgttatgggccatatgatagacagagctttctcacccatgctgtttcttccatagctcatgtactgcacattttagtccttcattaatgtggtattattctttttaaaatctgtgttatctcttcagcaacatttcaaggtctttaggatcaaggacttttatgccttccacaaagtctatgagagttgcaaggcacagatagtccgcttagaaagcaggcatgcagtaatcatacgcagtagatcccaagcccctctcatagccatatccctctacttgagttttagatgcactgtcatttcagctatgtgataggttccacagattttttttttttaaatgatggagaacaggtaaggggttaggaaaatatgaggtagaaaagttgtttttacgggtactctcttatttttaccatttcccctctttgaaccaagccactcctacctaggaactaaatgggtatatattgcctgttggattttggacagaagattcattgaatggcacctgcagaaggtatcctatttttaagcaatttggtttttgtagaatgaagtataaaactgttggctgctgtcaaacttctcccttctatttccactaaaaaaatggaattcttgagcacttgttttataatattctttacaaggcagaattttgtttggctcatctaaaatgtttctgtggtctctataaaccatgctcttaccattgttttatactttgagactggattattgtacttcataatgcaagatgtaaccctcacagtcttattcctgaagttcctttcatgtcttcctgtggctgtgtatccttcacaggaaaaaaaaaaatgttggatgttttaatagtaagacaggaagtgacaaagtcaagcatgtttgtgttttgtgttcttgtatagttgaaatattaacacgaagaggtatgcattatcttaaactctgcataggtagtctgactccgtgcacattaggtatatgattcttttgtaaagattcacttttaaatagactgtatgtccatagagataattacctttattacatgaagtatatttcatactttatcttcttatattttaacttaattataactagctaatatattttacaaattcacaatatagaactgtcttgcctgatttttgtaccattctaggaatatatagaacccatatttctctttctgaaattttcagactatcaaaagcatatcaaaacaagtatattagacatgcaaatgattatttgtgataaatggataatgtgatacattttttgactaacctggcttatatagtatttttttttctcttccatcaaaatgagttttagaactttgccctgatgttgacatttttcatttcgtaggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctggtaagacacgtctaataactgggttttactgttctgtgaaagttcgtgttgtgaggattaaggatgtataatgcattgaataattttcttagttggttagtttcagtataaagaccagagttatctcaagaatgtgtagctgtgttgtttctgtttcctgcctgcacccgtaccctagcttgtggtgtttgccctgcttggttgtttgaaagctgatgatgcactttattctcattctttgtgccattttcttttttcgatctttctcaggtagggaataatgaaatttggatcatttggtttaatattcttgacgttctgtgagtagttttgttggaaaatgtgaactacgatgggttagtgttttgccgattggatttgagttgtcctcatcttttcgcttttttcttaagcacaccctgaattgtacttcttaatattatttccaatcatttcttgaccagtgcttacatttggttttgtagaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtaagatatccgataaaaatgttgttaaaagagcttcaaacatagagataaaactgtactcgttgtccaaacaaagaaaattcttggttattactaagctcctattcatctcctaaaaataatttttttttcttcccaagagggaattgaatgaaaattattctccaggtatgttacagaatttccccaatgtagacttttaaggttagatagcagtagcttttaaaagctagtaagaattgaaatttagcatcacagttcttgaccagaatataattacagctacccctaatgtgagggtagggacagcgatgtgtgttatatccccagcatctagcagtgtcacagaagagacaaagacatttttctggtatgcatgagtataagaatggtttgcctaaataagactgtcctcatggctctgtgactgtgcctcttgtcaggtgtatgagtttagagtcaaagaatctagcatcatagctccagctcccgctgaggatgtggatactcctccaaggaaaaagaagaggaaacaccggtgagtctgtcatgaagctccttagagaagtcaacatagatagacactgcaggcaaggcccttcctaagggctgttgttggtttctacttagtctatttttaaaaaaccttaaactgccatcaaaaacagaactatggaatcttatttccactgggaattgctagtatttttctataagaatacagcagccttcagtatatttggtatgttaagagtcataaaaattacggttaccagactgcatttgggttaagaaaaacatatttagtagcttttgcagtaaaggttacgtaaacaattactttaacatcatgaaatgttctgtgtttctgatgtgagctctgggccagtttttagaagtatcttttattccaggagcttgaatttctaagttgtgtggcattgcattccgatactcctaatgggtaccctactttaagcttgtgtgaatgttattttcaccttagcgaatgttttcaggctgatttgatttgtaaatgtagaaccagaaaaattgatgagtggactatctgctctaatctccccatacctctacctcacatttttaaaaatgagaaacctaattgggtaggtaagggtgcagttagattatgcttcttaaattgtgtacaggctcatgttacattttcttactttaacaaaaataagtgtagggccaacccaatttagaatttttttaaagtcaagtttttatttttattttttaggctttaggagatgcttctgaaattttggttgaattatttctgaccacgtagttcacttgcttggtttatgatgcctgaaataagaggaagtgatcagtgtcatccacatcagaataaatcaacaggcagacacacctgctgctttgtgcagcattgggagctcaggagttctgaccaggagtcatggatgaacacaggaaatgacgtcttgccacacctggggcagcctgagccatcaagctgtttgagttgcttcaaactgatggcaaatataaactgggcagggcatgactcttggctttaacgcattcctgtattcatggcacagatagatccagttaagcagctctctgttggatttgtagcttcccgcagaaatttggtttaattttctttgtgtttttgcaggttgtgggctgcacactgcagaaagatacagctgaaaaagggttagcatctttccattcctctcattattagcttaacaatatctgctttgttttcatttgttttagaatagtaactggattagagcttgggttattctaatataattgccttctgtatgttaaaccaatttgaatggaggacgtctactgatgaatccccagtcaattctcttctgtaaagtggaggctgactggaaggtgtgagtccgagtcagttgaaggagaagtgctttgggatgggactgagatgtagccgtgctttagatggaactcataagcacttgggggtgggagagtattttttgccatcgttgtggaaaccggaatatgtgtaatgttatgcattcagtaaaggcaggccagctacactccacaggtagtagggaagaatgtactcgtgttaattgtgtatgatcgtttccatctccctggattcattggcctgcatgatgttatctttactcagacggctcctctaaccatgtttacaactatcaaccctgtgatcatccacggcagccttgtgacagttcgtgcccttgtgtgatagcacaaaatttttgtgaaaagttttgtcaatgtagttcagagtgtaagtatttgttgctttgatgcaattgcatgagaactaaataggtctttggttagcaatttaaaaaatcaaaaataagaacatgggagcactcccttgtttacttgacgaaactcatcaatttgacaagtatttattgagccccatacctggccctgtgccagtggccagtcagtcgcctgggtgttgtgtgtctgtgtgtgtgaggcaggcaaagttgtattaaaggaacctgaattttatttttttcttctttgatcatttaaattaagttgtattaaagttggcacagattgtaagaggaatctagaaggagctacccaaataatttttgggcaccagaaatcgtcagagttaggagggttttagtcttcagattaaaggaatgtagcataaacatagtagatttttataattgtttaatgagactgcacatgaagcttgggtattaagggagtgtttagaactaggggctattggaatggtcaaggaccatggatattgttccatccagaccctttattcacaccgactttaaatatatacactatttaacaaatgaaaaatggaaaaacaactcagtggtggtctttttcagtgcaaacagctataacttcgctgttaatacttccatttccttgtctatatagtagggtaataaggtggttgtgagggttgagagtcagtgagatgcccagtacagcccttgccacgtatctttctgcaagtttttgcatacggtgatgagtgaagaacctccaaacctctctctgaaggtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaaggtgagcaacaagtcacttctggaagattgtttgtagttagctatttagtgatgcaaatgtcaggattgccttagatgatttagctggggcaaaaagtaaaagtgatccttgaaaggtgcaactgatgagaaagtgttcataattttctctgggtaatgcacccttgggcagtctcttcagttttatatagagcaccatactctgatagtaggagaaaccagcaaattctgagaagtagccattcatggtatcagatttaggaataagaggtattttttctacctgtttaatagattatttgaatttgtcttttagttaatagtttatctagtatccttaatgcaaataaatggtgatttctatcaaaatatagactcattctcatcaaagagtatttatagattccttgctatgtagagtaaaagatgaaatttcccatggcaggtttttatagacttacttcctgtttgtttaataaccttgaaaaaaacccagctaggaactatatatttttagtctggggaattgtatgtaggtgagttaactctatcagccaggaagtaatgattgagcaaaagacacttcagcagcatgtatttgggaaatttcgtatcttttttttttttttagggcagatgataagttctttcttcagtcagttctttttataataatagctgacatttattaagcatgtaaaaataggcactgttgtgagtgctttagtggatttgttcattgattctcctaaccctgtgagtgaggagtaagtgttaacccccattttacgggtaaggaaacccaaagtcacaagacttgtaaaggagcaggagatcaaatcctccaggctcccattaacctgctcttatgctctgtgtgtgtgtgtgtgtgtgtgtgtgtgtgtgtatgtggggtgtagagtagacatatctttgttgttggaccatttatttataattgagaacacacttcttaccagcaagtccccttctaccagaaattctgctccaggcaaaacattgaagttctcttggttttgtttgtgtctttgaaccaaagtctgtgtttatggcagagagttgtgcagggtggtgggctttttgtttttagctttgacttttttcttttctgttagacatttagatgattttactctaggcagttatatgtccatttaggtcattggatcacagtgactctggttccgtttccgatggcatacctgctcagacctaaaaatctcaatttcccccccaaacaacttaccattgtactccttcctcactttgttcagaaagtgggcaattctattcatactgacatgtcctctttgtaagacattttttaactcttaaaaatgttaaactatgtagataccttttaccagttgggtttcatttgcatagcagtatttagctaatttagtttctgatcagaaggctatttgggaagttcacatgacaggtgagtggtaagatggtccagaataggaatagataggaagcctttgtaaaggtcacaagaaaattttggatgaaagcataatactgagccgtgatgaaaaattggcatatgttggggaacacatccctaaatacataatttatagcaatgatcatcttggcatttctgctgaaaatagactactggactgacaagatcatgataacacctcccggtagtttcttgtgttatcctgaggaggtggggtggaagaacaaatccaaagccaagctgctatgggcttcagagggaaccaacgccagtgaagaatcagggggtgtcctggggctgaaaatggaataactattaatagaatcacttggggaaagtagagggaaaggtatggagaggaagagtgatttggtggtgtccttatattaaattcaggttgaaagtgaattactcgaggaaatcaagggctgaagatacagggaagccaaaagtgcagcaggcctagagcaccttgctgaacgatggtcattgcagaggaccaacaccaccaaaaggttttctgtaagacagagattcttatctgctgtataaggaaaacataatgttcatagccattctcagcagctttcacgttgactgaagctgtgtgcccaattactgccttagaacaaacaggtctgaggatttacagtgatagcttttgttttcattctgtagtctactttgtccccagtccattttcaccctccttttttgatgatgtgattgtgttttattctctagcatctattgctggcaccatctgacgtggcaggctgggggatttttatcaaagatcctgtgcagaaaaatgaattcatctcagaatactgtggagaggtaaggcactgataacctgtattcaggtggcattgtatatactaactttactttattttagattgattttattaggtaagtctgtgggtttgattggaaatgaattgccataaactgccttttcagcctggacttctgcatgtttgtggatttgcatgcttagtaactggattgtgctgggcgcggtggccgactcctgcaatcccagcactttgggaggccgaggcaggtggattgcttgagctcaggagttggagaccagcatgggcaacatggcaagaccccattgctacaaaaaatgcaaaaattagccgggcgtggtggtgcatacttgtagtcccagctacttgggagctgaggcaggaagatcgcttgagcctggaagagcctgggaggttgaggctgcagtgagccatgtttgtgccactgcactccggcctgggcgacaaagtgagaccctgtctcagaggggaaaaaaagcaaaacaaaacaggattgcttggttctagccacatttagtttttctcgtctagggacaacagggagaacaagagctttaccatagagtggcctgcttaatccacaacctagttgataaggacctagaaacaacaacgtcattagttattcaccttttaaattgtcgacagtgttaccgaaaaatcataggcctagcttcagtttggaaccagctcacccaaattttttttttttttttttttttttttgagacagagtttcgctcttgttgcccaggccggagtgcaatggcgtgattttggctcaccgcaacctcagcctcccgggttcaagcgattctcctgcctcagtgtcccgagtagctgggattacaggcatgcgccaccgcgcccaggtgatctgcccgcctcggcctcccaaagtgctgggattacaggcgtgagccactgcagccggcttttttaaaaaactcagcaaaaagactttggttcctcactttctccaaggataattacttcaggatgtggaaatatttctgagttggggtccttagtatacagttattctttattactgcgcagaccgaatgggaaagagaacttggctgtagtgaccctttttgttgcgttttctccagaaggtccagtattcactctgtgcgcttttgtgtgttctgtcaggcttgatcacctttatccaaaagaattttctcctgtgtctttctttttagattatttctcaagatgaagctgacagaagagggaaagtgtatgataaatacatgtgcagctttctgttcaacttgaacaatggtatgtttcagagctgggaggcagtgagttcctgagttgcatgtggcagccttctgccggccgagtgttcagaaacgagggtagaggtcagtgaggtgaccagaaggaggatctccttagaggcatggggggctgcttgcttgaaaggaactggagcaaggaattcctcagggagcagatagactagatgtgcaggaaaccgcctctctggagtcccggctgtctgtgggtggcactatcccagcatctggctgtgaaggggacgttgtgctcttgctgctcacctcacctcttgtgttcaggcagctcagcacacctcctccagtcagtgcctctgagcggggaaggaggggaggagcactgcatttgcaaccatcttccagactcccctggatattctcaaaggactgagaggaaaggcagctgctttcatagctgatgttctggggtagaatgtaaggctgtgtagggatgtttctggtgggattatgtgaaaatggaggtaggcttaacatgtaagatattcagagaaactcttagttggaacaagtagaataggtttttttctatgaatgctttatagacattataatgaaaaaaactaattttaaatgtgaaaatgcctattcgtgatgtttggaagaattttgtcagatttttttttttttttgctatgttttgctcagtaatgtatgtttatagttgataattaatagcttttgagtcagataaccatcttgaattataggtgattaattttaacctggaacaatagtgtgttcttccaaatgtcatttcttactgaatgaagctgcttgatttatttgcttttgcaggcaaaccctgaagaactgtaaccagttgcatttacaaaatcataataacttggtttacttataactgaaattattcactgggctgtgcttacttttttctttttagattttgtggtggatgcaacccgcaagggtaacaaaattcgttttgcaaatcattcggtaaatccaaactgctatgcaaaaggtaggtacctttgacgtgagaattggaactcccttttcagtcctgtgatgatggacttgaatacttctgggatagttgtttgttatgtgaacaagtcagttaaccccccgtggcccgtcttcatgctcactgacaccagtgtgtctctttgcagttatgatggttaacggtgatcacaggataggtatttttgccaagagagccatccagactggcgaagagctgttttttgattacaggttggtaaagtacatttctagcatgatctctaagggctttttctactggattgtgaactctggacaaaggagggtttttagttctttgcttcttttgatgggtcactttgccatgagcattagtggggaattaggttacactttcctgttatgtatttattatccatttatatattatacaaggcatgcttatttttaaaatagagtaaaatccatgcagaaagccccatttctcaccctgctgttgacagctgggtgagtcctagaccttctcatatcatgccgcatgctgcatgctcactcgtggagcgttttccagataagtgcgatcacactgtcctatagatttttctgcaacatggttttacttgacaatatactatgcatatctttatggattagatctacttaatttattgtttgctataatacttattacacatatcattaccattttaaaaggggctgttggcaaatcgctgtctgatcaccctgtggggatgcctgggacacgtgggccaccaatggtatctgtgtggttaggcattaggctgccagttgacactgtcaagctctgtgggatacatttaagtcagccattgaatacagatttaagtgcagtcagatgactgtctagttaatgaactcctgtagccccgtgtacatcccatccagaagctgtctttgagtactgtgttcttgcatattcaggctggcctttaatggctgttgcacagcagcgagctctcagtgaggcagacagggtctgagtgaagggcccactttagcaaaggccgctgccttcactccaggctggctctgaatgaagagtgtggggtggagcttccttgggatgacccggtggcctcaccccccatggtgggcttcttgtgctcctctctgggtgtccgctgcactccagttcttgcttcacctgggggagcaactggcgggctgctctttggccctctttgctatggtcatcattttttccagaccatggttctattttgccttcccagcgtcaggcctaagcatcctgatgtggaccagccttcctgggaccacttgcttcagaggcataggggaggctggtcaggctctgggaatgcttgtagacaggtttgttcacttcagaacttggcagcgtctcaattagatggccagcaacacagacctatgcattgccttctatgaatgtgccgttatgcaggcagctgtgtaattggatgggttttttttttttttttttttttaaagacattttaatgcacccactatcttcagcaggctttgttgtgttaagtctcagcacatgttggatgggtggccatccagcggacatctccttcctgttgtttcagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttg

SEQ ID NO: 7 - DNA, 3256 bp, Artificial sequence
  synthetic sequence
gtgaggagcgatataaacgggcgcagaggccggctgcccgcccagttgttactcaggtgcgctagcctgcggagcccgtccgtgctgttctgcggcaaggcctttcccagtgtccccacgcggaaggcaactgcctgagaggcgcggcgtcgcaccgcccagagctgaggaagccggcgccagttcgcggggctccgggccgccactcagagctatgagctacggccgcccccctcccgatgtggagggtatgacctccctcaaggtggacaacctgacctaccgcacctcgcccgacacgctgaggcgcgtcttcgagaagtacgggcgcgtcggcgacgtgtacatcccgcgggaccgctacaccaaggagtcccgcggcttcgccttcgttcgctttcacgacaagcgcgacgctgaggacgctatggatgccatggacggggccgtgctggacggccgcgagctgcgggtgcaaatggcgcgctacggccgccccccggactcacaccacagccgccggggaccgccaccccgcaggtacgggggcggtggctacggacgccggagccgcaggtaaacggggctgaggggaccgcgggaggcggggcggggcgcgcgggaggcccgggcgacctcacaaaggtccgcggcgaagcacgtggtgcgggcccggacggggcgggggtgcacgccgcgtctcgcgaccctccggccaccccgcgagcttccgccgtctgcgacccgggagtggccggggtgtgggcggcgcggggcggaggaccccgcctcgcgactggggaaatggcgtctggcggcgagataatggcggcctgggcgggagcgcgcgggcggggccggccccgctgcctggaattaaccccgctgtgcttgctcgtcccgcccgcagccctaggcggcgtcgccgcagccgatcccggagtcggagtcgttccaggtctcgcagccgatctcgctacagccgctcgaagtctcggtcccgcactcgttctcgatctcggtcgacctccaagtccagatccgcacgaaggtccaagtccaagtcctcgtcggtctccagatctcgttcgcggtccaggtcccggtctcggtccaggagtcctcccccagtgtccaagagggaatccaaatccaggtcgcgatcgaagagtccccccaagtctcctgaagaggaaggagcggtgtcctcttaagaaaatggtaatgtctgggaatccgagacacataaccctaattcataaatgggatttggggtaggtctttttgagtcgtgttaatgtaagaatgactcctatcattaggagtgctgctcggaggttactcacctttgggagtaatactgaagagaggggtctgcagaaaggatgtgtatgaagcttagataataatggctgtttcgtaaactgtttgagacctattaatgaaaatgactatttcttgctgtttttatccaacgtctgcattttccccctttaaagctgcggtctcctgtttgataaaagaatattggccagtattgcagattttaactgatttggctgatcctccagggaccagtttctgtgggcgtgtattggagcaggtttgtctttaaatgttaaagatgcactatcctcttagagaaacaatcagttcaactattgttgtactgactgggacttcatattctaatggatgtggcaaaagaattgcaataagaagcagtgaacatttggaaccccaaaagaaagttacaggtattgcactgggtggggaaaggatagtgtgtctttaactcttaaattgtttggtcctattttttaaaaaggaaagggccctaagtagctcagatattaaagtagtattctcaattaccaaatgtttcatttgaaacaatttatcttaatgaaatatagaccaattctctgatctcgagttgtttttgtttggatacagcccttttttttttctttttttttcttccccttacctttcttcaccttggttatttggccaggaatacgtaaattcaaacttgtacatgctgatggtagcctttgtgaaattttcctaattgggccttttaaaaacatggctgggtggaacatttctgtaccctactggtttgaccagagccttagtaagtacgtgcctgaaactgaaaccatgtgcactttaatggaaggtaagctgaacttctttcttttcaaacctagatgtatcggcaagcagtgtaaacggaggacttggggaaaaaggaccacatagtccatcgaagaagagtccttggaacaagcaactggctattgaaaaggttattttgtaacatttgtctaactttttacttgtttaagctttgcctcagttggcaaacttcattttatgtgccattttgttgctgttattcaaatttcttgtaatttagtgaggtgaacgacttcagatttcattattggatttggatatttgaggtaaaatttcattttgttatatagtgctgactttttttgtttgaaattaaacagattggtaacctaatttgtggcctcctgacttttaaggaaaacgtgtgcagccattacacacagcctaaagctgtcaagagattgactcagcattgccttcattccttaaaattaaaaacctacaaaagttggtgtaaatttgtatatgttatttaccttcagatctaaatggtaatctgaacccaaatttgtataaagacttttcaggtgaaaagacttgattttttgaaaggattgtttatcaaacacaattctaatctcttctcttatgtatttttgtgcactaggcgcagttgtgtagcagttgagtaatgctggttagctgttaaggtggcgtgttgcagtgcagagtgcttggctgtttcctgttttctcccgattgctcctgtgtaaagatgccttgtcgtgcagaaacaaatggctgtccagtttattaaaatgcctgacaactgcacttccagtcacccgggccttgcatataaataacggagcatacagtgagcacatctagctgatgataaatacacctttttttccctcttccccctaaaaatggtaaatctgatcatatctacatgtatgaacttaacatggaaaatgttaaggaagcaaatggttgtaactttgtaagtacttataacatggtgtatctttttgcttatgaatattctgtattataaccattgtttctgtagtttaattaaaacattttcttggtgttagcttttctc

SEQ ID NO: 8 - DNA, 18907 bp, Artificial sequence
  synthetic sequence
ggcggggcctgggaggggacaaagccgggaagaggaaaagctcggacctaccctgtggtcccgggtttctgcagagtctacttcagaagcggaggcactgggagtccggtttgggattgccaggctgtggttgtgagtctgagcttgtgagcggctgtggcgccccaactcttcgccagcatatcatcccggcaggtaacctcaggctccaaggggcggccccggtccctggctgtggaggggtggctctaattccgcagaaggcaggaatggggtaaaggaaaaaagcgcagatcttgggtgtggagggagtcaaggcctgcgtgcaaagaagggaacaaatccaggtattgagaatgggaaaagaaaagggaaggaggggatgctggatgcttggatatgcagaaggtctgcttttgggcctgggagggagaaactgggcggtgaggaagttggatgcaacgacaaggggaaaatcaggtctgcacttgggttggaaaatcgaggggtggaagttgcaagaatgcatgtagaagactaagcctggagtgtaggaggaagaaaacgtgcagcagctgggggctggatgcaaagaaggctgtaagccgcagacgggcgtctgcaggcaactcaggcggcgaggaatagccttgaagccgatccctcgcgtccgccagggggcgtggaggggcggagctccagcggccttcagaggggtcgctaggccacacggggcgagagggggctgggggaaacacccccgccagttccgggtgcctggagtttaaaaggtcccagcaccagcgaggggaggggaggggaggggcgggcgtggagacctggaaggaggtaccgatccagccttgattgcagccctcttgcacaacggccataaggacgggtgcaggccaagagcaaggacatgcaaaccccaagacctgctaacgggcggcgaagcgggggcacgccctcgcacacgcagagataaattgtgctcccatgacctttatttggaaagtgcctgcgggcctaaaattggcctttgtcccaccgagtacactcagcactgtactttaaaccggataaactgggctgtctggcaggtactaaaaacagctaaattctcctgtgagtgttctgtatgttaacacttttattccttgttttgttttaggcgataaactacattcagttgagtctgcaagactgggaggaactggggtgataagaaatctattcactgtcaaggtgagaattagcaaattttcccccttcttctgtactttctttgatgtggcagtacagtgtttaactggtcttgggggcaggccgaggcactgcaagggaatgagtgaggaatatttccaacaacgaattccaacagcttccttgtttcttttaatacagtgtcttatctgcaagagatcctttttgcccagaaattattgtagttcatgaactcggcaaaaaacaatctggccaaaaacttgaggttgatgaaaataatattaaatggctcttttttttcattaaactactctgtattgttattccaggtttattttctggaattgcacaattatgttaggagataggattcagctacttcatatgagctggtgcctgctggtcttggaacagcccttccttctgaagttgggattgggttggagacaaaatctgaggggcaacaaaggtggagaagtcagtccagtggggcctcaagcccatcccatggaaatcagatccagaagaaatacaattatgttccacatggtagtaacaataaagcttcactgttttagttaaaagatggtaaaaatttcatggctaagtaatgtgaggaaaacctgaagcgtctgaagtatgcattatgagataaagaaagattactgcagagagtacagttgctttaactgagaaggaaatagtgaactaaaaagatatcagtcttcacattttaggtacaatatttgacctctattaactgtatttgccatcaagaataaattgccatgtcatgactgttgcatcatactatatagggacatggccatggaagtgtattatcaggaaccagataagttcactgcctccttttttgctttgttttaaaacaaggataggtgtgacagtggaatcaaacatgaactgaatcatatgctttcagctgtactacaaattatacttgacattacaaatgggaagtaattgactcatccacaccattctgttcatttcttttagtctagttcagttccagaaggttcccccaaaggaatctcctaaacactagtaagaagaattctagaattgcgaatttgggaagtgaaaatgggcctggggattggaaatacatccaggcttgtcaagaaacagacgcttactgtctactaatcattacatttttatttaaagaccaaattactttagctcaatttttcaagaaagactattttaggcttcttatctaataattctttaaagtcaattctcttgacttttttagtccttaagagaataaaagcaatttgcagctgtgttgttggattaaaaaagagtttcgattagagcatcattagaaggaatagataacttttagcaggctcagagcttcttctgcctctagggacaagctgggacagtttcatttcttccaaagcgtccaaccttgcactttctgagaatcagattgatttgtatatgctacccccacaccctatccttaaagtgctgtcttcctaggacaatgcacagtttgtcctatcccactcactagcagcttacccttgcctgtgaacagccagaagaagatgacagggacatgcttgggtcctgggcagtggccctactgtatagagattcttctaattgagaaatcactttgttgtatttagccccacatctccaatgtctgtgttatctctaggaaaacagttccaccaggaattcttcgtctcgcttttagacttaaagtttccctgaatatgggtcacaggaaccagctgaatctcctcatggctagttatttttattaaataacaaaaacagcctcttaaaggaggagaaatgctagcactcaccattctggacatcttgagagcccagtaaaatgatgagttggactgaagtgttcaataaacatgagattgtggaaataatctcatttcttaagacaaaccagtagttgagaataatgattgcattatgaagggcacgttatagggtcaggctctgccacgctctagctaggtggccttgggaaagctcttttttttttaacaaagtaaaatggggaaatgatggccatttttttggattgttatgagggctagctcagaaaccgcgtgtgaaacataacacaatactttgttcagagaagatactcaattctaatggtaaatagaattgctgttactagctttttattcaactacacataaattgaattgcatgaattgttcttttagtagtcactacgttgtttatataaacactacgttgtttatatatttcactttataatgaaataaatgacaaggcatgtattttttttttctttttaggtttattgaagtcaaaatgtccaaaaaaatcagtggcggttctgtggtagagatgcaaggagatgaaatgacacgaatcatttgggaattgattaaagagaaactcatttttccctacgtggaattggatctacataggtaaatgagttacccctccgtgtagcaaactcagaaaggataatctggctgggcatggtggtgcacacgtgaagtcccagctacttaggaggcagaggcaggaggatccctcaagaccaggaattcaagtccagcctgggcaacagagtgagaccccatcactttaaaaaaaaaaggaaaaaaaagggaaggagaaacaggaaaaaaaaaaagaatataatctgaattattttgaggtgaagttagctttttttatatagatatagctgtgttttgtgaataaagtttgttttaagcctaaatatattcagatgaatcttctaattttaacatactgctagaaaaactggatttatcatctaaaagtcaagaattataagggaaagaatgattttcagattcttctaaccttctgtttcctgtgaaggttttatttgtaataaaacagtggctaagaatagtacaatgtattgagaaaagggaaataagagaggatgtaagaaacagtaaactaaatgcagttaacacaattttgtcatttcaaactcaattttctatcactttacttagtcaatggacctgtttccttttcatgcctcaccttttgcccttttcatacaattgtagtgcagaaagaattcatgatgtttcttagttgaacccctcctttccagcaaaattcccttaaatcctgtgtccctttgccctgtgtgtaaagtgcctggggacagatgcaaaagtctatgtaattctctcatttttttcgctgtgtttttcataaacttgcttctgttctttgtacctttaggtagagatagaatttttaaaagggtaaagtggctgaagactggcatagaaaaatattgagcaacacgtttcagccaaactattggcaactctaagtgtgtttgcctcagcccagaattagtcatcaaaatttgtcctaaggacctgtttgagtggagacatttaattcacataatgtcattaaacatactttttaattgttctaaacctaagctgctgatgtctatattgaaatcatgataagatataaatacttgcagaagtcaaccaaacataagaaattaggcataaaaacatcttccaactaggttaaaaaaaaaaagcatcttaggaactcctgtttcaggactttacatgacagtaggtgaactggcagggttcttttccttataatggctgcaactaaagatgctttgagcctctttagtgtggaatcatgaggagttttaaaggggagtcactatgactaggaggaaaaaatttggtggctgcagactagataatttttggaggctcaaacaagttttaaatttttccaaaattcctattattatcactgactggttctttttcaggagaatggagcaggtagtttgttttatgtaggctggacagtggtctgggcaatttgttccttcctttcttatcttcttagaaatgttaagaagacaagcaaaaaatctaataatttgctgatagattctccttatgagaaatggttcctaggaacttgaaaactactgatcatacttcctgttggagttgggattggatcaacatttcctatgcaagaagataacaaaggacatgactttgttagatttatatgaattatctaaagtcgaagcagttggaagttatttttatttggaaatatgtcaagattactatctctttgtttgcatcatgagttgggtaggagatctaagaaaggaagataagggaaccaaaaagaaatctagaggcaggttttaagagctgaaaaaccttttaaatccttggagaacagaataaacaaaacattattttgttttcttgctttttaacaaactattgcatgcttcaaataaagctttgcagtcattttctatgctccctggacaactaacattctttcagctctgaatgacctgcaatctttgtgttggtgtatattaagctattactctctggtatgtagaaaaccattgatggattcctttacttacccaataaattgtaggcttgtttccagagcaaacacttgctcaagaattacagaaggaattagctgggcatggtggtgcatgcctgtagtccccgctactggggaggctgaggcgggggaatcacttgaacccaggaaggcagaggttgagtgagccaagatcgtgccactgcactccagcctgggcaacaaaacgagactcatctaaaaaaaaaaaaaaaagggaaaggctcattttatgactaactggcattaatgaagtagtcattcgcatttttggcatcaactgtcacatctttacctcccaccacttcaacgtcaaaatacaaaaaatcctttaacccctcccacccccttttatttttaaagagcaatatttactaagggaaaaaggtggtccttagacttacacttattatactaattatatggagagaaaccaggccattattgcctctatctggtgaaaaacaggaggcaaagggcagagtttgatggtttccattcattttcctgatagacagcttcccattacaagaggaggggcagcccagcagtctagcctctcttagttctctttgtagttggcacccatcttctgtgtttagggtgtgccagtgctaaaacttggcagattttggtgtactcagagccttcgctttctgcataatgagctctatatgccatcactgcagttgtaggttataactatccatttgtctgaaaaactttgcttctaatttttctctttcaagctatgatttaggcatagagaatcgtgatgccaccaacgaccaagtcaccaaggatgctgcagaagctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaagtaagtcatgttggcaataatgtgattttgcatgttttttttttcatggcccagaaatttccaacttgtatgtgttttattcttatcttttggtatctacacccattaagcaaggtatgaaattgagaaatgcatatatgtataactgtatatttacacacatttagctaaaggcaaatacaaataaacttacaaataggcgtccatctcaacacattttttttcaaacatgctgttttttttcctttatccttttattcagttataccatatgatattgccatttttatgttggtaatttcatatggttcaaccagatctgtggttttcaacactggctgcacaataggatccccttacaagtttttttggtggttttgttttgctttgcttgatttgtttctttgttttagtttcaatgcttgagtaccaccctacacaaattaaaatctgaattccatggggttcaggcattttaaagctccccaggtgaatctaatgtgcaaacttgagaaccaccaaagattgtattaaacatgatcccatcatgcataaaagaaaaaactggctgggtactatggttcacacctgtaatcctagcactttggaggccaatttggaagcactgcttgaggccaggagtttgagactagcctggacaacatagcagaaccctgtctctacaaaaatgaaaagtatttcaataattatgtcaattgttcatggaagagccagttttgtttattcatatacaaagtgagtaggcgaagctgagtggggtggcatgtgcctatagtcccagctacttggaaggctgaggcctgaggatcccttaagcctaggagttcaaggttacagctatgagctatgatcacaccactgcctacgcaacagagcaagaccctgtctcgaaaaagaaaaagaaaaaaaaaactgactcggtagttgaaagcagccttactaaagcattccttcctgcctggaaaaaaagtattgcttctttacttctgtaccagtacacttgttgccaaattaagcaaaaaaactgccaagtaaacaaaatcaaagttcagtgttgaaactgaggtttcagactttcacagaaagtagtttatgtcttaaaaggaccttaaaaacaggaatctctcatgctgaaatccagaggttttaatgtagccatcattgaaaacagtgtgttgagggtaaacagtttagtccttgaggggtcagttaactttgttccactctgtggctaaaactatcctttcatcctggccaaagccttcagaaatagctcccaggggcccaagcaagggaaggtagatggcagtggtgcaaactcgctaatcttgtaaaaatcaccctagattctttccagttcagtcgtgccttttttctgcgtatatcctttttgttttttgtttttgagacagagtctcgctctgtcgcccaggctggagtgcagtggtgtgatcttggctcactgcaacctctgcctcccgggttcaagcaattctcctgcctcagcctcttgagtagctgggattacaggcacatgctaccatgcccggctaatttttgtgtttttagtagagacggggtttcaccatgttggtcaggctggtctcaaactcctgaccttgtgatccgcccgcctccgcctccgcctcccaaagtgctgggattacaggcatgccaccatgcccggccttcctctttttttttttttttttaattattttggcatcagtcatttgtaaaaatggccattctatttattaaagtccagaaagagaatttcaaacctcaggttgattcatggactgtttaaatatacataaacctatacaaagcatttatggtgctttggtacctcagtttctcccttttcagaaatagtaataaattaccacccatatttactttatggattttatatgtcaaaaatgttctgtgtaaagtaggtgtttaatctttattgttacttactgctcttttaccagttgttattagtatttctctacacttatagatgtggccggcacctgctggtacctaactctgcaagtatctgattcaattcattgaagaagatacaatgcagcaaagacttatggatgtctagtatatgccaggcatattattccactgagtagctgaggcccatgagggaggtgcctgtccttgaacagccctttcctaaaaatcatcatcatcccattgtagctgctcccagaatgatttgattgctccatatgaacaagtcctataacactagtaacaatgataccatgagagaccgagccccgatggcctaagtaaggacgagacctcaagtcctttgaccctggatgcattatttctactatatcatagttccctccttgtaagactgatgtaagaatcaaatgagattatgcaagttaagcatttaaagcatctggcatagagtaaggacaaagcattagctacctcagtactacaattagtattgttatcatcaccctctagggagacttccctgacttccttccatggtcacctcttttcctctgtcatcctatagtttttttttttttggggggggggtctaataccctcttttggcatttgaaagctcttatattgcttttcacttatatatgtctttatagggttgtaaacttcctaaaggaaagtggcttatgttattctctgtcctcttcaaatcagtgctcagaaaactttcctatacctcgtataggcacctaataaatatttactgatttaggggaatgtctggacctcttcatccccatctcctaaaaaaaaaaaaaaaaaaagaagtttgacatactgccctatttgtctaggtgtcttctaggtctatattatatgaatattctccttacaattcctgctagggtattttttttttctttatacctttttcccctctacacagtacagagcaactgattttgttgttcctgggcctggaaaagtagagataacctacacaccaagtgacggaacccaaaaggtgacatacctggtacataactttgaaggtatgtatgactgtagcataagcttcttttttttttaaacaaatgatttagataatatttttattgctttttttgttagttatgtgactgcctaaaaatataatctgaatctacagtgtttttgcaaaacccgaaacttgacataaagaagtaatttgcttccataccttcctgtggcatgaataaaggcacaaaagggttacccacttaattggaaatgcctttaggcaaagacaaggagatgattatgcatttatcttaaagcacagagagaaggttggcagaaatctgctgtgcatgaattatatacattaaatattaatattctcaaatcacccagcatataatcctgccctttgctctggtagggctcttgtatctggaagaggtttccttatatatttgctgaagttttcttcttgcccttctgggtatttatcctgtgaaaaagtagaacatttgctttttgccttttctttcccagctaccctgctacctttgatggcaaatcccacccaaaagttgttgaaataattagtgggcccttttccttcactattgaagttgttgaaagcactcacacaaacaaaaatacccaacactcacctaccttttgtttttcttgcctcttccaaataatatctgttggaatctggttctgcaggcttcagcacctttcaccctgaacccaagttaaccctgagcattggcatggattcaagttctatctttaagtaataacaaagttcaataatagttgtaaaacacagtgattacatgtagcataattatttctacttatttctaccaactacagtaggagtgctcagtatcctccaggagattataagaattttcattcagtatttgaaatttaagtttttctcaataactgaggattgagtacacccctaaagtttaaaatagatgttgggaattgtaaaggtagggtgtttgtccattcttgcgttgctataaagaaatacctgaggctgcgtaagttataaagaaaagaggtttaattggctcatggttctgcaggctgtacaagcctggctccaacacctgttcctggtgagggcctcaggaagcttccaatcttggcagaaggcaaacagacaataggtgtatcacatggcaagagcgggaacaagagaaagagaagggggaggtcccagactcttaaacaaccagatttcaagtgaattaactgagcgagaactcacgtaatcaccaaaggggatggtcctaagccattcataagggatccaaccccatgttccaatctcctcccactgggccccacctccaacactgggaatcacatttcaacatgagatttggaagggacaaatatccaaaccatatcagtatgtaaaagaagtttcaaaattatttgccttttcttaaatgtatttctggttcatatcagtttttggcatttctgcatcttcaggattttaaagactgtatgttttttcatttctgctgtccttccaaaacaagttagaatgtccttatctagaactggcttttctctcattatgtgcctcagaaatttgttggaaacctgtctgggacttctgaaatctagtctcagatgtgagagctgagaagtaggtcatttggttgtggtgggtgattttagccttattacaactacattacatttattcttctcccttttttcctctcactggcttctcctctacagaaggtggtggtgttgccatggggatgtataatcaagataagtcaattgaagattttgcacacagttccttccaaatggctctgtctaagggttggcctttgtatctgagcaccaaaaacactattctgaagaaatatgatgggcgttttaaagacatctttcaggagatatatgacaagtaactatagttcttttttaactttttctttattctcaagtaagctgacatttgcatatagagtatgtatctccctatccctatgattctgggtaaattgagtgaaaatcatcactgtctcttaaagataagggcaaaactgaggtttagcaggagtgaaacaaaaccactctgacttgcttttataagataaacatgttcttaaataaggtccccacatgaattgttttcagagccaggttctttaatgtttaaataaacgtacgctgttagtgataagttctaatttttcatgtgtgagtttctaagtcagacccatttccattctttctgaagcccattgttagaatttaaaattctgttggccaggcacggtggctcatgcctgtaatcccagcactttgggaggccgaggcgggcggatcacgaggtcaggagatcgagaccatcctggctaacacggtgaaaccctgactctactaaaaataccaaaaattagccgggcgtggtggcgggcgcctctagtcccagttactggggaggctgaggcaggagaatggcgtgaacccgggaggcggaggttgcagtgagccgaaatcacgccactgcactgcagcctgggcgacagagcgagactccgtctcaaaaaaaaaaaaagaaaagaaaagaaaaaaaagaatttaaaattctgttttagtggagtcatttgaacttaagtctaagtttataacaacactggcttccacagcacaggaggtgagcatgtgttaatatttaagattggcataactccctttaggtgcaagtgttcaggccaaaatgttcctgaggcattttgattcctcctcctgctgcccatctataccaagcccagaaactgtctggaatatattttagtttcctgaatgacaccaagaagtagaacagtcttttcaaaaatgtattttaaaaataagctgaatctcaagaatctgatctatagtataatgaaaactgaaaagtgaagtagtcattgggatactctactgtctcacttaattctcacggcttccctgcaaggtgggtaaaattgttcctacagatagtcaaattgagttttacagttagaaaatgattgggctaggatttgagccaatgtctgtcagattcctgagtttctgctacttctactaaaatatgctgcttcttgtgtgtccggtcttctgtttgggacaagcagatgatatccctaacaaaatcaattttctttattattattctcttttaccttttgtttcccaggcagtacaagtcccagtttgaagctcaaaagatctggtatgagcataggctcatcgacgacatggtggcccaagctatgaaatcagagggaggcttcatctgggcctgtaaaaactatgatggtgacgtgcagtcggactctgtggcccaaggtacagaggtggagcctggcctgtaggaacagggtcattccagggtagttctctaatttgggaaggggagttttgtagtcctttgttgtaaaatcttggaatgttaaacctccaggtccaccgtcttacctgacagctaaggaaattaagctcagactagagaaatgatttactcaagaccacatgggaagtaaattagtggattgacactcctggctccagtgtcactccttgaagttttaaagcctacctgatctgaaaatcactccctttccctgccctccttgtcctttggtgaaaatgtaaaaaaccagggcactcaggcaaggagggataaagtgaggagcttgatcatgtcataaccaagctttggtgcctgcaaagctcatttattgggcagcaggacctactcatgtccctctgccagctgctgttctggtagaatataaaacaaatgtgtttggctgggcacagtggctcacgcctgtaatcctagcactttgagaggccaaggcagtggatcacttgaggccaggagttcaagaccagcctggccaacatagcaaaaccccatctctacttcaaaaaaaaaaaaaaaaaaaaattagccaggtgtggtcgcatgcctatagtccctggcactcaggaggctaaagcatgagaatttcttgagcctgggaagcggaggttgcagtgagctgagatcacgccactatactccagcctgggcaacagagcaagactcttgtctcaacaacaacaaagaaaaagtgtttttgtttactgcttagcttacacacaaatcaattgagcaaagaaccctaggggctgtttacagaaattccaggagaagtttcatgcatggggtaagggccaaatcaacagaaacacagcgcctggtttcctcagcaccaagttgcccttcagtttcagttgcccttggtagctgtatgaacttgaatctccccagatgaatgtttgttaactttaattatgtttgtttttcccaagaagtactactttttctgtctcttatcagtgttaaacatctcctctcagtttgaaaatcagtagatcaacctggttaaagataactttttgccagataaatgagttacacaactctccttaccatccttctgtttctctacatcctcacaatcatttgtactgaatgcaacagaggaaggagtgttattgctttgtgatgactccgtggttgattattaacttcacaggtgatgttgggaggtagcaaaaatggagaaaagaagaaaatcaattatatagatataagagttgtaatgtggttctttcaataagaaaccacagaagagaactgttgtactgagggtatgaaagagagcgaggaagggcaactgggatgttcttagatccaaccgatcctgtgtttacctgccttctcttcccagtcccagcaagtgtatgagcagcttgagggttcttaagagacagctctcgtccatctttatttccccagaacagcagagcattgcacataagaataacaataatagattgagctctgtgtccagtactattctaaggaatataaatatatattcatatatatttatgaatatatgtatatttatctaatcccacagcaacctagtgaagtaggtgctgttattactcccattttacagataagaaaactgagccgcagtagctatgaagtcatagaactagagtggaatgtaggcagttggaccctgaaccctccatcaaggagtaggtatccctgaaataagtgaggggaaaatgcagcctttccagacccattggctctgatatctggagcggggatcctgccttctgaaaggcagaattattggtgattccatgtgctcttgtttactttgcttaccaagacctatcaagattgagtcatttatgctctcatctctgagagactcagtgctcttcatgcagttggacccaacgcttcatgctccactacttacagggtatggctctctcggcatgatgaccagcgtgctggtttgtccagatggcaagacagtagaagcagaggctgcccacgggactgtaacccgtcactaccgcatgtaccagaaaggacaggagacgtccaccaatcccattggtaagatggtgtttacattgatttctcaccagagagtgctctccaaagcagcccagatgtgttcccttggctgctcagtgttttgtgtgcaaagtcatcaccaagtagctgatttgttctactagtaagatctatttgaatatgcttcttaaaatatacaatatttgcttttctgttttgtgggagaacataggttacagagaggaatgttatcaacaagcattttcctttacctctggtgcagtgaaatgtcacataaggctacaaatctaaaagtcacatggatagatccttttaacaagtttcaaagatagcacttcaaaaagagattacagcctggaaaaaggagaccaatatagacaagagatagaagagattgtgacctttttcaaattagaaaatgccttaagtttcaaacactagtaccatgccatgaaaatgtgtttttttaaagtttctgtttggtaaaatatttctaactctcctaaaacttctagcccatgtggttagtatctaaagaagtctgtcacctaaacaaaaaactattctgttttggggaactatgagacatttggaaacctctataactaggatgattggagtgttgtgttttttttcccctcttttagcttccatttttgcctggaccagagggttagcccacagagcaaagcttgataacaataaagagcttgccttctttgcaaatgctttggaagaagtctctattgagacaattgaggctggcttcatgaccaaggacttggctgcttgcattaaaggtttacccaagtaagtataagatgccctagtttctatggtcaaattaacacttatcctcactgggctcagagcatcggtgctttctggaattatataaattgttattcatcatctagttcaggagtcagcagatatctgtaaattgtcagatagtgaacattttaggctttgtggaacatatagtctttttgcaactacacaactattcaactttgcctctgccattgtagcaggaaagcagtcatagacaatacagaatcaaatggtgtggctggtaccaatcaaacttttatgttcaaaaccagcctcgatttggttcatgtgctgggctgtaatttgctgacatcccatctagttgataatcattgaacgaaaaggttctatttaattttaagaagcagattatagacaaatagaaacaaagatacttcagaggctattttattctactcctttattttctttaacttactgatgagaaaagtgagcagcacaagtttaaggtgtgcaattgattacagtcccttaagtatctgtaaccaatatttgggcaaatatttgacagcccatatgtttactgagaatttttctgagaacatagtagccagtatcctggggggattgtctccatatctcaaccagtgcatctgcagaggtcccaggtagggtcagtataccccataatggggatatacttggtcactgcatgtttcttaatttaaatttccaacaagattgcagtgttcgctgggtgctgtggctcatgcctgtaatcctagcactttgggaggccgaggtgggtggatcacgaggtcaggagatcaagaccatcctggctaacacagtgaaaccccatctccactaaaaatacaaaaaattaaccaggcgtggtggcgggcacctgtagtcccagctactcgggaggctgaggcaggagaatggcgtgaacctgggaggcggagcttgcagtgagccgagatggtgccactgcactccagcctgcgtgacagagcgagactccatctcaaaaaaaaaaaaaagattgcagtgttcaaaatggtcttatcagtggaatacctttgtagcctatttaacaggcacctgtcagcaggattatgttatcctgtcttttgtttttctttttttttctttttattgagacagagtctcgctttgcccaggctggagtgcagtggcgtgatcttcgctcactgcaacctccgcctcctgggttcaagtgattcttctgcctcagcctcccaagtagctgggacttgtgccacaacacccggctaatttttttttttttttttttgtatttttagtagagacggggtttcactttgttagccaggatggtctccatctcctgacctcatgatccacccgccttggccccccaaagtgctgggattataggcgtgagccaccaggcccggcctgtcttttgtttttcttaagacactgatagaaataacaacattataactttttttaaggacatgtaacggtttgtgggaaggtgggtaaaggtttggctatgccctgaataagttttgttatgtttaagaatgaggaagaagtgtgatgattttggtctcagcagcattagtctgaaccgaaactcaatgataatttcacccccaaagctacatagcaagaaacctaactgagtatgtatggctgtctctactgccggttgcctagagagcctgagtactgagcctgggtcagccccaggaaccctgaacatagtcattgtctatagattttgtagcttactaacatgaatgcgttttcttccagaaacaaatagaatactattttaagtagcatttctaggactttaccactacctgctaccatatcagagaccaactaaattttcttttttttttccttcccgttagtgtgcaacgttctgactacttgaatacatttgagttcatggataaacttggagaaaacttgaagatcaaactagctcaggccaaactttaagttcatacctgagctaagaaggataattgtcttttggtaactaggtctacaggtttacatttttctgtgttacactcaaggataaaggcaaaatcaattttgtaatttgtttagaagccagagtttatcttttctataagtttacagcctttttcttatatatacagttattgccacctttgtgaacatggcaagggacttttttacaatttttattttattttctagtaccagcctaggaattcggttagtactcatttgtattcactgtcactttttctcatgttctaattataaatgaccaaaatcaagattgctcaaaagggtaaatgatagccacagtattgctccctaaaatatgcataaagtagaaattcactgccttcccctcctgtccatgaccttgggcacagggaagttctggtgtcatagatatcccgttttgtgaggtagagctgtgcattaaacttgcacatgactggaacgaagtaggagtgcaactcaaatgtgttgaagatactgcagtcatttttgtaaagaccttgctgaatgtttccaatagactaaatactgtttaggccgcaggagagtttggaatccggaataaatactacctggaggtttgtcctctccatttttctctttctcctcctggcctggcctgaatattatactactctaaatagcatatttcatccaagtgcaataatgtaagctgaatcttttttggacttctgctggcctgttttatttcttttatataaatgtgatttctcagaaattgatattaaacactatcttatcttctcctgaactgttgattttaattaaaattaagtgctaattaccatt

SEQ ID NO: 9 - DNA, 19460 bp, Artificial sequence
  synthetic sequence
gtgcccgcgcggccagcgcccgccaggcccagcgttagcccgcggccaggcagccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactgtgagtgccgcgcggagggtccaggcaggcgcgcgcccccagctgcgacgtggcggcttcccaggctggggccgggaagcacgtcccgccagcccgcagcccggggacggcggggtcaggcgcacaaagggccgcggggcagcttcgctccgcgctcccccgcccggcgcttcccggccgtgccaggcgctggggtggggacttgagggacggtggcaactggtggcccgggcgacggccagctctgcgcgggcgggggctcggggctgccgcgcgcccctaggtatttcgggagggggtcctgagagtgtcccggacccgaagggtggcctgcccgccctgcgcccctggaacgagacgagcacccgtggggggctggagcaccccgcgcgctcccctggcgagagggagggtcgtggctcggcccctgctcagacaaaggctgggaggcgggagacatgcacttccccttccttttcagccaggcgcgcgctgataccaggcccacgtcagctatttttggagccttttacacgacagctggaggagcgtcctttttaattttccccttttgtttggccgcccccacccccaccccttcgccttcatcgctgcacttgaggctccatcctggggcctctccttgacttgacctgccttggcaggcacatgccctccctgcctggctcactcgccgcagagacctggcagcccgcgcaaaatgtcactttgcggaatcgttcccacggcttctgggtacccttagttccctgcttaggagggaagacagtagtcgggtcgtaataagcaagacttagcccgagcctccgttgccaacgcaggctgccttgcttggcgtgtgggcatcggcctgccccctcaccctggctacccaacacagctacaaaaggcagggaacaatgtaggtcccttggccctgcctaatgcctgttgccatggaaacccctatcctaatctggccaggagccccttgcagtgagccaggagagtgaggaagaggggatggggcccgctggccctgacctggccagaggaggtaatggttaaccggattgtgggagcagctgactagagccgggggggtagggaggcttgggccccagtcctaccttccctgccaaggagaaaggggcatgtctgcttttgtacctctgggaatctacctcagggatctgcccaacaactcccaggttccaagtgccccaggacccaaagattcccatatagcacaccaccttcagcaaaaatatggtggaagttagccacattctttaattctccctttttttctgccttgatggtaaaaaaaaaaaaaagaaacaaacaacccgaaaaacacacacaaacaggaacacaaagcgccttttaagggtcctgaatttggatacagaaaaagcagtcagttgcaggcccttctagaagtccccatgtgggttggttatgaatggggaaattgcttggtgttggagttggacatcagactctacagttgcgactctgccagcttgttacctggcaacgcagctggagacagaatacttggcattccccttgatgacaaggaaagaatattttggggctaaaagagccccgtgtctttgctgcctggtaattcctgcacgcatcttttcctattttgcaacgccataggcttccagcgactgctggtgatgtttctggtaagttagagcttggggcagtgcggaccagttctgtgaacggctcagtggcagaattacttatctagccttccttcactcctaacccagaaacttcttaattcttgccccttaagtgtgcagatctttctgccacgtcagataaggccctagctgccctgccagagctgattggggtggttgtgtttaggcctagtaggtactgaggaaaagcctctttggaatggctgaggcctgctcccacatcttctgtgtgaagttgagggcccttgggctggttccattcagggtatgaggtgctggaaactgctctcagattactggaagccagggtctgtggccttccctggggtttatactctgtaaacccatgttaaaaataccagcagattgcaaatattagtacactgtaggttgtaagcttcttcagggcatggggcctattgctggagagagatgccacccgtgtacatctggtgcaggctgctggtaagaactcactggcccctgtgtggcaatgcataccagatgggtgagaggggaggaaacacattcagtgaaacagaaggtgagggcagggcagccctgaccacagatggagttgacttcaacttgacctttatttatgtctttgacaaaagtagtatactcctactttgtgccagacaccatgccgggtgctggggatagagctgggaataaaaccagcagagactctttcaaaacccttaggagctcaaattctgatgtgggagggggcagacagtaagcaagatagatgttgactagtatgatatatgggtacaagagctaaggaaggccaggcactgtggcttacacctgtaatcccagaattttggggggctgaggtgggaggattgcttgaggctgggagctccagaccagcctgggcaatatagtgagaccccatctctacaaaaataaaaaataaaattagccaggcacgatggtgcatgtctgtggtcttagctactcaggaggatcatttgagcccaggaggtcaaggctgcagtgaggtacaattgtgccactgcactctagcctgggtgacaaagcaagaccctgcctcccccttctccccccaaaaagagaagagctaaggagaaacaggcaagaaaggtgtgtgcacaaaagtttgtatagggcagccagaaaagtcctcactgaaggaatcgctgaggaggggaggaagcaggccattttcggtattgggggaagagggactcgcaggtgccaagggcatgatggaagagcagagaggatggtgcgaaggctggctcagcaggcaccaggtggagtgacaggatgaggttcggcagtggggtctgtagagggctcacaggccatttttgagagctttcgcttttactttgcagggttttgagaggagtatcgttacctgatttccatcttatcgggagcattctagcttcagggacctggcaagagaggatgatggcttggacctgggtggtggcgctggggatggtgaagaagtggttggattctggatatattttgaaggaggagccagtagtggggaaaggttccaattcaggcgattttacataaaaatctagatacgcagcaggcactcacttatggcaagggtgggcttgtctgagggtggcttctggttttggtggtttggatgtgagagaagggaaggaatgaaggatgacaccaaggagtctactagagtttgttccaaaatggctgttctggccgcgtgcggtggctcccaggactttgggaggccgaagcaggcggatcagttggggccaggagttcgagacctgcctggccaccatggtgaaacccggtctctactaaaaatacaaaaattagctgggtgtggtggcaggcgcctgtagtcccagctactcaggggctgaggcaggagaattgcctgaacccaggaggtggagcttgcagtgagccgagatcacgccactgccctccagtgtaggcgacagagtgagactctgtctcaatacatacatacatacatacatacatacatgcatgcatacatacatacatacatacataaaatatacaaagttgctgtcctgccttcttgagcccccaggaattggaacctttccccactctctgatcttttgctgggacatgctgactgagcagcctttctgctgagagcatgggtaggaatgtggagggagcagttatgcctggggctggctgcctgcggctggacctctagcctcctgctgggagcagcaagaagccagggatgtgttttgaaatccttacttggtattgggtgctaggagccccagacagggaggagagtcagcctaacgtgcctctggtgactggtgtaattccatcctgcactctcgccaagaggacagtgtttgtgcaatgcatcagctgaagattcgtggtggctgtcacttcggaggttttcagggaggcctagctgctgctctgtgtatttcaggtagcttgtcaccacttatctctcagagaagggcagaccatggctgtatctccaccatacaggctgaacagcagacaggggtctcaaactccagtgccctgggccacactggagggcacttggctgtcatggagtgaagggagaggccgccttcagcccagcccatccttgccatgagtgagtgcctgctgcatttctagatttttatataaaatctcctgattttttttgactggtttaaatttttattattttggtaaaaaacgtataaaacttaccatcttagtcattgttcagtgtacagtattgttaagtacattcgcactgttgtgcagacattaccaccatccatcaccagaactttgtcaccttgcaaaaccgaaactctctacccatgaaacaatacctccccacttcccgtccccagtccctggcagccacctttttactttctgtttctatgaatttgactattctaggtacctcatgtaagtagaagcatatagtatttgtcttttgtgactggtttatttcacttagttagcataatgccccccaggttcatccactttgtagcaggtgtcagaatttcccttcttttcaaggctgaatcatattccattgtatgtagataacagtagtcccccttatccttggaggatgcatttcaagatccccagtggatacctgaaactgcaaagcctgtagatactatgtttttttcctatacatacagatctatgataaagtttaatttatgaattaggaacagtaagagattagcaataataactagtaataaaatagaacaattataacaatatactgtaatgaaagttacatgaatgtgatctcttaaaatatcttactgtactgtactcacctattttcagactatggttgactgtgggtaactgacaccacaggtaactgacgtcatggaaataaaacctttgataaggggggactgtatcacattgtctttatccattcatctgtgatggacacgtgggttgcttctatcttttgctgttgtgaacattgctgctatgaatgtgggcatacatatatctctttcagaccccgctttcgatatttttggatgtatatacagaagcagaattgctggatcatatagtaattcttgtccttttcttttctttttttttttcgagacggagtcttgctctctttcgcccatgctggagtgcagtggcgcgatctcagctcactgcaagctccgcctcccgggttcatgccattctcctgcctcagcctccccagtagctgggactacaggcgcctgccaccacgcctggctaattttttgtatttttagtagagtcagggtttcactgtgttagccaggatggtctcgatcttctgacctcgtgatccacccgcctcagccttccacagtgctgggattacaggtgcgagccaccgtgcccggcagtaattcttttccttttagctaaggaacacccatactgttttccacagcagctgcaccatttcacattcccaccaacagggcacaagagttccaatttctccacatccttgctagcactttattttctgttttgtttttcttttcctgatagtagctctcttagtggttgtgaggggatagtggggtgatatctcattatggttttgatttgcatttccctgatgattagtgatgttgagcatcttttcatgtgcttgttggccatttgtatatcatctttggacttgaaatgtctatttaagtcttttgtccattatatttatttatttattttgagatggagtcttcctgttacccaggctggagtgcagtggcatgatctctgctcactgcaacctccacctcctgggttcaagcaattctcctgcctcagcctcccaaatagctgggactacaggtgtgtgtcaccacaccaggctaattttttttctatttttagtagacacagagtttcaccattgtggccaggctggtctcgaactcctgacctcaagtgatctgccctcttcagccttccaaagtgctgggattacaggcatgagccacggtgcccagccagtcttctgtccattttaacgttgagttatttgttttattgttgagatttactgatttttaaaacttggtcaccaattaagatttttttaaaaaaccagtacctggaacaagcagaacaagtctctgctggccttggctcatgttgctgttctcagttcccctgggatgtttgcttccagggaggcctgggcactcctgactttggtgacagccctgcttgctctctctctgttacctgtaggtttccccttccacattcactgcctccgggcaggcactgttgtgttttaggcatttcagtatcccagtggggtactggataagtgcttagttagcacctgccggtgagtaaataatcaggtcctgtggtctctgagggtttgacacatcaggtctttgatcttgaccttgaactctcctagccatgcctgcctgaagcccaaccgtggagcccaggctgaggctgaaacctgaagaactggccccaagtgagtctctccttagggctcctgattatgatagttcatttttttttttttttaacaaatttgattagctgtcctcaaattaactcataggagatgctccccagaactaaactagctctgcaacagctcaacagactttccgcaggttctgcagctctttccactcaagcttcagaaaaaagtgaaggaatattgtagattgggttcaaaatccccatcgcgtcttgggaaaggcagtagagccagcagccagccaccttttgttctctagggaatggaatagtccaaggctgtgtttgtgttctgcttctctacatctgacccacgggtcttccagaggctgcatttgggtcagggacctgggctctatcttgcctctgatgcagaccacactctctgagtctcattattccccctaaatgatctctaagattatttttagattaaaaaaaaaaaacaaactctttgcatatctttggaaagtcagttgtttgaggtctgttaggctttaatcacttttgttttaaatgtgccgaggtggtttctaggcctgcttttcacaggtattctgtcccttgtcagttcttcccacccagttttttttgtttgtttgtttgtttttttgagatggagtctcgttctgttgcccaggctggagtgcagtgccgtgatcttggcttactgcaacctccacctcccgggttcaagcgattctcctgcctcagcctcctcggtagctgggattataggcacgtgccaccatgcctaatttttctatttttagtagagatggggtttcaccatgttggccaggctggtcttgaactccttaccttgagtgatccacctgcctcagcctcccaaagtgctaggattacaggcatgagccattacacccagccccaacccagtgttttcaaataatcattctcttcccttcacctgatttgctgtgtctaatctagagtggagctgaagacacatcaccacctctctaaggactgtgtgtaacacaagagtaattgggctgtaagtgaggctgaggagtgctgctttgaggaaagggcacagctctcagctgagctggtgagatttccctgtgactgaagttttattagatcttgttccctgctctgcatttgctgcttcttcccaggctgtgggcagaggagcaggctggtgccccggtgcccctatctgctgcaggctgaggttcaacaagccttgtccaaggcagctgtactgattcattgttaggtggccagttcctgagttttctttgaaattcacttcccagactgttgggtattcctgcgagtcagactctccttcagtagcgtcccctgccccctaccctccactggtctccctggacctctgatcagcccctcattgagtcctttgatgcttctctggtaggactgacccacctgcgtcggctgggatcaagggttctgagttccattcgcaggtgctgtaagcctgtccttcctgttgaatagggtcagagtcctcgtgactctgctgttttcaagctctgtaaccttgggcagtaagctgttaacctctctgagcctcagtttcccacacttaaaatggagataatggccaggcgtggtggctcacgcctgtaatcccagcactttgggaggccaaagtgggtggatcacctgaggctaggagttcgagaccagcctggccaacatggcaaaaccccgtctctactaaaaatacaaaaattagccgggcctggtggcgtgtgcctgtaatcccagctactcaggaagctgagacagcagaatcgcttgaacccgggaggcagaggttgcagtgagccgagatcacgccattgcattccagcctgggtgacagaccaaagactctgtctcaaaaaataaataaataaataaataaattaattaattaaataaaatggaggtaatgatacctccttggggttgtttttgggattaaatggaatcacattgtacagcattggtacactgtaaggcacatagtaggcactcaatacctatctatactatttcctttatcaccacaagttagttgagggctcttgggttgcacgcaacagacaaagttagtgactggttaacttggagaggattttattatgagctacacagcgtagctcatagaattgcagggaaaactataaagaactaagtcttggacaaaagtaatatcttggcatgggcctgtagccccagctactaaggaggctgaggtgcaaggattgcttgagccccggagtctgagcctggcttgggcaacagagagagataaagagaccctgactctaaacaataagtaaatacataaaagaggacccaggccctccttaggaactggatggcaggagcccctggacaacctcttactgatttggtgctggatgaggcagccgcagctagccagtgttctttgtgttgatctcctctgccttcatattccagggggagagggtctgacaggccagactggcccccaccctcttggccactggtgggctgggcatgtttttgggtagttctacctagccagattaaagtgggggtggggtggggagtccagccagcctgggcaacagggcaagactccatctctacaaaaaaattttaaaattaggctggcacggtggctcacgcctgtaatcccagcactttgcgggggtcaaggcaggtggatcacttgaggtcaggagtttgagaccagcctggccaacatggtgaaaccctgtctctactaaaaatacaaaaaattagccaggtgttgtggtgcacacctgaaatccccgctacttgggaggctgaggcccgagaatcacttgaactcgggaggcggaggttgcagtgggctgagatcatgccactgcactcccaccctgggggacaatagcgaaacttcgtctcagaaaaaaaaaaaattaaaaatagctgggtgtggtggcatgcgcctgtagtcccagctacttgggaggctgaggtcagaggatcacttgaagccaggaagtcaatgctgcagtaagctatgatggcatcagtgcattccagcctgaccaatggaacaggaccctgccaagaaagagagagagaaagggaggaaggaacgaaaggaaggaaggaaggaaagaaaggaaaggaaggaaagaaaggaaaggaagtaagtaaggatggatggatccaggtgctctaaaccagcagttcccaaagtttttggcaccaggggccagtttcatggaagacaatttttccacgggtggcagggtggtgggcagcggggatggttttgggatgagtcaagtgcattacatttattgtgcactttatttctattattattacattgtaatatataatgaaaaaattatatagctcaccataatgtacaatcggggggtcctgagcttgctttcctgcaactagacagtcccatctggaggtgctggaagacagtgacagatcatcaggcattagattctcataaggagtatgcaacctagatccctcgtgtgcgcagttcccaggcatgagctcaggtggtagtagtgctcactcacctgcagctctcctcctgctgtgtggcccagttcccaacagtccgtggacctgcactaaaggctgtggcctggtctgctgacctctctccagagcccacggtactagtccatggcctgggagttggggacccctgctctaagcagtgagggagctgatttgcagacgggggagtggacaccgggcagctggagccctggctttctgccactgtctcctggtgaagggcgggcctggctgggaggagagggcccccctccagcctccccaggggcatgatgcgctgtgtgtccctgcttctagatgccgacaaaaggatcaaggtggcgaagcccgtggtggagatggatggtgatgagatgacccgtattatctggcagttcatcaaggagaaggtagtgccccctcctgaagtgggtggctctccaggtgggctggccagggattgttctgtcccacagggtcttctggactgcaggtccctaggacccccccgttgtcctggtaggcagcagcagctctgtttctctccctctgtctacccttctttcacatctggtccactaattgttcagaagtcgtcaggcgggggcctgtccccagcagtccgtgttgtgatggtgccagatcccacacgttctgtcaactttgagaagcttcaaggttaatcctgtatcatttccatcttgatcccctacgttttgcctcacttcttgaaaaggagtgaaaagggaacagggctggtgtgtagggcagtgacccagcctgcagttgtgacgagccaccatggcaaaggggtcccagagtggccgcttggcttctccaaagttgccgtggtctatgtatgtccaggagagcatgtagtccttgtgaaggccccacactgtgtgtgcgttcatgggacaaaggttgagaactgtcacttccaactgtaagatctcaaagcacttgagaagaggaaaccagttgtatagagaaatctagatgtacttggggttggggtttggctgagttgatgggccatgtgaggggggcacacaggcacagtggaggaagaaccctccaaaagactgcggcctggcctgccaacctctctccaaagccccagcctctgccagctctggccaggccccactgagaaatggctgactccagctttctgctgcgcccctccactggccttcactcatccctgttttgactgactgtcccctgccatggcctgcagacttcttatcctgccctttgttgtcatgtccctgagtcactggggtgacgccttgctctggccctctgtccccagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaaggtgcgagggtgtggaggtgggcgggccagggagggtcacaggcttctcccaccggcctctccagacttggggtggggcagttggccatgtcactcatctcgggactgggaggttgggaggctccagagctggggctttcctgaggccaactgcacctgaggccagtctccctgtgtgtgcctggtggacagtggggcctcagaggtcacctttgatgaggctggggcaggcgtgggtaaggggctggcctcaggttccacggttagggcacagcagagctgagaacagaatccatgtcttccatctcccaggccagtggtgtttctgcttcactctggctctgacagtggtaggtgtcatcggtgacagggctgaggaggaggaggaggaggaaggtaatgctgatgtggtcacagtaaggatggcttctttgggcagtagtccattctggcgtggacagtcttgcaggcaggtatgcagagagattgtgcagaggagtgctgtgtgtaaggcctgtgtccttcagagcctcagcagcctctggggaacattgctgttgccctcctgccactggcttggccttgcaggtctgcctctgtccccatggtcatgcagggtcactgagaggtgcccccaccaggcctagactggcttctggtccttggctgttggggccgctctcgagggttccatgaagtcacagcagtgtgtgtgcagaggggccaggtttggataattcgagaaaggatggttgaggagttttctgagctctctcttcctactttccacagttggagggagctggcggaaagtactcgagatacatctgcaaattccccaggcctgcccctgtcctgcaggtcatgcctgacccacagcactgggatcatttgagtgttgagctcttcatttcacctgaaccttgggagttgtgtatccccatttatggacaaggcaggagagggtcacagccttaggaactggggtggcgttggggccaggaccggagcatgtggagtgggcagtgtgttgtcaggcttcgtcagacaagtccttcccccggactgtactccctccatgtgtgatgtgggaatcataatggccaacagagcacttagcatggaccgggcaccgttctaggccctttacacacgagctcatttcgtcctcacgacaacacttaggctggttatttttccagtaatttttttttacagctttggttgagccacttttgtaagcctcaccattaaaactggcagacttgctgggcatggtggctcacacctgtaatcccagcactttgggaagccgaggcgggtggatcacctgaggttgggaggtcgagaccagcctgaccaacatggagaaaccccatctctcctaaaaatacaaaattagcttggtgtggtgtcacatgcctgtaattccagctgctcgggaggctgaggcaggagaaccgcttgaacccaggaggcagaggttgtggtgagccgagatcgcgccattgcactccagcctgggcaataggagcaaaactctgtctcaaaaaaaaaaaaaaaaaaaaaaaaattggcagactccagagcccacacatttgcactctagactctactgccttcctcatgaagaattttaggacccccgtctggctgtgttgttgcttggggttcaaattctggttgaaagatggcggctgcagtgggaccactattatctctgtcctcacagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccaggtaggccagggtggagaggggatccactgacctgggcaccccccgactggagctcctcgcctagccatcctcttgtctctgcagtacaaggccacagactttgtggcagaccgggccggcactttcaaaatggtcttcaccccaaaagatggcagtggtgtcaaggagtgggaagtgtacaacttccccgcaggcggcgtgggcatgggcatgtacaacaccgacgaggtgaggctggcttgggcatcctgggcccctcctctcccagcttgtctcttcatctctgtctcgtggctttcctttcttctgtaatggcctcattcttaggccacaaagaggcagaaatggccaccagggctgggcatggtggctgacacttgtaatcccggcattttgggaggccaaggcaggattgcttgagaccagaagtttgagaccagcctgggcaacagtgagaccccatctcttgaaacaaaaagaaaagaaaagaaaagaaaagaaatggccacgagcagctccaggcgtactcctgcctgcttggctaccctatcagaagagaatgcctctttatccccagttccagctccaatgccaggctgagtcacatgaccacctctgagccaatcacagaggccctggggagaaaggcttctgattggataagccagggacacactgtcaaccccagcgctaggcctgggaattagccctgccccagccacatgtagtctaaggcggtgctttccaaaaaaaagttgtttccaaaaaaaaaaaaaaaaaggcggtgttttccaaaaaaaaaaaaaaggcggtgttttacaaagcaaagttgagagggagaggctgggccagcagaaacatcgtgtgcactgcacggaggctggtgttaaacagtcgcgtgggcggcggggtaccgttcctggagagctgggccttgccctgggaggtgggaggttgccggcaatcgccaggctagggcaccacgccagggccctgtctctccccctgcagtccatctcaggttttgcgcacagctgcttccagtatgccatccagaagaaatggccgctgtacatgagcaccaagaacaccatactgaaagcctacgatgggcgtttcaaggacatcttccaggagatctttgacaagtaaagcctcatccatgtactctgtggcctttcttcccttccccccatgctgttcccatcctaccctgggaaggtcgctattagagtgcatttggctcagctccgaggctcagggagggatccccaacctgtcagccttctgccctctccccataacagacctttttactcccaggcactataagaccgacttcgacaagaataagatctggtatgagcaccggctcattgatgacatggtggctcaggtcctcaagtcttcgggtggctttgtgtgggcctgcaagaactatgacggagatgtgcagtcagacatcctggcccagggtacgctgggaggacgctgctccctgtgggggtggactgttggtcttctctggctggagggattttcagacccttttggtagaaaactctagtggagcaggactcattgcagggtcctctgtggacagcagggggcaggagctccctggcatctgagtgagagaggatcacaaattaggtttggactggtgtatgactgaggctgctcttagagagaaaggagggcacaggaattccagggctaggttgacagccctaaggttgctccagtccatgatggagcctcatgactctttggttccagtaggaaaatgggatcaagcgggactggaggttctttaaactacagcgaatgcattttgcatagtgaacctaatggatggaagagaatgttggggaagcctgcaaacccccaagccctagggtgtcctaaaagacaaatcagattgcagtaaaaagtataggtgatgaggtcaggagtttgagaccagcgtggccaacatgacgaaaccccgtctctactaaaaatgcaaaaaaatttagctgggcatggaggcgggcacagtcatggacacctgtaatcccagctacttgagaggctgaggcaggagaatctcttgaacccaggaggtggaggttgcagtgagccgagattgcgccattgcactccagcctgggcaacaagggtgaaactccgtctccaaaaaaaataaaaaataaaaaataaagtataggtgagaccattagagaagaacagcgagaaatgggccaattgtgttgggaacaagagcaggaaaggtggtaggtgtgggaaacaggcaggacatagagagtgaaagagaaacctaagcttaccagggcagctcctggtatggggaaaagggaagcccaggacgggttctgatgaaaagcagagtcttgttactttgtgtaaacctttggctttactttggaaagttcagtggggatcctctcaccgaacaattcccatctaggatagaaggatgaaacgccatagcagatgaggcagaggcatgaaaatgaaccttaagtgtctaaggaatggtgtgaaggtgcaggtaaaatggagaagggattgcttacagccaaaattcttagaattcatagacaaagcattttcctgtagatgttgtccatgttttctttcttcaatccttattttggtgtcatgggggtgcctttgaagccctggagtccttgacaatagaaaagcttgctggagttgacatgtggatttgggagcattcagaaattaatcaaaaagcttgaaagtggcctaagagagtagctgcaaaagtgattgttgggaaatagctgaccacagctttattgttttccttgaatgtgcctccgggtcacgagccaggctgggtggcaggctgtgggggaccacaggactctgtggcctcttgggccctgtgggccacaggctgtggggcgcctggagctggtgacacagatggatgttaatgtgctggtgatagccaccctccagtgccctccagccctgtgctgggccctggagacccacaggagggtgaagagacctggaacagtccctgtcctcccagttgcagctgggggaggctgagtagagccacgaactatggcagctacaatattgggttgtagagggcagcagggctcagctgggtggccccaggagaggcgaggccctgagagaaaggctttctaccctccaggctttggctcccttggcctgatgacgtccgtcctggtctgccctgatgggaagacgattgaggctgaggccgctcatgggaccgtcacccgccactatcgggagcaccagaaggtgagtgcagggcatggggcctcacaatgcccctctaccccaggggcctaaagcccactgggctgagggctggaaggacgagcctgacccaccagcccaggggcgggcagctgtatctgggcagcctcctcactagctggctcggctcttgatctccctgcaacccccgtccccagggccggcccaccagcaccaaccccatcgccagcatctttgcctggacacgtggcctggagcaccgggggaagctggatgggaaccaagacctcatcaggtgagcatggagggagaggccggagctgcccaggacaggctctgggtcctgcccttgcacagatggggtctcattctgccccatccccataggtttgcccagatgctggagaaggtgtgcgtggagacggtggagagtggagccatgaccaaggacctggcgggctgcattcacggcctcagcaagtgcgtggcctggggtggtaggcagaccctggggccatgtgggaggaagccaggggacccctcctaaagtccccctcatgagtcagcttagctgcaagggggcctggggcaggacatgtccacagccctgagagactgctcaggccagcctctgtagaccaggtctagtgagttcctccccatccctgaggctggctactcagagctccttccgaccctgcccacacccctttcccagcttctctcaggagagcagttacctggcaagacagaccctgcgggtgcctgccgaatctcggtggcctcatggtagggggctatagctggcctctggcctgtagtctgggtggcaggagatggctgttctgatgcccaagctcaggctgttccctggaagggcctggctgatggccttggcaaggcctggagccacccctgatgtgagtggtgctctctctgcagtgtgaagctgaacgagcacttcctgaacaccacggacttcctcgacaccatcaagagcaacctggacagagccctgggcaggcagtagggggaggcgccacccatggctgcagtggaggggccagggctgagccggcgggtcctcctgagcgcggcagagggtgagcctcacagcccctctctggaggcctttctaggggatgtttttttataagccagatgtttttaaaagcatatgtgtgtttcccctcatggtgacgtgaggcaggagcagtgcgttttacctcagccagtcagtatgttttgcatactgtaatttatattgcccttggaacacatggtgccatatttagctactaaaaagctcttcacaaaattgtctgctgtgtttgtccctgaggggaggaggtagtgggaccctgaggcagaggccctgctagagctggcaggttcccctggggcagaccagagcacctcaggaaggggctgccacggcagggaagggaccaggcagccctgggagcccgcattccacaggggcccactgcggagttctcggacactcagggcacaggcctgtgggttccctggaattttctagcatgatccagtttctgtgtccagttctccattctgagagtcaatcagttcctgataggttgtcattgatttttttcttcgttggttttaaccttctaaacatctccaggccactttcttagcctttttctaggtactaaaaagaggtcctacccacacctgcctcacacttctcctttccaaggctgcctgagtttggaggggcttgggtgtgtgtgaacaagggccctgcattgtctaggcctgcagttcccaggcttgggttcactttcaccatgcattggcaaaactagaaaagtaagcttgtgacaaattgttctcggccgggcacagtggcgcacgcctataatccctgtactttgggaggctgaggtgggtggatcacttgaggccaggagttcgagaccagcctggccaacatggtgaaaccccatctctactcaaaatacaaaaattagccaggcgtggtgatgcgcacctgcagtcccagctactcgggaggctgaggcaggaaaatggcttgaacctgggaggcagaggttgcagtgagccgagactgcaccactgcactccagcctgggtgacagagcaagactctgtctcaaaaaaaaaaaaaaaaaaaaaaaaaaaaaaattgctctatgcaagttttttagccccatgtaaaaaggaaaaataaaagttgctctaaagctacatataatcaaaa